(19) United States
(12) Patent Application Publication
(10) Pub. No.: US 2024/0052061 A1
(43) Pub. Date: Feb. 15, 2024

(54) METHODS AND COMPOSITIONS FOR POTENTIATING ANTITUMORAL IMMUNE RESPONSES THROUGH TARGETING OF NTPDASE3

(71) Applicant: Purinomia Biotech, Inc., Woburn, MA (US)

(72) Inventor: Yan Wu Shang, North Reading, MA (US)

(21) Appl. No.: 18/025,072

(22) PCT Filed: Sep. 10, 2021
(86) PCT No.: PCT/US21/49770

§ 371 Date: Mar. 7, 2023

Related U.S. Application Data
(60) Provisional application No. 63/076,427, filed on Sep. 10, 2020.

(51) Int. Cl. C07K 16/40; A61K 45/06; A61K 39/395; A61K 47/68; A61P 35/00

(52) U.S. Cl. CPC C07K 16/40; A61K 45/06; A61K 39/3955; A61K 47/6871; A61P 35/00; C07K 2317/92; A61K 2039/505

(57) ABSTRACT

In combination with conventional therapies (e.g. targeted therapy, chemotherapy, and angiogenesis inhibitors etc.), immunotherapies targeting checkpoint molecules have shown promise in the treatment of solid or liquid tumors. However, the role of non-tumor cells in the intratumoral microenvironment has indicated that ablation of these cells may be a key to mounting an effective immune response against the tumor which includes tumor infiltration of cytotoxic T-cells and other anti-tumor cells of the immune system. The present invention not only brings up direct inhibition on the ectonucleotidase activity of NTPDase3 as an enzyme that generates adenosine, but also further utilizes NTPDase3 expression to bring about intratumoral cell ablation by NTPDase3-dependent ADCC.

### CROSS-REFERENCE TO RELATED APPLICATIONS

This application claims the benefit of priority to U.S. Provisional Application Ser. No. 63/076,427, filed on 10 Sep. 2020; the entire contents of said application are incorporated herein in their entirety by this reference.

### BACKGROUND OF THE INVENTION

For people with advanced cancers, hope can be a valuable but rare commodity. In recent years, a new class of drugs called immune checkpoint inhibitors has shown remarkable promise, keeping tumors at bay and preventing them from growing, and allowing some people who receive the treatments to essentially be cured. But these groundbreaking therapies have a substantial challenge. Despite the success of immunotherapies in advanced cancers based on inhibitory antibodies to programmed cell death protein 1 (PD1), PD1 ligand 1 (PDL1) and cytotoxic T lymphocyte antigen 4 (CTLA4) therapies in advanced cancer, a considerable proportion of patients remain unresponsive to these treatments.

With increasing attention on the immunosuppressive tumor microenvironment as a major driver of resistance, and characterization of “hot” and “cold” tumors depending on the level of immune cell infiltration, researchers have found several different mechanisms underlying the lack of efficacious responses to checkpoint monotherapies. Immunologically “hot” tumors contain high levels of infiltrating T cells and more antigens, making them more recognizable by the immune system and more likely to trigger a strong immune response. Amongst the cancers considered to be immunologically “hot” are bladder, head and neck, kidney, melanoma, and non-small cell lung cancers. However, even within these immunologically “hot” cancers, it is still only the minority of patients who derive benefit from immunotherapy. In contrast, immunologically “cold” tumors are cancers that for various reasons contain few infiltrating T cells, do not appear to be recognized as foreign and do not provoke a strong response by the immune system, making these cancers difficult to treat with current immunotherapies. Cancers that are classically immunologically “cold” include glioblastomas, as well as ovarian, prostate, pancreatic, and most breast cancers.

The microenvironment of tumors contains numerous cell types in addition to cancer cells, which include bone marrow-derived inflammatory cells, lymphocytes, blood vessels, pericytes, fibroblastic cells, and the extracellular matrix (ECM) composed of collagen and proteoglycans. Indeed, tumor drug responses are not exclusively determined by the tumor cell's intrinsic characteristics because tumor-associated stromal cells, including fibroblasts, mesenchymal stromal cells (MSCs), immuno-inflammatory cells, vascular endothelial cells, pericytes and the ECM combine in response to anti-cancer treatment.

Macrophages are widely distributed innate immune cells that play indispensable roles in the innate and adaptive immune response to pathogens and in-tissue homeostasis.

Macrophages can be activated by a variety of stimuli and polarized to functionally different phenotypes. Two distinct subsets of macrophages have been proposed, including classically activated (M1) and alternatively activated (M2) macrophages. M1 macrophages express a series of proinflammatory cytokines, chemokines, and effector molecules, such as IL-12, IL-23, TNF-α, iNOS and MHCI/II. In contrast, M2 macrophages express a wide array of anti-inflammatory molecules, such as IL-10, TGF-β, and arginase1. In most tumors, the infiltrated macrophages are considered to be of the M2 phenotype, which provides an immunosuppressive microenvironment for tumor growth. Furthermore, tumor-associated macrophages secrete many cytokines, chemokines, and proteases, which promote tumor angiogenesis, growth, metastasis, and immunosuppression. Inhibiting and/or decreasing intratumoral M2 and M2-like macrophage activity and/or levels are potential treatments for cancer.

### SUMMARY OF THE INVENTION

The ecto-nucleoside triphosphate diphosphohydrolase-1 (NTPDase1), also known as CD39, is the gene product of ENTPD1 and is a cell surface ecto-enzyme that scavenges and catalyzes extracellular nucleotides. This ectonucleotidase has been targeted to produce a decrease in the intratumoral level of the ecto-enzymatic activity associated with that protein. In so doing, this intervention is considered to boost the extracellular nucleotide concentrations that drive immune responses and decrease the intratumoral levels of the immunosuppressive nucleoside derivative, adenosine.

The ENTPD family member ecto-nucleoside triphosphate diphosphohydrolase-3 (NTPDase3), is abundantly expressed in some tissues such as pancreatic-cells, where it appears to play a role in regulating glucose-induced insulin secretion. See Saunders et al. (2019) Cell Metab 29(3):745-754, Lavoie et al. (2010) Am J Physiol Endocrinol Metab 299: E647-E656, Munkonda et al. (2009) FEBS J. 276:479-496, WO2018227176 and WO2006113237. In contrast to CD39, the proposed use of NTPDase3 antibodies in the prior art has been largely limited to potential anti-diabetic utility, or in the treatment of metabolic diseases more generally; as well as for diagnostic and imaging purposes. To the extent connected to immune-oncology, others have specifically taught away from the use of NTPDase3 antibodies, describing select anti-CD39 antibodies as selectively binding to NTPDase1 and not to NTPDase2 or NTPDase3. See, for example, WO2017157948, WO2017089334, WO2019096900 and WO2019243252.

Previously unknown in the art, NTPDase3 is also upregulated in the tumor microenvironment on cells and in a manner similar to NTPDase1 that also serves to generate immunosuppressive or an immune exclusionary environment. For example, we show NTPDase3 to be upregulated on M2 and M2-like macrophages that may confer potent immune suppressive signals in the tumor. Without wishing to be bound by any particular theory, the role of NTPDase3 in this context is potentially involved in promoting conversion of M1 macrophages (antitumor) to M2 macrophages (immunosuppressive). In the presence of ATP, M2 macrophages become potent immune suppressor cells by generating adenosine.

The present invention is based at least in part on the use of NTPDase3 antibodies and the antitumor activity of those antibodies possess though a mechanism of action impacting on the cellular makeup of a tumor. These actions are operational via targeted inhibition of the ectonucleotidase activity of NTPDase3 or by targeting cells that express the protein (such as M2 macrophage and/or pericytes or fibroblastic cells around the tumor vessels). This latter approach allows for ablation of these cells in the tumor using certain antibody-dependent cellular cytotoxicity (ADCC) competent anti-NTPDase3 antibodies. These novel reagents can be used to decrease M2 macrophage activities and/or levels in the tumor, and to enhance infiltration of cytotoxic T-cells and, in effect, convert “cold” tumors into immunologically “hot” ones. Without being bound by theory, it has been determined herein that NTPDase3 is expressed by pericytes and fibroblastic cells associated with tumor vessels, such that ablation of such cells (e.g., by ADCC) is believed to reduce hyperproliferation of the tumor by still a different mechanism, namely disruption of the tumor nutrient supply.

For example, in one aspect, an anti-NTPDase3 antibody, or antigen-binding fragment thereof, comprising at least one antigen binding domain that binds ectonucleoside triphosphate diphosphohydrolase-3 (NTPDase3) at a site such that the anti-NTPDase3 antibody forms a stable immune complex, and (a) an FcγRIIIa binding moiety that binds FcγRIIIa receptor and confers antibody-dependent cellular cytotoxicity (ADCC) activity against NTPDase3+ cells to the anti-NTPDase3 antibody; and/or (b) wherein the anti-NTPDase3 antibody, or antigen-binding fragment thereof, inhibits NTPDase3 enzymatic activity, is provided.

Numerous embodiments are further provided that may be applied to any aspect of the present invention and/or combined with any other embodiment described herein.

For example, in some embodiments, the anti-NTPDase3 antibody, or antigen-binding fragment thereof, has an EC50 of at least 2×10⁻⁶ molar (M) or lower in an in vitro ADCC assay, preferably wherein the EC50 is 1×10⁻⁶ M or lower, 0.5×10⁻⁶ M or lower, 1×10⁻⁷ M or lower, 7.5×10⁻⁸ M or lower, 5×10⁻⁸ M or lower, 2.5×10⁻⁸ M, 1×10⁻⁸ M or lower, 7.5×10⁻⁹ M or lower, 5×10⁻⁹ M or lower, 2.5×10⁻⁹ M or lower, 1×10⁻⁹ M or lower, 7.5×10⁻¹⁰ M or lower, 5×10⁻¹⁰ M or lower, 2.5×10⁻¹⁰ M or lower, 1×10⁻¹⁰ M or lower, 7.5×10⁻¹¹ M or lower, 5×10⁻¹¹ M or lower, 2.5×10⁻¹¹ M or lower, 1×10⁻¹¹ M or lower, 7.5×10⁻¹² M or lower, 5×10⁻¹² M or lower, 2.5×10⁻¹² M or lower, or 1×10⁻¹² M or lower, or any range in between, inclusive, including 1×10⁻⁶ M to 1×10⁻¹² M, 5×10⁻⁷ to 5×10⁻⁹ M, and 1×10⁻⁷ to 1×10⁻⁹ M.

In some embodiments, the anti-NTPDase3 antibody, or antigen-binding fragment thereof, the anti-NTPDase3 antibody, or antigen-binding fragment thereof, has an EC50 of at least 2×10⁻⁶ M or lower in an in vitro NTPD3 enzymatic activity inhibition assay, preferably wherein the EC50 is 1×10⁻⁶ M or lower, 0.5×10⁻⁶ M or lower, 1×10⁻⁷ M or lower, 7.5×10⁻⁸ M or lower, 5×10⁻⁸ M or lower, 2.5×10⁻⁸ M, 1×10⁻⁸ M or lower, 7.5×10⁻⁹ M or lower, 5×10⁻⁹ M or lower, 2.5×10⁻⁹ M or lower, 1×10⁻⁹ M or lower, 7.5×10⁻¹⁰ M or lower, 5×10⁻¹⁰ M or lower, 2.5×10⁻¹⁰ M or lower, 1×10⁻¹⁰ M or lower, 7.5×10⁻¹¹ M or lower, 5×10⁻¹¹ M or lower, 2.5×10⁻¹¹ M or lower, 1×10⁻¹¹ M or lower, 7.5×10⁻² M or lower, 5×10⁻² M or lower, 2.5×10⁻² M or lower, or 1×10⁻¹² M or lower, or any range in between, inclusive, including 1×10⁻⁶ M to 1×10⁻¹² M, 5×10⁻⁷ to 5×10⁻⁹ M, and 1×10⁻⁷ to 1×10⁻⁹ M, and with a maximal inhibition potency of at least 30%, 35%, 40%, 45%, 50%, 55%, 60%, 65%, 70%, 75%, 80%, 85%, 90%, 95%, 99%, or higher, or any range in between, inclusive, including 30% to 99%, as determined by an in vitro NTPD3 enzymatic activity inhibition assay.

In some embodiments, the anti-NTPDase3 antibody, or antigen-binding fragment thereof, has an EC50 of at least 4×10⁻⁶ M or lower in an in vitro ADCC assay, preferably wherein the EC50 is 2×10⁻⁶ M or lower, 1×10⁻⁶ M or lower, 0.5×10⁻⁶ M or lower, 1×10⁻⁷ M or lower, 7.5×10⁻⁸ M or lower, 5×10⁻⁸ M or lower, 2.5×10⁻⁸ M, 1×10⁻⁸ M or lower, 7.5×10⁻⁹ M or lower, 5×10⁻⁹ M or lower, 2.5×10⁻⁹ M or lower, 1×10⁻⁹ M or lower, 7.5×10⁻¹⁰ M or lower, 5×10⁻¹⁰ M or lower, 2.5×10⁻¹⁰ M or lower, 1×10⁻¹⁰ M or lower, 7.5×10⁻¹¹ M or lower, 5×10⁻¹¹ M or lower, 2.5×10⁻¹¹ M or lower, 1×10⁻¹¹ M or lower, 7.5×10⁻¹² M or lower, 5×10⁻¹² M or lower, 2.5×10⁻¹² M or lower, or 1×10⁻¹² M or lower, or any range in between, inclusive, including 1×10⁻⁶ M to 1×10⁻¹² M, 5×10⁻⁷ to 5×10⁻⁹ M, and 1×10⁻⁷ to 1×10⁻⁹ M; plus having an EC50 of at least 4×10⁻⁶ M or lower in an in vitro enzymatic activity inhibition assay, preferably wherein the EC50 is 2×10⁻⁶ M or lower, 1×10⁻⁶ M or lower, 0.5×10⁻⁶ M or lower, 1×10⁻⁷ M or lower, 7.5×10⁻⁸ M or lower, 5×10⁻⁸ M or lower, 2.5×10⁻⁸ M, 1×10⁻⁸ M or lower, 7.5×10⁻⁹ M or lower, 5×10⁻⁹ M or lower, 2.5×10⁻⁹ M or lower, 1×10⁻⁹ M or lower, 7.5×10⁻¹⁰ M or lower, 5×10⁻¹⁰ M or lower, 2.5×10⁻¹⁰ M or lower, 1×10⁻¹⁰ M or lower, 7.5×10⁻¹¹ M or lower, 5×10⁻¹¹ M or lower, 2.5×10⁻¹¹ M or lower, 1×10⁻¹¹ M or lower, 7.5×10⁻¹² M or lower, 5×10⁻¹² M or lower, 2.5×10⁻¹² M or lower, or 1×10⁻¹² M or lower, or any range in between, inclusive, including 1×10⁻⁶ M to 1×10⁻¹² M, 5×10⁻⁷ to 5×10⁻⁹ M, and 1×10⁻⁷ to 1×10⁻⁹ M, and with a maximal inhibition potency of at least 5%, 10%, 15%, 20%, 25%, 30%, 35%, 40%, 45%, 50%, 55%, 60%, 65%, 70%, 75%, 80%, 85%, 90%, 95%, 99%, or higher, or any range in between, inclusive, including 30% to 99%, as determined by an in vitro NTPD3 enzymatic activity inhibition assay.

A variety of assay setups for measuring NTPD3 functionality are well-known in the art. For example, target cells having differential levels of NTPD3 expression/enzyme activity; distinct function assay methods (e.g., Luciferase reporter assay versus direct NK-killing assay for measuring ADCC, CellTiter-Glo® luminescence assay versus malachite green phosphate assay for measuring enzyme activity or inhibition thereof, PBS versus Modified Ringer Buffer for measuring enzyme activity or inhibition thereof, and the like). Alternative assays may provide slightly different measured outputs (e.g., EC50, % enzyme inhibition, etc.), but methods for comparing and/or normalizing between assays are routine and well-known to the ordinarily skilled artisan.

Where the anti-NTPDase3 antibody includes an FcγRIIIa binding moiety, it can be selected from the group consisting of an Fc domain, an antibody or fragment thereof that binds to FcγRIIIa, and an FcγRIIIa binding peptide.

Where the anti-NTPDase3 antibody is an antigen binding domain, this can be also selected from the group consisting of Fab, Fab′, F(ab′)₂, Fv or single chain Fv (scFv), Fav, dsFv, sc(Fv)2, Fde, sdFv, single domain antibody (dAb), and diabodies.

Such an anti-NTPDase3 antibody can also be a monoclonal antibody.

In yet another embodiment, the anti-NTPDase3 antibody, or antigen-binding fragment thereof, is conjugated to an agent, optionally wherein the agent is selected from the group consisting of a binding protein, an enzyme, a drug, a chemotherapeutic agent, a biologic agent, a toxin, a radionuclide, an immunomodulatory agent, a detectable moiety, and a tag.

In another embodiment, the anti-NTPDase3 antibody, or antigen-binding fragment thereof, has a VH domain with an amino acid sequence that can be encoded by the nucleic acid sequence of or a nucleic acid that hybridizes under stringent conditions to a nucleic acid sequence described herein, such as the nucleic acid of SEQ ID No. 1, 9, 13, 17, 21, 25, 29, 33, 37, 41, 75, 79, or sequences listed in Table 2A, 2B, 2C, 2D, or 3, and a VL domain with an amino acid sequence that can be encoded by the nucleic acid sequence of or a nucleic acid that hybridizes under stringent conditions to a nucleic acid sequence described herein, such as the nucleic acid of SEQ ID No. 3, 11, 15, 19, 23, 27, 31, 35, 39, 43, 77, 81, or sequences listed in Table 2A, 2B, 2C, 2D, or 3 (such as hybridization under 6× sodium chloride/sodium citrate (SSC) at 45° C., and washing in 0.2×SSC/0.1% SDS at 50-65° C.).

In still another embodiment, the anti-NTPDase3 antibody, or antigen-binding fragment thereof, comprises a heavy chain having CDRs at least 60% identical (e.g., at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater) to the CDRs of a heavy chain variable sequence described herein, such as of SEQ ID No. 2, 10, 14, 18, 22, 26, 30, 34, 38, 42, 76, 80, or sequences listed in Table 2A, 2B, 2C, 2D, or 3, and a light chain having CDRs at least 60% identical (e.g., at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater) to the CDRs of a light chain variable sequence described herein, such as of SEQ ID No. 4, 12, 16, 20, 24, 28, 32, 36, 40, 44, 78, 82, or sequences listed in Table 2A, 2B, 2C, 2D, or 3.

In yet another embodiment, the anti-NTPDase3 antibody, or antigen-binding fragment thereof, comprises a variable heavy (VH) chain at least 60% identical (e.g., at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater) to a variable heavy chain sequence described herein, such as of SEQ ID No. 2, 10, 14, 18, 22, 26, 30, 34, 38, 42, 76, 80, or sequences listed in Table 2A, 2B, 2C, 2D, or 3, and a variable light (VL) chain at least 60% identical (e.g., at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater) to a variable light chain sequence described herein, such as of SEQ ID No. 4, 12, 16, 20, 24, 28, 32, 36, 40, 44, 78, 82, or sequences listed in Table 2A, 2B, 2C, 2D, or 3.

In another embodiment, the anti-NTPDase3 antibody, or antigen-binding fragment thereof, comprises a heavy chain having a CDRH1, CDRH2, and CDRH3 amino acid sequence and a light chain having a CDRL1, CDRL2, and CDRL3, each of which is at least 80% identical (e.g., at least 81%, 82%, 83%, 84%, 85%, 86%, 87%, 88%, 89%, 90%, 91%, 92%, 93%, 94%, 95%, 96%, 97%, 98%, 99%, or greater) to the respective CDR sequence of a respective variable heavy chain and variable light chain sequence described herein, such as for clones PBI #30, 3E9, 4F9, 8E1 (including h8E1), 16D4, 37H1, 38D5 (including h38D5), 38D12, 42D8, and 44H5, and variants thereof. For example, (i) a heavy chain having a CDR1 amino acid sequence at least 80% identical (e.g., at least 81%, 82%, 83%, 84%, 85%, 86%, 87%, 88%, 89%, 90%, 91%, 92%, 93%, 94%, 95%, 96%, 97%, 98%, 99%, or greater) to SEQ ID No. 45, a CDR2 amino acid sequence at least 80% (e.g., at least 81%, 82%, 83%, 84%, 85%, 86%, 87%, 88%, 89%, 90%, 91%, 92%, 93%, 94%, 95%, 96%, 97%, 98%, 99%, or greater) identical to SEQ ID No. 46, and a CDR3 amino acid sequence at least 80% identical (e.g., at least 81%, 82%, 83%, 84%, 85%, 86%, 87%, 88%, 89%, 90%, 91%, 92%, 93%, 94%, 95%, 96%, 97%, 98%, 99%, or greater) to SEQ ID No. 47; and (ii) a light chain having a CDR1 amino acid sequence at least 80% identical (e.g., at least 81%, 82%, 83%, 84%, 85%, 86%, 87%, 88%, 89%, 90%, 91%, 92%, 93%, 94%, 95%, 96%, 97%, 98%, 99%, or greater) to SEQ ID No. 48, a CDR2 amino acid sequence at least 80% identical (e.g., at least 81%, 82%, 83%, 84%, 85%, 86%, 87%, 88%, 89%, 90%, 91%, 92%, 93%, 94%, 95%, 96%, 97%, 98%, 99%, or greater) to SEQ ID No. 49, and a CDR3 amino acid sequence at least 80% identical (e.g., at least 81%, 82%, 83%, 84%, 85%, 86%, 87%, 88%, 89%, 90%, 91%, 92%, 93%, 94%, 95%, 96%, 97%, 98%, 99%, or greater) to SEQ ID No. 50.

In still another embodiment, the anti-NTPDase3 antibody, or antigen-binding fragment thereof, comprises a heavy chain having CDRs selected from the group consisting of CDRs of a variable heavy chain sequence described herein, such as of SEQ ID No. 2, 10, 14, 18, 22, 26, 30, 34, 38, 42, 76, 80, and sequences listed in Tables 2A, 2B, 2C, 2D, and 3, and a light chain having CDRs selected from the group consisting of CDRs of a variable light chain sequence described herein, such as of SEQ ID No. 4, 12, 16, 20, 24, 28, 32, 36, 40, 44, 78, 82, and sequences listed in Tables 2A, 2B, 2C, 2D, and 3, and human framework sequences to form humanized heavy and light chains with an antigen binding site able to specifically bind human NTPDase3.

In yet another embodiment, the anti-NTPDase3 antibody, or antigen-binding fragment thereof, comprises an Fc domain of an IgG1, IgG3, IgG2, or IgG4 isotype, optionally wherein the Fc domain is human, preferably the isotype is an IgG1 or IgG3 that is ADCC active.

In another embodiment, the anti-NTPDase3 antibody, or antigen-binding fragment thereof, is hypo-fucosylated or afucosylated.

In still another embodiment, the anti-NTPDase3 antibody, or antigen-binding fragment thereof, is human or is humanized.

In yet another embodiment, the anti-NTPDase3 antibody, or antigen-binding fragment thereof, is bispecific: including at least one additional antigen binding site for a tumor antigen, immune checkpoint, or costimulatory receptor. Wherein if the additional antigen binding site is for an immune checkpoint, it functions as a checkpoint inhibitor and wherein if the additional antigen binding site is for a costimulatory receptor, it functions as a costimulatory agonist. In another embodiment, the additional antigen binding site binds to a checkpoint protein, such as selected from the group consisting of CD39, PD-1, PD-L1, CTLA-4/B7-1/B7-2, PD-L2, NKG2A, KIR, LAG-3, TIM-3, CD96, VISTA, TIGIT and Siglec-15.

In still another embodiment, the additional antigen binding site binds a checkpoint protein upregulated on T-cells and associated with T-cell exhaustion. In yet another embodiment, the additional antigen binding site binds to an immune costimulatory receptor, such as selected from the group consisting of MHCI molecules, BTLA receptor, OX40, CD27, CD28, CDS, ICAM-1, LFA-1 (CD11a/CD18), ICOS (CD278) and 4-1BB (CD137).

In another embodiment, the additional antigen binding site binds to an inducer/potentiator of innate immunity, such as CD47, SIRPα, CD24, Siglec-15 or Siglec-10.

In certain embodiments, the anti-NTPDase3 antibody, or antigen-binding fragment thereof, is a therapeutic antibody, or antigen-binding fragment thereof.

In certain embodiments, the anti-NTPDase3 antibody, or antigen-binding fragment thereof, has complement dependent cytotoxicity (CDC) activity against NTPDase3+ cells.

In certain embodiments, the anti-NTPDase3 antibody, or antigen-binding fragment thereof, has ADCC activity against NTPDase3+ intratumoral cells, such as NTPDase3+ immune cells, cancer cells, and/or pericytes and/or fibroblasts around tumor vessels.

In certain embodiments, the anti-NTPDase3 antibody, or antigen-binding fragment thereof, can induce antibody-mediated target cytosis of NTPDase3 on NTPDase3+ immune cells, such as M2 macrophage and/or other types of NTPDase3 expressing cells inside the tumor.

In certain embodiments, the anti-NTPDase3 antibody, or antigen-binding fragment thereof, can inhibit NTPDase3 enzymatic activity on NTPDase3+ immune cells, such as M2 macrophage and/or other types of NTPDase3 expressing cells inside the tumor.

In certain embodiments, the anti-NTPDase3 antibody, or antigen-binding fragment thereof, binds to NTPDase3 in a manner that is competitive, non-competitive, or partially competitive with an NTPDase3 monoclonal antibody clone binding to NTPDase3, wherein the NTPDase3 monoclonal antibody clone is selected from the group consisting of NTPDase3 clones described herein, including, without limitation, PBI #30 and its affinity maturation variants, 3E9, 4F9, 8E1 and its humanized counterparts, 16D4, 37H1, 38D5 and its humanized counterparts with or without point mutation in their backbone sequences, 38D12, 42D8, and 44H5.

In another aspect, a pharmaceutical preparation comprising a therapeutically effective amount of at least one anti-NTPDase3 antibody, or antigen-binding fragment thereof, described herein, and one or more pharmaceutically acceptable excipients, buffers or solutions, is provided. For example, the pharmaceutical preparation can be for improving anti-tumor T cell immunity and suitable for administration to a subject having a tumor, comprising an effective amount of the anti-NTPDase3 antibody, or antigen-binding fragment thereof, and one or more pharmaceutically acceptable excipients, buffers or solutions, wherein administration of the anti-NTPDase3 antibody to the subject results in a reduction in numbers of intratumoral NTPDase3+ cells, such as M2 and M2-like macrophage, and may i) enhance T-cell infiltration into the tumor, ii) T-cell exhaustion in the tumor, and/or iii) disrupt tumor-associated vasculature thereby resulting in tumor starvation.

In still another aspect, a pharmaceutical preparation comprising a therapeutically effective amount of at least one anti-NTPDase3 antibody, or antigen-binding fragment thereof, described herein, and one or more pharmaceutically acceptable excipients, buffers or solutions, is provided. For example, the pharmaceutical preparation can be for inhibiting tumor angiogenesis and suitable for administration to a subject having a tumor, comprising an effective amount of the anti-NTPDase3 antibody, or antigen-binding fragment thereof, and one or more pharmaceutically acceptable excipients, buffers or solutions, wherein administration of the anti-NTPDase3 antibody to the subject results in a reduction in numbers of intratumoral NTPDase3+ cells, such as pericytes and/or fibroblastic cells, and may disrupt the integrity of tumor-associated vascular structure to limit tumor growth.

In still another aspect, an isolated nucleic acid molecule that i) hybridizes, under stringent conditions, with the complement of a nucleic acid encoding an immunoglobulin heavy and/or light chain polypeptide of an anti-NTPDase3 antibody, or antigen-binding fragment thereof, described herein; ii) has a sequence with at least about 90% identity across its full length to a nucleic acid encoding an immunoglobulin heavy and/or light chain polypeptide of an anti-NTPDase3 antibody, or antigen-binding fragment thereof, described herein; or iii) encodes an immunoglobulin heavy and/or light chain polypeptide of an anti-NTPDase3 antibody, or antigen-binding fragment thereof, described herein, is provided.

In yet another aspect, an isolated immunoglobulin heavy and/or light chain polypeptide encoded by a nucleic acid descried herein, is provided.

In another aspect, a vector comprising an isolated nucleic acid described herein, is provided, optionally wherein the vector is an expression vector.

In still another aspect, a host cell which comprises an isolated nucleic acid described herein, that: a) expresses an anti-NTPDase3 antibody, or antigen-binding fragment thereof, described herein; b) comprises the immunoglobulin heavy and/or light chain polypeptide of a polypeptide described herein; or c) comprises a vector described herein.

In yet another aspect, a device or kit comprising at least one anti-NTPDase3 antibody, or antigen-binding fragment thereof, described herein is provided. The device or kit optionally comprises a label to detect the at least one anti-NTPDase3 antibody, or antigen-binding fragment thereof, or a complex comprising the anti-NTPDase3 antibody, or antigen-binding fragment thereof.

In another aspect, a device or kit comprising a pharmaceutical composition, isolated nucleic acid molecule, isolated immunoglobulin heavy and/or light chain polypeptide, vector, and/or host cell described herein, is provided.

In still another aspect, a method of producing at least one anti-NTPDase3 antibody, or antigen-binding fragment thereof, which method comprises the steps of: (i) culturing a transformed host cell which has been transformed by a nucleic acid comprising a sequence encoding the at least one anti-NTPDase3 antibody, or antigen-binding fragment thereof, under conditions suitable to allow expression of said anti-NTPDase3 antibody, or antigen-binding fragment thereof; and (ii) recovering the expressed anti-NTPDase3 antibody, or antigen-binding fragment thereof, is provided.

In yet another aspect, a method of detecting the presence or level of NTPDase3 polypeptide comprising obtaining a sample and detecting said polypeptide in the sample by use of at least one anti-NTPDase3 antibody, or antigen-binding fragment thereof, described herein, is provided. For example, at least one anti-NTPDase3 antibody, or antigen-binding fragment thereof, can form a complex with the NTPDase3 polypeptide and the complex can be detected in the form of an enzyme linked immunosorbent assay (ELISA), radioimmune assay (RIA), immunochemical assay, Western blot, mass spectrometry assay, nuclear magnetic resonance assay, or using an intracellular flow assay.

In another aspect, a method for improving anti-tumor therapies by depleting intratumoral NTPDase3+ cells (such as M2 and M2-like macrophages, pericytes, and/or fibroblasts), comprising administering to a subject having a tumor an effective amount of a pharmaceutical composition of an anti-NTPDase3 antibody, or antigen-binding fragment thereof, described herein, wherein administration of the anti-NTPDase3 antibody, or antigen-binding fragment thereof, results in a reduction in numbers of intratumoral NTPDase3+ cells, is provided.

In still another aspect, a method for promoting immune cell infiltration into tumors, comprising administering to a subject having a tumor an effective amount of a pharmaceutical composition of an anti-NTPDase3 antibody, or antigen-binding fragment thereof, described herein, wherein administration of the anti-NTPDase3 antibody, or antigen-binding fragment thereof, results in ablation and reduction of NTPDase3+ cells in the tumor, is provided.

In yet another aspect, a method for reducing type II NKT cells suppression of intratumoral immune cell function, comprising administering to a subject having a tumor an effective amount of a pharmaceutical composition of an anti-NTPDase3 antibody, or antigen-binding fragment thereof, described herein, optionally wherein administration of the anti-NTPDase3 antibody, or antigen-binding fragment thereof, results in ablation and reduction of M2 macrophage in the tumor, is provided.

In another aspect, a method for reducing regulatory T cells (Treg) suppression of intratumoral immune cell function, comprising administering to a subject having a tumor an effective amount of a pharmaceutical composition of an anti-NTPDase3 antibody, or antigen-binding fragment thereof, described herein, optionally wherein administration of the anti-NTPDase3 antibody, or antigen-binding fragment thereof, results in reduced immunosuppressive activity of M2 macrophage in the tumor.

In still another aspect, a method for promoting an anti-tumor immune response comprising administering to a subject having a tumor an anti-NTPDase3 antibody, or antigen-binding fragment thereof, described herein, in an amount sufficient to result in a reduction of NTPDase3 expressing cells in the tumor, is provided.

In yet another aspect, a method for promoting T-cell mediated immune function in a tumor of a subject, comprising (i) identifying a cancer subject having a degree of tumor infiltrated tumor-reactive lymphocytes that is below a predetermined threshold so as to be characterized as being a non-infiltrated or under-infiltrated tumor phenotype; and (ii) administering to the subject an anti-NTPDase3 antibody, or antigen-binding fragment thereof, described herein in an amount that increases tumor-reactive T-cell activity in the tumor, is provided.

In exemplary embodiments, the anti-NTPDase3 antibody, or antigen-binding fragment thereof, is administered as part of an antitumor therapy, such as anti-tumor therapies for treating solid tumor, optionally wherein the solid tumor is pancreatic cancer, liver cancer, lung cancer, stomach cancer, esophageal cancer, head and neck squamous cell carcinoma, prostate cancer, colon cancer, breast cancer, lymphoma, gallbladder cancer, renal cancer, multiple myeloma, ovarian cancer, cervical cancer or glioma.

In still another embodiment, the anti-NTPDase3 antibody, or antigen-binding fragment thereof, is administered as part of an anti-tumor therapy for treating a liquid tumor, optionally wherein the liquid tumor is a leukemia.

In yet another embodiment, the anti-NTPDase3 antibody, or antigen-binding fragment thereof, is administered as part of a therapy involving one or more chemotherapeutic agents, anti-angiogenetic agents, immuno-oncology agents and/or radiation.

In another embodiment, the therapy includes administering one or more inhibitors (antagonists) of one or more checkpoint molecules, optionally wherein the one or more checkpoint molecules is selected from the group consisting of a PD-1 antagonists, a CTLA-4 antagonist, a LAG-3 antagonist, a TIM-3 antagonist, a TIGIT antagonist and a Siglec-15 antagonist. In still another embodiment, the therapy includes administering one or more activators (agonists) of one or more costimulatory molecules, optionally wherein the one or more costimulatory molecules is selected from the group consisting of a GITR agonist, a CD27 agonist, a 4-1BB agonist, an OX40 agonist, a CD137 agonist, an ICOS agonist and a CD28 agonist.

In yet another embodiment, the therapy includes administering one or more of a VEGFR or VEGF antagonist, an EGFR or EGF antagonist, an IDO inhibitor, an IDO1 inhibitor, an HDAC inhibitor, a PI3K delta inhibitor, an IL-15 agonist, a CXCR4 antagonist, a CXCL12 antagonist, a DNMT inhibitor, interleukin-21, an anti-KIR antibody, an anti-CSF-1R antibody, an anti-CCR4 antibody, GMCSF, an anti-PS antibody, an anti-CD30 antibody-auristatin E conjugate, an anti-CD19 antibody, an anti-CEA IL-2 antibody, an anti-NY-ESO-1 antibody, an anti-NKG2A antibody, a STING agonist, a TRL7/8 agonist, a RIG-1 agonist and/or NRLP3 inhibitor, an anti-CD73 antibody (such as MED19447), a P2X7 antagonist, an adenosine A2a receptor antagonist, or an anti-CD39 antibody.

In another embodiment, the therapy includes administering one or more innate immune inducers, optionally wherein the one or more innate immune inducers is selected from the group consisting of an inhibitor of the CD47-SIRPα axis (e.g., antibodies or other binding moieties that bind to CD47 or SIRPα, and inhibit the interaction of the two molecules), an inhibitor of the CD24-Siglec-10 axis (e.g., antibodies or other binding moieties that bind to CD24 or Siglec-10 and inhibit the interaction of the two molecules), an NGK2A checkpoint inhibitor that blocks HLA-E driven inhibition of NK and CD8+ cells, a STING agonist, a TLR7/8 agonist and an RIG-1 agonist.

In still another embodiment, the anti-NTPDase3 antibody, or antigen-binding fragment thereof, is administered as a part of therapy including a tumor vaccine, adoptive cell therapy (including CAR-T and ACTR therapy), antitumor gene therapy, inhibitory nucleic acid therapy (such as siRNA, shRNA, antisense, CRISPR and TALEN therapy) and/or oncolytic viral therapy.

In yet another embodiment, the subject is an animal model of cancer. In another embodiment, the subject is a mammal, optionally wherein the mammal is a human or a rodent.

### BRIEF DESCRIPTION OF THE FIGURES

FIG. 1. Affinity of PBI #30 measured by flow cytometry using human ENTPD3 (hENTPD3) positive CHO cells (stably transfected cells). PBI #30 was serially diluted as indicated (top dose 333 nM) and incubated with CHO-hENTPD3 cells for 30 minutes at 4° C., followed by secondary antibody staining (anti-human IgG (Fc specific), Alexa Fluor® 488) for 30 minutes at 4° C. and flow cytometry analysis. Kd was calculated as 5.53 nM.

FIG. 2. Affinity of PBI #30 measured by flow cytometry using hENTPD3 positive COS-7 cells (stably transfected cells). PBI #30 was serially diluted as indicated (top dose 133 nM) and incubated with COS7-hENTPD3 cells for 30 minutes at 4° C., followed by secondary antibody staining (anti-human IgG (Fc specific), Alexa Fluor® 488) for 30 minutes at 4° C. and flow cytometry analysis. Kd was calculated as 11.9 nM.

FIG. 3. Affinity of PBI #30 measured by flow cytometry using hENTPD3 positive HEK293T cells (stably transfected cells). PBI #30 was serially diluted as indicated (top dose 133 nM) and incubated with HEK293T-hENTPD3 cells for 30 minutes at 4° C., followed by secondary antibody staining (anti-human IgG (Fc specific), Alexa Fluor® 488) for 30 minutes at 4° C. and flow cytometry analysis. Kd was calculated as 8.4 nM.

FIG. 4. Affinity of PBI #30 measured by flow cytometry using RT4 bladder cancer cells that endogenously express hENTPD3. PBI #30 was serially diluted as indicated (top dose 133 nM) and incubated with RT4 cells for 30 minutes at 4° C., followed by secondary antibody staining (anti-human IgG (Fc specific), Alexa Fluor® 488) for 30 minutes at 4° C. and flow cytometry analysis. Kd is calculated as 10.1 nM.

FIG. 5. PBI #30 inhibits hENTPD3 enzyme activity on CHO cell membrane. CHO-hENTPD3 cells were incubated with 10 μg/mL of human IgG1 isotype Ultra-LEAF, mouse anti-hENTPD3 clone hN3-B3s, or PBI #30 for 30 minutes at 37° C., followed by incubation with ATP (250 μM) for 15 minutes at room temperature. Supernatants were then collected and ATP levels were detected by luminescence using CellTiter-Glo®. Cells with no antibody (Cells+ATP) or ATP alone in the absence of cells were also detected in parallel in order to calculate % of enzymatic activity inhibition as described in Materials and Methods.

FIG. 6. PBI #30 fully inhibits hENTPD3 enzyme activity on CHO cell membrane at saturation dose. CHO-hENTPD3 cells were incubated with serially diluted human IgG1 isotype Ultra-LEAF or PBI #30 (top dose 50 μg/mL) for 30 minutes at 37° C., followed by incubation with ATP (250 μM) for 15 minutes at room temperature. Supernatants were then collected and ATP levels were detected by luminescence using CellTiter-Glo®. Cells with no antibody (Cells+ATP) or ATP alone in the absence of cells were also detected in parallel and presented in the graphic for comparison. EC50 was calculated as 17.30 μg/mL.

FIG. 7. PBI #30 inhibits hENTPD3 enzyme activity on RT4 cell membrane. RT4 cells, both attached and in suspension, were incubated with 10 μg/mL of human IgG1 isotype Ultra-LEAF or PBI #30 for 30 minutes at 37° C., followed by incubation with ATP (25 μM, attached; or 50 μM, suspension) for 45 minutes at 37° C. Supernatants were then collected and ATP levels were detected by luminescence using CellTiter-Glo®. Cells with no antibody (Cells+ATP) and ATP alone (25 or 50 μM) in the absence of cells were also detected in parallel in order to calculate % of enzymatic activity inhibition as described in Materials and Methods.

FIG. 8. PBI #30 demonstrates ADCC activity against CHO-hENTPD3 cells. CHO-hENTPD3 cells were used as target cells. Jurkat cells stably expressing luciferase and hCD16a-158V were used as effector cells. Target cells were pre-incubated with serially diluted PBI #30 as indicated for 30 minutes at 37° C. in 5% CO₂, followed by co-culture with effector cells (T:E=1:6) for 6 hours. ADCC activity was indicated by an increase of luciferase activity over background. RLU: Relative Luminescence Unit. EC50 was calculated as 0.092 μg/mL.

FIG. 9. Affinity of Human/Rabbit chimeric anti-human ENTPD3 clones measured by flow cytometry using CHO-hENTPD3 cells. Rabbit anti-human ENTPD3 monoclonal antibodies chimerized with a human IgG1 Fc (Human/Rabbit chimeric clones; Hu/Ra) were serially diluted as indicated (top dose 33 nM) and incubated with CHO-hENTPD3 cells for 30 minutes at 4° C., followed by secondary antibody staining (anti-human IgG (Fc specific), Alexa Fluor® 488) for 30 minutes at 4° C. and flow cytometry analysis. Kd was calculated for each clone and presented.

FIG. 10. Affinity of Human/Rabbit chimeric anti-human ENTPD3 clones measured by flow cytometry using COS7-hENTPD3 cells. Hu/Ra chimeric anti-human ENTPD3 clones (Hu/Ra) were serially diluted as indicated (top dose 133 nM) and incubated with COS7-hENTPD3 cells for 30 minutes at 4° C., followed by secondary antibody staining (anti-human IgG (Fc specific), Alexa Fluor® 488) for 30 minutes at 4° C. and flow cytometry analysis. Kd was calculated for each clone and presented.

FIG. 11. Affinity of Human/Rabbit chimeric anti-human ENTPD3 clones measured by flow cytometry using HEK293T-hENTPD3 cells. Hu/Ra chimeric anti-human ENTPD3 clones were serially diluted as indicated (top dose 133 nM) and incubated with HEK293T-hENTPD3 cells for 30 minutes at 4° C., followed by secondary antibody staining (anti-human IgG (Fc specific), Alexa Fluor® 488) for 30 minutes at 4° C. and flow cytometry analysis. Kd was calculated for each clone and presented.

FIG. 12. Affinity of Human/Rabbit chimeric anti-human ENTPD3 clones measured by flow cytometry using hENTPD3 positive RT4 bladder cancer cells. Human/Rabbit chimeric anti-human ENTPD3 clones (Hu/Ra), except 3E9 which is a rabbit monoclonal antibody, were serially diluted as indicated (top dose 133 nM) and incubated with RT4 cells for 30 minutes at 4° C., followed by secondary antibody staining (anti-human or anti-rabbit IgG, Alexa Fluor® 488) for 30 minutes at 4° C. and flow cytometry analysis. Kd was calculated for each clone and presented.

FIG. 13. Clone 38D5 exerts the highest inhibition capacity of hENTPD3 enzyme activity on CHO cell membrane among all chimeric clones. CHO-hENTPD3 cells were incubated with human 10 μg/mL IgG1 isotype Ultra-LEAF, mouse anti-hENTPD3 clone hN3-B3s or Hu/Ra chimeric anti-human ENTPD3 clones for 30 minutes at 37° C., followed by incubation with ATP (250 μM) for 15 minutes at room temperature. Supernatants were then collected and ATP levels were detected by luminescence using CellTiter-Glo®. Cells with no antibody (Cells+ATP) or ATP alone in the absence of cells were also detected in parallel in order to calculate % of enzymatic activity inhibition as described in Materials and Methods.

FIG. 14. Clone 38D5 partially inhibits hENTPD3 enzyme activity on CHO cell membrane even at the saturated dose. CHO-hENTPD3 cells were incubated with serially diluted human IgG1 isotype Ultra-LEAF or Hu/Ra 38D5 clone (top dose 50 μg/mL) for 30 minutes at 37° C., followed by incubation with ATP (250 μM) for 15 minutes at room temperature. Supernatants were then collected and ATP levels were detected by luminescence using CellTiter-Glo®. Cells with no antibody (Cells+ATP) or ATP alone in the absence of cells were also detected in parallel and presented in the graphic for comparison. EC50 was calculated as 15.92 μg/mL.

FIG. 15. Clone 38D5 exerts the highest inhibition capacity of hENTPD3 enzyme activity on RT4 cell membrane among all chimeric clones. RT4 cells, both attached and in suspension, were incubated with 10 μg/mL of human IgG1 isotype Ultra-LEAF or Hu/Ra chimeric anti-human ENTPD3 clones, except 3E9 which is a rabbit monoclonal antibody, for 30 min at 37° C., followed by incubation with ATP (25 μM, attached; or 50 μM, suspension) for 45 minutes at 37° C. Supernatants were then collected and ATP levels were detected by luminescence using CellTiter-Glo®. Cells with no antibody (Cells+ATP) or ATP alone (25 or 50 μM) in the absence of cells were also detected in parallel in order to calculate % of enzymatic activity inhibition as described in Materials and Methods.

FIG. 16. ADCC activity of Human/Rabbit chimeric clones against CHO-hENTPD3 cells. CHO-hENTPD3 cells were used as target cells. Jurkat cells stably expressing luciferase and hCD16a-158V were used as effector cells. Target cells were pre-incubated with serially diluted Hu/Ra chimeric anti-human ENTPD3 antibodies as indicated for 30 minutes at 37° C. in 5% CO₂, followed by co-culture with effector cells (T:E=1:6) for 6 hours. ADCC activity was indicated by an increase of luciferase activity over background. RLU: Relative Luminescence Unit. EC50 was calculated for each clone and presented.

FIG. 17. Epitope competition assay for all anti-human ENTPD3 monoclonal antibodies in CHO-hENTPD3 cells. CHO-hENTPD3 cells were incubated with 20 μg/ml of unconjugated human IgG1 isotype Ultra-LEAF or anti-human ENTPD3 antibodies, at 4° C. for 30 minutes. Cells were then incubated with Alexa Fluor® 647-conjugated clones 3E9, 38D5, 38D12, 44H5, or PBI #30 for 30 minutes at 4° C., washed twice and analyzed by flow cytometry. Fold change in AF647 MFI detection in relation to isotype control was calculated (No epitope overlap=1).

FIG. 18. Anti-tumor efficacy of Human/Rabbit chimeric clones 38D5 and 8E1 in vivo. Six-to 8-week-old C57BL6 hENTPD3 KI mice were inoculated subcutaneously with MC38 colorectal cancer cells (5×10⁵) in 150 μl of RPMI 1640 media. Mice were then randomized into three groups (n=6). On day 4, tumor-bearing mice received 20 mg/kg of chimeric anti-hENTPD3 antibody 38D5 or 8E1, or 200 μl of saline, via intraperitoneal injection. On days 7, 10 and 14, tumor-bearing mice received 10 mg/kg of 38D5 or 8E1 antibody, or 200 μl of saline. Tumor length (L) and width (W) were measured using a digital caliper twice weekly. Tumor volume (mm³) was determined as L*W*W*0.52.

FIG. 19. Affinity of the fully human anti-hENTPD3 monoclonal antibody PBI #30 hIgG1 measured by flow cytometry using CHO-hENTPD3 and COS7-hENTPD3 cells. Generally, FIG. 19 and following are directed to analyses of anti-hENTPD3 monoclonal antibody clones that have been further optimized exemplary (e.g., affinity maturation and isotype switching for PBI #30 and humanization of 8E1 and 38D5) and related detection methods. For example, FIG. 19 shows results of assays, wherein PBI #30 hIgG1 was serially diluted as indicated (top dose 33 nM) and incubated with cells for 2 hours at 37° C., followed by secondary antibody staining (anti-human IgG (Fc specific), Alexa Fluor® 488) for 30 minutes at 4° C. and subsequent flow cytometry analysis. Kd was calculated as 2.96 nM for CHO-hENTPD3 cells and 1.82 nM for COS7-hENTPD3 cells.

FIG. 20. PBI #30 hIgG1 antibody inhibits hENTPD3 enzyme activity on both hENTPD3 positive CHO and COS7 cell membrane. Cells were incubated with 2 μg/mL of human IgG1 isotype Ultra-LEAF or PBI #30 hIgG1 for 2 hours at 37° C., followed by incubation with ATP (250 μM) for 15 minutes at 37° C. Supernatants were then collected and ATP levels were detected by luminescence using CellTiter-Glo®. Cells with no antibody (Cells+ATP) or ATP alone in the absence of cells were also detected in parallel in order to calculate % of enzymatic activity inhibition as described in Materials and Methods below.

FIG. 21. PBI #30 hIgG1 has no CDC activity toward CHO-hENTPD3 cells. PBI #30 hIgG1 were serially diluted as indicated and pre-incubated with CHO-hENTPD3 target cells for 30 minutes at 37° C., followed by incubation with 10% Normal Human Serum (NHS) for 2 hours. Target cell lysis was analyzed by flow cytometry and % P/I⁺ cells (% of cytotoxicity) was calculated. No CDC activity was noted so EC50 was listed as N/A (not applicable).

FIG. 22. Assaying PBI #30 hIgG1 for anti-tumor activity in vivo. Six- to 8-week-old C57BL6 hENTPD3 KI mice were inoculated subcutaneously with MC38 colorectal cancer cells (5×10⁵) in 150 μl of RPMI 1640 media. Mice were then randomized into two groups (n=6). On day 4, tumor-bearing mice received 20 mg/kg of PBI #30 hIgG1, or 200 μl of saline, via intraperitoneal injection. On days 7, 10 and 14, tumor-bearing mice received 10 mg/kg of PBI #30 hIgG1, or 200 μl of saline. Tumor length (L) and width (W) were measured using a digital caliper twice weekly. Tumor volume (mm³) was determined as L*W*W*0.52.

FIG. 23. Affinity of PBI #30 maturation variants measured by flow cytometry using CHO-hENTPD3 cells. The original clone PBI #30 hIgG1 was subjected to affinity maturation as well as isotype switching processes resulting in eight new variants. PBI #30 maturation variants were serially diluted as indicated (top dose 66 nM) and incubated with CHO-hENTPD3 cells for 2 hours at 37° C., followed by secondary antibody staining (anti-human IgG (Fc specific), Alexa Fluor® 488) for 30 minutes at 4° C. and flow cytometry analysis. The original PBI #30 hIgG1 antibody was used as the reference. Kd was calculated for each variant and presented.

FIG. 24. Affinity of PBI #30 maturation variants measured by flow cytometry using COS7-hENTPD3 cells. PBI #30 maturation variants and the original PBI #30 hIgG1 antibody were serially diluted as indicated (top dose 66 nM) and incubated with COS7-hENTPD3 cells for 2 hours at 37° C., followed by secondary antibody staining (anti-human IgG (Fc specific), Alexa Fluor® 488) for 30 minutes at 4° C. and flow cytometry analysis. Kd was calculated for each variant and presented.

FIG. 25. PBI #30 maturation variants strongly inhibit hENTPD3 enzyme activity on CHO-hENTPD3 cell membrane. CHO-hENTPD3 cells were incubated with 2 μg/mL of PBI #30 maturation variants for 2 hours at 37° C., followed by incubation with ATP (250 μM) for 15 minutes at 37° C. Supernatants were then collected and ATP levels were detected by luminescence using CellTiter-Glo®. Cells with no antibody (Cells+ATP) or ATP alone in the absence of cells were also detected in parallel in order to calculate % of enzymatic activity inhibition as described in Materials and Methods. The original PBI #30 hIgG1 antibody was used as the reference.

FIG. 26. PBI #30 hIgG1 maturation variants fully inhibit hENTPD3 enzyme activity on CHO cell membrane at lower dose if incubated for longer time. Cells were incubated with serially diluted isotype control or antibodies (top dose 10 μg/mL) for 2 hours at 37° C., followed by incubation with ATP (250 μM) for 15 minutes at room temperature. Supernatants were then collected and ATP levels were detected by luminescence using CellTiter-Glo®. Cells with no antibody (Cells+ATP) or ATP alone in the absence of cells were also detected in parallel and presented in the graphic for comparison. EC50 was calculated and presented. Note the difference in EC50 of the original clone PBI #30 hIgG1 from FIG. 6 where cells were incubated with the antibody for 30 min at 4° C.

FIG. 27. PBI #30 maturation variant the Fc-competent af4 hIgG1 but not the Fc-silent af4 hIgG4 has high ADCC activity against CHO-hENTPD3 cells. CHO-hENTPD3 cells were used as target cells. Jurkat cells stably expressing luciferase and hCD16a-158V were used as effector cells. Target cells were pre-incubated with serially diluted isotype control or PBI #30 maturation variants as indicated for 30 minutes at 37° C., followed by co-culture with effector cells (T:E=1:6) for 6 hours. ADCC activity was indicated by an increase of luciferase activity over background. RLU: Relative Luminescence Unit. EC50 was calculated as 1.443 μg/mL for PBI #30 hIgG1 and 0.434 for PBI #30af4 hIgG1. ADCC fold increase in relation to PBI #30 hIgG1 was determined as: RLU of maturation variant/RLU of PBI #30 hIgG1 at 1 μg/mL. Note: PBI #30af4 hIgG1 maturation variant shows an 2.8-fold increase in ADCC activity over the parental PBI #30 hIgG1 clone.

FIG. 28. PBI #30 maturation variants have no CDC activity toward CHO-hENTPD3 cells. PBI #30 hIgG1 and its maturation variants were serially diluted as indicated and pre-incubated with CHO-hENTPD3 target cells for 30 minutes at 37° C., followed by incubation with 10% Normal Human Serum (NHS) for 2 hours. Target cell lysis was analyzed by flow cytometry and % P/I⁺ cells (% of cytotoxicity) was calculated. No CDC activity was noted so EC50 was listed as N/A (not applicable). PC: in-house positive control.

FIG. 29. Anti-tumor efficacy of two exemplary PBI #30 maturation variants (af4 hIgG1 and af4 hIgG4) in vivo. 13- to 16-week-old C57BL6 hENTPD3 KI female mice were inoculated subcutaneously with MC38 colorectal cancer cells (1×10⁵) in 150 μl of RPMI 1640 media. Mice were then randomized into three groups (n=8 per group). Tumor-bearing mice received 3 mg/kg of fully human anti-hENTPD3 antibody PBI #3af4 hIgG1 or PBI #3af4 hIgG4, or 200 μl of saline, on days 8, 12, 15, 18 and 21, via intraperitoneal injection. Tumor length (L) and width (W) were measured using a digital caliper every two days. Tumor volume (mm³) was determined as L*W*W*0.52.

FIG. 30. Plasma clearance of PBI #30 maturation variants (af4 hIgG1 and af4 hIgG4) in vivo after repeated dose exposure. 13- to 16-week-old C57BL6 hENTPD3 KI female mice were inoculated subcutaneously with MC38 colorectal cancer cells (1×10⁵) in 150 μl of RPMI 1640 media. Tumor-bearing mice received 3 mg/kg of PBI #3af4 hIgG1 or PBI #3af4 hIgG4, on days 8, 12, 15, 18, and 21 via intraperitoneal injection. Plasma samples were collected on days 20, 21, 22 and 24, and free plasma antibody level was detected by binding analysis using COS7-hENTPD3 cells by flow cytometry. PBI #30af4 hIgG1 and hIgG4 (2 μg/mL) were used as respective positive controls to indicate maximal cell binding. Sample name is designated as: animal number+collection date. Note that after repeated dose exposure, PBI #30af4 hIgG1 antibody is quickly cleared in the mouse blood (plasma antibody level is only detected from 3 out of 8 mice), while PBI #30af4 hIgG4 remains stable (100% of mice have detectable plasma antibody levels with good in vitro binding kinetics).

FIG. 31. Affinity of 8E1 Human/Rabbit chimeric clone and its humanized (hIgG1) counterpart measured by flow cytometry using hENTPD3 positive CHO, COS7 and HEK293T cells. 8E1 rabbit anti-human ENTPD3 monoclonal antibody chimerized with a human IgG1 Fc (Human/Rabbit chimeric clones; 8E1 Hu/Ra) and its humanized counterpart (8E1 hIgG1) were serially diluted as indicated (top dose 66 nM) and incubated with cells for 2 hours at 37° C., followed by secondary antibody staining (anti-human IgG (Fc specific), Alexa Fluor® 488) for 30 minutes at 4° C. and flow cytometry analysis. Kd was calculated for each clone and presented in the table. Note: these three cell lines express differential levels of hENTPD3: HEK293T-hENTPD3 (very high) >CHO-hENTPD3 (high) >COS7-hENTPD3 (moderate). HEK293T-hENTPD3 cells contain very high levels of hENTPD3 expression/enzyme activity and are not considered to be physiologically relevant. Expression levels on COS7-hENTPD3 cells are similar to that on RT4 bladder cancer cells (endogenously expressing hENTPD3). Due to the technical difficulty with culturing/working with RT4 cells, two physiologically relevant cell lines COS7-hENTPD3 and CHO-hENTPD3 matched for ENTPD3 expression were chosen for in vitro binding and functional assays, representing intratumoral ENTPD3+ and ENTPD3^{high} cells, respectively.

FIG. 32. 8E1 Hu/Ra clone and its humanized (hIgG1) counterpart show minimal inhibition of hENTPD3 enzyme activity on hENTPD3 positive CHO, COS7 and HEK293T cell membrane. Cells were incubated with 2 μg/mL of 8E1 Hu/Ra or its humanized counterpart (8E1 hIgG1) for 2 hours at 37° C., followed by incubation with ATP (250 μM) for 15 minutes at 37° C. Supernatants were then collected and ATP levels were detected by luminescence using CellTiter-Glo®. Cells with no antibody (Cells+ATP) or ATP alone in the absence of cells were also detected in parallel in order to calculate % of enzymatic activity inhibition as described in Materials and Methods. Note: HEK293T-hENTPD3 cells contain very high hENTPD3 enzyme activity thus degrade exogenous ATP very quickly. This function feature is not considered to be physiologically relevant.

FIG. 33. 8E1 Hu/Ra clone and its humanized (hIgG1) counterpart show minimal inhibition of hENTPD3 enzyme activity on CHO cell membrane even at higher doses. Cells were incubated with serially diluted isotype control or antibodies (top dose 10 μg/mL) for 2 hours at 37° C., followed by incubation with ATP (250 μM) for 15 minutes at room temperature. Supernatants were then collected and ATP levels were detected by luminescence using CellTiter-Glo®. Cells with no antibody (Cells+ATP) or ATP alone in the absence of cells were also detected in parallel and presented in the graphic for comparison. No EC50 could be calculated so it was listed as N/A (not applicable).

FIG. 34. Humanized 8E1 clone (8E1 hIgG1) demonstrates markedly higher ADCC activity against CHO-hENTPD3 cells. CHO-hENTPD3 cells were used as target cells. Jurkat cells stably expressing luciferase and hCD16a-158V were used as effector cells. Target cells were pre-incubated with serially diluted 8E1 Hu/Ra or its humanized counterpart (8E1 hIgG1) as indicated for 30 minutes at 37° C., followed by co-culture with effector cells (T:E=1:6) for 6 hours. ADCC activity was indicated by an increase of luciferase activity over background. RLU: Relative Luminescence Unit. EC50 was calculated as 0.075 μg/mL for 8E1 Hu/Ra and 0.047 for 8E1 hIgG1. ADCC fold increase in relation to 8E1 Hu/Ra was determined as: RLU of humanized clone/RLU of chimeric clone at 1 μg/mL. Note that humanized 8E1 hIgG1 shows an 1.5-fold increase in ADCC activity over the parental 8E1 Hu/Ra chimeric clone.

FIG. 35. Humanized 8E1 clone (8E1 hIgG1) exhibits markedly higher CDC activity toward CHO-hENTPD3 cells. 8E1 human/rabbit chimeric clone (8E1 Hu/Ra) and its respective humanized clone (8E1 hIgG1) were serially diluted as indicated and pre-incubated with CHO-hENTPD3 cells target cells for 30 minutes at 37° C., followed by incubation with 10% Normal Human Serum (NHS) for 2 hours. Target cell lysis was analyzed by flow cytometry and EC50 and % P/I⁺ cells (% of cytotoxicity) were calculated. % of maximal cytotoxicity over background was determined as: % of maximal cytotoxicity at 1 μg/mL-% of background cytotoxicity (at 10⁻³ μg/mL) for each clone. Note that humanized 8E1 hIgG1 shows a 2-fold increase in the maximal CDC cytotoxicity over the parental 8E1 Hu/Ra chimeric clone.

FIG. 36. Affinity of 38D5 Human/Rabbit chimeric clone and its humanized (hIgG1) counterpart measured by flow cytometry using hENTPD3 positive CHO, COS7 and HEK293T cells. 38D5 rabbit anti-human ENTPD3 monoclonal antibody chimerized with a human IgG1 Fc (Human/Rabbit chimeric clones; 38D5 Hu/Ra) and its humanized counterpart (38D5 hIgG1) were serially diluted as indicated (top dose 66 nM) and incubated with cells for 2 hours at 37° C., followed by secondary antibody staining (anti-human IgG (Fc specific), Alexa Fluor® 488) for 30 minutes at 4° C. and flow cytometry analysis. Kd was calculated for each clone and presented.

FIG. 37. 38D5 Hu/Ra clone and its humanized (hIgG1 and hIgG4) counterparts exert differential enzyme activity inhibition potency on cells that express different levels of hENTPD3 on the membrane. CHO, COS7 and HEK293T cells expressing high, moderate and very high hENTPD3 membrane levels, respectively, were incubated with 2 μg/mL of 38D5 Hu/Ra or its humanized counterparts (38D51 hIgG1 or 38D5 hIgG4) for 2 hours at 37° C., followed by incubation with ATP (250 μM) for 15 minutes at 37° C. Supernatants were then collected and ATP levels were detected by luminescence using CellTiter-Glo®. Cells with no antibody (Cells+ATP) or ATP alone in the absence of cells were also detected in parallel in order to calculate % of enzymatic activity inhibition as described in Materials and Methods.

FIG. 38. 38D5 Hu/Ra clone and its humanized (hIgG1) counterpart moderately inhibit hENTPD3 enzyme activity on CHO cell membrane at the same rate regardless of doses. Cells were incubated with serially diluted isotype control or antibodies (top dose 10 μg/mL) for 2 hours at 37° C., followed by incubation with ATP (250 μM) for 15 minutes at room temperature. Supernatants were then collected and ATP levels were detected by luminescence using CellTiter-Glo®. Cells with no antibody (Cells+ATP) or ATP alone in the absence of cells were also detected in parallel and presented in the graphic for comparison. EC50 was calculated as 0.31 μg/mL for 38D5 Hu/Ra and 0.22 μg/mL for 38D5 hIgG1.

FIG. 39. Humanized 38D5 clone (38D5 hIgG1) demonstrates higher ADCC activity against CHO-hENTPD3 cells. CHO-hENTPD3 cells were used as target cells. Jurkat cells stably expressing luciferase and hCD16a-158V were used as effector cells. Target cells were pre-incubated with serially diluted 38D5 Hu/Ra or its humanized counterpart (38D5 hIgG1) as indicated for 30 minutes at 37° C., followed by co-culture with effector cells (T:E=1:6) for 6 hours. ADCC activity was indicated by an increase of luciferase activity over background. RLU: Relative Luminescence Unit. EC50 was calculated as 0.1041 μg/mL for 38D5 Hu/Ra and 0.1036 μg/mL for 38D5 hIgG1. ADCC fold increase in relation to 38D5 Hu/Ra was determined as: RLU of humanized clone/RLU of chimeric clone at 1 μg/mL. Note that humanized 38D5 hIgG1 shows an 1.2-fold increase in ADCC activity over the parental 38D5 Hu/Ra chimeric clone.

FIG. 40. Humanized 38D5 hIgG1 exhibits higher CDC activity toward CHO-hENTPD3 cells. 38D5 human/rabbit chimeric clone (38D5 Hu/Ra) and its respective humanized clone (38D5 hIgG1) were serially diluted as indicated and pre-incubated with CHO-hENTPD3 cells target cells for 30 minutes at 37° C., followed by incubation with 10% Normal Human Serum (NHS) for 2 hours. Target cell lysis was analyzed by flow cytometry and % P/I⁺ cells (% of cytotoxicity) was calculated. EC50 was calculated for each clone and presented. % of maximal cytotoxicity over background was determined as: % of maximal cytotoxicity at 1 μg/mL-% of background cytotoxicity (at 10⁻³ μg/mL) for each clone.

FIG. 41. Affinity of humanized 38D5 hIgG1 backbone point maturation variants measured by flow cytometry using CHO-hENTPD3 cells. Humanized 38D5 (38D5 hIgG1) was subjected to point mutations at the backbone of light (L) or heavy (H) chain resulting in eight new variants. 38D5 hIgG1 clone variants were serially diluted as indicated (top dose 66 nM) and incubated with cells for 2 hours at 37° C., followed by secondary antibody staining (anti-human IgG (Fc specific), Alexa Fluor® 488) for 30 minutes at 4° C. and flow cytometry analysis. The original humanized 38D5 hIgG1 antibody was used as the reference. Kd was calculated for each clone and presented.

FIG. 42. Affinity of humanized 38D5 hIgG1 clone variants measured by flow cytometry using COS7-hENTPD3 cells. 38D5 humanized (38D5 hIgG1) clone variants were serially diluted as indicated (top dose 66 nM) and incubated with cells for 2 hours at 37° C., followed by secondary antibody staining (anti-human IgG (Fc specific), Alexa Fluor® 488) for 30 minutes at 4° C. and flow cytometry analysis. The original humanized 38D5 hIgG1 antibody was used as the reference. Kd was calculated for each clone and presented.

FIG. 43. Humanized 38D5 hIgG1 clone variants show moderate hENTPD3 enzyme activity inhibition potency on CHO-hENTPD3 cell membrane. Cells were incubated with 2 μg/mL of human IgG1 isotype Ultra-LEAF or 38D5 hIgG1 variants or the original humanized 38D5 hIgG1 antibody for 2 hours at 37° C., followed by incubation with ATP (250 μM) for 15 minutes at 37° C. Supernatants were then collected and ATP levels were detected by luminescence using CellTiter-Glo®. Cells with no antibody (Cells+ATP) or ATP alone in the absence of cells were also detected in parallel in order to calculate % of enzymatic activity inhibition as described in Materials and Methods.

FIG. 44. Humanized 38D5 hIgG1 clone variants partially inhibit hENTPD3 enzyme activity on COS7-hENTPD3 cell membrane. Cells were incubated with 2 μg/mL of human IgG1 isotype Ultra-LEAF or 38D5 hIgG1 variants or the original humanized 38D5 hIgG1 antibody for 2 hours at 37° C., followed by incubation with ATP (250 μM) for 15 minutes at 37° C. Supernatants were then collected and ATP levels were detected by luminescence using CellTiter-Glo®. Cells with no antibody (Cells+ATP) or ATP alone in the absence of cells were also detected in parallel in order to calculate % of enzymatic activity inhibition as described in Materials and Methods.

FIG. 45. Anti-tumor efficacy of the Fc-competent humanized 38D5 hIgG1 in vivo. Six- to 8-week-old C57BL6 hENTPD3 KI female mice were inoculated subcutaneously with MC38 colorectal cancer cells (5×10⁵) in 150 μl of RPMI 1640 media. Mice were then randomized into two groups (n=5 per group). On day 4, tumor-bearing mice received 20 mg/kg of humanized 38D5 hIgG1 antibody, or 200 μl of saline, via intraperitoneal injection. On days 7, 10 and 14, tumor-bearing mice received 10 mg/kg of 38D5 hIgG1 antibody, or 200 μl of saline. Tumor length (L) and width (W) were measured using a digital caliper twice weekly. Tumor volume (mm³) was determined as L*W*W*0.52.

FIG. 46. Analyzing Fc-silent humanized 38D5 hIgG4 effects in an in vivo tumor-killing assay. Six- to 8-week-old C57BL6 hENTPD3 KI female mice were inoculated subcutaneously with MC38 colorectal cancer cells (5×10⁵) in 150 μl of RPMI 1640 media. Mice were then randomized into two groups (n=5 per group). On day 4, tumor-bearing mice received 20 mg/kg of humanized 38D5 hIgG4 antibody, or 200 μl of saline, via intraperitoneal injection. On days 7, 10 and 14, tumor-bearing mice received 10 mg/kg of 38D5 hIgG4 antibody, or 200 μl of saline. Tumor length (L) and width (W) were measured using a digital caliper twice weekly. Tumor volume (mm³) was determined as L*W*W*0.52.

FIG. 47. The Fc-competent humanized 38D5 hIgG1 is stable in vivo after single dose exposure. 9-week-old C57BL6 hENTPD3 KI tumor-free female mice received one dose of 1 or 10 mg/kg of 38D5 hIgG1 via intraperitoneal injection. Plasma samples were then collected from each mouse 24 and 48 h later, and free plasma antibody level was detected by binding analysis using COS7-hENTPD3 cells by flow cytometry. 38D5 hIgG1 (2 μg/mL) was used as the positive control to indicate maximal cell binding. A total of two mice were used in this experiment, one for each dose. Sample name was designated as: dose injected+collection time.

FIG. 48. The Fc-competent humanized 38D5 hIgG1 remains stable in vivo after repeated dose exposure. 9-week-old C57BL6 hENTPD3 KI female mice were inoculated subcutaneously with MC38 colorectal cancer cells (1×10⁵) in 150 μl of RPMI 1640 media. Tumor-bearing mice received 3 mg/kg of 38D5 hIgG1, on days 12, 15, 18, and 21 via intraperitoneal injection. Plasma samples were collected on days 19, 21 and 23, and free plasma antibody was detected by binding analysis using COS7-hENTPD3 cells by flow cytometry. 38D5 hIgG1 (2 μg/mL) was used as the positive control to indicate maximal cell binding. Sample name is designated as: animal number+collection date.

FIG. 49. Different binding kinetics profiles of 8E1, 38D5 and PBI #30 hIgG1 affect cell affinity. Antibodies were serially diluted as indicated (top dose 33 nM) and incubated with CHO-hENTPD3 cells for 20 min at 4° C. or 2 hours at 37° C., followed by secondary antibody staining (anti-human IgG (Fc specific), Alexa Fluor® 488) for 30 minutes at 4° C. and subsequent flow cytometry analysis. Kd was calculated for each clone and presented. Note the significant increase in Kd for 38D5 and PBI #30 clones, but not for clone 8E1 after elongation of the incubation time.

FIG. 50. Stability of antibody:antigen immune complex of the exemplary Fc-competent anti-ENTPD3 monoclonal antibodies on COS7-hENTPD3 cells. The exemplary anti-human ENTDP3 antibodies in this invention as indicated (6 μg/ml) were incubated with attached COS7-hENTPD3 cells for 24 hours or 2 hours at 37° C. in 5% CO₂, followed by secondary antibody staining (anti-human IgG (Fc specific), Alexa Fluor 488) for 30 minutes at 4° C. Cells were then washed, trypsinized, and analyzed by flow cytometry. The difference in AF488 MFI between 2- and 24-hour treatment represents the loss of human ENTPD3 on cell membrane that was calculated as described in Materials and Methods. 8E1 and 38D5 Hu/Ra: human/rabbit chimeric antibodies; 8E1 and 38D5 hIgG1: humanized rabbit antibodies; PBI #30 hIgG1 and PBI #30af4 hIgG1: fully human antibody and its affinity maturation variant.

FIG. 51. Stability of antibody:antigen immune complex of other subject ADCC-high monoclonal anti-ENTPD3 antibodies on CHO-hENTPD3 cells. Human/Rabbit chimeric antibodies (2 μg/ml) were incubated with CHO-hENTPD3 cells for 24 hours at 37° C. in 5% CO₂ or 20 min at 4° C., followed by secondary antibody staining (anti-human IgG (Fc specific), Alexa Fluor 488) for 30 minutes at 4° C. Cells were then washed and analyzed by flow cytometry. The difference in AF488 MFI between 20 minutes and 24 hours treatment represents the loss of human ENTPD3 on cell membrane that was calculated as described in Materials and Methods.

### DETAILED DESCRIPTION OF THE INVENTION

### I. Overview

The tumor microenvironment is considered as a highly significant therapeutic target as it contributes to the proliferation and survival of malignant cells, angiogenesis, metastasis, abnormal immunity, and decreased responses to hormones and chemotherapeutic agents.

In many studies, it has been demonstrated that the tumor-associated macrophage (TAM) is a major factor in the tumor microenvironment and an important regulator of angiogenesis, which is essential for tumor progression by supplying oxygen and nutrients to hypoxic tumor sites. Therefore, when a large number of tumor-associated macrophages exist around tumors in cancer patients, it has been reported that the prognosis and survival rate of patients are poor. The role of tumor-associated macrophages in the tumor microenvironment is still highly controversial.

The tumor-associated macrophages are classified into two phenotypes of tumor suppressor M1 or tumor support M2 macrophages. The M1-type tumor-associated macrophage has a strong ability to present an antigen and generally presents CD86 and TNF-α. In contrast, the M2-type tumor-associated macrophage has a low antigen-presentation ability and high potential for phagocytosis.

The M2-type macrophages are known to promote immunosuppression, tumorigenesis, and vasculogenesis by releasing various extracellular matrix components, angiogenesis and chemotaxis factors. The M2-type tumor-associated macrophages are distinguished from the M1-type tumor-associated macrophages by expressing some markers such as CD163, CD204, CD206, and IL-10. In most tumors, such as breast, ovarian, prostate, lung cancer and skin melanoma, the tumor microenvironment includes IL-10 capable of inducing the introduction of CSF-1, VEGF, CCL2, IL-4, IL-13, TGF-β and monocytes and inducing differentiation with a similar phenotype to M2.

Previous studies have shown that depletion of macrophages by encapsulated clodronates may limit angiogenesis in tumor tissues. In addition, since the infiltration of the macrophages is prevented through CSF-1R and CCR2 antibodies, it is possible to limit and decrease tumor-initiating properties and increase the activity of cytotoxic T lymphocytes. Therefore, when high levels of M2-type tumor-associated macrophages are present in the tumor microenvironment, the growth, differentiation, and metastasis of the tumor are all activated. Hence, targeting innovative elements of M2-type tumor-associated macrophages may provide potential therapies to prevent tumor growth and metastasis.

The present invention is based at least in part on the discovery that certain defined antibodies to NTPDase3 are capable of selectively targeting and ablating, such as by ADCC, such NTPDase3 expressing cells in the tumor microenvironment. This occurs more efficiently by targeting NTPDase3+ macrophage (especially M2/M2-like macrophage) and/or other NTPDase3+ cellular makeup of the tumor, such as NTPDase3+ intratumoral cells like cancer cells, as well as pericytes and/or fibroblastic cells around tumor vessels. The resulting decrease in numbers of intratumoral NTPDase3+ cells can lead to such changes in the inflammatory phenotype of the tumor as enhanced T-cell infiltration occurs into the tumor. These is also potential for decreased levels of T-cell exhaustion in the tumor, reduced type II NKT cells suppression of intratumoral immune cell function and/or limitation of regulatory T cells (Treg) suppression of intratumoral immune cell function, and/or disruption of tumor-associated vasculature limiting nutrient supplies to the tumor (e.g., tumor starvation).

Another aspect of the present invention is based at least in part on the discovery that certain antibodies to NTPDase3 are capable of inhibiting the NTPDase activity of that enzyme and thereby reducing the intratumoral concentration of adenosine at specific sites. Extracellular adenosine has been known as an inhibitor of immune functions. While intracellular adenosine is involved in energy metabolism, nucleic acid metabolism, and the methionine cycle, in the tumor microenvironment extracellular adenosine plays an important role in suppressing immune signaling. Immunosuppressive adenosine 3′5′-monophosphate (cAMP)-mediated pathway, signaling through adenosine A2A receptor (A2AR), can inhibit T lymphocytes and natural killer (NK) cells in hypoxic, inflamed, and cancerous microenvironment (Ohta et al. (2006) Proc Natl Acad Sci USA, 103:13132-7).

Preclinical proof, along with recent and evolving positive clinical trial data, demonstrates that the administration of A2AR inhibitors can be a potential novel strategy for immunotherapy. In addition, blocking the adenosine-generating pathway involving CD39/CD73 also induces regression of breast cancer, colorectal cancer and melanoma in experimental animal models. In the case of anti-CD39 and anti-CD73 antibody therapies, the focus is predominantly on the inhibition or reduction of ATP and derivative nucleotide catabolism, ultimately to adenosine through binding to these cell surface adenosine generating enzymes (ectonucleotidases) and inhibiting the enzymatic activity or removing the enzymatic activity from the cell surface.

As described in greater detail in the Exemplary Methods and illustrated in the Figures, anti-ENTPD3 antibodies described herein, including those that are fully human antibodies or humanized antibodies that were specifically designed to have human constant regions with an IgG1 domain, were generated and assayed for a variety of functions. The IgG1 domain-based design confers FcγRIIIa receptor-dependent cellular activities, e.g., antibody-dependent cellular cytotoxicity (ADCC) and complement-dependent cytotoxicity (CDC) against ENTPD3+ cells, and/or antibody-mediated target cytosis of intratumoral ENTPD3+ cells, to the anti-ENTPD3 antibodies of the instant invention. Consequently, such cellular activities result in ablation and reduction of ENTPD3^{high} cells in the tumor. In addition, some of our anti-ENTPD3 antibodies are also capable to inhibit ENTPD3 enzymatic activity on ENTPD3+ cells, alone or accompanied with ADCC/CDC/target cytosis activity, which therefore lead to global decreases in ENTPD3 enzymatic activity inside the tumors.

Exemplary features of the subject anti-ENTPD3 monoclonal antibodies, features for use in therapeutic anti-ENTPD3 monoclonal antibodies which are taught away from the literature and have not been described in the prior art up to date, are summarized, listed in Table 1, and discussed further below.

TABLE 1
Exemplary Clones

| | | | | Rabbit 8E1 | | Rabbit 38D5 | | | Fully Human PBI#30 | | |
|---|---|---|---|---|---|---|---|---|---|---|---|
| | | | | | | | 38D5 | 38D5 | | PBI#30af4 | PBI#30af4 |
| | | | | 8E1 | | 38D5 | hIgG1 | hIgG4 | PBI#30 | hIgG1 | hIgG4 |
| | | | | Hu/Ra | | Hu/Ra | Human- | Human- | hIgG1 | Affinity | Affinity |
| | | | | (hIgG1) | | (hIgG1) | ized | ized | Parental | maturated | maturated |
| Clone name | | | | Parental | 8E1 hIgG1 | Parental | (Fc- | (Fc- | (Fc- | (Fc- | (Fc- |
| Note | | | | Chimeric | Humanized | Chimeric | Competent) | Silent) | Competent) | Competent) | Silent) |
| Func- | In | ADCC | EC50 | 0.075 | 0.047 | 0.1041 | 0.1036 | N/A | 1.443 | 0.434 | N/A |
| tional | Vitro | | (μg/mL) | | | | | | | | |
| Activ- | | | Fold increase | N/A | 1.5 | N/A | 1.2 | N/A | N/A | 2.8 | N/A |
| ities | | | in relation to | | | | | | | | |
| | | | parental clone | | | | | | | | |
| | | | at 1 μg/mL | | | | | | | | |
| | | | Category | Very High | Very High | High | High | No | High | High | No |
| | | CDC | EC50 | 0.239 | 0.867 | 1.732 | 0.694 | N/A | N/A | N/A | N/A |
| | | | (μg/mL) | | | | | | | | |
| | | | % of | 13 | 25 | 1 | 4 | N/A | N/A | N/A | N/A |
| | | | Maximal | | | | | | | | |
| | | | Cytotoxicity | | | | | | | | |
| | | | Category | High | High | Moderate | Moderate | No | No | No | No |
| | | Enzyme | EC50 | N/A | N/A | 0.31 | 0.22 | N/A | 2.51 | 0.82 | 1.33 |
| | | Activity | (μg/mL) | | | | | | | | |
| | | Inhibition | % of | 5 | 15 | 44 | 43 | 41 | 76 | 91 | 95 |
| | | Capacity | Maximal | | | | | | | | |
| | | | Inhibition | | | | | | | | |
| | | | Category | Minimal | Minimal | Moderate | Moderate | Moderate | High | Very High | Very High |

| | | | | | | | | | | |
|---|---|---|---|---|---|---|---|---|---|---|
| | In | Anti-tumor Activity | Yes | N/A* | Yes | Yes | No | No** | No*** | Yes |
| | Vivo | against MC38 tumor in | | | | | | | | |
| | | hENTDP3 KI mice | | | | | | | | |
| | | Plasma Clearance | N/A | N/A | N/A | Stable | N/A | N/A | Quick*** | Stable |
| Bio- | In | Binding Kinetics | Fast | Fast | Slow | Slow | Slow | Slow | Slow | Slow |
| physical | Vitro | (Association Rate) | | | | | | | | |

| Charac- | Epitope Competition | Not with 38D5 | Not with 8E1 | Partially with 8E1 |
|---|---|---|---|---|
| teristics | | Partially with PBI#30 | Partially with PB##30 | and 38D5 |

Other Subject ADCC-high Clones (Hu/Ra; hIgG1; Fc-Competent)

| | | | | 3E9 | 4F9 | 16D4 | 37H1 | 38D12 | 42 D8 | 44H5 |
|---|---|---|---|---|---|---|---|---|---|---|
| Clone Name | | | | Parental | Parental | Parental | Parental | Parental | Parental | Parental |
| Note | | | | Chimeric | Chimeric | Chimeric | Chimeric | Chimeric | Chimeric | Chimeric |
| Func- | In | ADCC | EC50 | 0.044 | 0.064 | 0.053 | 0.044 | 0.059 | 0.072 | 0.045 |
| tional | Vitro | | (μg/mL) | | | | | | | |
| Activ- | | | Fold increase | N/A | N/A | N/A | N/A | N/A | N/A | N/A |
| ities | | | in relation to | | | | | | | |
| | | | parental clone | | | | | | | |
| | | | at 1 μg/mL | | | | | | | |
| | | | Category | High | High | High | High | High | High | High |
| | | CDC | EC50 | N/A | N/A | N/A | N/A | N/A | N/A | N/A |
| | | | (μg/mL) | | | | | | | |
| | | | % of | N/A | N/A | N/A | N/A | N/A | N/A | N/A |
| | | | Maximal | | | | | | | |
| | | | Cytotoxicity | | | | | | | |
| | | | Category | N/A | N/A | N/A | N/A | N/A | N/A | N/A |
| | | Enzyme | EC50 | N/A | N/A | N/A | N/A | N/A | N/A | N/A |
| | | Activity | (μg/mL) | | | | | | | |
| | | Inhibition | % of | 6 | 12 | 9 | 9 | 9 | 12 | 15 |
| | | Capacity | Maximal | | | | | | | |
| | | | Inhibition | | | | | | | |
| | | | Category | Minimal | Minimal | Minimal | Minimal | Minimal | Minimal | Minimal |

| | | | | | | | | | |
|---|---|---|---|---|---|---|---|---|---|
| | In | Anti-tumor Activity | N/A | N/A | N/A | N/A | N/A | N/A | N/A |
| | Vivo | against MC38 tumor in | | | | | | | |
| | | hENTDP3 KI mice | | | | | | | |
| | | Plasma Clearance | N/A | N/A | N/A | N/A | N/A | N/A | N/A |
| Bio- | In | Binding Kinetics | Fast | Fast | Fast | Fast | Slow | Slow | Moderate |
| physical | Vitro | (Association Rate) | | | | | | | |

| | | | | | | |
|---|---|---|---|---|---|---|
| Charac- | Epitope Competition | Not with | Not with 38D5 and 44H5; | Not with | Not with | Fully with 3E9 |
| teristics | | 38D5, | Partially with 38D12 and PBI#30; | 38D5; | 44H5; | and 38D5; |
| | | 38D12 and | Fully with 3E9 | Fully with | Fully | Not with the |
| | | PBI#30; | | PBI#30 | with 3E9 | rest clones |
| | | Fully with | | | | |
| | | 44H5 | | | | |

“N/A” indicates not applicable.
*Not tested in vivo, but it is believed and expected that this clone has favorable in vivo tumor-killing activity.
**Under particular conditions assayed, the parental PBI#30 hIgG1 antibody did not present in vivo anti-tumor activity due to its unsatisfying binding kinetics, so affinity maturation variants were generated to optimize binding affinity and functionality.
***Affinity maturation variant PBI#30af4 lgG1 is cleared out quickly in the mouse blood limiting its anti-tumor activity in vivo. It is believed and expected that it would have in vivo tumor-killing activity if engineered to be more stable in vivo.

Physiologically Relevant Cell Lines Matched for ENTPD3 Expression/Enzyme Activity were Used for Binding and Functional Assays in this Invention.
Three stably transfected cell lines, including CHO-hENTPD3, COS7-hENTPD3 and HEK293T-hENTPD3 cells, were established for use in binding and functional assays as described further herein. These three cell lines contain differential levels of expression/enzyme activities of the target antigen hENTPD3: HEK293T-hENTPD3 (very high) >CHO-hENTPD3 (high) >COS7-hENTPD3 (moderate). HEK293T-hENTPD3 cell line expresses very high levels of hENTPD3 enzyme activity thus degrading exogenous ATP very quickly, which is not considered to be physiologically relevant. hENTPD3 expression/enzyme activity levels on COS7-hENTPD3 cells are similar to those on RT4 human bladder cancer cells (endogenously expressing hENTPD3). CHO-hENTPD3 cells are representative of the ADCC target ENTPD3^{high} cells in the tumor microenvironment. Due to the technical difficulty with culturing/working with RT4 cells, COS7-hENTPD3 and CHO-hENTPD3 cells, two physiologically relevant transfectant cell lines that are matched for ENTPD3 expression/enzyme activity, were therefore chosen for in vitro binding and functional assays to represent intratumoral ENTPD3+ and ENTPD3^{high} cells, respectively.

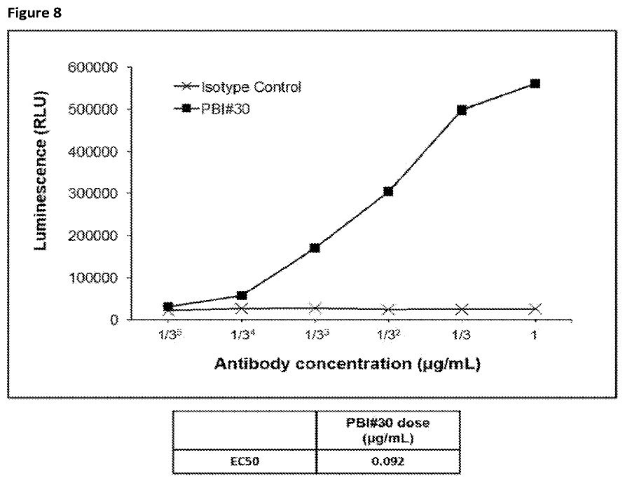

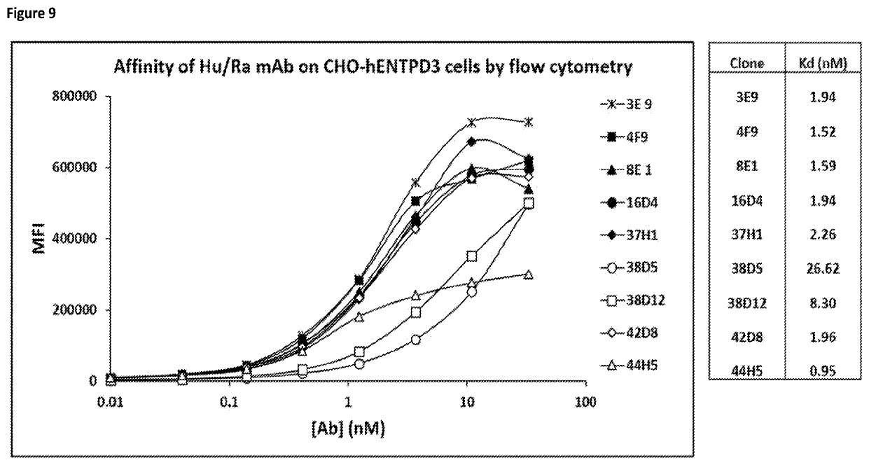

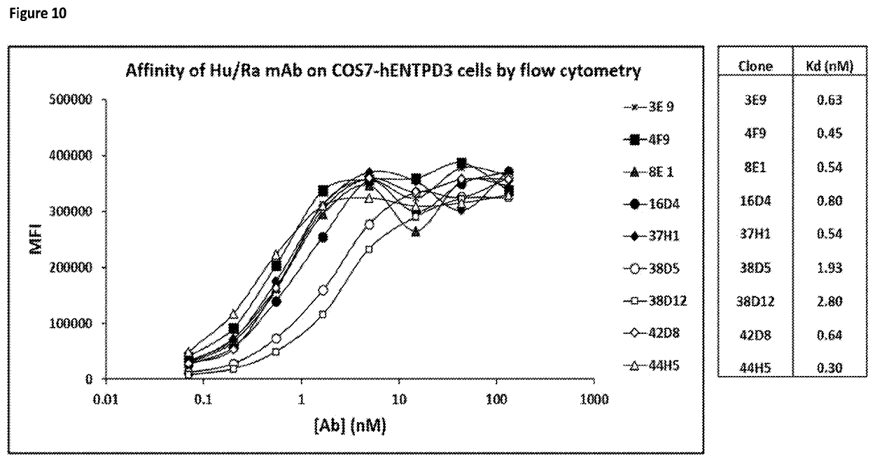

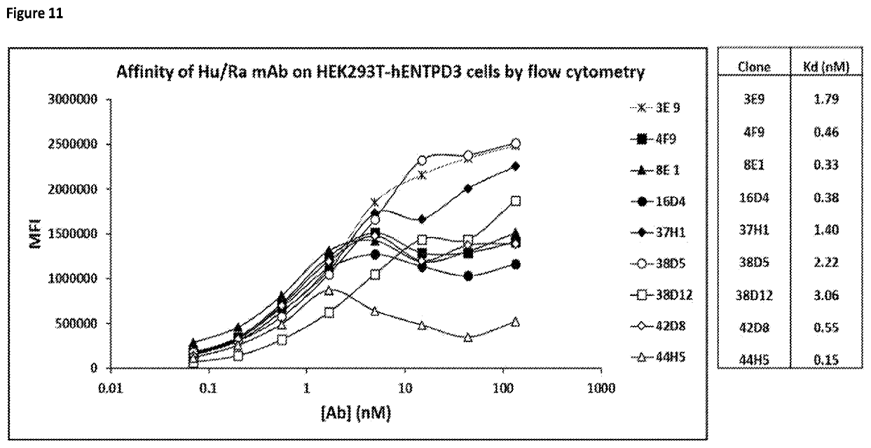

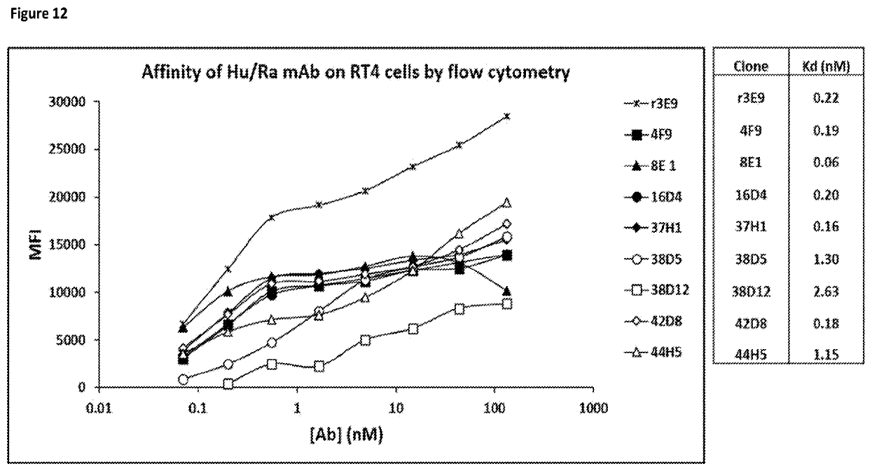

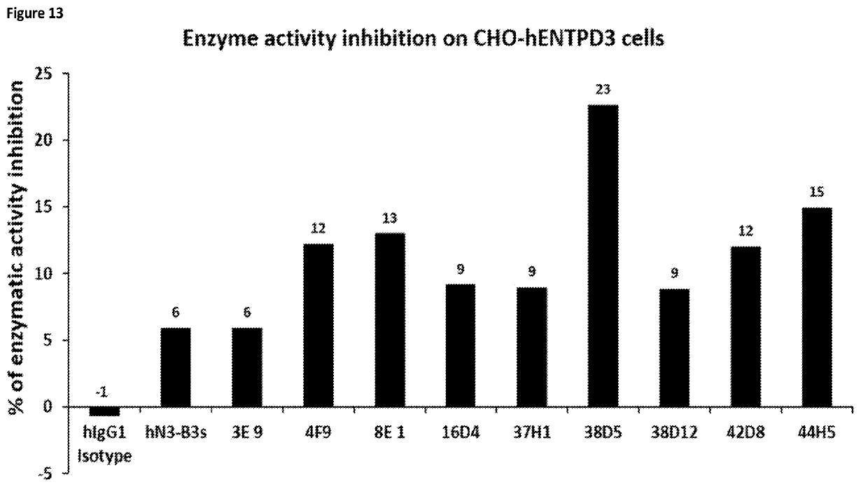

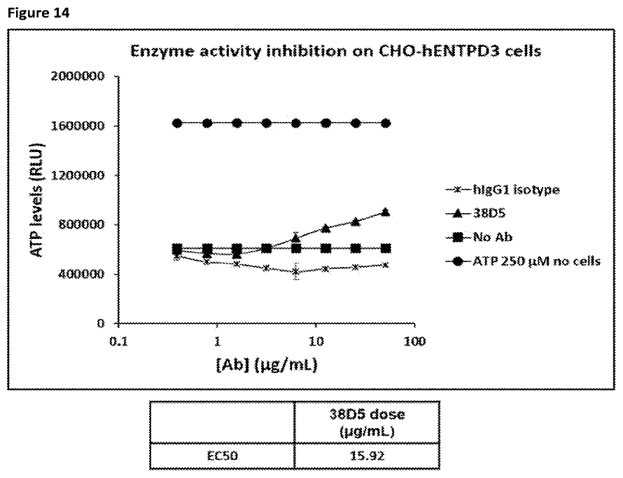

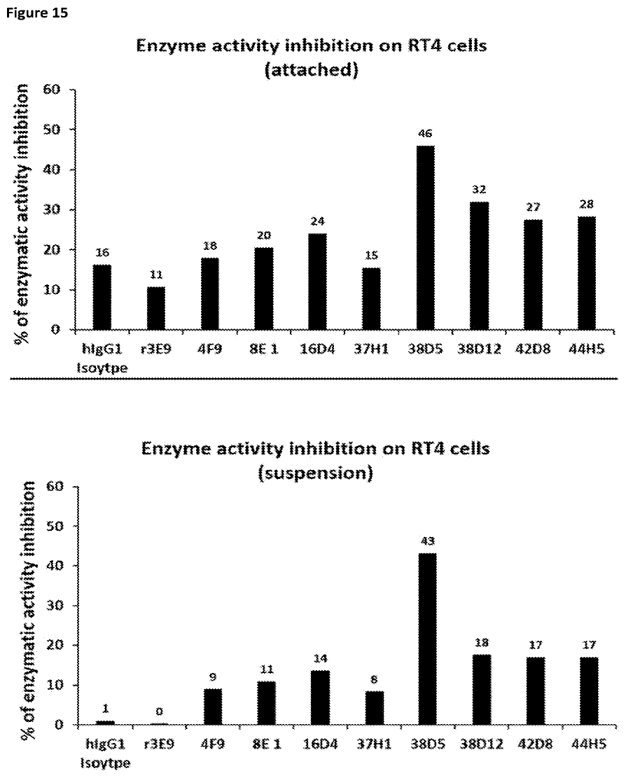

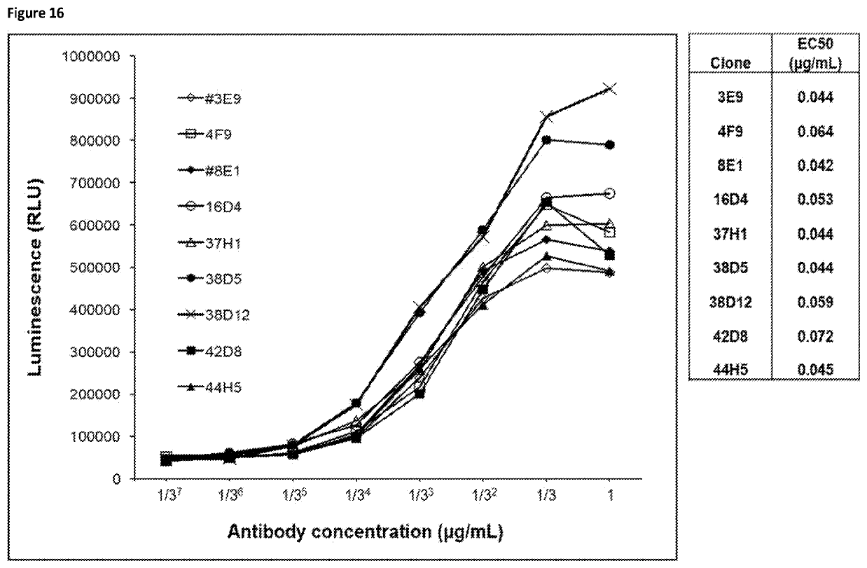

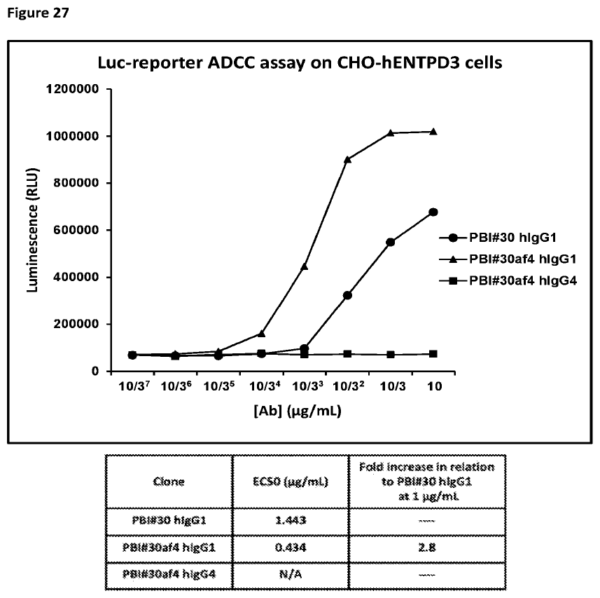

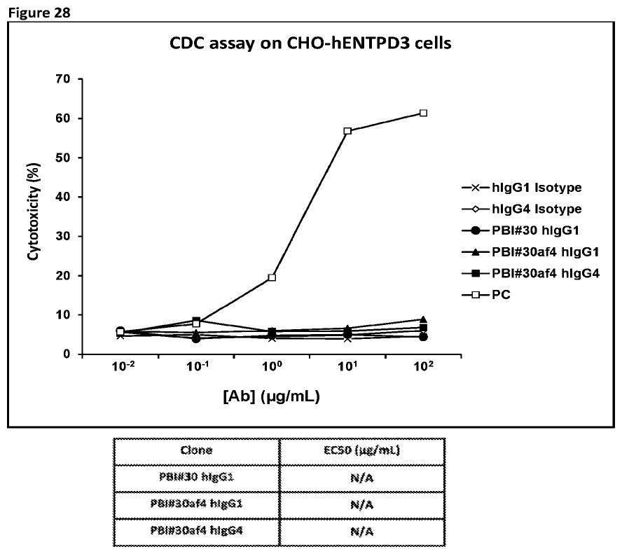

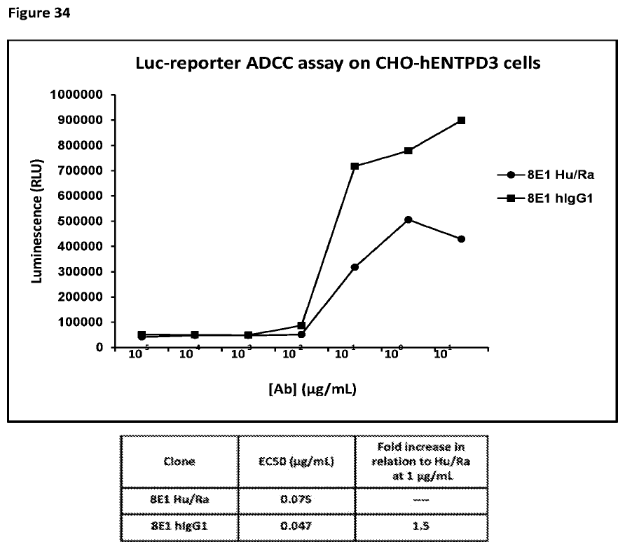

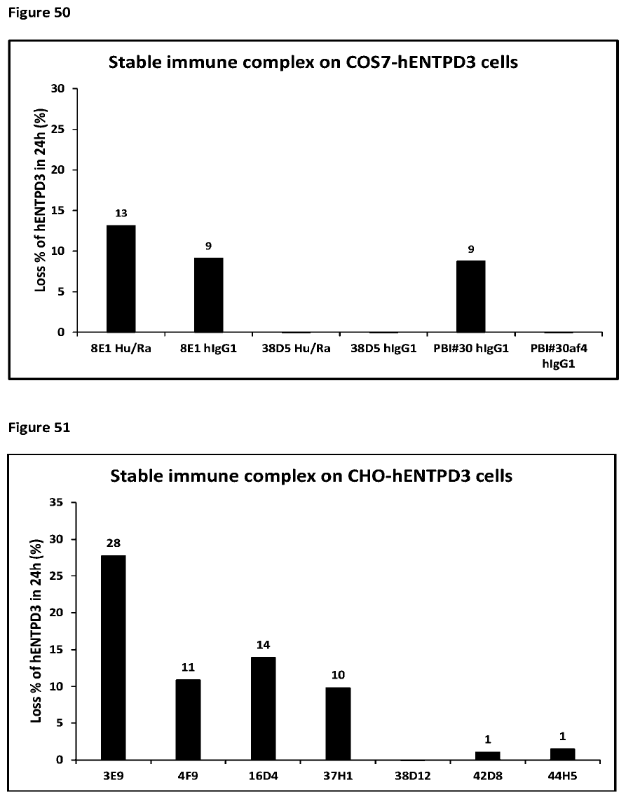

Formation of a Stable Immune Complex of Anti-ENTPD3 Antibodies with the Antigen on Target Cell Membrane Confers High ADCC Activity to the Antibodies.
As shown in FIGS. 50 and 51, the stability of the antibody:antigen immune complex on target cell surface was examined for all parental subject anti-ENTPD3 antibodies described herein, which were initially selected based on their high ADCC activity (FIGS. 8 and 16). All tested clones form a stable immune complex with the antigen ENTPD3 on target cell membrane (i.e., the highest loss of human ENTPD3 on cell membrane is no greater than 30%) (FIGS. 50 and 51). Moreover, the engineered clone variants with improved ADCC functionality (e.g., FIGS. 27 and 34 for clone 8E1 and PBI #30, respectively) demonstrate favorably increased immune complex stability over the respective parental clone (FIG. 50; 8E1 Hu/Ra versus humanized 8E1 hIgG1 and PBI #30 hIgG1 versus maturation variant PBI #30af4 hIgG1). Our data evidently show the positive correlation of an antibody's ADCC activity with its immune complex stability on target cell membrane.

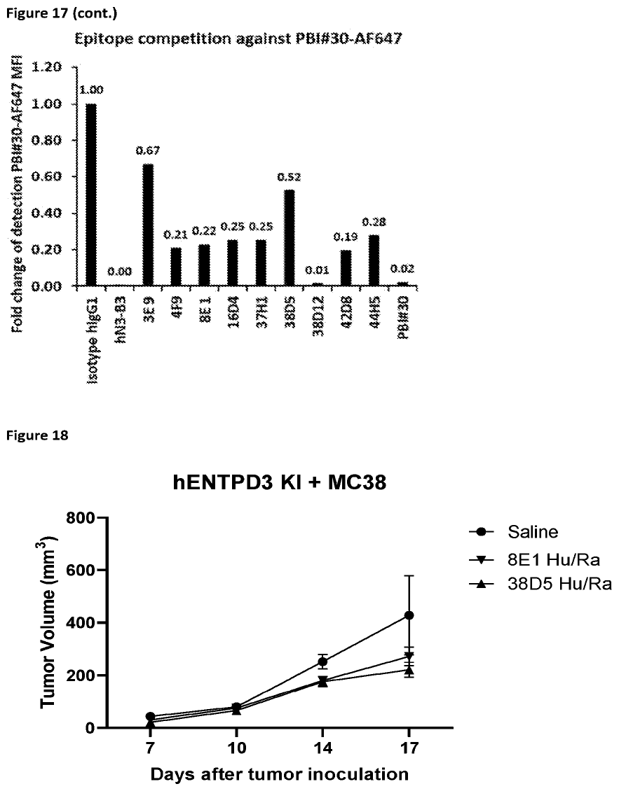

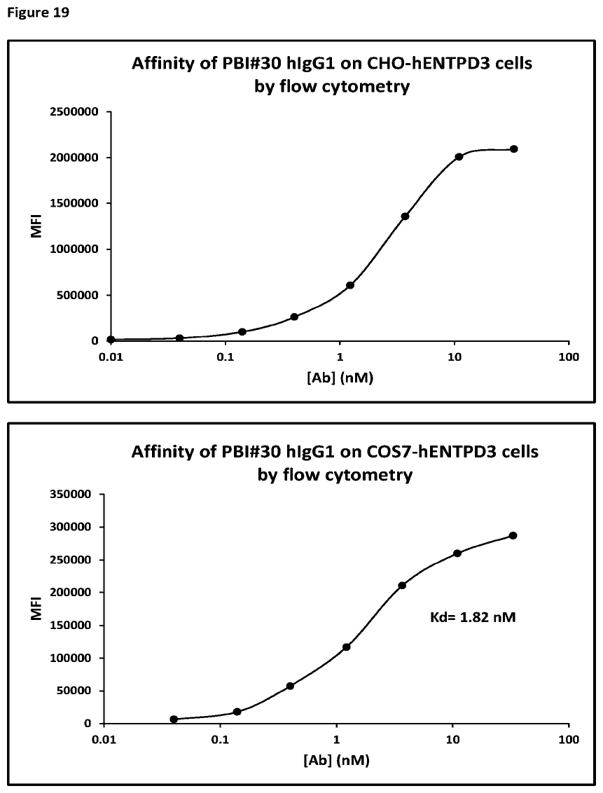

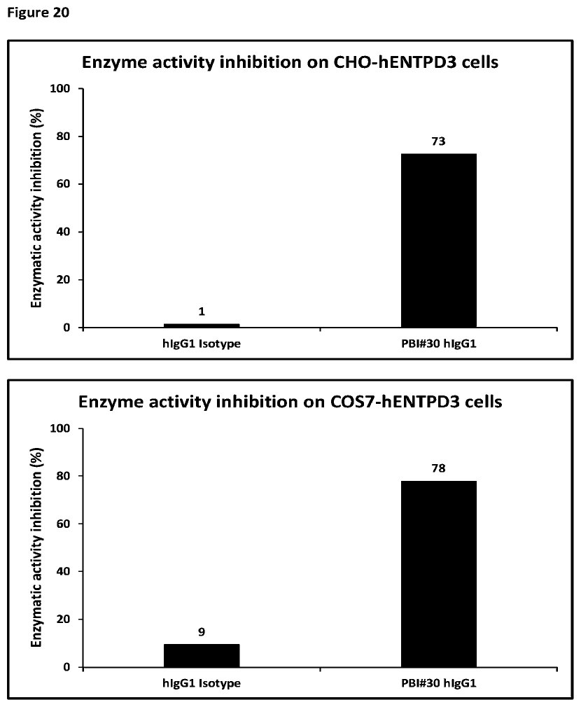

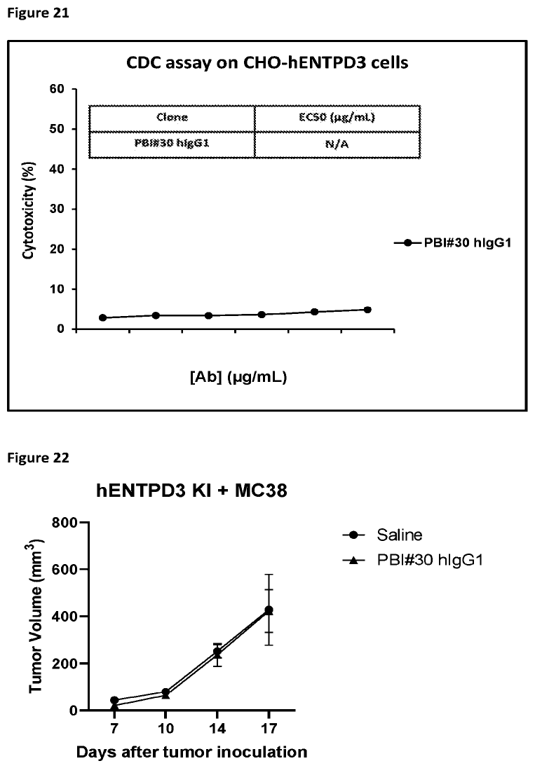

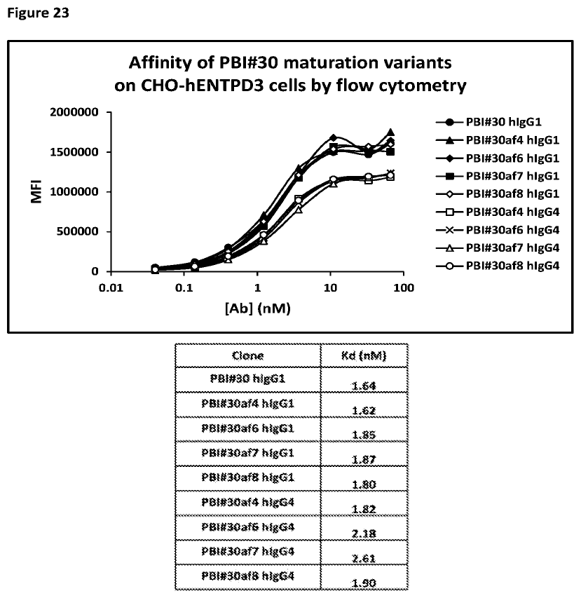

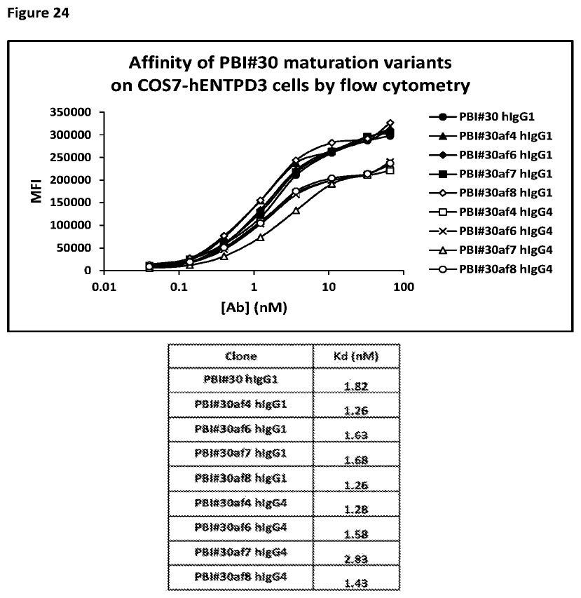

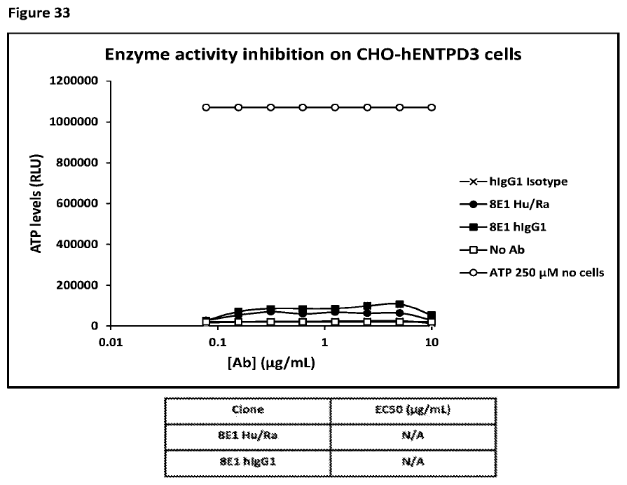

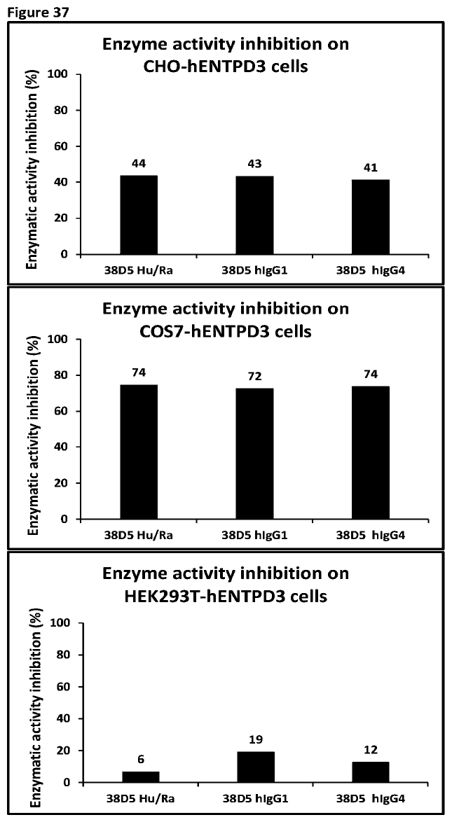

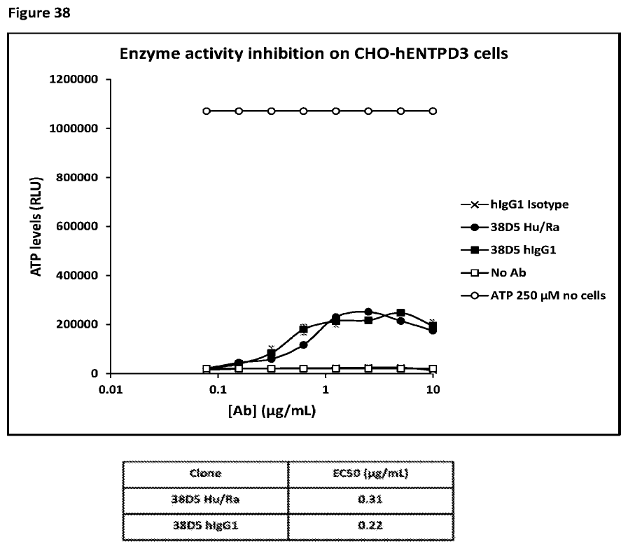

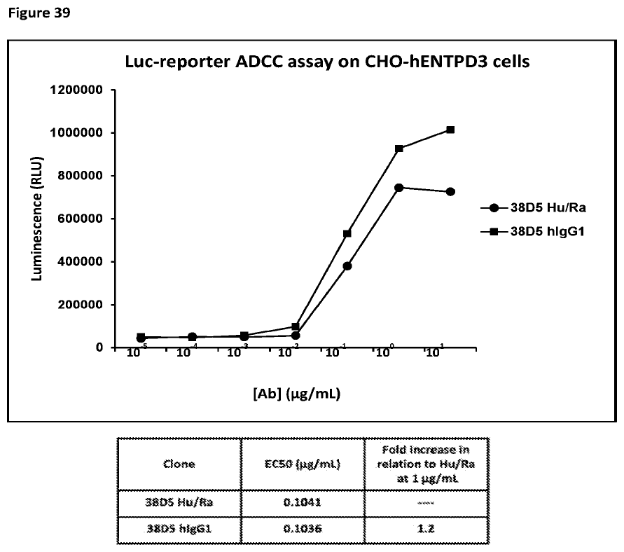

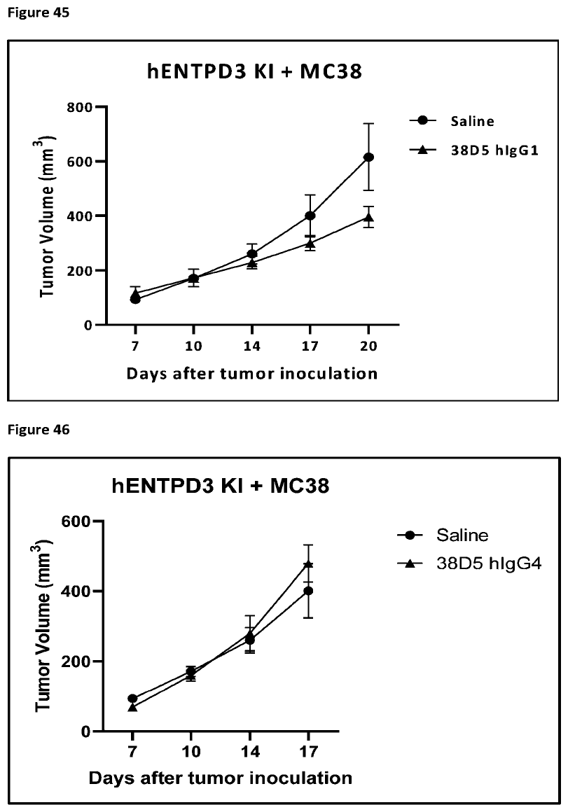

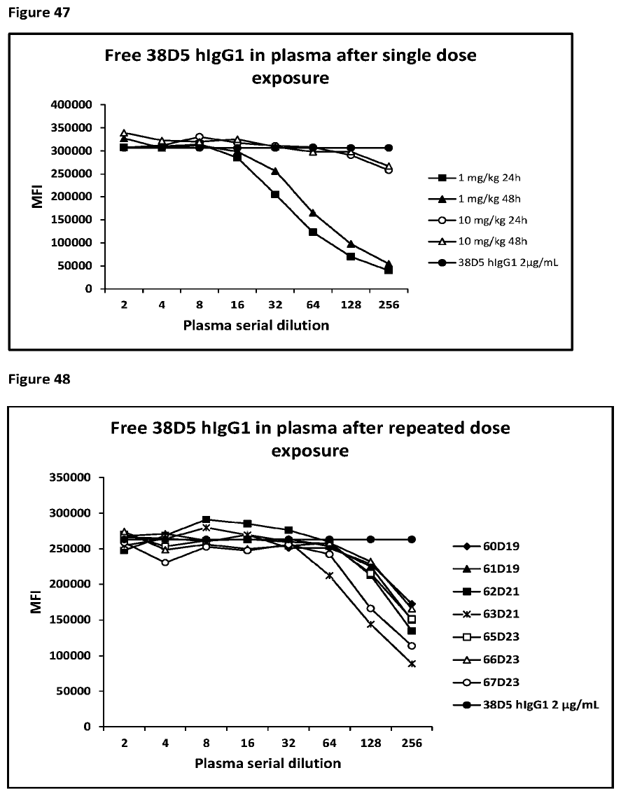

The Subject Monoclonal Antibodies Target ENTPD3+ Cells in the Tumor Through FcγRIIIa Receptor-Dependent Cellular Activities (e.g., ADCC).
As a working example, the original 8E1 Human/Rabbit chimeric clone (8E1 Hu/Ra, an hIgG1 isotype) shows minimal inhibition of ENTPD3 enzyme activity (FIG. 33) but has very high ADCC activity against ENTPD3^{high} cells in vitro (FIG. 34; EC50=0.075 μg/ml) which is sufficient to block tumor growth in vivo (FIG. 18). A representative humanized version of this clone (8E1 hIgG1) designed to reduce any possible immunogenicity for potential therapeutic use has ADCC activity showing a 1.5-fold increase over the parental chimeric clone 8E1 Hu/Ra (FIG. 34; EC50=0.047 μg/ml). Without being bound by theory, it is thus believed that humanized 8E1 hIgG1 has favorable tumor-killing activity in vivo. Indeed, for therapeutic use of antibodies with ADCC killing activity, IgG Fc engineering of antibodies e.g. defucosylation of the Fc domain is preferred as this approach can enhance the antibody's ADCC effector functions to approximately 100 fold (Shields et al. (2002) JBC. 277 (30):26733-26740; Yamane-Ohnuki et al. (2004) Biotechnol Bioeng. September 5, 87(5):614-622; Mori et al. (2007) Cytotechnology 55:109-114; and Lonza's Potelligent® CHOK1SC Cell Line for manufacture of afucosylated therapeutic antibodies). Therefore significant augmentation of both in vitro and in vivo ADCC tumor-killing activity of an afucosylated 8E1 hIgG1 is expected.
Another working example is clone 38D5: the original Human/Rabbit chimeric clone (38D5 Hu/Ra, an hIgG1 isotype) contains high ADCC activity (FIG. 39; EC50=0.1041 μg/ml) and moderate enzyme activity inhibition capacity (FIGS. 37 and 38; EC50=0.31 μg/ml with approximately 40% of maximal inhibition) toward ENTPD3^{high} cells in vitro, associated with anti-tumor activity in vivo (FIG. 18). Humanization of this clone (38D5 hIgG1) improves its in vitro ADCC activity (FIG. 39; EC50=0.1036 μg/ml with an 1.2-fold increase over the parental chimeric clone 38D5 Hu/Ra), while retaining its enzyme activity inhibition capacity (FIG. 38). Humanized 38D5 hIgG1 displays favorable in vivo tumor-killing activity (FIG. 45) and in vivo plasma stability (FIGS. 47 and 48). Interestingly, a switch from the Fc-competent 38D5 IgG1 to the Fc-silent IgG4 version, 38D5 hIgG4 antibody fails to block tumor growth in vivo (FIG. 46), yet its in vitro enzyme activity inhibition capability is retained (FIG. 37).
These data again validate that high ADCC activity alone (at an approximate EC50 of 0.1 μg/ml) is sufficient to confer the antibody with tumor-killing activity both in vitro and in vivo, regardless of its ENTPD3 enzyme activity inhibition capacity; and an antibody containing moderate levels of enzyme activity inhibition capability alone is not sufficient to have in vivo anti-tumor activity.

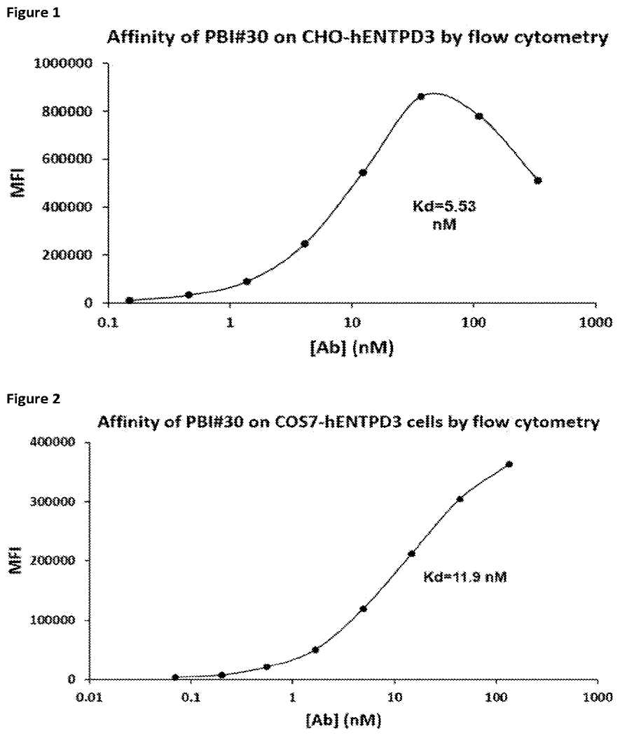

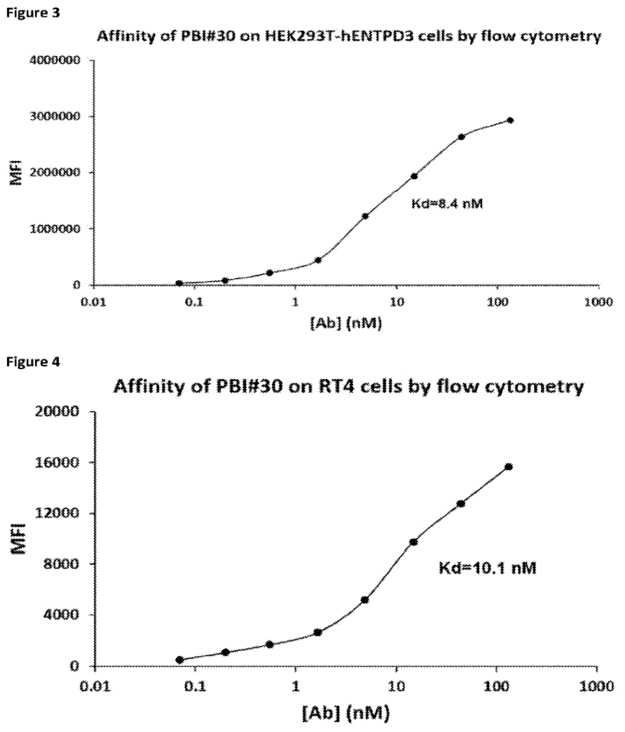

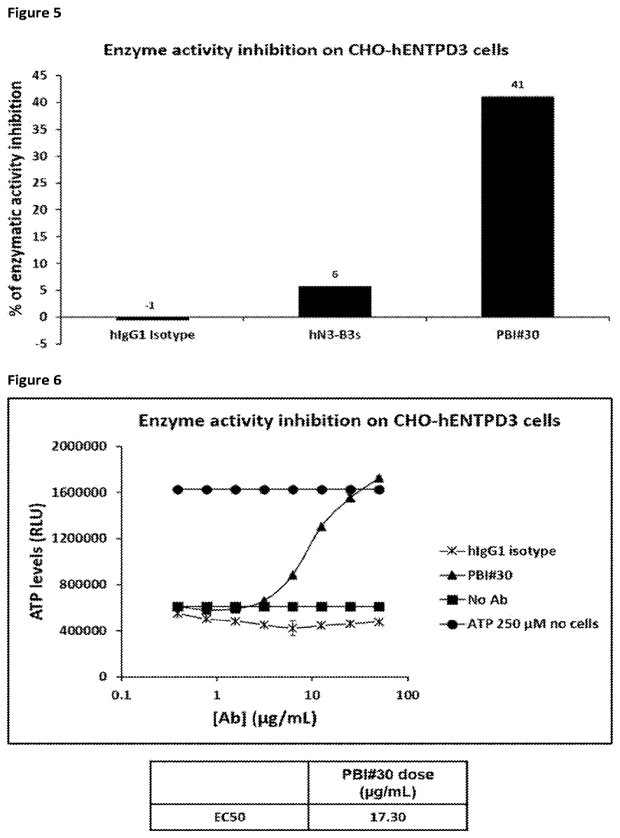

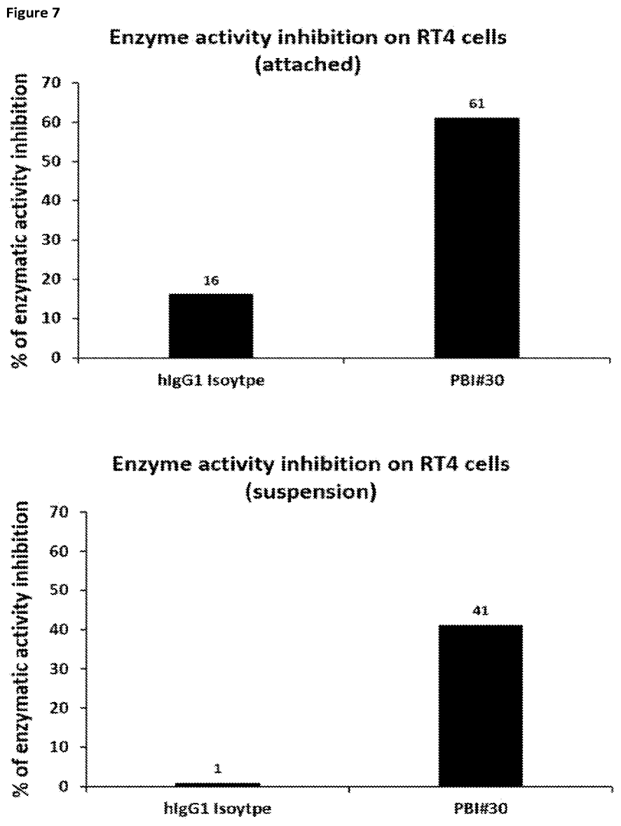

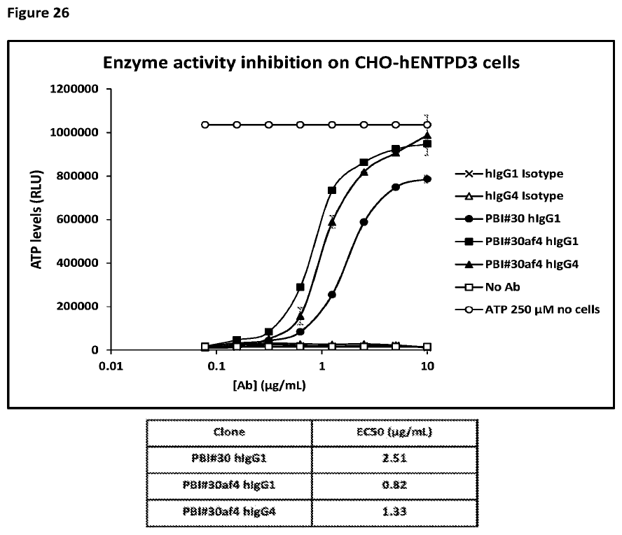

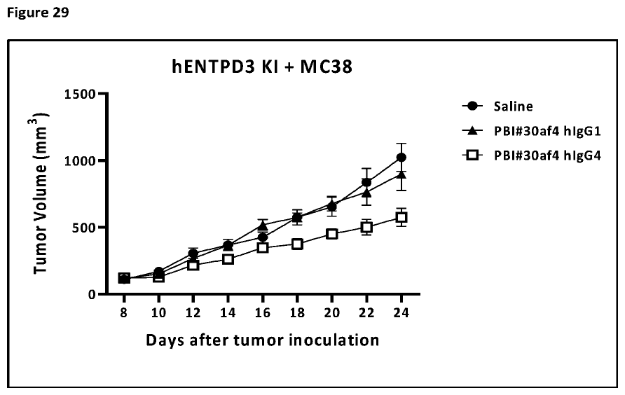

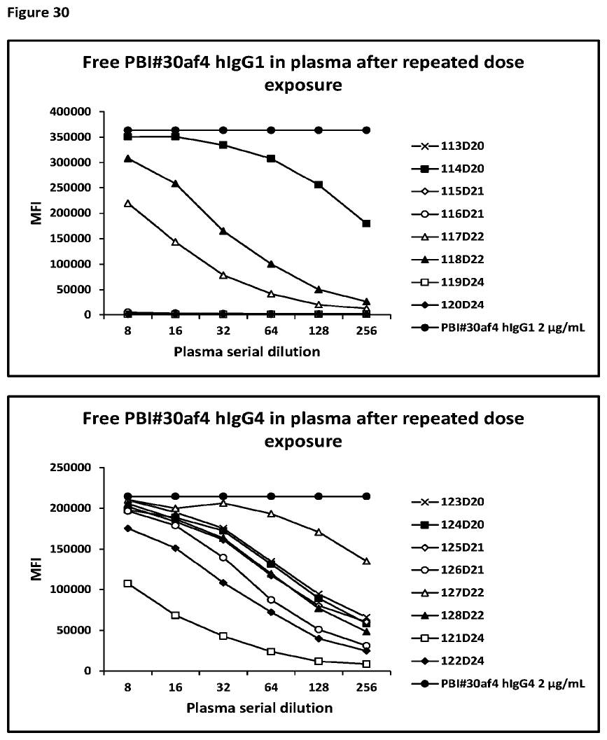

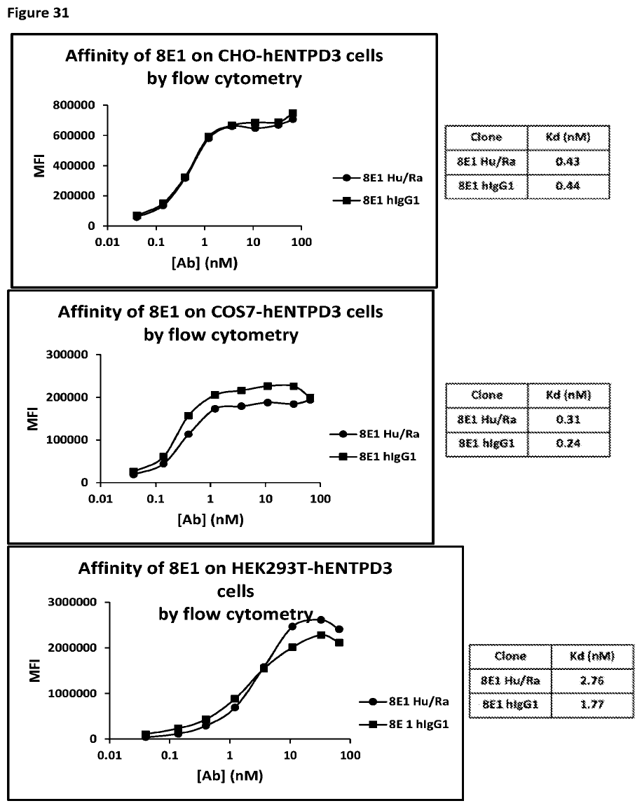

The Subject Monoclonal Antibodies Target ENTPD3+ Cells in the Tumor Through Direct Inhibition of ENTPD3 Enzymatic Activity.
A working example is the fully human clone PBI #30: the original clone PBI #30 hIgG1 shows high levels of ADCC activity in vitro (FIG. 27; EC50=1.443 ag/ml) and also displays high enzyme activity inhibition potency (FIG. 26; EC50=2.51 μg/ml with approximate 80% of maximal inhibition) against ENTPD3^{high} cells in vitro. However, it does not have tumor-killing activity when tested in one setup in vivo (FIG. 22) due to its unsatisfying binding kinetics (FIGS. 1-4).
Affinity maturation variants of PBI #30 hIgG1 (inclusive of PBI #30af4, 6, 7, and 8 hIgG1), as well as isotype switched versions thereof (e.g., Fc-competent IgG1 isotypes switched to the Fc-silent IgG4 versions) (e.g. PBI #30af4, 6, 7, and 8 hIgG4), were generated. Using the variant PBI #30af4 as an example, affinity maturation (PBI #30af4 hIgG1) improves the parental PBI #30 hIgG1 antibody's functional performance such as enzyme activity inhibition capacity (FIG. 26; EC50=0.82 μg/ml with approximate 100% of maximal inhibition) and ADCC activity (FIG. 27; EC50=0.434 μg/ml). As expected, the Fc-silent IgG4 version (PBI #30af4 hIgG4) does not have any ADCC activity (FIG. 27) but its enzyme activity inhibition capacity still remains at a very high level (FIG. 26; EC50=1.33 μg/ml with approximate 100% of maximal inhibition).
Interestingly, when PBI #30af4 hIgG1 and PBI #30af4 hIgG4 antibodies are tested in vivo, the hIgG4 version antibody demonstrates favorable anti-tumor efficacy (FIG. 29), suggesting that, in some cases, enzyme activity inhibition capacity alone, if remained at very high levels, is sufficient to exert in vivo tumoricidal activity.
Of another important note, it was further observed that PBI #30af4 hIgG1 is rapidly cleared in the mouse blood thus limiting antibody availability in tumor tissues (FIG. 30) and ultimately counteracting its in vivo anti-tumor activity (FIG. 29). Therefore it is believed that if PBI #30af4 hIgG1 were engineered to be more stable in vivo, it would confer favorable in vivo ADCC tumor-killing activity to this antibody.

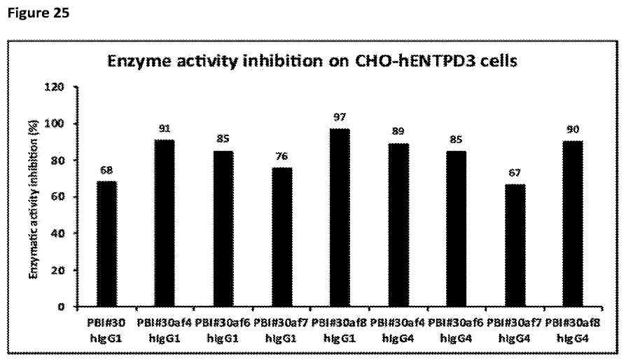

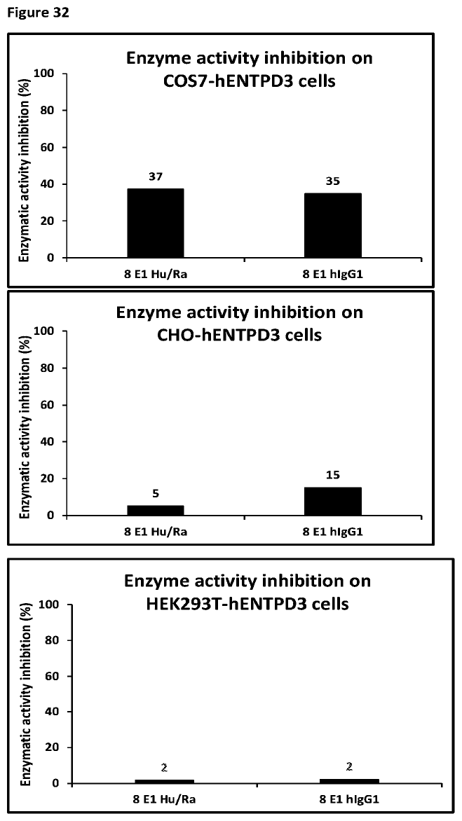

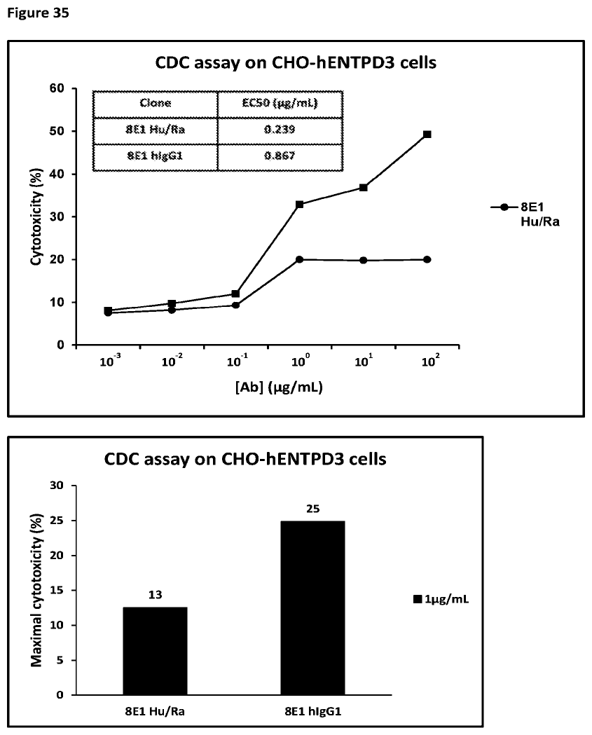

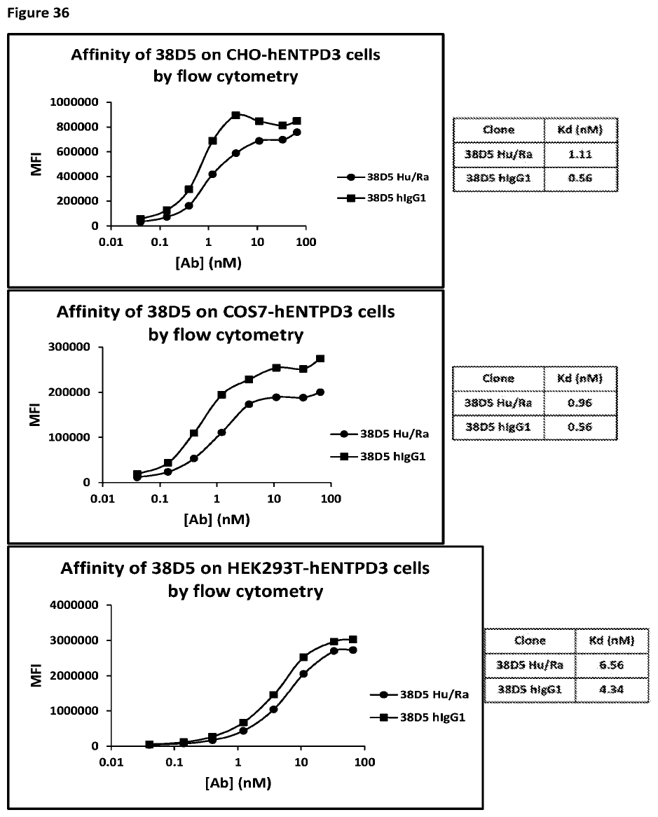

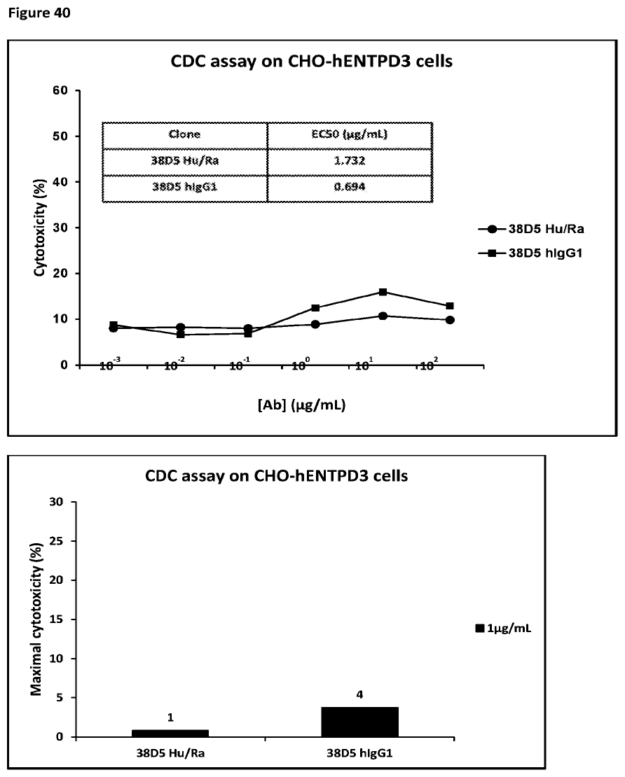

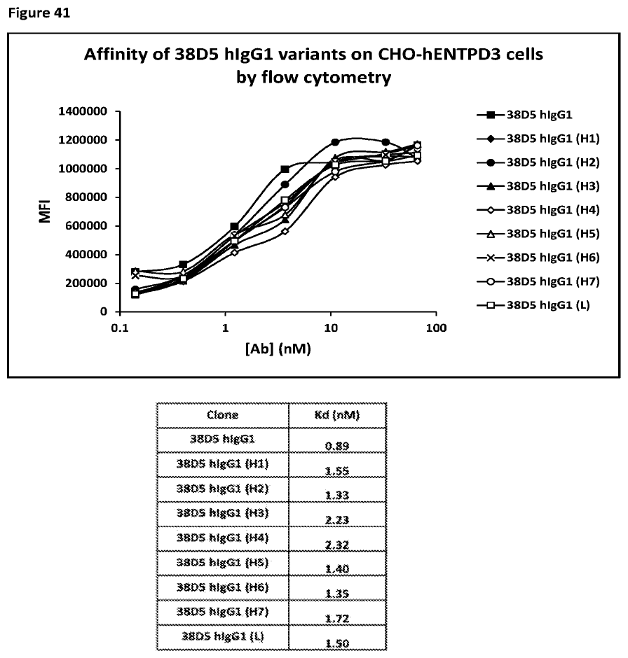

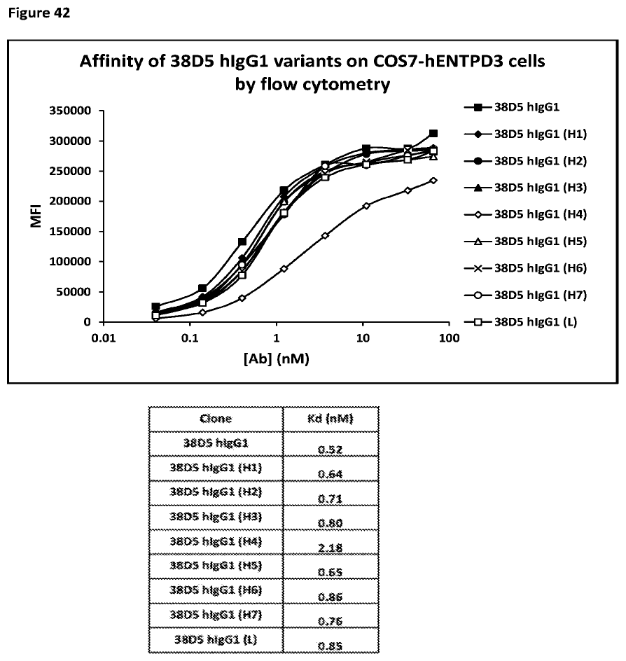

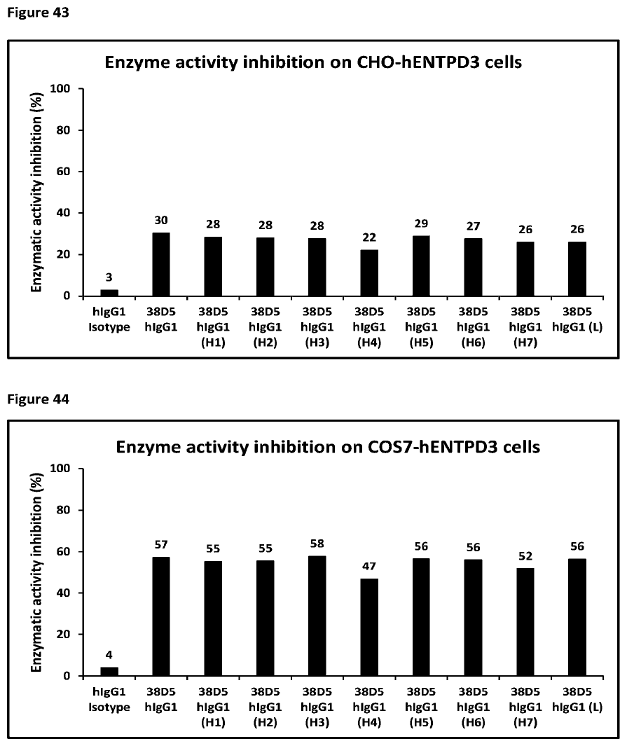

The Subject Anti-ENTPD3 Monoclonal Antibodies are Engineered to Enhance Functionality and/or Reduce Immunogenicity to Humans for Potential Therapeutic Uses.
As a representative example, the fully human clone PBI #30 was engineered by affinity maturation through introducing point mutations into the antibody CDR regions, without disrupting the antibody framework region. Its affinity maturation variants show improvements on function results (FIGS. 25-27).
As another example, rabbit clones 8E1 and 38D5 were humanized (through humanization of the antibody framework region without disrupting the antibody CDR regions) so that the antibody's functionality are not markedly impaired or even improved (FIGS. 32-35 and 37-40), while retaining the specificity and affinity of the parental antibodies. This antibody engineering approach is to reduce the antibody's immunogenicity to humans.
As another example, humanized 38D5 antibody was further engineered to further reduce the potential immunogenicity to humans for therapeutic use, e.g., through introducing point mutation in the antibody backbone sequence without disrupting the variable domains of the heavy and light chains of the antibody nor dramatically damaging the antibody's biophysical and functional attributes (FIGS. 41-44).

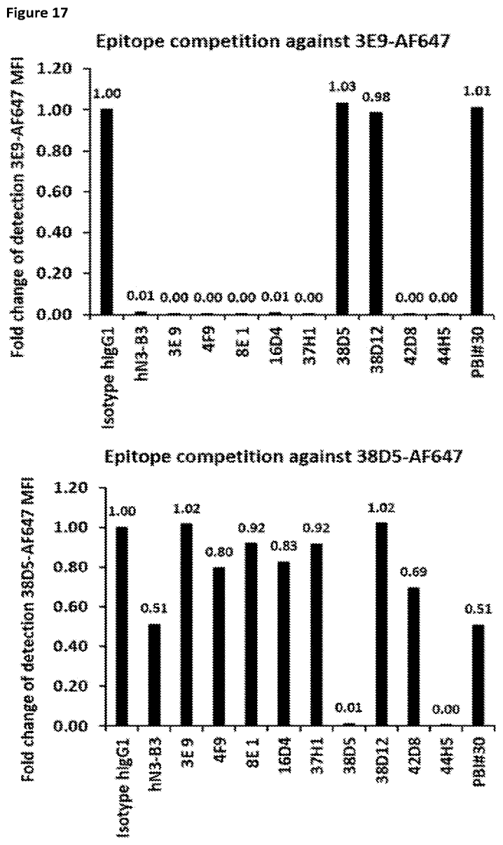

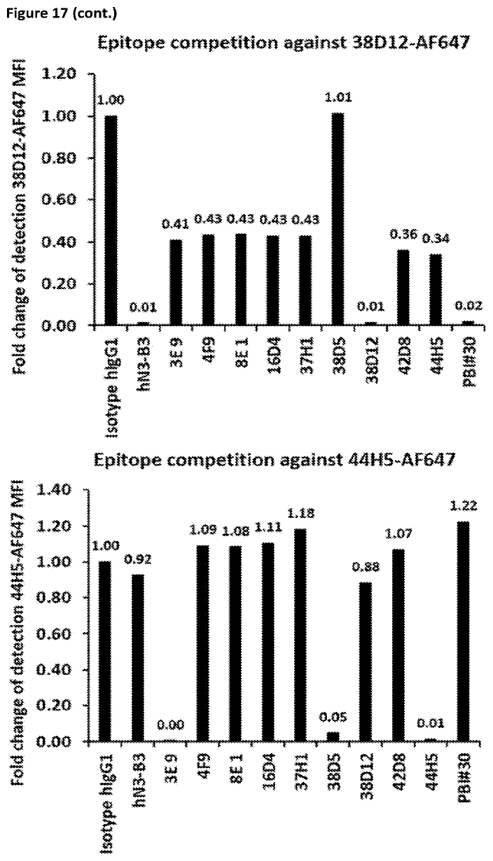

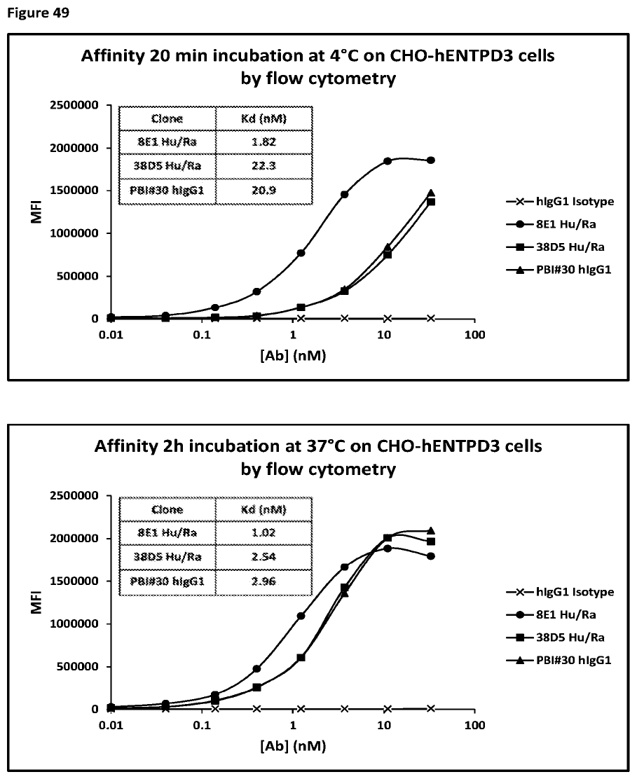

The Subject Monoclonal Antibodies are Comprised of Diverse Anti-ENTPD3 Antibodies that Bind to Different Epitopes and Display Different Cell Binding Kinetics.
As examples, FIG. 17 shows that our anti-ENTPD3 antibody pool in this invention consists of a variety of different antibodies that bind to different epitopes, viz., clone PBI #30 completely competes with clone 38D12 whereas partially competes with all the other clones. In contrast, clone 38D5 only competes completely with clone 44H5, partially competes with clones PBI #30 and 42D8, while does not compete with the rest of the clones. 8E1 completely competes with clone 3E9, partially competes with clones PBI #30 and 38D12, and does not compete with clones 38D5 and 44H5.
Our anti-ENTPD3 antibody pool also contains antibodies with different binding kinetics profiles. As an example shown in FIG. 49, some clones have slow association rates (e.g. 38D5 and PBI #30) whereas some have fast association rates (e.g. 8E1).
Such biophysical diversity of our anti-ENTPD3 monoclonal antibody pool provides greater opportunities to select the best lead candidates for potential therapeutic uses.

### II. Definitions

To facilitate an understanding of the present invention, a number of terms and phrases are defined below.

“NTPDase3” is encoded by the ENTPD3 gene, and is a plasma membrane-bound ectonucleotidase that hydrolyzes extracellular NTPs to NMPs via corresponding NDP intermediates. Representative human NTPDase3 cDNA and protein sequences are well-known in the art and are publicly available from the National Center for Biotechnology Information (NCBI). For example, a human NTPDase3 sequence that contains two cytoplasmic domains, two transmembrane domains and a large extracellular region is provided in UniProtKB as 075355 (ENTP3_HUMAN). Two potential isoforms of human NTPDase3 containing only one cytoplasmic domain and one transmembrane domain (the short forms) have also been reported in UniProtKB as C9J0J3 (C9J0J3_HUMAN) and A0A3B3|T06 (A0A3B3|T06_HUMAN).

The crystal structure and the structure-function relationship of human NTPDase3 proteins is largely unknown in the art. The only available information in this regard is a 3D model of human NTPDase3 by threading the primary sequence of the extracellular portion of NTPDase3 through the crystal structure of the extracellular portion of rat NTPDase2 (Munkonda et al. (2009) FEBS J. 276:479-496; Ivanenkov et al. (2010) Protein Engineering, Design & Selection 23(7):579-588).

The modulation (e.g., decrease) in NTPDase3 activity can be measured in any number of ways (e.g., according to measures described herein, including using controls, ratios, comparisons to baselines, and the like). For example, a NTPDase3 activity modulator can decrease the catalytic activity of the ectonucleotidase or overall NTPDase3 activity as compared to the level of such ectonucleotidase in the presence of a test agent. In one embodiment, NTPDase3 activity is determined by analyzing the concentration of adenosine in a sample. The concentration can be assessed over time. In another embodiment, ATP is added in the sample tested and the concentration of ATP, ADP, AMP or adenosine is determined or assessed. A modulation in this context, such as a decrease, can mean a decrease of 1%, 5%, 10%>, 20%, 25%, 30%, 35%, 40%, 45%, 50%, 55%, 60%, 65%, 70%, 75%, 80%, 85%, 90%, 95%, 100%, 120%, 150%, 200%, 500%, 1000%, or more. In an embodiment, said increase is detected over time.

A “NTPDase3 Antibody” (alternatively an “anti-NTPDase3 antibody”) refers to an antibody that selectively binds to one or more epitopes of the NTPDase3 protein, and includes monoparatopic antibodies, as well as biparatopic and other multiparatopic format antibodies.

a. Antibodies and Other Polypeptides

The term “antibody” as used herein refers to an immunoglobulin molecule that recognizes and specifically binds a target, such as a protein, polypeptide, peptide, carbohydrate, polynucleotide, lipid, or a combination of any of the foregoing, through at least one antigen-binding site wherein the antigen-binding site is usually within the variable region of the immunoglobulin molecule. As used herein, the term encompasses intact polyclonal antibodies, intact monoclonal antibodies, antibody fragments (such as Fab, Fab′, F(ab′)2, and Fv fragments), single chain Fv (scFv) antibodies provided those fragments have been formatted to include an Fc or other FcγRIII binding domain, multispecific antibodies, bispecific antibodies, monospecific antibodies, monovalent antibodies, chimeric antibodies, humanized antibodies, human antibodies, fusion proteins comprising an antigen-binding site of an antibody (formatted to include an Fc or other FcγRIII binding domain), and any other modified immunoglobulin molecule comprising an antigen-binding site as long as the antibodies exhibit the desired biological activity.

“Antibody-mediated target cytosis” in the context of the present invention refers to antibody-mediated depletion of NTPDase3 from the surface of NTPDase3+ cells without a substantial decrease in the number of NTPDase3+ cells, i.e., through a process other than induction of NTPDase3+ cell death.

The term “antigen-binding portion” or antigen-binding fragment” of an antibody, as used herein, refers to one or more fragments of an antibody that retain the ability to specifically bind to an antigen (e.g., human NTPDase3). It has been shown that the antigen-binding function of an antibody can be performed by fragments of a full-length antibody. Examples of binding fragments encompassed within the term “antigen-binding portion” of an antibody, e.g., an anti-NTPDase3 antibody described herein, include (i) a Fab fragment, a monovalent fragment consisting of the V_{L}, V_{H}, CL and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) or (vii) a combination of two or more isolated CDRs which may optionally be joined by a synthetic linker. Such single chain antibodies are also intended to be encompassed within the term “antigen-binding portion” of an antibody. These and other potential constructs are described at Chan & Carter (2010) Nat. Rev. Immunol. 10:301. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The term “variable region” of an antibody refers to the variable region of an antibody light chain, or the variable region of an antibody heavy chain, either alone or in combination. Generally, the variable region of heavy and light chains each consist of four framework regions (FR) and three complementarity determining regions (CDRs), also known as “hypervariable regions”. The CDRs in each chain are held together in close proximity by the framework regions and, with the CDRs from the other chain, contribute to the formation of the antigen-binding sites of the antibody. There are at least two techniques for determining CDRs: (1) an approach based on cross-species sequence variability (i.e., Kabat et al., 1991, Sequences of Proteins of Immunological Interest, 5th Edition, National Institutes of Health, Bethesda Md.), and (2) an approach based on crystallographic studies of antigen-antibody complexes (Al Lazikani et al., 1997, J. Mol. Biol., 273:927-948). In addition, combinations of these two approaches are sometimes used in the art to determine CDRs.

While the antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively, the preferred NTPDase3 antibody is an IgG1 and IgG3 isotype in order to engage FcγRIII most effectively (i.e., with a Kd of 10⁻⁷ or smaller).

In certain embodiments, the antibody is “hypofucosylated” and may even be “afucosylated”. A “hypofucosylated” antibody preparation refers to an antibody preparation in which less than 50% of the oligosaccharide chains contain α-1,6-fucose. Typically, less than about 40%, less than about 30%, less than about 20%, less than about 10%, or less than 5% or less than 1% of the oligosaccharide chains contain α-1,6-fucose in a “hypofucosylated” antibody preparation. An “afucosylated” antibody lacks α-1,6-fucose in the carbohydrate attached to the CH2 domain of the IgG heavy chain.

The term “monoclonal antibody,” as used herein, refers to an antibody that displays a single binding specificity and affinity for a particular epitope or a composition of antibodies in which all antibodies display a single binding specificity and affinity for a particular epitope. Typically such monoclonal antibodies will be derived from a single cell or nucleic acid encoding the antibody, and will be propagated without intentionally introducing any sequence alterations. Accordingly, the term “human monoclonal antibody” refers to a monoclonal antibody that has variable and optional constant regions derived from human germline immunoglobulin sequences. In one embodiment, human monoclonal antibodies are produced by a hybridoma, for example, obtained by fusing a B cell obtained from a transgenic or transchromosomal non-human animal (e.g., a transgenic mouse having a genome comprising a human heavy chain transgene and a light chain transgene), to an immortalized cell.

The term “humanized antibody” as used herein refers to forms of non-human (e.g., murine) antibodies that are specific immunoglobulin chains, chimeric immunoglobulins, or fragments thereof that contain minimal non-human sequences. Typically, humanized antibodies are human immunoglobulins in which residues of the CDRs are replaced by residues from the CDRs of a non-human species (e.g., mouse, rat, rabbit, or hamster) that have the desired specificity, affinity, and/or binding capability. In some instances, the Fv framework region residues of a human immunoglobulin are replaced with the corresponding residues in an antibody from a non-human species. The humanized antibody can be further modified by the substitution of additional residues either in the Fv framework region and/or within the replaced non-human residues to refine and optimize antibody specificity, affinity, and/or binding capability. The humanized antibody may comprise variable domains containing all or substantially all of the CDRs that correspond to the non-human immunoglobulin whereas all or substantially all of the framework regions are those of a human immunoglobulin sequence. In some embodiments, the variable domains comprise the framework regions of a human immunoglobulin sequence. In some embodiments, the variable domains comprise the framework regions of a human immunoglobulin consensus sequence. The humanized antibody can also comprise at least a portion of an immunoglobulin constant region or domain (Fc), typically that of a human immunoglobulin. A humanized antibody is usually considered distinct from a chimeric antibody.

The term “human antibody” as used herein refers to an antibody produced by a human or an antibody having an amino acid sequence corresponding to an antibody produced by a human made using any of the techniques known in the art.

The term “chimeric antibody” as used herein refers to an antibody wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and/or binding capability, while the constant regions are homologous to the sequences in antibodies derived from another species (usually human) to avoid eliciting an immune response in that species.

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIB) receptor.

An “FcγRIII binding moiety” is a peptide, protein, nucleic acid or other moiety which, when associated with an antigen binding site of an anti-NTPDase3 antibody, is able to bind to FcγRIII (CD16) and mediate antibody-dependent cellular cytotoxicity (ADCC). The heavy chain Fc fragment containing the CH2 and CH3 domains of IgG1 and IgG3 isotypes are FcγRIII binding moiety.

The terms “epitope” and “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation.

As use herein, the term “specifically binds to” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of less than or equal to 1 μM, 100 nM, 10 nM, 1 nM, or even 0.1 nM. In certain embodiments, an antibody specifically binds to an epitope on a protein that is conserved among the protein from different species. In another embodiment, specific binding can include, but does not require exclusive binding.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides encompassed by the present invention may be based upon antibodies or other members of the immunoglobulin superfamily, in certain embodiments, the polypeptides can occur as single chains or as associated chains.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides encompassed by the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a nucleotide sequence encoding a protein of interest.

A “conservative amino acid substitution” is one in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble proteins, and/or antibodies encompassed by the present invention do not abrogate the binding of the polypeptide, soluble protein, or antibody containing the amino acid sequence, to the target binding site. Methods of identifying amino acid conservative substitutions which do not eliminate binding are well-known in the art.

A polypeptide, soluble protein, antibody, polynucleotide, vector, cell, or composition which is “isolated” is a polypeptide, soluble protein, antibody, polynucleotide, vector, cell, or composition which is in a form not found in nature. Isolated polypeptides, soluble proteins, antibodies, polynucleotides, vectors, cells, or compositions include those which have been purified to a degree that they are no longer in a form in which they are found in nature. In some embodiments, a polypeptide, soluble protein, antibody, polynucleotide, vector, cell, or composition which is isolated is substantially pure.

The term “substantially pure” as used herein refers to material which is at least 50% pure (i.e., free from contaminants), at least 90% pure, at least 95% pure, at least 98% pure, or at least 99% pure.

The term “fusion protein” or “fusion polypeptide” as used herein refers to a hybrid protein expressed by a nucleic acid molecule comprising nucleotide sequences of at least two genes.

The term “linker” or “linker region” as used herein refers to a linker inserted between a first polypeptide (e.g., an anti-NTPDase3 antibody) and a second polypeptide (e.g., an Fc or other FcγRIII binding moiety; an scFV, Vhh domain or the like the binds a different protein to create a bispecific antibody format maintaining the bivalency for NTPDase3). In some embodiments, the linker is a peptide linker. Linkers should not adversely affect the expression, secretion, or bioactivity of the polypeptides. Preferably, linkers are not antigenic and do not elicit an immune response.

b. Nucleic Acids

The terms “polynucleotide” and “nucleic acid” and “nucleic acid molecule” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

As used herein, the term “nucleic acid molecule encoding”, “DNA sequence encoding,” and “DNA encoding” refer to the order or sequence of nucleotides along a strand of deoxyribonucleic acid deoxyribonucleotides. The order of these deoxyribonucleotides determines the order of amino acids along the polypeptide (protein) chain. Thus, a nucleic acid sequence encoding the amino acid sequence.

When used in reference to nucleotide sequences, “sequence” as used herein, the term grammatical and other forms may comprise DNA or RNA, and may be single or double stranded. Nucleic acid sequences may be mutated. Nucleic acid sequence may have any length, for example 2 to 1,000,000 or more nucleotides (or any integral value above or between) a nucleic acid, for example a length of from about 100 to about 10,000, or from about 200 nucleotides to about 500 nucleotides.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

As used herein, the term “transfection” refers to an exogenous nucleic acid into a eukaryotic cell. Transfection can be achieved by various means known in the art, including calcium phosphate-DNA co-precipitation, DEAE-dextran-mediated transfection, polybrene-mediated transfection, electroporation, microinjection, liposome fusion, lipofection, protoplast fusion, retroviral infection, and biolistics technology (biolistics).

The term “carrier” as used herein is an isolated nucleic acid comprising the isolated nucleic acid can be used to deliver a composition to the interior of the cell. It is known in the art a number of carriers including, but not limited to the linear polynucleotides, polynucleotides associated with ionic or amphiphilic compounds, plasmids, and viruses. Thus, the term “vector” includes an autonomously replicating plasmid or virus. The term should also be construed to include facilitate transfer of nucleic acid into cells of the non-plasmid and non-viral compounds, for example, polylysine compounds, liposomes, and the like. Examples of viral vectors include, but are not limited to adenoviral vectors, adeno-associated virus vectors, retroviral vectors and the like.

As used herein, the term “expression vector” refers to a vector comprising a recombinant polynucleotide comprising expression control sequence and a nucleotide sequence to be expressed operably linked. The expression vector comprises sufficient cis-acting elements (cis-acting elements) used for expression; other elements for expression can be supplied by the host cell or in vitro expression system. Expression vectors include all those known in the art, such as cosmids, plasmids (e.g., naked or contained in liposomes) and viruses (e.g., lentivirus, retroviruses, adenoviruses and adeno-associated viruses).

As used herein, the term “operably linked” refers to functional linkage between the regulatory sequence and a heterologous nucleic acid sequence is connected to a connection results in the expression of the latter. For example, when the first nucleic acid sequence and a second nucleic acid sequence is a functional relationship between the first nucleic acid sequence and the second nucleic acid sequence is operably linked. For example, if the promoter affects the transcription or expression of the coding sequence, the promoter is operably linked to a coding sequence. Typically, DNA sequencing operably linked are contiguous, and to join two protein coding regions in the same reading frame as necessary.

As used herein, the term “promoter” is defined as a promoter DNA sequence recognized by the synthetic machinery required for the synthesis machinery of the cell specific transcription of a polynucleotide sequence or introduced.

The term “constitutive expression” as used herein refers to all expressed under physiological conditions.

The term “inducible expression” as used herein refers to expression under certain conditions, the conditions such as occurs when a T cell antigen binding. How those skilled in the routine “induce expression.”

The term “electroporation” refers to the use of a transmembrane electric field pulse to induce microscopic pathways (pores) in a bio-membrane; their presence allows biomolecules such as plasmids or other oligonucleotide to pass from one side of the cellular membrane to the other.

c. Checkpoint Inhibitors, Co-stimulatory Agonists, Innate Immune Inducers and Chemotherapeutics

A “checkpoint molecule” refers to proteins that are expressed by tissues and/or immune cells and reduce the efficacy of an immune response in a manner dependent on the level of expression of the checkpoint molecule. When these proteins are blocked, the “brakes” on the immune system are released and, for example, T cells are able to kill cancer cells more effectively. Examples of checkpoint proteins found on T cells or cancer cells include PD-1/PD-L1 and CTLA-4/B7-1/B7-2, PD-L2, NKG2A, KIR, LAG-3, TIM-3, CD96, VISTA, TIGIT, CD39 and Siglec-15.

A “checkpoint inhibitor” refers to a drug entity that reverses the immunosuppressive signaling from a checkpoint molecule.

A “costimulatory molecule” refers to an immune cell such as a T cell cognate binding partner which specifically binds to costimulatory ligands thereby mediating co-stimulation, such as, but not limited to proliferation. Costimulatory molecules are cell surface molecules other than the antigen receptor or ligand which facilitate an effective immune response. Co-stimulatory molecules include, but are not limited to MHCI molecules, BTLA receptor and Toll ligands, and OX40, CD27, CD28, CDS, ICAM-1, LFA-1 (CD11a/CD18), ICOS (CD278) and 4-1BB (CD137). Examples of costimulatory molecules include but are not limited to: CDS, ICAM-1, GITR, BAFFR, HVEM (LIGHTR), SLAMF7, NKp80 (KLRF1), NKp44, NKp30, NKp46, CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244,2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and CD83 ligand.

A “costimulatory agonist” refers to a drug entity that activates (agonizes) the costimulatory molecule, such as costimulatory ligand would do, and produces an immunostimulatory signal or otherwise increases the potency or efficacy of an immune response.

An “innate immune inducer” is an agent that mimetics the innate immune response, including activation of inflammatory activities and/or deactivation of anti-inflammatory activities of macrophage, NK cells, dendritic cells, monocytes, neutrophils and the like.

Innate immune inducers include inhibitors of the CD47-SIRPα axis, such as antibodies or other binding moieties that bind to CD47 or SIRPα and inhibit the interaction of the two molecules in order to promote antitumor macrophage activity. Innate immune inducers include inhibitors of the CD24-Siglec-10 axis, such as antibodies or other binding moieties that bind to CD24 or Siglec-10 and inhibit the interaction of the two molecules in order to promote antitumor macrophage activity. In other embodiments, the innate immune activator can be an NGK2A checkpoint inhibitor that blocks HLA-E driven inhibition of NK and CD8+ cells. Small molecule inducers of innate immunity include such agents STING agonist, TLR7/8 agonists and RIG-1 agonists.

A “chemotherapeutic agent” is a chemical compound useful in the treatment of cancer. Examples of chemotherapeutic agents include alkylating agents such as thiotepa and cyclophosphamide (CYTOXAN); alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL); beta-lapachone; lapachol; colchicines; betulinic acid; a camptothecin (including the synthetic analogue topotecan (HYCAMTIN), CPT-11 (irinotecan, CAMPTOSAR), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omega1I (see, e.g., Nicolaou et al., Angew. Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including ADRIAMYCIN, morpholino-doxorubicin, cyanomorpholino-doxorubicin, 2-pyrrolino-doxorubicin, doxorubicin HCl liposome injection (DOXIL) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR), tegafur (UFTORAL), capecitabine (XELODA), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative), as well as other c-Kit inhibitors; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; eflornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE, FILDESIN); dacarbazine; mannomustine; mitobronitol; mitolactol; pipobroman; gacytosine; arabinoside (“Ara-C”); thiotepa; taxoids, e.g., paclitaxel (TAXOL), albumin-engineered nanoparticle formulation of paclitaxel (ABRAXANE), and doxetaxel (TAXOTERE); chlorambucil; 6-thioguanine; mercaptopurine; methotrexate; platinum analogs such as cisplatin and carboplatin; vinblastine (VELBAN); platinum; etoposide (VP-16); ifosfamide; mitoxantrone; vincristine (ONCOVIN); oxaliplatin; leucovovin; vinorelbine (NAVELBINE); novantrone; edatrexate; daunomycin; aminopterin; ibandronate; topoisomerase inhibitor RFS 2000; difluoromethylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN) combined with 5-FU and leucovovin.

Also included in this definition are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX tamoxifen), raloxifene (EVISTA), droloxifene, 4-hydroxytamoxifen, trioxifene, keoxifene, LY117018, onapristone, and toremifene (FARESTON); anti-progesterones; estrogen receptor down-regulators (ERDs); estrogen receptor antagonists such as fulvestrant (FASLODEX); agents that function to suppress or shut down the ovaries, for example, leutinizing hormone-releasing hormone (LHRH) agonists such as leuprolide acetate (LUPRON and ELIGARD), goserelin acetate, buserelin acetate and tripterelin; anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE), exemestane (AROMASIN), formestanie, fadrozole, vorozole (RIVISOR), letrozole (FEMARA), and anastrozole (ARIMIDEX). In addition, such definition of chemotherapeutic agents includes bisphosphonates such as clodronate (for example, BONEFOS or OSTAC), etidronate (DIDROCAL), NE-58095, zoledronic acid/zoledronate (ZOMETA), alendronate (FOSAMAX), pamidronate (AREDIA), tiludronate (SKELID), or risedronate (ACTONEL); as well as troxacitabine (a 1,3-dioxolane nucleoside cytosine analog); anti-sense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE vaccine and gene therapy vaccines, for example, ALLOVECTIN vaccine, LEUVECTIN vaccine, and VAXID vaccine; topoisomerase 1 inhibitor (e.g., LURTOTECAN); an anti-estrogen such as fulvestrant; a Kit inhibitor such as imatinib or EXEL-0862 (a tyrosine kinase inhibitor); EGFR inhibitor such as erlotinib or cetuximab; an anti-VEGF inhibitor such as bevacizumab; arinotecan; rmRH (e.g., ABARELIX); lapatinib and lapatinib ditosylate (an ErbB-2 and EGFR dual tyrosine kinase small-molecule inhibitor also known as GW572016); 17AAG (geldanamycin derivative that is a heat shock protein (Hsp) 90 poison), and pharmaceutically acceptable salts, acids or derivatives of any of the above.

As used herein, the term “cytokine” refers generically to proteins released by one cell population that act on another cell as intercellular mediators or have an autocrine effect on the cells producing the proteins. Examples of such cytokines include lymphokines, monokines; interleukins (“ILs”) such as IL-1, IL-1a, IL-2, IL-3, IL-4, IL-5, IL-6, IL-7, IL-8, IL-9, IL10, IL-11, IL-12, IL-13, IL-15, IL-17A-F, IL-18 to IL-29 (such as IL-23), IL-31, including PROLEUKIN rIL-2; a tumor-necrosis factor such as TNF-α or TNF-β, TGF-β1-3; and other polypeptide factors including leukemia inhibitory factor (“LIF”), ciliary neurotrophic factor (“CNTF”), CNTF-like cytokine (“CLC”), cardiotrophin (“CT”), and kit ligand (“KL”).

As used herein, the term “chemokine” refers to soluble factors (e.g., cytokines) that have the ability to selectively induce chemotaxis and activation of leukocytes. They also trigger processes of angiogenesis, inflammation, wound healing, and tumorigenesis. Example chemokines include IL-8, a human homolog of murine keratinocyte chemoattractant (KC).

d. Treatments

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage and tumor clearance.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2.0×, 2.5×, or 3.0× length of the treatment duration.

The terms “cancer” and “cancerous” as used herein refer to or describe the physiological condition in mammals in which a population of cells are characterized by unregulated cell growth. Examples of cancer include, but are not limited to, carcinoma, blastoma, sarcoma, and hematologic cancers such as lymphoma and leukemia.

The terms “tumor” and “neoplasm” as used herein refer to any mass of tissue that results from excessive cell growth or proliferation, either benign (noncancerous) or malignant (cancerous) including pre-cancerous lesions. Tumor growth is generally uncontrolled and progressive, does not induce or inhibit the proliferation of normal cells. Tumor can affect a variety of cells, tissues or organs, including but not limited to selected from bladder, bone, brain, breast, cartilage, glial cells, esophagus, fallopian tube, gall bladder, heart, intestine, kidney, liver, lung, lymph node, neural tissue, ovary, pancreas, prostate, skeletal muscle, skin, spinal cord, spleen, stomach, testis, thymus, thyroid, trachea, urethra, ureter, urethra, uterus, vagina organ or tissue or the corresponding cells. Tumors include cancers, such as sarcoma, carcinoma, plasmacytoma or (malignant plasma cells). Tumors encompassed by the present invention, may include, but are not limited to leukemias (e.g., acute leukemia, acute lymphoblastic leukemia, acute myeloid leukemia, acute myeloid leukemia, acute promyelocytic leukemia, acute myeloid-monocytic leukemia, acute monocytic leukemia, acute leukemia, chronic leukemia, chronic myeloid leukemia, chronic lymphocytic leukemia, polycythemia vera), lymphomas (Hodgkin's disease, non-Hodgkin's disease), primary macroglobulinemia disease, heavy chain disease, and solid tumors such as sarcomas cancer (e.g., fibrosarcoma, myxosarcoma, liposarcoma, chondrosarcoma, osteosarcoma, chordoma, endothelium sarcoma, lymphangiosarcoma, angiosarcoma, lymphangioendothelio sarcoma, synovioma vioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinoma, carcinoma, bronchogenic carcinoma, medullary carcinoma, renal cell carcinoma, hepatoma, Nile duct carcinoma, choriocarcinoma, spermatogonia Tumor, embryonal carcinoma, Wilms' tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, retinoblastoma), esophageal cancer, gallbladder, kidney cancer, multiple myeloma. Preferably, a “tumor” includes, but is not limited to: pancreatic cancer, liver cancer, lung cancer, stomach cancer, esophageal cancer, head and neck squamous cell carcinoma, prostate cancer, colon cancer, breast cancer, lymphoma, gallbladder cancer, renal cancer, leukemia, multiple myeloma, ovarian cancer, cervical cancer and glioma.

The term “metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures.

The terms “cancer cell” and “tumor cell” refer to the total population of cells derived from a cancer or tumor or pre-cancerous lesion, including both non-tumorigenic cells, which comprise the bulk of the cancer cell population, and tumorigenic stem cells (cancer stem cells). As used herein, the terms “cancer cell” or “tumor cell” will be modified by the term “non-tumorigenic” when referring solely to those cells lacking the capacity to renew and differentiate to distinguish those tumor cells from cancer stem cells.

The term “effective amount” as used herein refers to an amount to provide therapeutic or prophylactic benefit.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD recorded since the treatment started or the presence of one or more new lesions.

As used herein, “progression free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” (ORR) refers to the sum of complete response (CR) rate and partial response (PR) rate.

As used herein, “overall survival” refers to the percentage of individuals in a group who are likely to be alive after a particular duration of time.

The term “treatment” as used herein refers to the individual trying to change the process or treatment of a clinical disease caused by intervention of a cell, may be either preventive intervention course of clinical pathology. Including but not limited to treatment to prevent the occurrence or recurrence of disease, alleviation of symptoms, reducing the direct or indirect pathological consequences of any disease, preventing metastasis, slow the rate of disease progression, amelioration or remission of disease remission or improved prognosis.

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The terms “agonist” and “agonistic” as used herein refer to or describe a therapeutic moiety that is capable of, directly or indirectly, substantially inducing, activating, promoting, increasing, or enhancing the biological activity of a target and/or a pathway. The term “agonist” is used herein to include any agent that partially or fully induces, activates, promotes, increases, or enhances the activity of a protein or other target of interest.

The terms “antagonist” and “antagonistic” as used herein refer to or describe a therapeutic moiety that is capable of, directly or indirectly, partially or fully blocking, inhibiting, reducing, or neutralizing a biological activity of a target and/or pathway. The term “antagonist” is used herein to include any agent that partially or fully blocks, inhibits, reduces, or neutralizes the activity of a protein or other target of interest.

The terms “modulation” and “modulate” as used herein refer to a change or an alteration in a biological activity. Modulation includes, but is not limited to, stimulating an activity or inhibiting an activity. Modulation may be an increase in activity or a decrease in activity, a change in binding characteristics, or any other change in the biological, functional, or immunological properties associated with the activity of a protein, a pathway, a system, or other biological targets of interest.

The term “immune response” as used herein includes responses from both the innate immune system and the adaptive immune system. It includes both cell-mediated and/or humoral immune responses. It includes both T-cell and B-cell responses, as well as responses from other cells of the immune system such as natural killer (NK) cells, monocytes, macrophages, etc.

The term “pharmaceutically acceptable” refers to a substance approved or approvable by a regulatory agency of the Federal government or a state government or listed in the U.S.

Pharmacopeia or other generally recognized pharmacopeia for use in animals, including humans.

The terms “pharmaceutically acceptable excipient, carrier or adjuvant” or “acceptable pharmaceutical carrier” refer to an excipient, carrier or adjuvant that can be administered to a subject, together with at least one agent of the present disclosure, and which does not destroy the pharmacological activity thereof and is nontoxic when administered in doses sufficient to deliver a therapeutic effect. In general, those of skill in the art and the U.S. FDA consider a pharmaceutically acceptable excipient, carrier, or adjuvant to be an inactive ingredient of any formulation.

The terms “effective amount” or “therapeutically effective amount” or “therapeutic effect” refer to an amount of an anti-NTPDase3 antibody effective to “treat” a disease or disorder in a subject such as, a mammal. In the case of cancer or a tumor, the therapeutically effective amount of an anti-NTPDase3 antibody has a therapeutic effect and as such can boost the immune response, boost the anti-tumor response, increase cytolytic activity of immune cells, increase killing of tumor cells by immune cells, reduce the number of tumor cells; decrease tumorigenicity, tumorigenic frequency or tumorigenic capacity; reduce the number or frequency of cancer stem cells; reduce the tumor size; reduce the cancer cell population; inhibit or stop cancer cell infiltration into peripheral organs including, for example, the spread of cancer into soft tissue and bone; inhibit and stop tumor or cancer cell metastasis; inhibit and stop tumor or cancer cell growth; relieve to some extent one or more of the symptoms associated with the cancer; reduce morbidity and mortality; improve quality of life; or a combination of such effects.

The terms “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus those in need of treatment include those already with the disorder; those prone to have the disorder; and those in whom the disorder is to be prevented. In the case of cancer or a tumor, a subject is successfully “treated” according to the methods encompassed by the present invention if the patient shows one or more of the following: an increased immune response, an increased anti-tumor response, increased cytolytic activity of immune cells, increased killing of tumor cells by immune cells, a reduction in the number of or complete absence of cancer cells; a reduction in the tumor size; inhibition of or an absence of cancer cell infiltration into peripheral organs including the spread of cancer cells into soft tissue and bone; inhibition of or an absence of tumor or cancer cell metastasis; inhibition or an absence of cancer growth; relief of one or more symptoms associated with the specific cancer; reduced morbidity and mortality; improvement in quality of life; reduction in tumorigenicity; reduction in the number or frequency of cancer stem cells; or some combination of effects.

e. Miscellaneous

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

As used herein, reference to “about” or “approximately” a value or parameter includes (and describes) embodiments that are directed to that value or parameter. For example, description referring to “about X” includes description of “X”.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### III. Anti-NTPDase3 Antibodies

a. Monoclonal Antibodies

The anti-NTPDase3 antibodies may be monoclonal antibodies. Such monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the NTPDase3 polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against the polypeptide. Preferably, the binding specificity of monoclonal antibodies produced by the hybridoma cells is determined by immunoprecipitation or by an in vitro binding assay, such as radioimmunoassay (RIA) or enzyme-linked immunosorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies encompassed by the invention can be readily isolated and sequenced using conventional procedures (e.g., by using oligonucleotide probes that are capable of binding specifically to genes encoding the heavy and light chains of murine antibodies). The hybridoma cells encompassed by the present invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences [U.S. Pat. No. 4,816,567; Morrison et al., supra] or by covalently joining to the immunoglobulin coding sequence all or part of the coding sequence for a non-immunoglobulin polypeptide. Such a non-immunoglobulin polypeptide can be substituted for the constant domains of an antibody encompassed by the present invention, or can be substituted for the variable domains of one antigen-combining site of an antibody encompassed by the present invention to create a chimeric bivalent antibody.

b. Human and Humanized Antibodies

The anti-NTPDase3 antibodies encompassed by the present invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

The antibodies may also be affinity matured using known selection and/or mutagenesis methods as described above. Preferred affinity matured antibodies have an affinity which is five times, more preferably 10 times, even more preferably 20 or 30 times greater than the starting antibody (generally murine, humanized or human) from which the matured antibody is prepared.

c. Bispecific Antibodies

Anti-NTPDase3 antibodies described herein include bispecific molecules. An anti-NTPDase3 antibody, or antigen-binding portions thereof, can be derivatized or linked to another functional molecule, e.g., another peptide or protein (e.g., another antibody or ligand for a receptor) to generate a bispecific molecule that binds to at least two different binding sites or target molecules. The antibody described herein may in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule described herein, an antibody described herein can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, provided herein are bispecific molecules comprising at least one first binding specificity for NTPDase3 and a second binding specificity for a second target epitope. In an embodiment described herein in which the bispecific molecule is multi-specific, the molecule can further include a third binding specificity.

In certain embodiments, the subject bispecific (or multi-specific as the case may be) includes one or more binding domains for immune checkpoints, e.g., which are checkpoint inhibitors, such as PD-1, PD-L1, CTLA-4/B7-1/B7-2, PD-L2, KIR, LAG-3, TIM-3, CD96, VISTA, TIGIT, CD39 and/or Siglec-15. In certain embodiments, the multi-specific includes binding domains that bind checkpoint proteins on T-cells, especially checkpoints associated with T-cell exhaustion such as LAG-3, TIM-3, TIGIT or CD39. In certain embodiments, the multi-specific binds to NTPDase3 and one or more other T-cell associated checkpoints and leads to antibody-dependent cellular cytotoxicity of cells expressing each or both of NTPDase3 and the other checkpoint proteins to which it binds.

In certain embodiments, the subject bispecific (or multi-specific as the case may be) includes one or more binding domains for immune costimulatory receptors, e.g., which are costimulatory agonists (activators), such as agonists of MHCI molecules, BTLA receptor and Toll ligands, and OX40, CD27, CD28, CDS, ICAM-1, LFA-1 (CD11a/CD18), ICOS (CD278) and 4-1BB (CD137). Examples of costimulatory molecules that can be included in the multi-specific include but are not limited to: CDS, ICAM-1, GITR, BAFFR, HVEM (LIGHTR), SLAMF7, NKp80 (KLRF1), NKp44, NKp30, NKp46, CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and CD83 ligand.

In certain embodiments, the subject bispecific (or multi-specific as the case may be) includes one or more binding domains which serve as innate immune activators, such as binding moieties for CD47, SIRPα, CD24, Siglec-10 or NKG2A.

In one embodiment, the bispecific molecules described herein comprise as a binding specificity at least one antibody, or an antibody fragment thereof, including, e.g., an Fab, Fab′, F(ab′)₂, Fv, or a single chain Fv. The antibody may also be a light chain or heavy chain dimer, or any minimal fragment thereof such as a Fv or a single chain (scFv) construct.

Binding of the bispecific molecules to their specific targets can be confirmed using art-recognized methods, such as enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest.

Methods for making bispecific antibodies are known in the art. Traditionally, the recombinant production of bispecific antibodies is based on the co-expression of two immunoglobulin heavy-chain/light-chain pairs, where the two heavy chains have different specificities [Milstein and Cuello, Nature, 305:537-539 (1983)]. Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various technique for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. As one nonlimiting example, trispecific antibodies can be prepared. See, e.g., Tutt et al., J. Immunol. 147:60 (1991).

d. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

e. Effector Function Engineering

It may be desirable to modify the antibody encompassed by the present invention with respect to effector function, so as to enhance, e.g., the effectiveness of the anti-NTPDase3 antibody in treating cancer. For example, cysteine residue(s) may be introduced into the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). In certain preferred embodiments, the effector function being engineered is the ability of the anti-NTPDase3 antibody to induce FcγRIII binding-dependent removal (such as by anti-NTPDase3 antibody mediated target cytosis) of NTPDase3 from immune cells, i.e., without depleting the immune cell population by way of cell killing.

Homodimeric antibodies with enhanced anti-tumor activity may also be prepared using heterobifunctional cross-linkers as described in Wolff et al. Cancer Research, 53: 2560-2565 (1993). Alternatively, an antibody can be engineered that has dual Fc regions and may thereby have enhanced NTPDase3 trogocytosis capabilities. See Stevenson et al., Anti-Cancer Drug Design, 3: 219-230 (1989).

f. Representative Anti-NTPDase3 Antibody Sequences

In certain embodiments, the anti-NTPDase3 antibody is a fully human antibody, such as generated from a human antibody library. An exemplary fully human anti-NTPDase3 antibody is clone PBI #30, the heavy and light variable domains (VH and VL) sequences provided as follows:

| | Nucleic Acid Sequence | Amino Acid Sequence |
|---|---|---|
| VH domain | SEQ ID No. 1 (VH) | SEQ ID No. 2 (VH) |
| VL domain | SEQ ID No. 3 (VL) | SEQ ID No. 4 (VL) |

The full length sequences for the heavy and light chains of clone PBI #30, including constant domains are provided as follows:

| | Nucleic Acid Sequence | Amino Acid Sequence |
|---|---|---|
| VH domain | SEQ ID No. 5 (VH) | SEQ ID No. 6 (VH) |
| VL domain | SEQ ID No. 7 (VL) | SEQ ID No. 8 (VL) |

For the PBI #30 clone, the CDRs (amino acid sequences) for each of the VH and VL domains are:

| | CDR1 | CDR2 | CDR3 |
|---|---|---|---|
| VH | SEQ ID No. 45 | SEQ ID No. 46 | SEQ ID No. 47 |
| VL | SEQ ID No. 48 | SEQ ID No. 49 | SEQ ID No. 50 |

In certain embodiments, the anti-NTPDase3 antibody is an engineered fully human antibody e.g. affinity maturation variants. An exemplary engineered fully human anti-NTPDase3 antibody is clone PBI #30 affinity maturation variants through introducing point mutations into the antibody CDR regions, without disrupting the antibody framework region.

Exemplary sequences of the CDRs of four PBI #30 affinity maturation variant clones for each of the VH and VL domains (amino acid sequences) are provided as follows in Tables 2A-2D:

TABLE 2A

| PBI#30af4 | CDR1 | CDR2 | CDR3 |
|---|---|---|---|
| VH | SEQ ID No. 51 | SEQ ID No. 52 | SEQ ID No. 53 |
| VL | SEQ ID No. 54 | SEQ ID No. 55 | SEQ ID No. 56 |

TABLE 2B

| PBI#30af6 | CDR1 | CDR2 | CDR3 |
|---|---|---|---|
| VH | SEQ ID No. 57 | SEQ ID No. 58 | SEQ ID No. 59 |
| VL* | SEQ ID No. 60 | SEQ ID No. 61 | SEQ ID No. 62 |

Note:
*For the PBI#30af6 maturation variant, due to a PCR amplication error, one amino acid residue in the framework of VL domain was changed from Val(ine) to I(soleucine), but this change has no impacts on function results.

TABLE 2C

| PBI#30af7 | CDR1 | CDR2 | CDR3 |
|---|---|---|---|
| VH | SEQ ID No. 63 | SEQ ID No. 64 | SEQ ID No. 65 |
| VL | SEQ ID No. 66 | SEQ ID No. 67 | SEQ ID No. 68 |

TABLE 2D

| PBI#30af8 | CDR1 | CDR2 | CDR3 |
|---|---|---|---|
| VH | SEQ ID No. 69 | SEQ ID No. 70 | SEQ ID No. 71 |
| VL | SEQ ID No. 72 | SEQ ID No. 73 | SEQ ID No. 74 |

In some embodiments, the anti-NTPDase3 antibody or antigen-binding fragment thereof comprises at least one heavy chain variable domain that is at least 60% identical to a VH domain sequence described herein, such as SEQ ID No. 2 and even more preferably at least 65%, 70%, 75%, 80%, 85% or even 90% identical to a VH domain sequence described herein, such as SEQ ID No. 2, and able to specifically bind human NTPDase3.

In some embodiments, the anti-NTPDase3 antibody or antigen-binding fragment thereof comprises at least one light chain variable domain that is at least 60% identical to a VL domain sequence described herein, such as SEQ ID No. 4, and even more preferably at least 65%, 70%, 75%, 80%, 85% or even 90% identical to a VL domain sequence described herein, such as SEQ ID No. 4 and able to specifically bind human NTPDase3.

In certain embodiments, the anti-NTPDase3 antibody is a human or a humanized antibody comprising a VH domain having human framework sequences associated with CDRs of a VH domain described herein, such as in SEQ ID Nos. 45, 46 and 47, and the CDRs of the corresponding VL domain described herein, such as in SEQ ID Nos. 48, 49 and 50. The CDRs of an anti-NTPDase3 antibody or antigen-binding fragment thereof are preferably identical to CDRs described herein, but may vary by 1, 2 or 3 amino acids across each CDR so long as the resulting antibody binds human NTPDase3.

In certain embodiments, the heavy and light chains of the anti-NTPDase3 antibody have variable domains that can be encoded by a nucleic acid which is identical to, or hybridizes under stringent conditions (such as the 6× sodium chloride/sodium citrate (SSC) at 45° C., and washing in 0.2×SSC/0.1% SDS at 50-65° C.) to VH and VL domain (correspondingly) coding sequences described herein, such as those shown in SEQ ID No. 1 (VH) and SEQ ID No. 3 (VL).

In some embodiments, anti-NTPDase3 antibodies were generated in rabbits, and the variable domains of the heavy and light chains of these antibodies are rabbit sequence.

Exemplary sequences for the VH and VL domains of rabbit anti-NTPDase3 antibodies are:

| | Nucleic Acid | Amino Acid | |
|---|---|---|---|
| Clone | Sequence | Sequence | CDR Sequences |
| 3E9 | SEQ ID No. 9 (VH) | SEQ ID No. 10 (VH) | SEQ ID NO: 10 (VH) CDRs |
| | SEQ ID No. 11 (VL) | SEQ ID No. 12 (VL) | CDR1: GFSFSSSDW |
| | | | CDR2: IYAGSIGGA |
| | | | CDR3: ARSSSNGRLKLDQFDL |
| | | | SEQ ID NO: 12 (VL) CDRs |
| | | | CDR1: QSIGGW |
| | | | CDR2: KAS |
| | | | CDR3: QQGSAADVANT |
| 4F9 | SEQ ID No. 13 (VH) | SEQ ID No. 14 (VH) | SEQ ID NO: 14 (VH) CDRs |
| | SEQ ID No. 15 (VL) | SEQ ID No. 16 (VL) | CDR1: GFSLSSYA |
| | | | CDR2: ITYNGNT |
| | | | CDR3: ARARYGGYSTNSYYLNI |
| | | | SEQ ID NO: 16 (VL) CDRs |
| | | | CDR1: ENIYRI |
| | | | CDR2: GAS |
| | | | CDR3: QGVLYNSNDST |
| 8E1 | SEQ ID No. 17 (VH) | SEQ ID No. 18 (VH) | SEQ ID NO: 18 (VH) CDRs |
| | SEQ ID No. 19 (VL) | SEQ ID No. 20 (VL) | CDR1: GFSLSSYA |
| | | | CDR2: ISYSGNT |
| | | | CDR3: ARARYGGYSTNSYYLNI |
| | | | SEQ ID NO: 20 (VL) CDRs |
| | | | CDR1: ENIYRV |
| | | | CDR2: GAS |
| | | | CDR3: QGVVYNSDDSA |
| 16D4 | SEQ ID No. 21 (VH) | SEQ ID No. 22 (VH) | SEQ ID NO: 22 (VH) CDRs |
| | SEQ ID No. 23 (VL) | SEQ ID No. 24 (VL) | CDR1: GFSLSSYA |
| | | | CDR2: ITYNGGT |
| | | | CDR3: ARARYGGYSTNSYYLNI |
| | | | SEQ ID NO: 24 (VL) CDRs |
| | | | CDR1: ENIYRV |
| | | | CDR2: GAS |
| | | | CDR3: QGVLYDNNDST |
| 37H1 | SEQ ID No. 25 (VH) | SEQ ID No. 26 (VH) | SEQ ID NO: 26 (VH) CDRs |
| | SEQ ID No. 27 (VL) | SEQ ID No. 28 (VL) | CDR1: GFSLSSYA |
| | | | CDR2: ITYNGAT |
| | | | CDR3: ARARYGGYSTNSYYLNI |
| | | | SEQ ID NO: 28 (VL) CDRs |
| | | | CDR1: ENIYRI |
| | | | CDR2: GAS |
| | | | CDR3: QGVLWNSNDST |
| 38D5 | SEQ ID No. 29 (VH) | SEQ ID No. 30 (VH) | SEQ ID NO: 30 (VH) CDRs |
| | SEQ ID No. 31 (VL) | SEQ ID No. 32 (VL) | CDR1: GIDFNNYG |
| | | | CDR2: KYPGFGIR |
| | | | CDR3: ARGARYRHDDYGALNL |
| | | | SEQ ID NO: 32 (VL) CDRs |
| | | | CDR1: QSVTNL |
| | | | CDR2: GAS |
| | | | CDR3: QSGYYSANT |
| 38D12 | SEQ ID No. 33 (VH) | SEQ ID No. 34 (VH) | SEQ ID NO: 34 (VH) CDRs |
| | SEQ ID No. 35 (VH) | SEQ ID No. 36 (VH) | CDR1: GFSLSSYV |
| | | | CDR2: ISSSGST |
| | | | CDR3: ARGADGAFISDYFNI |
| | | | SEQ ID NO: 36 (VL) CDRs |
| | | | CDR1: ENIDSW |
| | | | CDR2: YAS |
| | | | CDR3: QSYDTISDYGVG |
| 42D8 | SEQ ID No. 37 (VH) | SEQ ID No. 38 (VH) | SEQ ID NO: 38 (VH) CDRs |
| | SEQ ID No. 39(VL) | SEQ ID No. 40 (VL) | CDR1: GFSLSSYA |
| | | | CDR2: ITYNSNT |
| | | | CDR3: ARARYGGYSTNSYYLNI |
| | | | SEQ ID NO: 40 (VL) CDRs |
| | | | CDR1: ENIYRI |
| | | | CDR2: GAS |
| | | | CDR3: QGVLYNSNDST |
| 44H5 | SEQ ID No. 41 (VH) | SEQ ID No. 42 (VH) | SEQ ID NO: 42 (VH) CDRs |
| | SEQ ID No. 43 (VL) | SEQ ID No. 44 (VL) | CDR1: GFDSSSTYY |
| | | | CDR2: IYTGGLT |
| | | | CDR3: ASYIGSRFYMPGFSL |
| | | | SEQ ID NO: 44 (VL) CDRs |
| | | | CDR1: ESIYSNNR |
| | | | CDR2: LAS |
| | | | CDR3: AGYKGSSTDGTA |

In some embodiments, anti-NTPDase3 antibodies were humanized rabbit antibodies (through humanization of the antibody framework region without disrupting the antibody CDR regions), and the variable domains of the heavy and light chains of these antibodies are human sequences. Exemplary sequences for the VH and VL domains of humanized anti-NTPDase3 antibodies are provided as follows:

| | Nucleic Acid | Amino Acid | |
|---|---|---|---|
| Clone | Sequence | Sequence | CDR Sequences |
| h8E1 | SEQ ID No. 75 (VH) | SEQ ID No. 76 (VH) | SEQ ID NO: 76 (VH) CDRs |
| | SEQ ID No. 77 (VL) | SEQ ID No. 78 (VL) | CDR1: GFSLSSYA |
| | | | CDR2: ISYSGNT |
| | | | CDR3: ARARYGGYSTNSYYLNI |
| | | | SEQ ID NO: 78 (VL) CDRs |
| | | | CDR1: ENIYRV |
| | | | CDR2: GAS |
| | | | CDR3: QGVVYNSDDSA |
| h38D5 | SEQ ID No. 79 (VH) | SEQ ID No. 80 (VH) | SEQ ID NO: 80 (VH) CDRs |
| | SEQ ID No. 81 (VL) | SEQ ID No. 82 (VL) | CDR1: GIDFNNYG |
| | | | CDR2: KYPGFGIR |
| | | | CDR3: ARGARYRHDDYGALNL |
| | | | SEQ ID NO: 82 (VL) CDRs |
| | | | CDR1: QSVTNL |
| | | | CDR2: GAS |
| | | | CDR3: QSGYYSANT |

In some embodiments, anti-NTPDase3 antibodies were further engineered humanized antibodies (through introducing point mutation in the antibody backbone sequence without disrupting the variable domains of the heavy and light chains of the antibody to further reduce the potential immunogenicity to humans for therapeutic use. Exemplary point mutations introduced into the VH and VL domains of the humanized 38D5 clone are shown in Table 3 below:

TABLE 3

| Engineered | | | | | | | | |
|---|---|---|---|---|---|---|---|---|
| region | LC.1 | HC.1 | HC.2 | HC.3 | HC.4 | HC.5 | HC.6 | HC.7 |
| Point | P43A | Q2V | 148V | A49S | S71R | V78L | F91Y | P105R |
| mutation | | | | | | | | |

Note:
LC and HC stands for light chain and heavy chain of the humanized 38D5 clone, respectively.

In some embodiments, an anti-NTPDase3 antibody, or antigen-binding fragment thereof, provided herein promotes: (i) complement dependent cytotoxicity (CDC) activity against NTPDase3+ cells; (ii) antibody-mediated target cytosis of NTPDase3 on NTPDase3+ immune cells (preferably M2 macrophage); (iii) NTPDase3 enzymatic activity inhibition on NTPDase3+ cells; and/or (iv) binding to NTPDase3 in a manner that is competitive, non-competitive, or partially competitive with an NTPDase3 monoclonal antibody clone binding to NTPDase3, wherein the NTPDase3 monoclonal antibody clone is selected from the group consisting of PBI #30 and its affinity maturation variants, 3E9, 4F9, 8E1 and its humanized counterparts, 16D4, 37H1, 38D5 and its humanized counterparts with or without point mutation in their backbone sequences, 38D12, 42D8, and 44H5.

Representative anti-NTPDase3 antibody sequences described above according to sequence identification number correspond to the following (where line <210> corresponds to sequence identification number, such as <210>1 corresponds to SEQ ID NO: 1, <210>2 corresponds to SEQ ID NO: 2, etc.):

| <210> 1 | |
|---|---|
| <211> 384 | |
| <212> DNA | |
| <213> Homo sapiens | |
| <220> | |
| <221> CDS | |
| <222> (1) . . . (384) | |
| <400> 1 | |
| cag gtg cag ctg cag gag tcg ggg gga ggt ttg ata cag cct ggg gga | 48 |
| Gln Val Gln Leu Gln Glu Ser Gly Gly Gly Leu Ile Gln Pro Gly Gly | |
| 1 5 10 15 | |
| tcc ctg aga ctc tcc tgc gca gcc tct gga ttc acc tct agt tct agt | 96 |
| Ser Leu Arg Leu Ser Cys Ala Ala Ser Gly Phe Thr Ser Ser Ser Ser | |
| 20 25 30 | |
| gat tat gcc atg agt tgg gtc cgc cag gct cca ggg aag ggg ctg gag | 144 |
| Asp Tyr Ala Met Ser Trp Val Arg Gln Ala Pro Gly Lys Gly Leu Glu | |
| 35 40 45 | |
| tgg gtc tcg tct att agt gga agt ggc ggt agc aca tac tac gca gac | 192 |
| Trp Val Ser Ser Ile Ser Gly Ser Gly Gly Ser Thr Tyr Tyr Ala Asp | |
| 50 55 60 | |
| tcc gtg aag ggc cgc ttc acc atc tcc aga gac aat tcc aag aac aca | 240 |
| Ser Val Lys Gly Arg Phe Thr Ile Ser Arg Asp Asn Ser Lys Asn Thr | |
| 65 70 75 80 | |
| ctc tat gtg caa atg acc agc ctg aca gcc gag gac acg gcc gta tat | 288 |
| Leu Tyr Val Gln Met Thr Ser Leu Thr Ala Glu Asp Thr Ala Val Tyr | |
| 85 90 95 | |
| tac tgt gcg aaa gtc gtc gga tac agt atg tac gac tac tac tac cac | 336 |
| Tyr Cys Ala Lys Val Val Gly Tyr Ser Met Tyr Asp Tyr Tyr Tyr His | |
| 100 105 110 | |
| tac gct ttg gac gtc tgg ggc caa ggg acc acg gtc acc gtc tcc tca | 384 |
| Tyr Ala Leu Asp Val Trp Gly Gln Gly Thr Thr Val Thr Val Ser Ser | |
| 115 120 125 | |
| <210> 2 | |
| <211> 128 | |
| <212> PRT | |
| <213> Homo sapiens | |
| <400> 2 | |
| Gln Val Gln Leu Gln Glu Ser Gly Gly Gly Leu Ile Gln Pro Gly Gly | |
| 1 5 10 15 | |
| Ser Leu Arg Leu Ser Cys Ala Ala Ser Gly Phe Thr Ser Ser Ser Ser | |
| 20 25 30 | |
| Asp Tyr Ala Met Ser Trp Val Arg Gln Ala Pro Gly Lys Gly Leu Glu | |
| 35 40 45 | |
| Trp Val Ser Ser Ile Ser Gly Ser Gly Gly Ser Thr Tyr Tyr Ala Asp | |
| 50 55 60 | |
| Ser Val Lys Gly Arg Phe Thr Ile Ser Arg Asp Asn Ser Lys Asn Thr | |
| 65 70 75 80 | |
| Leu Tyr Val Gln Met Thr Ser Leu Thr Ala Glu Asp Thr Ala Val Tyr | |
| 85 90 95 | |
| Tyr Cys Ala Lys Val Val Gly Tyr Ser Met Tyr Asp Tyr Tyr Tyr His | |
| 100 105 110 | |
| Tyr Ala Leu Asp Val Trp Gly Gln Gly Thr Thr Val Thr Val Ser Ser | |
| 115 120 125 | |
| <210> 3 | |
| <211> 324 | |
| <212> DNA | |
| <213> Homo sapiens | |
| <220> | |
| <221> CDS | |
| <222> (1) . . . (324) | |
| <400> 3 | |
| cag cct gtg ctg act cag cca cct tca gcg tct ggg acc ccc ggg cag | 48 |
| Gln Pro Val Leu Thr Gln Pro Pro Ser Ala Ser Gly Thr Pro Gly Gln | |
| 1 5 10 15 | |
| agg gtc acc atc tct tgt tct gga agc agc tcc aac atc gga agt aat | 96 |
| Arg Val Thr Ile Ser Cys Ser Gly Ser Ser Ser Asn Ile Gly Ser Asn | |
| 20 25 30 | |
| act gta aac tgg tac cag cag ctc cca gga acg gcc ccc aaa ctc ctc | 144 |
| Thr Val Asn Trp Tyr Gln Gln Leu Pro Gly Thr Ala Pro Lys Leu Leu | |
| 35 40 45 | |
| atc tat agt aat aat cag cgg ccc tca ggg gtc cct gac cga ttc tct | 192 |
| Ile Tyr Ser Asn Asn Gln Arg Pro Ser Gly Val Pro Asp Arg Phe Ser | |
| 50 55 60 | |
| ggc tcc aag tct ggc acc tca gcc tcc ctc acc atc tct gga ctg aag | 240 |
| Gly Ser Lys Ser Gly Thr Ser Ala Ser Leu Thr Ile Ser Gly Leu Lys | |
| 65 70 75 80 | |
| act gag gac gag gct ggc tac tac tgt cag tct tat gat acc agc aat | 288 |
| Thr Glu Asp Glu Ala Gly Tyr Tyr Cys Gln Ser Tyr Asp Thr Ser Asn | |
| 85 90 95 | |
| gtg gta ttc ggc gga ggg acc aag gtg acc gtc ctc | 324 |
| Val Val Phe Gly Gly Gly Thr Lys Val Thr Val Leu | |
| 100 105 | |
| <210> 4 | |
| <211> 108 | |
| <212> PRT | |
| <213> Homo sapiens | |
| <400> 4 | |
| Gln Pro Val Leu Thr Gln Pro Pro Ser Ala Ser Gly Thr Pro Gly Gln | |
| 1 5 10 15 | |
| Arg Val Thr Ile Ser Cys Ser Gly Ser Ser Ser Asn Ile Gly Ser Asn | |
| 20 25 30 | |
| Thr Val Asn Trp Tyr Gln Gln Leu Pro Gly Thr Ala Pro Lys Leu Leu | |
| 35 40 45 | |
| Ile Tyr Ser Asn Asn Gln Arg Pro Ser Gly Val Pro Asp Arg Phe Ser | |
| 50 55 60 | |
| Gly Ser Lys Ser Gly Thr Ser Ala Ser Leu Thr Ile Ser Gly Leu Lys | |
| 65 70 75 80 | |
| Thr Glu Asp Glu Ala Gly Tyr Tyr Cys Gln Ser Tyr Asp Thr Ser Asn | |
| 85 90 95 | |
| Val Val Phe Gly Gly Gly Thr Lys Val Thr Val Leu | |
| 100 105 | |
| <210> 5 | |
| <211> 1377 | |
| <212> DNA | |
| <213> Homo sapiens | |
| <220> | |
| <221> CDS | |
| <222> (1) . . . (1377) | |
| <400> 5 | |
| cag gtg cag ctg cag gag tcg ggg gga ggt ttg ata cag cct ggg gga | 48 |
| Gln Val Gln Leu Gln Glu Ser Gly Gly Gly Leu Ile Gln Pro Gly Gly | |
| 1 5 10 15 | |
| tcc ctg aga ctc tcc tgc gca gcc tct gga ttc acc tct agt tct agt | 96 |
| Ser Leu Arg Leu Ser Cys Ala Ala Ser Gly Phe Thr Ser Ser Ser Ser | |
| 20 25 30 | |
| gat tat gcc atg agt tgg gtc cgc cag gct cca ggg aag ggg ctg gag | 144 |
| Asp Tyr Ala Met Ser Trp Val Arg Gln Ala Pro Gly Lys Gly Leu Glu | |
| 35 40 45 | |
| tgg gtc tcg tct att agt gga agt ggc ggt agc aca tac tac gca gac | 192 |
| Trp Val Ser Ser Ile Ser Gly Ser Gly Gly Ser Thr Tyr Tyr Ala Asp | |
| 50 55 60 | |
| tcc gtg aag ggc cgc ttc acc atc tcc aga gac aat tcc aag aac aca | 240 |
| Ser Val Lys Gly Arg Phe Thr Ile Ser Arg Asp Asn Ser Lys Asn Thr | |
| 65 70 75 80 | |
| ctc tat gtg caa atg acc agc ctg aca gcc gag gac acg gcc gta tat | 288 |
| Leu Tyr Val Gln Mel Thr Ser Leu Thr Ala Glu Asp Thr Ala Val Tyr | |
| 85 90 95 | |
| tac tgt gcg aaa gtc gtc gga tac agt atg tac gac tac tac tac cac | 336 |
| Tyr Cys Ala Lys Val Val Gly Tyr Ser Met Tyr Asp Tyr Tyr Tyr His | |
| 100 105 110 | |
| tac gct ttg gac gtc tgg ggc caa ggg acc acg gtc acc gtc tcc tca | 384 |
| Tyr Ala Leu Asp Val Trp Gly Gln Gly Thr Thr Val Thr Val Ser Ser | |
| 115 120 125 | |
| gcc tcc act aag ggc cca tcc gtg ttc cca ctg gca ccc tct agt aag | 432 |
| Ala Ser Thr Lys Gly Pro Ser Val Phe Pro Leu Ala Pro Ser Ser Lys | |
| 130 135 140 | |
| agc aca tct ggg ggt act gcc gct ctg gga tgt ctg gtg aag gat tac | 480 |
| Ser Thr Ser Gly Gly Thr Ala Ala Leu Gly Cys Leu Val Lys Asp Tyr | |
| 145 150 155 160 | |
| ttc cca gag cca gtc acc gtg tcc tgg aac agc ggg gcc ctg act tcc | 528 |
| Phe Pro Glu Pro Val Thr Val Ser Trp Asn Ser Gly Ala Leu Thr Ser | |
| 165 170 175 | |
| ggt gtc cat acc ttt cca gct gtg ctg cag tca tcc ggc ctg tac agc | 576 |
| Gly Val His Thr Phe Pro Ala Val Leu Gln Ser Ser Gly Leu Tyr Ser | |
| 180 185 190 | |
| ctg agc tct gtg gtc acc gtc ccc agt tca tcc ctg gga aca cag act | 624 |
| Leu Ser Ser Val Val Thr Val Pro Ser Ser Ser Leu Gly Thr Gln Thr | |
| 195 200 205 | |
| tat atc tgc aac gtg aat cac aag cca tcc aat aca aaa gtc gac aag | 672 |
| Tyr Ile Cys Asn Val Asn His Lys Pro Ser Asn Thr Lys Val Asp Lys | |
| 210 215 220 | |
| aaa gtg gaa ccc aag agc tgt gat aaa acc cat aca tgc ccc cct tgt | 720 |
| Lys Val Glu Pro Lys Ser Cys Asp Lys Thr His Thr Cys Pro Pro Cys | |
| 225 230 235 240 | |
| cct gct cca gag ctg ctg gga gga cca tcc gtg ttc ctg ttt cca ccc | 768 |
| Pro Ala Pro Glu Leu Leu Gly Gly Pro Ser Val Phe Leu Phe Pro Pro | |
| 245 250 255 | |
| aag cct aaa gac act ctg atg att tct cga acc ccc gaa gtc aca tgc | 816 |
| Lys Pro Lys Asp Thr Leu Met Ile Ser Arg Thr Pro Glu Val Thr Cys | |
| 260 265 270 | |
| gtg gtc gtg gac gtg tcc cac gag gat cct gaa gtc aag ttc aac tgg | 864 |
| Val Val Val Asp Val Ser His Glu Asp Pro Glu Val Lys Phe Asn Trp | |
| 275 280 285 | |
| tac gtg gat ggc gtc gag gtg cat aat gcc aag aca aaa cca cga gag | 912 |
| Tyr Val Asp Gly Val Glu Val His Asn Ala Lys Thr Lys Pro Arg Glu | |
| 290 295 300 | |
| gaa cag tac aac agt acc tat cgt gtc gtg tca gtc ctg aca gtg ctg | 960 |
| Glu Gln Tyr Asn Ser Thr Tyr Arg Val Val Ser Val Leu Thr Val Leu | |
| 305 310 315 320 | |
| cac cag gac tgg ctg aac ggg aag gaa tat aag tgc aaa gtg agc aat | 1008 |
| His Gln Asp Trp Leu Asn Gly Lys Glu Tyr Lys Cys Lys Val Ser Asn | |
| 325 330 335 | |
| aag gca ctg ccc gcc cct atc gag aaa aca att tct aag gct aaa gga | 1056 |
| Lys Ala Leu Pro Ala Pro Ile Glu Lys Thr Ile Ser Lys Ala Lys Gly | |
| 340 345 350 | |
| cag cct agg gaa cca cag gtg tac act ctg cct cca tca cgg gac gag | 1104 |
| Gln Pro Arg Glu Pro Gln Val Tyr Thr Leu Pro Pro Ser Arg Asp Glu | |
| 355 360 365 | |
| ctg aca aag aac cag gtc agt ctg act tgt ctg gtg aaa ggg ttc tat | 1152 |
| Leu Thr Lys Asn Gln Val Ser Leu Thr Cys Leu Val Lys Gly Phe Tyr | |
| 370 375 380 | |
| cct tct gat atc gcc gtg gag tgg gaa agt aat ggt cag cca gag aac | 1200 |
| Pro Ser Asp Ile Ala Val Glu Trp Glu Ser Asn Gly Gln Pro Glu Asn | |
| 385 390 395 400 | |
| aat tac aag acc aca ccc cct gtc ctg gac tct gat ggg agt ttc ttt | 1248 |
| Asn Tyr Lys Thr Thr Pro Pro Val Leu Asp Ser Asp Gly Ser Phe Phe | |
| 405 410 415 | |
| ctg tat tcc aag ctg acc gtg gat aaa agc cgg tgg cag cag ggt aat | 1296 |
| Leu Tyr Ser Lys Leu Thr Val Asp Lys Ser Arg Trp Gln Gln Gly Asn | |
| 420 425 430 | |
| gtc ttt agt tgt tca gtg atg cac gag gca ctg cac aat cac tac acc | 1344 |
| Val Phe Ser Cys Ser Val Met His Glu Ala Leu His Asn His Tyr Thr | |
| 435 440 445 | |
| cag aaa tca ctg tca ctg tca cca ggt aaa tga | 1377 |
| Gln Lys Ser Leu Ser Leu Ser Pro Gly Lys | |
| 450 455 | |
| <210> 6 | |
| <211> 458 | |
| <212> PRT | |
| <213> Homo sapiens | |
| <400> 6 | |
| Gln Val Gln Leu Gln Glu Ser Gly Gly Gly Leu Ile Gln Pro Gly Gly | |
| 1 5 10 15 | |
| Ser Leu Arg Leu Ser Cys Ala Ala Ser Gly Phe Thr Ser Ser Ser Ser | |
| 20 25 30 | |
| Asp Tyr Ala Met Ser Trp Val Arg Gln Ala Pro Gly Lys Gly Leu Glu | |
| 35 40 45 | |
| Trp Val Ser Ser Ile Ser Gly Ser Gly Gly Ser Thr Tyr Tyr Ala Asp | |
| 50 55 60 | |
| Ser Val Lys Gly Arg Phe Thr Ile Ser Arg Asp Asn Ser Lys Asn Thr | |
| 65 70 75 80 | |
| Leu Tyr Val Gln Met Thr Ser Leu Thr Ala Glu Asp Thr Ala Val Tyr | |
| 85 90 95 | |
| Tyr Cys Ala Lys Val Val Gly Tyr Ser Mel Tyr Asp Tyr Tyr Tyr His | |
| 100 105 110 | |
| Tyr Ala Leu Asp Val Trp Gly Gln Gly Thr Thr Val Thr Val Ser Ser | |
| 115 120 125 | |
| Ala Ser Thr Lys Gly Pro Ser Val Phe Pro Leu Ala Pro Ser Ser Lys | |
| 130 135 140 | |
| Ser Thr Ser Gly Gly Thr Ala Ala Leu Gly Cys Leu Val Lys Asp Tyr | |
| 145 150 155 160 | |
| Phe Pro Glu Pro Val Thr Val Ser Trp Asn Ser Gly Ala Leu Thr Ser | |
| 165 170 175 | |
| Gly Val His Thr Phe Pro Ala Val Leu Gln Ser Ser Gly Leu Tyr Ser | |
| 180 185 190 | |
| Leu Ser Ser Val Val Thr Val Pro Ser Ser Ser Leu Gly Thr Gln Thr | |
| 195 200 205 | |
| Tyr Ile Cys Asn Val Asn His Lys Pro Ser Asn Thr Lys Val Asp Lys | |
| 210 215 220 | |
| Lys Val Glu Pro Lys Ser Cys Asp Lys Thr His Thr Cys Pro Pro Cys | |
| 225 230 235 240 | |
| Pro Ala Pro Glu Leu Leu Gly Gly Pro Ser Val Phe Leu Phe Pro Pro | |
| 245 250 255 | |
| Lys Pro Lys Asp Thr Leu Met Ile Ser Arg Thr Pro Glu Val Thr Cys | |
| 260 265 270 | |
| Val Val Val Asp Val Ser His Glu Asp Pro Glu Val Lys Phe Asn Trp | |
| 275 280 285 | |
| Tyr Val Asp Gly Val Glu Val His Asn Ala Lys Thr Lys Pro Arg Glu | |
| 290 295 300 | |
| Glu Gln Tyr Asn Ser Thr Tyr Arg Val Val Ser Val Leu Thr Val Leu | |
| 305 310 315 320 | |
| His Gln Asp Trp Leu Asn Gly Lys Glu Tyr Lys Cys Lys Val Ser Asn | |
| 325 330 335 | |
| Lys Ala Leu Pro Ala Pro Ile Glu Lys Thr Ile Ser Lys Ala Lys Gly | |
| 340 345 350 | |
| Gln Pro Arg Glu Pro Gln Val Tyr Thr Leu Pro Pro Ser Arg Asp Glu | |
| 355 360 365 | |
| Leu Thr Lys Asn Gln Val Ser Leu Thr Cys Leu Val Lys Gly Phe Tyr | |
| 370 375 380 | |
| Pro Ser Asp Ile Ala Val Glu Trp Glu Ser Asn Gly Gln Pro Glu Asn | |
| 385 390 395 400 | |
| Asn Tyr Lys Thr Thr Pro Pro Val Leu Asp Ser Asp Gly Ser Phe Phe | |
| 405 410 415 | |
| Leu Tyr Ser Lys Leu Thr Val Asp Lys Ser Arg Trp Gln Gln Gly Asn | |
| 420 425 430 | |
| Val Phe Ser Cys Ser Val Met His Glu Ala Leu His Asn His Tyr Thr | |
| 435 440 445 | |
| Gln Lys Ser Leu Ser Leu Ser Pro Gly Lys | |
| 450 455 | |
| <210> 7 | |
| <211> 645 | |
| <212> DNA | |
| <213> Homo sapiens | |
| <220> | |
| <221> CDS | |
| <222> (1) . . . (645) | |
| <400> 7 | |
| cag cct gtg ctg act cag cca cct tra geg tct ggg acc ccc ggg cag | 48 |
| Gln Pro Val Leu Thr Gln Pro Pro Ser Ala Ser Gly Thr Pro Gly Gln | |
| 1 5 10 15 | |
| agg gtc acc atc tct tgt tct gga agc agc tcc aac atc gga agt aat | 96 |
| Arg Val Thr Ile Ser Cys Ser Gly Ser Ser Ser Asn Ile Gly Ser Asn | |
| 20 25 30 | |
| act gta aac tgg tac cag cag ctc cca gga acg gcc ccc aaa ctc ctc | 144 |
| Thr Val Asn Trp Tyr Gln Gln Leu Pro Gly Thr Ala Pro Lys Leu Leu | |
| 35 40 45 | |
| atc tat agt aat aat cag cgg ccc tca ggg gtc cct gac cga ttc tct | 192 |
| Ile Tyr Ser Asn Asn Gln Arg Pro Ser Gly Val Pro Asp Arg Phe Ser | |
| 50 55 60 | |
| ggc tcc aag tct ggc acc tca gcc tcc ctc acc atc tct gga ctg aag | 240 |
| Gly Ser Lys Ser Gly Thr Ser Ala Ser Leu Thr Ile Ser Gly Leu Lys | |
| 65 70 75 80 | |
| act gag gac gag gct ggc tac tac tgt cag tct tat gat acc agc aat | 288 |
| Thr Glu Asp Glu Ala Gly Tyr Tyr Cys Gln Ser Tyr Asp Thr Ser Asn | |
| 85 90 95 | |
| gtg gta ttc ggc gga ggg acc aag gtg acc gtc ctc ggc cag cct aaa | 336 |
| Val Val Phe Gly Gly Gly Thr Lys Val Thr Val Leu Gly Gln Pro Lys | |
| 100 105 110 | |
| gcc aac cca aca gtg act ctg ttt cca ccc tcc agc gag gaa ctg cag | 384 |
| Ala Asn Pro Thr Val Thr Leu Phe Pro Pro Ser Ser Glu Glu Leu Gln | |
| 115 120 125 | |
| gct aat aag gca act ctg gtc tgt ctg att tct gac ttc tac cct gga | 432 |
| Ala Asn Lys Ala Thr Leu Val Cys Leu Ile Ser Asp Phe Tyr Pro Gly | |
| 130 135 140 | |
| gct gtg acc gtc gca tgg aag gct gat ggt tcc ccc gtg aaa gca ggc | 480 |
| Ala Val Thr Val Ala Trp Lys Ala Asp Gly Ser Pro Val Lys Ala Gly | |
| 145 150 155 160 | |
| gtc gag acc aca aag cct tct aaa cag agt aac aat aag tac gcc gct | 528 |
| Val Glu Thr Thr Lys Pro Ser Lys Gln Ser Asn Asn Lys Tyr Ala Ala | |
| 165 170 175 | |
| tct agt tat ctg tct ctg aca cca gaa cag tgg aaa agt cat agg tcc | 576 |
| Ser Ser Tyr Leu Ser Leu Thr Pro Glu Gln Trp Lys Ser His Arg Ser | |
| 180 185 190 | |
| tat agc tgt cag gtc act cac gaa ggc tca act gtg gaa aaa act gtg | 624 |
| Tyr Ser Cys Gln Val Thr His Glu Gly Ser Thr Val Glu Lys Thr Val | |
| 195 200 205 | |
| gct cct acc gaa tgc tca tag | 645 |
| Ala Pro Thr Glu Cys Ser | |
| 210 | |
| <210> 8 | |
| <211> 214 | |
| <212> PRT | |
| <213> Homo sapiens | |
| <400> 8 | |
| Gln Pro Val Leu Thr Gln Pro Pro Ser Ala Ser Gly Thr Pro Gly Gln | |
| 1 5 10 15 | |
| Arg Val Thr Ile Ser Cys Ser Gly Ser Ser Ser Asn Ile Gly Ser Asn | |
| 20 25 30 | |
| Thr Val Asn Trp Tyr Gln Gln Leu Pro Gly Thr Ala Pro Lys Leu Leu | |
| 35 40 45 | |
| Ile Tyr Ser Asn Asn Gln Arg Pro Ser Gly Val Pro Asp Arg Phe Ser | |
| 50 55 60 | |
| Gly Ser Lys Ser Gly Thr Ser Ala Ser Leu Thr Ile Ser Gly Leu Lys | |
| 65 70 75 80 | |
| Thr Glu Asp Glu Ala Gly Tyr Tyr Cys Gln Ser Tyr Asp Thr Ser Asn | |
| 85 90 95 | |
| Val Val Phe Gly Gly Gly Thr Lys Val Thr Val Leu Gly Gln Pro Lys | |
| 100 105 110 | |
| Ala Asn Pro Thr Val Thr Leu Phe Pro Pro Ser Ser Glu Glu Leu Gln | |
| 115 120 125 | |
| Ala Asn Lys Ala Thr Leu Val Cys Leu Ile Ser Asp Phe Tyr Pro Gly | |
| 130 135 140 | |
| Ala Val Thr Val Ala Trp Lys Ala Asp Gly Ser Pro Val Lys Ala Gly | |
| 145 150 155 160 | |
| Val Glu Thr Thr Lys Pro Ser Lys Gln Ser Asn Asn Lys Tyr Ala Ala | |
| 165 170 175 | |
| Ser Ser Tyr Leu Ser Leu Thr Pro Glu Gln Trp Lys Ser His Arg Ser | |
| 180 185 190 | |
| Tyr Ser Cys Gln Val Thr His Glu Gly Ser Thr Val Glu Lys Thr Val | |
| 195 200 205 | |
| Ala Pro Thr Glu Cys Ser | |
| 210 | |
| <210> 9 | |
| <211> 369 | |
| <212> DNA | |
| <213> Artificial Sequence | |
| <220> | |
| <223> human-rabbit hybrid | |
| <220> | |
| <221> CDS | |
| <222> (1) . . . (369) | |
| <400> 9 | |
| cag tcg ttg gag gag tcc ggg gga gac ctg gtc aag cct ggg gca tcc | 48 |
| Gln Ser Leu Glu Glu Ser Gly Gly Asp Leu Val Lys Pro Gly Ala Ser | |
| 1 5 10 15 | |
| ctg aca ctc acc tgc acg gcc tct gga ttc tcc ttc agt agc agc gac | 92 |
| Leu Thr Leu Thr Cys Thr Ala Ser Gly Phe Ser Phe Ser Ser Ser Asp | |
| 20 25 30 | |
| tgg ata tgc tgg gtc cgc cag gct cca ggg aag ggg ctg gag tgg atc | 144 |
| Trp Ile Cys Trp Val Arg Gln Ala Pro Gly Lys Gly Leu Glu Trp Ile | |
| 35 40 45 | |
| gca tgc att tat gct ggt agt att ggt ggc gct tac ttc gcg agc tgg | 192 |
| Ala Cys Ile Tyr Ala Gly Ser Ile Gly Gly Ala Tyr Phe Ala Ser Trp | |
| 50 55 60 | |
| gtg aat ggc cga ttc acc atc tcc aga gcc tcg tcg acc acg gtg act | 240 |
| Val Asn Gly Arg Phe Thr Ile Ser Arg Ala Ser Ser Thr Thr Val Thr | |
| 65 70 75 80 | |
| ctg caa atg acc agt ctg aca gcc gcg gac acg gcc acc tat ttc tgt | 288 |
| Leu Gln Met Thr Ser Leu Thr Ala Ala Asp Thr Ala Thr Tyr Phe Cys | |
| 85 90 95 | |
| gcg aga tca agt agt aat ggt cgt ttg aaa ttg gat caa ttc gac ttg | 336 |
| Ala Arg Ser Ser Ser Asn Gly Arg Leu Lys Leu Asp Gln Phe Asp Leu | |
| 100 105 110 | |
| tgg ggc cca ggc acc ctg gtc acc gtc tcc tca | 369 |
| Trp Gly Pro Gly Thr Leu Val Thr Val Ser Ser | |
| 115 120 | |
| <210> 10 | |
| <211> 123 | |
| <212> PRT | |
| <213> Artificial Sequence | |
| <220> | |
| <223> Synthetic Construct | |
| <400> 10 | |
| Gln Ser Leu Glu Glu Ser Gly Gly Asp Leu Val Lys Pro Gly Ala Ser | |
| 1 5 10 15 | |
| Leu Thr Leu Thr Cys Thr Ala Ser Gly Phe Ser Phe Ser Ser Ser Asp | |
| 20 25 30 | |
| Trp Ile Cys Trp Val Arg Gln Ala Pro Gly Lys Gly Leu Glu Trp Ile | |
| 35 40 45 | |
| Ala Cys Ile Tyr Ala Gly Ser Ile Gly Gly Ala Tyr Phe Ala Ser Trp | |
| 50 55 60 | |
| Val Asn Gly Arg Phe Thr Ile Ser Arg Ala Ser Ser Thr Thr Val Thr | |
| 65 70 75 80 | |
| Leu Gln Met Thr Ser Leu Thr Ala Ala Asp Thr Ala Thr Tyr Phe Cys | |
| 85 90 95 | |
| Ala Arg Ser Ser Ser Asn Gly Arg Leu Lys Leu Asp Gln Phe Asp Leu | |
| 100 105 110 | |
| Trp Gly Pro Gly thr Leu Val thr Val Ser Ser | |
| 115 120 | |
| <210> 11 | |
| <211> 330 | |
| <212> DNA | |
| <213> Artificial Sequence | |
| <220> | |
| <223> human-rabbit hybrid | |
| <220> | |
| <221> CDS | |
| <222> (1) . . . (330) | |
| <400> 11 | |
| gcc tat gat atg acc cag act cca gcc tct gtg gag gta gct gtg gga | 48 |
| Ala Tyr Asp Met Thr Gln Thr Pro Ala Ser Val Glu Val Ala Val Gly | |
| 1 5 10 15 | |
| ggc aca gtc acc atc aat tgc cag gcc agt cag agc att ggc ggt tgg | 96 |
| Gly Thr Val Thr Ile Asn Cys Gln Ala Ser Gln Ser Ile Gly Gly Trp | |
| 20 25 30 | |
| tta tcc tgg tat cgg cag aaa cca ggg cag cct ccc agc ctc ctg atc | 144 |
| Leu Ser Trp Tyr Arg Gln Lys Pro Gly Gln Pro Pro Ser Leu Leu Ile | |
| 35 40 45 | |
| tac aag gct tcc act ctg gca tct ggg gtc tca tcg cgg ttc agc ggc | 192 |
| Tyr Lys Ala Ser Thr Leu Ala Ser Gly Val Ser Ser Arg Phe Ser Gly | |
| 50 55 60 | |
| act gga tct agg aca cac ttc act ctc acc atc aac ggc gtg cag tgt | 240 |
| Thr Gly Ser Arg Thr His Phe Thr Leu Thr Ile Asn Gly Val Gln Cys | |
| 65 70 75 80 | |
| gcc gat gct gcc act tac tac tgt caa cag ggt agt gct gct gat gtt | 288 |
| Ala Asp Ala Ala Thr Tyr Tyr Cys Gln Gln Gly Ser Ala Ala Asp Val | |
| 85 90 95 | |
| gct aat act ttc ggc gga ggg acc gag gtg gtg gtc aaa ggt | 330 |
| Ala Asn Thr Phe Gly Gly Gly Thr Glu Val Val Val Lys Gly | |
| 100 105 110 | |
| <210> 12 | |
| <211> 110 | |
| <212> PRT | |
| <213> Artificial Sequence | |
| <220> | |
| <223> Synthetic Construct | |
| <400> 12 | |
| Ala Tyr Asp Met Thr Gln Thr Pro Ala Ser Val Glu Val Ala Val Gly | |
| 1 5 10 15 | |
| Gly Thr Val Thr Ile Asn Cys Gln Ala Ser Gln Ser Ile Gly Gly Trp | |
| 20 25 30 | |
| Leu Ser Trp Tyr Arg Gln Lys Pro Gly Gln Pro Pro Ser Leu Leu Ile | |
| 35 40 45 | |
| Tyr Lys Ala Ser Thr Leu Ala Ser Gly Val Ser Ser Arg Phe Ser Gly | |
| 50 55 60 | |
| Thr Gly Ser Arg Thr His Phe Thr Leu Thr Ile Asn Gly Val Gln Cys | |
| 65 70 75 80 | |
| Ala Asp Ala Ala Thr Tyr Tyr Cys Gln Gln Gly Ser Ala Ala Asp Val | |
| 85 90 95 | |
| Ala Asn Thr Phe Gly Gly Gly Thr Glu Val Val Val Lys Gly | |
| 100 105 110 | |
| <210> 13 | |
| <211> 363 | |
| <212> DNA | |
| <213> Artificial Sequence | |
| <220> | |
| <223> human-rabbit hybrid | |
| <220> | |
| <221> CDS | |
| <222> (1) . . . (363) | |
| <400> 13 | |
| cag tcg gtg gag gag tcc ggg ggt cgc ctg gtc acg cct ggg aca ccc | 48 |
| Gln Ser Val Glu Glu Ser Gly Gly Arg Leu Val Thr Pro Gly Thr Pro | |
| 1 5 10 15 | |
| ctg aca ctc acc tgc aca gtc tct gga ttc tcc ctc agt agc tat gca | 96 |
| Leu Thr Leu Thr Cys Thr Val Ser Gly Phe Ser Leu Ser Ser Tyr Ala | |
| 20 25 30 | |
| atg ggc tgg gtc cgc cag gct cca ggg aag ggg ctg gaa ttc atc gga | 144 |
| Met Gly Trp Val Arg Gln Ala Pro Gly Lys Gly Leu Glu Phe Ile Gly | |
| 35 40 45 | |
| atc att act tat aat ggt aac aca tac tac gcg agt tgg gcg aaa ggc | 192 |
| Ile Ile Thr Tyr Asn Gly Asn Thr Tyr Tyr Ala Ser Trp Ala Lys Gly | |
| 50 55 60 | |
| cga ttc ccc atc tcc aag acc tcg tcg acc acg gtg gat ctg aaa atg | 240 |
| Arg Phe Pro Ile Ser Lys Thr Ser Ser Thr Thr Val Asp Leu Lys Met | |
| 65 70 75 80 | |
| acc agt ccg aca acc gaa gac acg gcc act tat ttc tgt gcc aga gcc | 288 |
| Thr Ser Pro Thr Thr Glu Asp Thr Ala Thr Tyr Phe Cys Ala Arg Ala | |
| 85 90 95 | |
| cgt tat ggt ggt tat agt act aat tcc tac tat ctt aat atc tgg ggc | 336 |
| Arg Tyr Gly Gly Tyr Ser Thr Asn Ser Tyr Tyr Leu Asn Ile Trp Gly | |
| 100 105 110 | |
| cca ggc acc ctg gtc acc gtc tcc tca | 363 |
| Pro Gly Thr Leu Val Thr Val Ser Ser | |
| 115 120 | |
| <210> 14 | |
| <211> 121 | |
| <212> PRT | |
| <213> Artificial Sequence | |
| <220> | |
| <223> Synthetic Construct | |
| <400> 14 | |
| Gln Ser Val Glu Glu Ser Gly Gly Arg Leu Val Thr Pro Gly Thr Pro | |
| 1 5 10 15 | |
| Leu Thr Leu Thr Cys Thr Val Ser Gly Phe Ser Leu Ser Ser Tyr Ala | |
| 20 25 30 | |
| Met Gly Trp Val Arg Gln Ala Pro Gly Lys Gly Leu Glu Phe Ile Gly | |
| 35 40 45 | |
| Ile Ile Thr Tyr Asn Gly Asn Thr Tyr Tyr Ala Ser Trp Ala Lys Gly | |
| 50 55 60 | |
| Arg Phe Pro Ile Ser Lys Thr Ser Ser Thr Thr Val Asp Leu Lys Met | |
| 65 70 75 80 | |
| Thr Ser Pro Thr Thr Glu Asp Thr Ala Thr Tyr Phe Cys Ala Arg Ala | |
| 85 90 95 | |
| Arg Tyr Gly Gly Tyr Ser Thr Asn Ser Tyr Tyr Leu Asn Ile Trp Gly | |
| 100 105 110 | |
| Pro Gly Thr Leu Val Thr Val Ser SEr | |
| 115 120 | |
| <210> 15 | |
| <211> 333 | |
| <212> DNA | |
| <213> Artificial Sequence | |
| <220> | |
| <223> human-rabbit hybrid | |
| <220> | |
| <221> CDS | |
| <222> (1) . . . (333) | |
| <400> 15 | |
| gcc gac gtc gtg atg acc cag act cca gcc tcc gtg tct gca gct gtg | 48 |
| Ala Asp Val Val Met Thr Gln Thr Pro Ala Ser Val Ser Ala Ala Val | |
| 1 5 10 15 | |
| ggc ggc aca gtc acc atc aac tgc cag gcc agt gag aat att tat agg | 96 |
| Gly Gly Thr Val Thr Ile Asn Cys Gln Ala Ser Glu Asn Ile Tyr Arg | |
| 20 25 30 | |
| att ttg gcc tgg tat cag cag aaa cca ggg cag cgt ccc aaa ctc ctg | 144 |
| Ile Leu Ala Trp Tyr Gln Gln Lys Pro Gly Gln Arg Pro Lys Leu Leu | |
| 35 40 45 | |
| atc tat ggt gca tcc aat ctg gaa act ggg gtc cca tca cgg ttc aaa | 192 |
| Ile Tyr Gly Ala Ser Asn Leu Glu Thr Gly Val Pro Ser Arg Phe Lys | |
| 50 55 60 | |
| ggc agt gga tct ggg aca gaa tac act ctc acc atc agc gac ctg gag | 240 |
| Gly Ser Gly Ser Gly Thr Glu Tyr Thr Leu Thr Ile Ser Asp Leu Glu | |
| 65 70 75 80 | |
| tgt gac gat gct gcc act tac tat tgt caa ggt gtt ctt tat aat agt | 288 |
| Cys Asp Asp Ala Ala Thr Tyr Tyr Cys Gln Gly Val Leu Tyr Asn Ser | |
| 85 90 95 | |
| aat gat agt act ttc ggc gga ggg acc gag gtg gtg gtc aaa ggt | 333 |
| Asn Asp Ser Thr Phe Gly Gly Gly Thr Glu Val Val Val Lys Gly | |
| 100 105 110 | |
| <210> 16 | |
| <211> 111 | |
| <212> PRT | |
| <213> Artificial Sequence | |
| <220> | |
| <223> Synthetic Construct | |
| <400> 16 | |
| Ala Asp Val Val Met Thr Gln Thr Pro Ala Ser Val Ser Ala Ala Val | |
| 1 5 10 15 | |
| Gly Gly Thr Val Thr Ile Asn Cys Gln Ala Ser Glu Asn Ile Tyr Arg | |
| 20 25 30 | |
| Ile Leu Ala Trp Tyr Gln Gln Lys Pro Gly Gln Arg Pro Lys Leu Leu | |
| 35 40 45 | |
| Ile Tyr Gly Ala Ser Asn Leu Glu Thr Gly Val Pro Ser Arg Phe Lys | |
| 50 55 60 | |
| Gly Ser Gly Ser Gly Thr Glu Tyr Thr Leu Thr Ile Ser Asp Leu Glu | |
| 65 70 75 80 | |
| Cys Asp Asp Ala Ala Thr Tyr Tyr Cys Gln Gly Val Leu Tyr Asn Ser | |
| 85 90 95 | |
| Asn Asp Ser Thr Phe Gly Gly Gly Thr Glu Val Val Val Lys Gly | |
| 100 105 110 | |
| <210> 17 | |
| <211> 363 | |
| <212> DNA | |
| <213> Artificial Sequence | |
| <220> | |
| <223> human-rabbit hybrid | |
| <220> | |
| <221> CDS | |
| <222> (1) . . . (363) | |
| <400> 17 | |
| cag tcg gtg gag gag tcc ggg ggt cgc ctg gtc acg cct ggg aca ccc | 48 |
| Gln Ser Val Glu Glu Ser Gly Gly Arg Leu Val Thr Pro Gly Thr Pro | |
| 1 5 10 15 | |
| ctg aca ctc acc tgc aca gtc tct gga ttc tcc ctc agt agc tat gca | 96 |
| Leu Thr Leu Thr Cys Thr Val Ser Gly Phe Ser Leu Ser Ser Tyr Ala | |
| 20 25 30 | |
| atg ggc tgg gtc cgc cag gct cca ggg aag ggg ctg gaa ttc atc gga | 144 |
| Met Gly Trp Val Arg Gln Ala Pro Gly Lys Gly Leu Glu Phe Ile Gly | |
| 35 40 45 | |
| atc att agt tat agt ggt aac aca tac tac gcg agt tgg gcg aaa ggc | 192 |
| Ile Ile Ser Tyr Ser Gly Asn Thr Tyr Tyr Ala Ser Trp Ala Lys Gly | |
| 50 55 60 | |
| cga ttc ccc atc tcc aaa acc tcg tcg acc acg gtg gat ctg aaa atg | 240 |
| Arg Phe Pro Ile Ser Lys Thr Ser Ser Thr Thr Val Asp Leu Lys Met | |
| 65 70 75 80 | |
| acc agt ccg aca acc gaa gac acg gcc aat tat ttc tgt gcc aga gcc | 288 |
| Thr Ser Pro Thr Thr Glu Asp Thr Ala Asn Tyr Phe Cys Ala Arg Ala | |
| 85 90 95 | |
| cgt tat ggt ggt tat agt act aat tcc tac tat ctt aat atc tgg ggc | 336 |
| Arg Tyr Gly Gly Tyr Ser Thr Asn Ser Tyr Tyr Leu Asn Ile Trp Gly | |
| 100 105 110 | |
| cca ggc acc ctg gtc acc gtc tcc tca | 363 |
| Pro Gly Thr Leu Val Thr Val Ser Ser | |
| 115 120 | |
| <210> 18 | |
| <211> 121 | |
| <212> PRT | |
| <213> Artificial Sequence | |
| <220> | |
| <223> Synthetic Construct | |
| <400> 18 | |
| Gln Ser Val Glu Glu Ser Gly Gly Arg Leu Val Thr Pro Gly Thr Pro | |
| 1 5 10 15 | |
| Leu Thr Leu Thr Cys Thr Val Ser Gly Phe Ser Leu Ser Ser Tyr Ala | |
| 20 25 30 | |
| Met Gly Trp Val Arg Gln Ala Pro Gly Lys Gly Leu Glu Phe Ile Gly | |
| 35 40 45 | |
| Ile Ile Ser Tyr Ser Gly Asn Thr Tyr Tyr Ala Ser Trp Ala Lys Gly | |
| 50 55 60 | |
| Arg Phe Pro Ile Ser Lys Thr Ser Ser Thr Thr Val Asp Leu Lys Met | |
| 65 70 75 80 | |
| Thr Ser Pro Thr Thr Glu Asp Thr Ala Asn Tyr Phe Cys Ala Arg Ala | |
| 85 90 95 | |
| Arg Tyr Gly Gly Tyr Ser Thr Asn Ser Tyr Tyr Leu Asn Ile Trp Gly | |
| 100 105 110 | |
| Pro Gly Thr Leu Val Thr Val Ser Ser | |
| 115 120 | |
| <210> 19 | |
| <211> 333 | |
| <212> DNA | |
| <213> Artificial Sequence | |
| <220> | |
| <223> human-rabbit hybrid | |
| <220> | |
| <221> CDS | |
| <222> (1) . . . (333) | |
| <400> 19 | |
| gcc gac gtc gtg atg acc cag act cca gcc tcc gtg gag gca gct gtg | 48 |
| Ala Asp Val Val Met Thr Gln Thr Pro Ala Ser Val Glu Ala Ala Val | |
| 1 5 10 15 | |
| gga ggc aca gtc acc atc aac tgc cag gcc agt gag aat att tat aga | 96 |
| Gly Gly Thr Val Thr Ile Asn Cys Gln Ala Ser Glu Asn Ile Tyr Arg | |
| 20 25 30 | |
| gtt ttg gcc tgg tat cag cag aaa cca ggg cag cgt ccc aaa ctc ctg | 144 |
| Val Leu Ala Trp Tyr Gln Gln Lys Pro Gly Gln Arg Pro Lys Leu Leu | |
| 35 40 45 | |
| atc tat ggt gca tcc aat ctg gaa tct ggg gtc cca tca cgg ttc aaa | 192 |
| Ile Tyr Gly Ala Ser Asn Leu Glu Ser Gly Val Pro Ser Arg Phe Lys | |
| 50 55 60 | |
| ggc agt gga tct ggg aca gac tac act ctc acc atc agc gac ctg gag | 240 |
| Gly Ser Gly Ser Gly Thr Asp Tyr Thr Leu Thr Ile Ser Asp Leu Glu | |
| 65 70 75 80 | |
| tgt gac gat gct gcc act tac tat tgt caa ggt gtt gtt tat aat agt | 288 |
| Cys Asp Asp Ala Ala Thr Tyr Tyr Cys Gln Gly Val Val Tyr Asn Ser | |
| 85 90 95 | |
| gat gat agt gct ttc ggc gga ggg acc gag gtg gtg gtc aaa ggt | 333 |
| Asp Asp Ser Ala Phe Gly Gly Gly Thr Glu Val Val Val Lys Gly | |
| 100 105 110 | |
| <210> 20 | |
| <211> 111 | |
| <212> PRT | |
| <213> Artificial Sequence | |
| <220> | |
| <223> Synthetic Construct | |
| <400> 20 | |
| Ala Asp Val Val Met Thr Gln Thr Pro Ala Ser Val Glu Ala Ala Val | |
| 1 5 10 15 | |
| Gly Gly Thr Val Thr Ile Asn Cys Gln Ala Ser Glu Asn Ile Tyr Arg | |
| 20 25 30 | |
| Val Leu Ala Trp Tyr Gln Gln Lys Pro Gly Gln Arg Pro Lys Leu Leu | |
| 35 40 45 | |
| Ile Tyr Gly Ala Ser Asn Leu Glu Ser Gly Val Pro Ser Arg Phe Lys | |
| 50 55 60 | |
| Gly Ser Gly Ser Gly Thr Asp Tyr Thr Leu Thr Ile Ser Asp Leu Glu | |
| 65 70 75 80 | |
| Cys Asp Asp Ala Ala Thr Tyr Tyr Cys Gln Gly Val Val Tyr Asn Ser | |
| 85 90 95 | |
| Asp Asp Ser Ala Phe Gly Gly Gly Thr Glu Val Val Val Lys Gly | |
| 100 105 110 | |
| <210> 21 | |
| <211> 363 | |
| <212> DNA | |
| <213> Artificial Sequence | |
| <220> | |
| <223> human-rabbit hybrid | |
| <220> | |
| <221> CDS | |
| <222> (1) . . . (363) | |
| <400> 21 | |
| cag tcg gtg gag gag tcc ggg ggt cgc ctg gtc acg cct ggg aca ccc | 48 |
| Gln Ser Val Glu Glu Ser Gly Gly Arg Leu Val Thr Pro Gly Thr Pro | |
| 1 5 10 15 | |
| ctg aca ctc acc tgc aca gtc tct gga ttc tcc ctc agt agc tat gca | 96 |
| Leu Thr Leu Thr Cys Thr Val Ser Gly Phe Ser Leu Ser Ser Tyr Ala | |
| 20 25 30 | |
| atg ggc tgg gtc cgc cag gct cca ggg aag ggg ctg gaa ttc atc gga | 144 |
| Met Gly Trp Val Arg Gln Ala Pro Gly Lys Gly Leu Glu Phe Ile Gly | |
| 35 40 45 | |
| atc att act tat aat ggt ggc aca tac tac gcg agt tgg gcg aaa ggc | 192 |
| Ile Ile Thr Tyr Asn Gly Gly Thr Tyr Tyr Ala Ser Trp Ala Lys Gly | |
| 50 55 60 | |
| cga ttc ccc atc tcc aag acc tcg tcg acc acg gtg gat ctg aaa atg | 240 |
| Arg Phe Pro Ile Ser Lys Thr Ser Ser Thr Thr Val Asp Leu Lys Met | |
| 65 70 75 80 | |
| acc agt ccg aca acc gaa gac acg gcc act tat ttc tgt gcc aga gcc | 288 |
| Thr Ser Pro Thr Thr Glu Asp Thr Ala Thr Tyr Phe Cys Ala Arg Ala | |
| 85 90 95 | |
| cgt tat ggt ggt tat agt act aat tcc tac tat ctt aat atc tgg ggc | 336 |
| Arg Tyr Gly Gly Tyr Ser Thr Asn Ser Tyr Tyr Leu Asn Ile Trp Gly | |
| 100 105 110 | |
| cca ggc acc ctg gtc acc gtc tcc tca | 363 |
| Pro Gly Thr Leu Val Thr Val Ser Ser | |
| 115 120 | |
| <210> 22 | |
| <211> 121 | |
| <212> PRT | |
| <213> Artificial Sequence | |
| <220> | |
| <223> Synthetic Construct | |
| <400> 22 | |
| Gln Ser Val Glu Glu Ser Gly Gly Arg Leu Val Thr Pro Gly Thr Pro | |
| 1 5 10 15 | |
| Leu Thr Leu Thr Cys Thr Val Ser Gly Phe Ser Leu Ser Ser Tyr Ala | |
| 20 25 30 | |
| Met Gly Trp Val Arg Gln Ala Pro Gly Lys Gly Leu Glu Phe Ile Gly | |
| 35 40 45 | |
| Ile Ile Thr Tyr Asn Gly Gly Thr Tyr Tyr Ala Ser Trp Ala Lys Gly | |
| 50 55 60 | |
| Arg Phe Pro Ile Ser Lys Thr Ser Ser Thr Thr Val Asp Leu Lys Met | |
| 65 70 75 80 | |
| Thr Ser Pro Thr Thr Glu Asp Thr Ala Thr Tyr Phe Cys Ala Arg Ala | |
| 85 90 95 | |
| Arg Tyr Gly Gly Tyr Ser Thr Asn Ser Tyr Tyr Leu Asn Ile Trp Gly | |
| 100 105 110 | |
| Pro Gly Thr Leu Val Thr Val Ser Ser | |
| 115 120 | |
| <210> 23 | |
| <211> 333 | |
| <212> DNA | |
| <213> Artificial Sequence | |
| <220> | |
| <223> human-rabbit hybrid | |
| <220> | |
| <221> CDS | |
| <222> (1) . . . (333) | |
| <400> 23 | |
| gcc gac gtc gtg atg acc cag act cca gcc tcc gtg tct gca gct gtg | 48 |
| Ala Asp Val Val Met Thr Gln Thr Pro Ala Ser Val Ser Ala Ala Val | |
| 1 5 10 15 | |
| ggc ggc aca gtc acc atc aac tgc cag gcc agt gag aat att tat agg | 96 |
| Gly Gly Thr Val Thr Ile Asn Cys Gln Ala Ser Glu Asn Ile Tyr Arg | |
| 20 25 30 | |
| gtt ttg gcc tgg tat cag cag aaa cca ggg cag cgt ccc aaa ctc ctg | 144 |
| Val Leu Ala Trp Tyr Gln Gln Lys Pro Gly Gln Arg Pro Lys Leu Leu | |
| 35 40 45 | |
| atc tat ggt gca tcc aat ctg gaa act ggg gtc cca tca cgg ttc aaa | 192 |
| Ile Tyr Gly Ala Ser Asn Leu Glu Thr Gly Val Pro Ser Arg Phe Lys | |
| 50 55 60 | |
| ggc agt gga tct ggg aca gaa tac act ctc acc atc agc gac ctg gag | 240 |
| Gly Ser Gly Ser Gly Thr Glu Tyr Thr Leu Thr Ile Ser Asp Leu Glu | |
| 65 70 75 80 | |
| tgt gac gat gct gcc act tac tat tgt caa ggt gtt ctt tat gat aat | 288 |
| Cys Asp Asp Ala Ala Thr Tyr Tyr Cys Gln Gly Val Leu Tyr Asp Asn | |
| 85 90 95 | |
| aat gat agt act ttc ggc gga ggg acc gag gtg gtg gtc aaa ggt | 333 |
| Asn Asp Ser Thr Phe Gly Gly Gly Thr Glu Val Val Val Lys Gly | |
| 100 105 110 | |
| <210> 24 | |
| <211> 111 | |
| <212> PRT | |
| <213> Artificial Sequence | |
| <220> | |
| <223> Synthetic Construct | |
| <400> 24 | |
| Ala Asp Val Val Met Thr Gln Thr Pro Ala Ser Val Ser Ala Ala Val | |
| 1 5 10 15 | |
| Gly Gly Thr Val Thr Ile Asn Cys Gln Ala Ser Glu Asn Ile Tyr Arg | |
| 20 25 30 | |
| Val Leu Ala Trp Tyr Gln Gln Lys Pro Gly Gln Arg Pro Lys Leu Leu | |
| 35 40 45 | |
| Ile Tyr Gly Ala Ser Asn Leu Glu Thr Gly Val Pro Ser Arg Phe Lys | |
| 50 55 60 | |
| Gly Ser Gly Ser Gly Thr Glu Tyr Thr Leu Thr Ile Ser Asp Leu Glu | |
| 65 70 75 | |
| Cys Asp Asp Ala Ala Thr Tyr Tyr Cys Gln Gly Val Leu Tyr Asp Asn | |
| 85 90 95 | |
| Asn Asp Ser Thr Phe Gly Gly Gly Thr Glu Val Val Val Lys Gly | |
| 100 105 110 | |
| <210> 25 | |
| <211> 363 | |
| <212> DNA | |
| <213> Artificial Sequence | |
| <220> | |
| <223> human-rabbit hybrid | |
| <220> | |
| <221> CDS | |
| <222> (1) . . . (363) | |
| <400> 25 | |
| cag tcg gtg gag gag tcc ggg ggt cgc ctg gtc acg cct ggg aca ccc | 48 |
| Gln Ser Val Glu Glu Ser Gly Gly Arg Leu Val Thr Pro Gly Thr Pro | |
| 1 5 10 15 | |
| ctg aca ctc acc tgc aca gtc tct gga ttc tcc ctc agt agc tat gcg | 96 |
| Leu Thr Leu Thr Cys Thr Val Ser Gly Phe Ser Leu Ser Ser Tyr Ala | |
| 20 25 30 | |
| gtg ggc tgg gtc cgc cag gct cca ggg aag ggg ctg gaa ttc atc gga | 144 |
| Val Gly Trp Val Arg Gln Ala Pro Gly Lys Gly Leu Glu Phe Ile Gly | |
| 35 40 45 | |
| atc atc act tat aat ggt gcc aca tac tac gcg agt tgg gcg gaa ggc | 192 |
| Ile Ile Thr Tyr Asn Gly Ala Thr Tyr Tyr Ala Ser Trp Ala Glu Gly | |
| 50 55 60 | |
| cga ttc ccc atc tcc aag acc tcg tcg acc acg gtg gat ctg aaa atg | 240 |
| Arg Phe Pro Ile Ser Lys Thr Ser Ser Thr Thr Val Asp Leu Lys Met | |
| 65 70 75 80 | |
| acc agt ccg aca acc gaa gac acg gcc act tat ttc tgt gcc aga gcc | 288 |
| Thr Ser Pro Thr Thr Glu Asp Thr Ala Thr Tyr Phe Cys Ala Arg Ala | |
| 85 90 95 | |
| cgt tat ggt ggt tat agt act aat tcc tac tat ctt aat atc tgg ggc | 336 |
| Arg Tyr Gly Gly Tyr Ser Thr Asn Ser Tyr Tyr Leu Asn Ile Trp Gly | |
| 100 105 110 | |
| cca ggc acc ctg gtc acc gtc tcc tca | 363 |
| Pro Gly Thr Leu Val Thr Val Ser Ser | |
| 115 120 | |
| <210> 26 | |
| <211> 121 | |
| <212> PRT | |
| <213> Artificial Sequence | |
| <220> | |
| <223> Synthetic Construct | |
| <400> 26 | |
| Gln Ser Val Glu Glu Ser Gly Gly Arg Leu Val Thr Pro Gly Thr Pro | |
| 1 5 10 15 | |
| Leu Thr Leu Thr Cys Thr Val Ser Gly Phe Ser Leu Ser Ser Tyr Ala | |
| 20 25 30 | |
| Val Gly Trp Val Arg Gln Ala Pro Gly Lys Gly Leu Glu Phe Ile Gly | |
| 35 40 45 | |
| Ile Ile Thr Tyr Asn Gly Ala Thr Tyr Tyr Ala Ser Trp Ala Glu Gly | |
| 50 55 60 | |
| Arg Phe Pro Ile Ser Lys Thr Ser Ser Thr Thr Val Asp Leu Lys Met | |
| 65 70 75 80 | |
| Thr Ser Pro Thr Thr Glu Asp Thr Ala Thr Tyr Phe Cys Ala Arg Ala | |
| 85 90 95 | |
| Arg Tyr Gly Gly Tyr Ser Thr Asn Ser Tyr Tyr Leu Asn Ile Trp Gly | |
| 100 105 110 | |
| Pro Gly Thr Leu Val Thr Val Ser SEr | |
| 115 120 | |
| <210> 27 | |
| <211> 333 | |
| <212> DNA | |
| <213> Artificial Sequence | |
| <220> | |
| <223> human-rabbit hybrid | |
| <220> | |
| <221> CDS | |
| <222> (1) . . . (333) | |
| <400> 27 | |
| gcc gac gtc gtg atg acc cag act cca gcc tcc gtg tct gca gct gtg | 48 |
| Ala Asp Val Val Met Thr Gln Thr Pro Ala Ser Val Ser Ala Ala Val | |
| 1 5 10 15 | |
| ggc ggc aca gtc acc atc aac tgc cag gcc agt gag aat att tat agg | 96 |
| Gly Gly Thr Val Thr Ile Asn Cys Gln Ala Ser Glu Asn Ile Tyr Arg | |
| 20 25 30 | |
| att ttg gcc tgg tat cag cag aaa cca ggg cag cgt ccc aag ctc ctg | 144 |
| Ile Leu Ala Trp Tyr Gln Gln Lys Pro Gly Gln Arg Pro Lys Leu Leu | |
| 35 40 45 | |
| atc tat ggt gca tcc aat ctg gaa act ggg gtc cca tca cgg ttc aaa | 192 |
| Ile Tyr Gly Ala Ser Asn Leu Glu Thr Gly Val Pro Ser Arg Phe Lys | |
| 50 55 60 | |
| ggc agt gga tct ggg aca gaa tac act ctc acc atc agc gac ctg gag | 240 |
| Gly Ser Gly Ser Gly Thr Glu Tyr Thr Leu Thr Ile Ser Asp Leu Glu | |
| 65 70 75 80 | |
| tgt gac gat gct gcc act tac tat tgt caa ggt gtt ctt tgg aat agt | 288 |
| Cys Asp Asp Ala Ala Thr Tyr Tyr Cys Gln Gly Val Leu Trp Asn Ser | |
| 85 90 95 | |
| aat gat agt act ttc ggc gga ggg acc gag gtg gtg gtc aaa ggt | 333 |
| Asn Asp Ser Thr Phe Gly Gly Gly Thr Glu Val Val Val Lys Gly | |
| 100 105 110 | |
| <210> 28 | |
| <211> 111 | |
| <212> PRT | |
| <213> Artificial Sequence | |
| <220> | |
| <223> Synthetic Construct | |
| <400> 28 | |
| Ala Asp Val Val Met Thr Gln Thr Pro Ala Ser Val Ser Ala Ala Val | |
| 1 5 10 15 | |
| Gly Gly Thr Val Thr Ile Asn Cys Gln Ala Ser Glu Asn Ile Tyr Arg | |
| 20 25 30 | |
| Ile Leu Ala Trp Tyr Gln Gln Lys Pro Gly Gln Arg Pro Lys Leu Leu | |
| 35 40 45 | |
| Ile Tyr Gly Ala Ser Asn Leu Glu Thr Gly Val Pro Ser Arg Phe Lys | |
| 50 55 60 | |
| Gly Ser Gly Ser Gly Thr Glu Tyr Thr Leu Thr Ile Ser Asp Leu Glu | |
| 65 70 75 80 | |
| Cys Asp Asp Ala Ala Thr Tyr Tyr Cys Gln Gly Val Leu Trp Asn Ser | |
| 85 90 95 | |
| Asn Asp Ser Thr Phe Gly Gly Gly Thr Glu Val Val Val Lys Gly | |
| 100 105 110 | |
| <210> 29 | |
| <211> 366 | |
| <212> DNA | |
| <213> Artificial Sequence | |
| <220> | |
| <223> human-rabbit hybrid | |
| <220> | |
| <221> CDS | |
| <222> (1) . . . (366) | |
| <400> 29 | |
| cag gaa cta gtg gag tct gga ggg ggt ctg gtc cag ccg ggg gga tcc | 48 |
| Gln Glu Leu Val Glu Ser Gly Gly Gly Leu Val Gln Pro Gly Gly Ser | |
| 1 5 10 15 | |
| ctg aaa ctc tcc tgc aaa gcc tct gga atc gac ttc aat aac tat ggc | 96 |
| Leu Lys Leu Ser Cys Lys Ala Ser Gly Ile Asp Phe Asn Asn Tyr Gly | |
| 20 25 30 | |
| att agc tgg gtc cgc cag gct cca ggg aag ggg ctg gag tgg atc gca | 144 |
| Ile Ser Trp Val Arg Gln Ala Pro Gly Lys Gly Leu Glu Trp Ile Ala | |
| 35 40 45 | |
| tac aag tat cct ggt ttt ggt atc aga aac tac gcg aac tct gtg aag | 192 |
| Tyr Lys Tyr Pro Gly Phe Gly Ile Arg Asn Tyr Ala Asn Ser Val Lys | |
| 50 55 60 | |
| ggc cga ttc acc atc tcc agc gac aac gcc cag aac acg gtg ttt ctg | 240 |
| Gly Arg Phe Thr Ile Ser Ser Asp Asn Ala Gln Asn Thr Val Phe Leu | |
| 65 70 75 80 | |
| caa atg acc agt ctg gca gcc tcg gac acg gcc acc tat ttc tgt gca | 288 |
| Gln Met Thr Ser Leu Ala Ala Ser Asp Thr Ala Thr Tyr Phe Cys Ala | |
| 85 90 95 | |
| aga gga gcg cga tat agg cac gat gac tat ggt gct ttg aac ttg tgg | 336 |
| Arg Gly Ala Arg Tyr Arg His Asp Asp Tyr Gly Ala Leu Asn Leu Trp | |
| 100 105 110 | |
| ggc cca ggc acc ctg gtc acc gtc tcc tca | 366 |
| Gly Pro Gly Thr Leu Val Thr Val Ser Ser | |
| 115 120 | |
| <210> 30 | |
| <211> 122 | |
| <212> PRT | |
| <213> Artificial Sequence | |
| <220> | |
| <223> Synthetic Construct | |
| <400> 30 | |
| Gln Glu Leu Val Glu Ser Gly Gly Gly Leu Val Gln Pro Gly Gly Ser | |
| 1 5 10 15 | |
| Leu Lys Leu Ser Cys Lys Ala Ser Gly Ile Asp Phe Asn Asn Tyr Gly | |
| 20 25 30 | |
| Ile Ser Trp Val Arg Gln Ala Pro Gly Lys Gly Leu Glu Trp Ile Ala | |
| 35 40 45 | |
| Tyr Lys Tyr Pro Gly Phe Gly Ile Arg Asn Tyr Ala Asn Ser Val Lys | |
| 50 55 60 | |
| Gly Arg Phe Thr Ile Ser Ser Asp Asn Ala Gln Asn Thr Val Phe Leu | |
| 65 70 75 80 | |
| Gln Met Thr Ser Leu Ala Ala Ser Asp Thr Ala Thr Tyr Phe Cys Ala | |
| 85 90 95 | |
| Arg Gly Ala Arg Tyr Arg His Asp Asp Tyr Gly Ala Leu Asn Leu Trp | |
| 100 105 110 | |
| Gly Pro Gly Thr Leu Val Thr Val Ser Ser | |
| 115 120 | |
| <210> 31 | |
| <211> 324 | |
| <212> DNA | |
| <213> Artificial Sequence | |
| <220> | |
| <223> human-rabbit hybrid | |
| <220> | |
| <221> CDS | |
| <222> (1) . . . (324) | |
| <400> 31 | |
| gac atc gtg atg acc cag act cca ccc tct gtg tct gca gct gtg gga | 48 |
| Asp Ile Val Met Thr Gln Thr Pro Pro Ser Val Ser Ala Ala Val Gly | |
| 1 5 10 15 | |
| ggc aca gtc acc atc aat tgt cag gcc agt cag agt gtt acc aac ctc | 96 |
| Gly Thr Val Thr Ile Asn Cys Gln Ala Ser Gln Ser Val Thr Asn Leu | |
| 20 25 30 | |
| tta gcc tgg tat cag cag aaa cca ggg cag cct ccc aag ctc ctg att | 144 |
| Leu Ala Trp Tyr Gln Gln Lys Pro Gly Gln Pro Pro Lys Leu Leu Ile | |
| 35 40 45 | |
| tat ggt gca tct aat ctg gaa tct ggg gtc cca tcg cgt ttc cgt ggc | 192 |
| Tyr Gly Ala Ser Asn Leu Glu Ser Gly Val Pro Ser Arg Phe Arg Gly | |
| 50 55 60 | |
| agt gga tct ggg aca gag ttc act ctc acc atc agt ggc atg aag gct | 240 |
| Ser Gly Ser Gly Thr Glu Phe Thr Leu Thr Ile Ser Gly Met Lys Ala | |
| 65 70 75 80 | |
| gaa gat gct gcc act tat tac tgt caa agt ggt tat tat agt gct aat | 288 |
| Glu Asp Ala Ala Thr Tyr Tyr Cys Gln Ser Gly Tyr Tyr Ser Ala Asn | |
| 85 90 95 | |
| act ttc ggt gcg ggg acc aag gtg ggg atc aaa cgt | 324 |
| Thr Phe Gly Ala Gly Thr Lys Val Gly Ile Lys Arg | |
| 100 105 | |
| <210> 32 | |
| <211> 108 | |
| <212> PRT | |
| <213> Artificial Sequence | |
| <220> | |
| <223> Synthetic Construct | |
| <400> 32 | |
| Asp Ile Val Met Thr Gln Thr Pro Pro Ser Val Ser Ala Ala Val Gly | |
| 1 5 10 15 | |
| Gly Thr Val Thr Ile Asn Cys Gln Ala Ser Gln Ser Val Thr Asn Leu | |
| 20 25 30 | |
| Leu Ala Trp Tyr Gln Gln Lys Pro Gly Gln Pro Pro Lys Leu Leu Ile | |
| 35 40 45 | |
| Tyr Gly Ala Ser Asn Leu Glu Ser Gly Val Pro Ser Arg Phe Arg Gly | |
| 50 55 60 | |
| Ser Gly Ser Gly Thr Glu Phe Thr Leu Thr Ile Ser Gly Met Lys Ala | |
| 65 70 75 80 | |
| Glu Asp Ala Ala Thr Tyr Tyr Cys Gln Ser Gly Tyr Tyr Ser Ala Asn | |
| 85 90 95 | |
| Thr Phe Gly Ala Gly Thr Lys Val Gly Ile Lys Arg | |
| 100 105 | |
| <210> 33 | |
| <211> 357 | |
| <212> DNA | |
| <213> Artificial Sequence | |
| <220> | |
| <223> human-rabbit hybrid | |
| <220> | |
| <221> CDS | |
| <222> (1) . . . (357) | |
| <400> 33 | |
| cag tcg gtg gag gag tcc ggg ggt cgc ctg gtc acg cct ggg aca ccc | 48 |
| Gln Ser Val Glu Glu Ser Gly Gly Arg Leu Val Thr Pro Gly Thr Pro | |
| 1 5 10 15 | |
| ctg aca ctc acc tgc aca gtc tct gga ttc tcc ctc agt agt tat gta | 96 |
| Leu Thr Leu Thr Cys Thr Val Ser Gly Phe Ser Leu Ser Ser Tyr Val | |
| 20 25 30 | |
| ata aat tcg gtc cgc cag gct cca ggg aag ggg ctg gaa tcg atc gga | 144 |
| Ile Asn Trp Val Arg Gln Ala Pro Gly Lys Gly Leu Glu Trp Ile Gly | |
| 35 40 45 | |
| acc att agt agt agt ggt agc aca tac tac gcg agc tgg gcg aaa ggc | 192 |
| Thr Ile Ser Ser Ser Gly Ser Thr Tyr Tyr Ala Ser Trp Ala Lys Gly | |
| 50 55 60 | |
| cga ttc acc atc tcc aca acc tcg tcg acc acg gtg gat ctg aaa atc | 240 |
| Arg Phe Thr Ile Ser Thr Thr Ser Ser Thr Thr Val Asp Leu Lys Ile | |
| 65 70 75 80 | |
| acc agt ccg aca acc gag gac acg gcc acc tat ttc tgt gcc aga ggg | 288 |
| Thr Ser Pro Thr Thr Glu Asp Thr Ala Thr Tyr Phe Cys Ala Arg Gly | |
| 85 90 95 | |
| gct gat ggt gct ttt att agt gac tac ttt aac atc tgg ggc cca ggc | 336 |
| Ala Asp Gly Ala Phe Ile Ser Asp Tyr Phe Asn Ile Trp Gly Pro Gly | |
| 100 105 110 | |
| acc ctg gtc acc gtc tcc tca | 357 |
| Thr Leu Val Thr Val Ser Ser | |
| 115 | |
| <210> 34 | |
| <211> 119 | |
| <212> PRT | |
| <213> Artificial Sequence | |
| <220> | |
| <223> Synthetic Construct | |
| <400> 34 | |
| Gln Ser Val Glu Glu Ser Gly Gly Arg Leu Val Thr Pro Gly Thr Pro | |
| 1 5 10 15 | |
| Leu Thr Leu Thr Cys Thr Val Ser Gly Phe Ser Leu Ser Ser Tyr Val | |
| 20 25 30 | |
| Ile Asn Trp Val Arg Gln Ala Pro Gly Lys Gly Leu Glu Trp Ile Gly | |
| 35 40 45 | |
| Thr Ile Ser Ser Ser Gly Ser Thr Tyr Tyr Ala Ser Trp Ala Lys Gly | |
| 50 55 60 | |
| Arg Phe Thr Ile Ser Thr Thr Ser Ser Thr Thr Val Asp Leu Lys Ile | |
| 65 70 75 80 | |
| Thr Ser Pro Thr Thr Glu Asp Thr Ala Thr Tyr Phe Cys Ala Arg Gly | |
| 85 90 95 | |
| Ala Asp Gly Ala Phe Ile Ser Asp Tyr Phe Asn Ile Trp Gly Pro Gly | |
| 100 105 110 | |
| Thr Leu Val Thr Val Ser Ser | |
| 115 | |
| <210> 35 | |
| <211> 336 | |
| <212> DNA | |
| <213> Artificial Sequence | |
| <220> | |
| <223> human-rabbit hybrid | |
| <220> | |
| <221> CDS | |
| <222> (1) . . . (336) | |
| <400> 35 | |
| gcc gac atc gtg atg acc cag act cca tcc tcc gtg tct gca gct gtg | 48 |
| Ala Asp Ile Val Met Thr Gln Thr Pro Ser Ser Val Ser Ala Ala Val | |
| 1 5 10 15 | |
| gga ggc aca gtc acc atc aag tgc cag gcc aat gag aat att gac agt | 96 |
| Gly Gly Thr Val Thr Ile Lys Cys Gln Ala Asn Glu Asn Ile Asp Ser | |
| 20 25 30 | |
| tgg tta gcc tgg tat cag cag aaa cca ggg cag cgt ccc aag ctc cta | 144 |
| Trp Leu Ala Trp Tyr Gln Gln Lys Pro Gly Gln Arg Pro Lys Leu Leu | |
| 35 40 45 | |
| atc tat tat gca tcc act ctg gca tct ggg gtc cca tcg cgg ttc aaa | 192 |
| Ile Tyr Tyr Ala Ser Thr Leu Ala Ser Gly Val Pro Ser Arg Phe Lys | |
| 50 55 60 | |
| ggc agt gga tct ggg aca gag tac act ctc acc atc agc gac ctg gag | 240 |
| Gly Ser Gly Ser Gly Thr Glu Tyr Thr Leu Thr Ile Ser Asp Leu Glu | |
| 65 70 75 80 | |
| tgt gcc gat ggt gcc act tac tac tgt caa agc tat gat act att agt | 288 |
| Cys Ala Asp Gly Ala Thr Tyr Tyr Cys Gln Ser Tyr Asp Thr Ile Ser | |
| 85 90 95 | |
| gac tat ggt gtt ggt ttc ggc gga ggg acc gag gtg gtg gtc aaa ggt | 336 |
| Asp Tyr Gly Val Gly Phe Gly Gly Gly Thr Glu Val Val Val Lys Gly | |
| 100 105 110 | |
| <210> 36 | |
| <211> 112 | |
| <212> PRT | |
| <213> Artificial Sequence | |
| <220> | |
| <223> Synthetic Construct | |
| <400> 36 | |
| Ala Asp Ile Val Met Thr Gln Thr Pro Ser Ser Val Ser Ala Ala Val | |
| 1 5 10 15 | |
| Gly Gly Thr Val Thr Ile Lys Cys Gln Ala Asn Glu Asn Ile Asp Ser | |
| 20 25 30 | |
| Trp Leu Ala Trp Tyr Gln Gln Lys Pro Gly Gln Arg Pro Lys Leu Leu | |
| 35 40 45 | |
| Ile Tyr Tyr Ala Ser Thr Leu Ala Ser Gly Val Pro Ser Arg Phe Lys | |
| 50 55 60 | |
| Gly Ser Gly Ser Gly Thr Glu Tyr Thr Leu Thr Ile Ser Asp Leu Glu | |
| 65 70 75 80 | |
| Cys Ala Asp Gly Ala Thr Tyr Tyr Cys Gln Ser Tyr Asp Thr Ile Ser | |
| 85 90 95 | |
| Asp Tyr Gly Val Gly Phe Gly Gly Gly Thr Glu Val Val Val Lys Gly | |
| 100 105 110 | |
| <210> 37 | |
| <211> 363 | |
| <212> DNA | |
| <213> Artificial Sequence | |
| <220> | |
| <223> human-rabbit hybrid | |
| <220> | |
| <221> CDS | |
| <222> (1) . . . (363) | |
| <400> 37 | |
| cag tcg gtg gag gag tcc ggg ggt cgc ctg gtc acg cct ggg aca ccc | 48 |
| Gln Ser Val Glu Glu Ser Gly Gly Arg Leu Val Thr Pro Gly Thr Pro | |
| 1 5 10 15 | |
| ctg aca ctc acc tgc aca gtc tct gga ttc tcc ctc agt agc tat gca | 96 |
| Leu Thr Leu Thr Cys Thr Val Ser Gly Phe Ser Leu Ser Ser Tyr Ala | |
| 20 25 30 | |
| atg ggc tgg gtc cgc cag gct cca ggg aag ggg ctg gaa ttc atc gga | 144 |
| Met Gly Trp Val Arg Gln Ala Pro Gly Lys Gly Leu Glu Phe Ile Gly | |
| 35 40 45 | |
| atc atc act tat aat agt aac aca tac tac gcg aat tgg gcg aaa ggc | 192 |
| Ile Ile Thr Tyr Asn Ser Asn Thr Tyr Tyr Ala Asn Trp Ala Lys Gly | |
| 50 55 60 | |
| cga ttc ccc atc tcc aag acc tcg tcg acc acg gtg gat ctg aaa atg | 240 |
| Arg Phe Pro Ile Ser Lys Thr Ser Ser Thr Thr Val Asp Leu Lys Met | |
| 65 70 75 80 | |
| acc agt ccg aca acc gaa gac acg gcc act tat ttc tgt gcc aga gcc | 288 |
| Thr Ser Pro Thr Thr Glu Asp Thr Ala Thr Tyr Phe Cys Ala Arg Ala | |
| 85 90 95 | |
| cgt tat ggt ggt tat agt act aat tcc tac tat ctt aat atc tgg ggc | 336 |
| Arg Tyr Gly Gly Tyr Ser Thr Asn Ser Tyr Tyr Leu Asn Ile Trp Gly | |
| 100 105 110 | |
| cca ggc acc ctg gtc acc gtc tcc tca | 363 |
| Pro Gly Thr Leu Val Thr Val Ser Ser | |
| 115 120 | |
| <210> 38 | |
| <211> 121 | |
| <212> PRT | |
| <213> Artificial Sequence | |
| <220> | |
| <223> Synthetic Construct | |
| <400> 38 | |
| Gln Ser Val Glu Glu Ser Gly Gly Arg Leu Val Thr Pro Gly Thr Pro | |
| 1 5 10 15 | |
| Leu Thr Leu Thr Cys Thr Val Ser Gly Phe Ser Leu Ser Ser Tyr Ala | |
| 20 25 30 | |
| Met Gly Trp Val Arg Gln Ala Pro Gly Lys Gly Leu Glu Phe Ile Gly | |
| 35 40 45 | |
| Ile Ile Thr Tyr Asn Ser Asn Thr Tyr Tyr Ala Asn Trp Ala Lys Gly | |
| 50 55 60 | |
| Arg Phe Pro Ile Ser Lys Thr Ser Ser Thr Thr Val Asp Leu Lys Met | |
| 65 70 75 80 | |
| Thr Ser Pro Thr Thr Glu Asp Thr Ala Thr Tyr Phe Cys Ala Arg Ala | |
| 85 90 95 | |
| Arg Tyr Gly Gly Tyr Ser Thr Asn Ser Tyr Tyr Leu Asn Ile Trp Gly | |
| 100 105 110 | |
| Pro Gly Thr Leu Val Thr Val Ser Ser | |
| 115 120 | |
| <210> 39 | |
| <211> 333 | |
| <212> DNA | |
| <213> Artificial Sequence | |
| <220> | |
| <223> human-rabbit hybrid | |
| <220> | |
| <221> CDS | |
| <222> (1) . . . (333) | |
| <400> 39 | |
| gcc gac gtc gtg atg acc cag act cca gcc tcc gtg tct gca gct gtg | 48 |
| Ala Asp Val Val Met Thr Gln Thr Pro Ala Ser Val Ser Ala Ala Val | |
| 1 5 10 15 | |
| ggc ggc aca gtc acc atc aac tgc cag gcc agt gag aat att tat agg | 96 |
| Gly Gly Thr Val Thr Ile Asn Cys Gln Ala Ser Glu Asn Ile Tyr Arg | |
| 20 25 30 | |
| att ttg gcc tgg tat cag cag aaa cca ggg cag cct ccc aag ctc ctg | 144 |
| Ile Leu Ala Trp Tyr Gln Gln Lys Pro Gly Gln Pro Pro Lys Leu Leu | |
| 35 40 45 | |
| atc tat ggt gca tcc aat ctg gaa act ggg gtc cca tca cgg ttc aaa | 192 |
| Ile Tyr Gly Ala Ser Asn Leu Glu Thr Gly Val Pro Ser Arg Phe Lys | |
| 50 55 60 | |
| ggc agt gga tct ggg aca gaa tat act ctc acc atc agc gac ctg gag | 240 |
| Gly Ser Gly Ser Gly Thr Glu Tyr Thr Leu Thr Ile Ser Asp Leu Glu | |
| 65 70 75 80 | |
| tgt gac gat gct gcc act tac tat tgt caa ggt gtt ctt tat aat agt | 288 |
| Cys Asp Asp Ala Ala Thr Tyr Tyr Cys Gln Gly Val Leu Tyr Asn Ser | |
| 85 90 95 | |
| aat gat agt act ttc ggc gga ggg acc gag gtg gtg gtc aaa ggt | 333 |
| Asn Asp Ser Thr Phe Gly Gly Gly Thr Glu Val Val Val Lys Gly | |
| 100 105 110 | |
| <210> 40 | |
| <211> 111 | |
| <212> PRT | |
| <213> Artificial Sequence | |
| <220> | |
| <223> Synthetic Construct | |
| <400> 40 | |
| Ala Asp Val Val Met Thr Gln Thr Pro Ala Ser Val Ser Ala Ala Val | |
| 1 5 10 15 | |
| Gly Gly Thr Val Thr Ile Asn Cys Gln Ala Ser Glu Asn Ile Tyr Arg | |
| 20 25 30 | |
| Ile Leu Ala Trp Tyr Gln Gln Lys Pro Gly Gln Pro Pro Lys Leu Leu | |
| 35 40 45 | |
| Ile Tyr Gly Ala Ser Asn Leu Glu Thr Gly Val Pro Ser Arg Phe Lys | |
| 50 55 60 | |
| Gly Ser Gly Ser Gly Thr Glu Tyr Thr Leu Thr Ile Ser Asp Leu Glu | |
| 65 70 75 80 | |
| Cys Asp Asp Ala Ala Thr Tyr Tyr Cys Gln Gly Val Leu Tyr Asn Ser | |
| 85 90 95 | |
| Asn Asp Ser Thr Phe Gly Gly Gly Thr Glu Val Val Val Lys Gly | |
| 100 105 110 | |
| <210> 41 | |
| <211> 363 | |
| <212> DNA | |
| <213> Artificial Sequence | |
| <220> | |
| <223> human-rabbit hybrid | |
| <220> | |
| <221> CDS | |
| <222> (1) . . . (363) | |
| <400> 41 | |
| cag gag cag ctg gag gag tcc ggg gga gac ctg gtc aag cct gag gga | 48 |
| Gln Glu Gln Leu Glu Glu Ser Gly Gly Asp Leu Val Lys Pro Glu Gly | |
| 1 5 10 15 | |
| tcc ctg aca ctc acc tgc act gcc tct gga ttt gac tcc agt agc acc | 96 |
| Ser Leu Thr Leu Thr Cys Thr Ala Ser Gly Phe Asp Ser Ser Ser Thr | |
| 20 25 30 | |
| tac tac atg tgc tgg gtc cgc cag gct cca ggg aag ggg ctg gag tgg | 144 |
| Tyr Tyr Met Cys Trp Val Arg Gln Ala Pro Gly Lys Gly Leu Glu Trp | |
| 35 40 45 | |
| agc gga tgc att tat act ggt ggt ctt aca tac tac gcg agc tgg gcg | 192 |
| Ser Gly Cys Ile Tyr Thr Gly Gly Leu Thr Tyr Tyr Ala Ser Trp Ala | |
| 50 55 60 | |
| aaa ggc cga ttc acc atc tcc aaa acc tcg tcg acc acg gtg act ctg | 240 |
| Lys Gly Arg Phe Thr Ile Ser Lys Thr Ser Ser Thr Thr Val Thr Leu | |
| 65 70 75 80 | |
| caa atg acc agt ctg aca gcc gcg gac acg gcc acc tat ttc tgt gcg | 288 |
| Gln Met Thr Ser Leu Thr Ala Ala Asp Thr Ala Thr Tyr Phe Cys Ala | |
| 85 90 95 | |
| agt tat att ggt agt cgt ttt tat atg ccc ggt ttt agc ttg tgg ggc | 336 |
| Ser Tyr Ile Gly Ser Arg Phe Tyr Met Pro Gly Phe Ser Leu Trp Gly | |
| 100 105 110 | |
| cca ggc acc ctg gtc acc gtc tcc tca | 363 |
| Pro Gly Thr Leu Val Thr Val Ser Ser | |
| 115 120 | |
| <210> 42 | |
| <211> 121 | |
| <212> PRT | |
| <213> Artificial Sequence | |
| <220> | |
| <223> Synthetic Construct | |
| <400> 42 | |
| Gln Glu Gln Leu Glu Glu Ser Gly Gly Asp Leu Val Lys Pro Glu Gly | |
| 1 5 10 15 | |
| Ser Leu Thr Leu Thr Cys Thr Ala Ser Gly Phe Asp Ser Ser Ser Thr | |
| 20 25 30 | |
| Tyr Tyr Met Cys Trp Val Arg Gln Ala Pro Gly Lys Gly Leu Glu Trp | |
| 35 40 45 | |
| Ser Gly Cys Ile Tyr Thr Gly Gly Leu Thr Tyr Tyr Ala Ser Trp Ala | |
| 50 55 60 | |
| Lys Gly Arg Phe Thr Ile Ser Lys Thr Ser Ser Thr Thr Val Thr Leu | |
| 65 70 75 80 | |
| Gln Met Thr Ser Leu Thr Ala Ala Asp Thr Ala Thr Tyr Phe Cys Ala | |
| 85 90 95 | |
| Ser Tyr Ile Gly Ser Arg Phe Tyr Met Pro Gly Phe Ser Leu Trp Gly | |
| 100 105 110 | |
| Pro Gly Thr Leu Val Thr Val Ser Ser | |
| 115 120 | |
| <210> 43 | |
| <211> 339 | |
| <212> DNA | |
| <213> Artificial Sequence | |
| <220> | |
| <223> human-rabbit hybrid | |
| <220> | |
| <221> CDS | |
| <222> (1) . . . (339) | |
| <400> 43 | |
| gcc atc gtg atg acc cag act cca tct tcc aag tct gtc cct gtg gga | 48 |
| Ala Ile Val Met Thr Gln Thr Pro Ser Ser Lys Ser Val Pro Val Gly | |
| 1 5 10 15 | |
| gac aca gtc acc atc aat tgc cag gcc agt gag agt att tac agt aac | 96 |
| Asp Thr Val Thr Ile Asn Cys Gln Ala Ser Glu Ser Ile Tyr Ser Asn | |
| 20 25 30 | |
| aat cgc tta gcc tgg ttt caa caa aaa cca ggg cag cct ccc aag ctc | 144 |
| Asn Arg Leu Ala Trp Phe Gln Gln Lys Pro Gly Gln Pro Pro Lys Leu | |
| 35 40 45 | |
| ctg atc tat ctg gca tcc act ctg gca tct ggg gtc cca tcg cgg ttc | 192 |
| Leu Ile Tyr Leu Ala Ser Thr Leu Ala Ser Gly Val Pro Ser Arg Phe | |
| 50 55 60 | |
| aaa ggc agt gga tct ggg aca cag ttc act ctc acc atc agc gat gtg | 240 |
| Lys Gly Ser Gly Ser Gly Thr Gln Phe Thr Leu Thr Ile Ser Asp Val | |
| 65 70 75 80 | |
| gtg tgt gac gat gct gcc act tac tac tgt gca gga tat aaa ggt agt | 288 |
| Val Cys Asp Asp Ala Ala Thr Tyr Tyr Cys Ala Gly Tyr Lys Gly Ser | |
| 85 90 95 | |
| agt act gat ggt act gcg ttc ggc gga ggg acc gag gtg gtg gtc aaa | 336 |
| Ser Thr Asp Gly Thr Ala Phe Gly Gly Gly Thr Glu Val Val Val Lys | |
| 100 105 110 | |
| ggt | 339 |
| Gly | |

```
<210> 44
<211> 113
<212> PRT
<213> Artificial Sequence
<220>
<223> Synthetic Construct
<400> 44
Ala Ile Val Met Thr Gln Thr Pro Ser Ser Lys Ser Val Pro Val Gly
1 5 10 15
Asp Thr Val Thr Ile Asn Cys Gln Ala Ser Glu Ser Ile Tyr Ser Asn
                        20 25 30
Asn Arg Leu Ala Trp Phe Gln Gln Lys Pro Gly Gln Pro Pro Lys Leu
                35 40 45
Leu Ile Tyr Leu Ala Ser Thr Leu Ala Ser Gly Val Pro Ser Arg Phe
        50 55 60
Lys Gly Ser Gly Ser Gly Thr Gln Phe Thr Leu Thr Ile Ser Asp Val
65 70 75 80
Val Cys Asp Asp Ala Ala Thr Tyr Tyr Cys Ala Gly Tyr Lys Gly Ser
                                85 90 95
Ser Thr Asp Gly Thr Ala Phe Gly Gly Gly Thr Glu Val Val Val Lys
                        100 105 110
Gly
<210> 45
<211> 10
<212> PRT
<213> Homo sapiens
<400> 45
Gly Phe Thr Ser Ser Ser Ser Asp Tyr Ala
1 5 10
<210> 46
<211> 8
<212> PRT
<213> Homo sapiens
<400> 46
Ile Ser Gly Ser Gly Gly Ser Thr
1 5
<210> 47
<211> 19
<212> PRT
<213> Homo sapiens
<400> 47
Ala Lys Val Val Gly Tyr Ser Met Tyr Asp Tyr Tyr Tyr His Tyr Ala
1 5 10 15
Leu Asp Val
<210> 48
<211> 8
<212> PRT
<213> Homo sapiens
<400> 48
Ser Ser Asn Ile Gly Ser Asn Thr
1 5
<210> 49
<211> 3
<212> PRT
<213> Homo sapiens
<400> 49
Ser Asn Asn
1
<210> 50
<211> 9
<212> PRT
<213> Homo sapiens
<400> 50
Gln Ser Tyr Asp Thr Ser Asn Val Val
1 5
PBI#30af4-VH domain
Nucleic Acid Sequence:
CAGGTGCAGCTGCAGGAGTCTGGAGGTGGCTTGATACAGCCTGGTGGATCTCTGAGACTCTCCTGTGCAGCCT
CTGGATTCACCTCTAGTTCTAGTGATTATGCCATGAGTTGGGTCAGGCAGGCTCCAGGGAAGGGGCTGGAGTG
GGTCTCGTCCATCAGTGGTAGTGGCGTTAGCACATACTACGCAGACTCCGTGAAGGGTCGCTTCACCATCTCC
AGAGACAATTCCAAGAACACACTCTATGTGCAAATGACCAGCCTGACAGCCGAGGACACGGCCGTGTACTACT
GTGCGAAGGTCGTCGGTTACAGTGTTTACGACTACTACTACCACTACGCTTTGGACGTCTGGGGCCAAGGGAC
CACGGTCACCGTCTCCTCA
CDR1: GGATTCACCTCTAGTTCTAGTGATTATGCC
CDR2: ATCAGTGGTAGTGGCGTTAGCACA
CDR3: GCGAAGGTCGTCGGTTACAGTGTTTACGACTACTACTACCACTACGCTTTGGACGTC
Amino Acid Sequence:
QVQLQESGGGLIQPGGSLRLSCAASGFTSSSSDYAMSWVRQAPGKGLEWVSSISGSGVSTYY
ADSVKGRFTISRDNSKNTLYVQMTSLTAEDTAVYYCAKVVGYSVYDYYYHYALDVWGQGT
TVTVSS
SEQ ID No. 51
(VH)
CDR1: GFTSSSSDYA
SEQ ID No. 52
(VH)
CDR2: ISGSGVST
SEQ ID No. 53
(VH)
CDR3: AKVVGYSVYDYYYHYALDV
PBI#30af4-VL domain
Nucleic Acid Sequence:
CAGCCTGTGCTGACTCAGCCACCTTCAGCGTCTGGGACCCCCGGGCAGAGGGTCACCATCTCTTGTTCTGGAA
GCAGCTCCAACATCGGTAGTAATACTGTGAACTGGTACCAGCAGCTTCCAGGAACGGCTCCTAAGCTCCTCAT
CTATAGTGATAATCAGAGACCCTCAGGTGTCCCTGACCGATTCTCTGGCTCCAAGTCTGGCACCTCAGCCTCC
CTCACCATCTCTGGACTGAAGACTGAGGACGAGGCTGGCTACTACTGTCAGTCTGATGATACCAGCAATGTGG
TATTCGGTGGAGGCACCAAGGTGACCGTCCTC
CDR1: AGCTCCAACATCGGTAGTAATACT
CDR2: AGTGATAAT
CDR3: CAGTCTGATGATACCAGCAATGTGGTA
Amino acid sequence:
QPVLTQPPSASGTPGQRVTISCSGSSSNIGSNTVNWYQQLPGTAPKLLIYSDNQRPSGVP
DRFSGSKSGTSASLTISGLKTEDEAGYYCQSDDTSNVVFGGGTKVTVL
SEQ ID No. 54
(VL)
CDR1: SSNIGSNT
SEQ ID No. 55
(VL)
CDR2: SDN
SEQ ID No. 56
(VL)
CDR3: QSDDTSNVV
PBI#30af6-VH domain
Nucleic Acid Sequence:
CAGGTGCAGCTGCAGGAGTCTGGAGGTGGCTTGATACAGCCTGGTGGATCTCTGAGACTCTCCTGTGCAGCCT
CTGGATTCACCTCTAGTTCTAGTGATTATGCCATGAGTTGGGTCAGGCAGGCTCCAGGGAAGGGGCTGGAGTG
GGTCTCGTCCATCAGTGGTAGTGGCGGTAGCACATACTACGCAGACTCCGTGAAGGGTCGCTTCACCATCTCC
AGAGACAATTCCAAGAACACACTCTATGTGCAAATGACCAGCCTGACAGCCGAGGACACGGCCGTGTACTACT
GTGCGAAGGTCTTCGGTTACAGTATGTACGACTACTACTACCACTACGCTTTGGACGTCTGGGGCCAAGGGAC
CACGGTCACCGTCTCCTCA
CDR1: GGATTCACCTCTAGTTCTAGTGATTATGCC
CDR2: ATCAGTGGTAGTGGCGGTAGCACA
CDR3: GCGAAGGTCTTCGGTTACAGTATGTACGACTACTACTACCACTACGCTTTGGACGTC
Amino Acid Sequence:
QVQLQESGGGLIQPGGSLRLSCAASGFTSSSSDYAMSWVRQAPGKGLEWVSSISGSGGSTYY
ADSVKGRFTISRDNSKNTLYVQMTSLTAEDTAVYYCAKVFGYSMYDYYYHYALDVWGQGT
TVTVSS
SEQ ID No. 57
(VH)
CDR1: GFTSSSSDYA
SEQ ID No. 58
(VH)
CDR2: ISGSGGST
SEQ ID No. 59
(VH)
CDR3: AKVFGYSMYDYYYHYALDV
PBI#30af6-VL domain
Nucleic Acid Sequence:
CAGCCTGTGCTGACTCAGCCACCTTCAGCGTCTGGGACCCCCGGGCAGAGGATCACCATCTCTTGTTCTGGAA
GCAGCTCCAACATCGGTAGTAATACTGTGAACTGGTACCAGCAGCTTCCAGGAACGGCTCCTAAGCTCCTCAT
CTATAGTAATAATCAGAGACCCTCAGGTGTCCCTGACCGATTCTCTGGCTCCAAGTCTGGCACCTCAGCCTCC
CTCACCATCTCTGGACTGAAGACTGAGGACGAGGCTGGCTACTACTGTCAGTCTGATGATACCAGCAATGTGG
TATTCGGTGGAGGCACCAAGGTGACCGTCCTC
CDR1: AGCTCCAACATCGGTAGTAATACT
CDR2: AGTAATAAT
CDR3: CAGTCTGATGATACCAGCAATGTGGTA
Amino Acid Sequence:
QPVLTQPPSASGTPGQRITISCSGSSSNIGSNTVNWYQQLPGTAPKLLIYSNNQRPSGVP
DRFSGSKSGTSASLTISGLKTEDEAGYYCQSDDTSNVVFGGGTKVTVL
SEQ ID No. 60
(VL)
CDR1: SSNIGSNT
SEQ ID No. 61
(VL)
CDR2: SNN
SEQ ID No. 62
(VL)
CDR3: QSDDTSNVV
PBI#30af7-VH domain
Nucleic Acid Sequence:
CAGGTGCAGCTGCAGGAGTCTGGAGGTGGCTTGATACAGCCTGGTGGATCTCTGAGACTCTCCTGTGCAGCCT
CTGGATTCACCACTAGTTCTAGTGATTATGCCATGAGTTGGGTCAGGCAGGCTCCAGGGAAGGGGCTGGAGTG
GGTCTCGTCCATCAGTGGTAGTGGGGGTAGCACATACTACGCAGACTCCGTGAAGGGTCGCTTCACCATCTCC
AGAGACAATTCCAAGAACACACTCTATGTGCAAATGACCAGCCTGACAGCCGAGGACACGGCCGTGTACTACT
GTGCGAAGGACGTCGGTTACAGTACGTACGACTACTACTACCACTACGCTTTGGACGTCTGGGGCCAAGGGAC
CACGGTCACCGTCTCCTCA
CDR1: GGATTCACCACTAGTTCTAGTGATTATGCC
CDR2: ATCAGTGGTAGTGGGGGTAGCACA
CDR3: GCGAAGGACGTCGGTTACAGTACGTACGACTACTACTACCACTACGCTTTGGACGTC
Amino Acid Sequence:
QVQLQESGGGLIQPGGSLRLSCAASGFTTSSSDYAMSWVRQAPGKGLEWVSSISGSGGSTYY
ADSVKGRFTISRDNSKNTLYVQMTSLTAEDTAVYYCAKDVGYSTYDYYYHYALDVWGQGT
TVTVSS
SEQ ID No. 63
(VH)
CDR1: GFTTSSSDYA
SEQ ID No. 64
(VH)
CDR2: ISGSGGST
SEQ ID No. 65
(VH)
CDR3: AKDVGYSTYDYYYHYALDV
PBI#30af7-VL domain
Nucleic Acid Sequence:
CAGCCTGTGCTGACTCAGCCACCTTCAGCGTCTGGGACCCCCGGGCAGAGGGTCACCATCTCTTGTTCTGGAA
GCAGCTCCAACGTCGGTAGTAATACTGTGAACTGGTACCAGCAGCTTCCAGGAACGGCTCCTAAGCTCCTCAT
CTATAGTAATAATCAGAGACCCTCAGGTGTCCCTGACCGATTCTCTGGCTCCAAGTCTGGCACCTCAGCCTCC
CTCACCATCTCTGGACTGAAGACTGAGGACGAGGCTGGCTACTACTGTCAGTCTTATGATACCAGCAATGTGG
TATTCGGTGGAGGCACCAAGGTGACCGTCCTC
CDR1: AGCTCCAACGTCGGTAGTAATACT
CDR2: AGTAATAAT
CDR3: CAGTCTTATGATACCAGCAATGTGGTA
Amino Acid Sequence:
QPVLTQPPSASGTPGQRVTISCSGSSSNVGSNTVNWYQQLPGTAPKLLIYSNNQRPSGVP
DRFSGSKSGTSASLTISGLKTEDEAGYYCQSYDTSNVVFGGGTKVTVL
SEQ ID No. 66
(VL)
CDR1: SSNVGSNT
SEQ ID No. 67
(VL)
CDR2: SNN
SEQ ID No. 68
(VL)
CDR3: QSYDTSNVV
PBI#30af8-VH domain
Nucleic Acid Sequence:
CAGGTGCAGCTGCAGGAGTCTGGAGGTGGCTTGATACAGCCTGGTGGATCTCTGAGACTCTCCTGTGCAGCCT
CTGGATTCCAGTCTGATTCTAGTGATTATGCCATGAGTTGGGTCAGGCAGGCTCCAGGGAAGGGGCTGGAGTG
GGTCTCGTCCATCAGTGGTACTGGGGGTAGCACATACTACGCAGACTCCGTGAAGGGTCGCTTCACCATCTCC
AGAGACAATTCCAAGAACACACTCTATGTGCAAATGACCAGCCTGACAGCCGAGGACACGGCCGTGTACTACT
GTGCGAAGGTGGTCGGTTTCAGTACGTACGACTACTACTACCACTACGCTTTGGACGTCTGGGGCCAAGGGAC
CACGGTCACCGTCTCCTCA
CDR1: GGATTCCAGTCTGATTCTAGTGATTATGCC
CDR2: ATCAGTGGTACTGGCGGTAGCACA
CDR3: GCGAAGGTGGTCGGTTTCAGTACGTACGACTACTACTACCACTACGCTTTGGACGTC
Amino Acid Sequence:
QVQLQESGGGLIQPGGSLRLSCAASGFQSDSSDYAMSWVRQAPGKGLEWVSSISGTGGSTYY
ADSVKGRFTISRDNSKNTLYVQMTSLTAEDTAVYYCAKVVGFSTYDYYYHYALDVWGQGT
TVTVSS
SEQ ID No. 69
(VH)
CDR1: GFQSDSSDYA
SEQ ID No. 70
(VH)
CDR2: ISGTGGST
SEQ ID No. 71
(VH)
CDR3: AKVVGFSTYDYYYHYALDV
PBI#30af8-VL domain
Nucleic Acid Sequence:
CAGCCTGTGCTGACTCAGCCACCTTCAGCGTCTGGGACCCCCGGGCAGAGGGTCACCATCTCTTGTTCTGGAA
GCAGCTCCAACATCGGTAGTAATACTGTGAACTGGTACCAGCAGCTTCCAGGAACGGCTCCTAAGCTCCTCAT
CTATAGTGATGATCAGAGACCCTCAGGTGTCCCTGACCGATTCTCTGGCTCCAAGTCTGGCACCTCAGCCTCC
CTCACCATCTCTGGACTGAAGACTGAGGACGAGGCTGGCTACTACTGTCAGTCTTATGATACCAGCAATGTGG
TATTCGGTGGAGGCACCAAGGTGACCGTCCTC
CDR1: AGCTCCAACATCGGTAGTAATACT
CDR2: AGTGATGAT
CDR3: CAGTCTTATGATACCAGCAATGTGGTA
Amino Acid Sequence:
QPVLTQPPSASGTPGQRVTISCSGSSSNIGSNTVNWYQQLPGTAPKLLIYSDDQRPSGVP
DRFSGSKSGTSASLTISGLKTEDEAGYYCQSYDTSNVVFGGGTKVTVL
SEQ ID No. 72
(VL)
CDR1: SSNIGSNT
SEQ ID No. 73
(VL)
CDR2: SDD
SEQ ID No. 74
(VL)
CDR3: QSYDTSNVV
Humanized 8E1-VH domain
Nucleic Acid Sequence:
SEQ ID No. 75
(VH)
GAGCAGCAGCTGCTGGAGAGCGGCGGAGGCCTGGTGCAGCCAGGAGGAAGCCTGAGGCTGTCCTGCGCCGTGA
GCGGCTTTTCCCTGAGCAGCTACGCCATGGGCTGGGTGAGGCAGGCCCCTGGAAAGGGCCTGGAGTTCATCGG
CATCATCTCCTACTCCGGCAACACATACTACGCCAGCTGGGCCAAGGGCAGGTTCACAATCAGCAAGGATAGC
TCCAAGAACACCGTGTACCTGCAGATGAACAGCCTGAGGGCCGAGGACACAGCCGTGTACTTCTGTGCCAGGG
CCAGGTACGGCGGCTACTCCACCAATAGCTACTACCTGAACATCTGGGGCCCCGGCACACTGGTGACAGTGAG
CTCC
CDR1: GGCTTTTCCCTGAGCAGCTACGCC
CDR2: ATCTCCTACTCCGGCAACACA
CDR3: GCCAGGGCCAGGTACGGCGGCTACTCCACCAATAGCTACTACCTGAACATC
Amino Acid Sequence:
SEQ ID No. 76
(VH)
EQQLLESGGGLVQPGGSLRLSCAVSGFSLSSYAMGWVRQAPGKGLEFIGIISYSGNTYYASWAKGRFTISKDS
SKNTVYLQMNSLRAEDTAVYFCARARYGGYSTNSYYLNIWGPGTLVTVSS
CDR1: GFSLSSYA
CDR2: ISYSGNT
CDR3: ARARYGGYSTNSYYLNI
Humanized 8E1-VL domain
Nucleic Acid Sequence:
SEQ ID No. 77
(VL)
GATGTGCAGATGACCCAGTCCCCTTCCACCCTGTCCGCCAGCGTGGGCGATAGAGTGACAATCACCTGTCAGG
CCAGCGAGAATATCTACAGGGTGCTGGCCTGGTACCAGCAGAAGCCTGGCAAGAGGCCTAAGCTGCTGATCTA
CGGCGCCAGCAATCTGGAGTCCGGCGTGCCCAGCAGATTTTCCGGCTCCGGCAGCGGCACCGAGTACACACTG
ACAATCAGCTCCCTGCAGCCTGACGATTTCGCCACCTACTACTGCCAGGGCGTGGTGTACAACAGCGACGATA
GCGCCTTCGGCGGCGGCACAAAGGTGGAGATCAAG
CDR1: GAGAATATCTACAGGGTG
CDR2: GGCGCCAGC
CDR3: CAGGGCGTGGTGTACAACAGCGACGATAGCGCC
Amino Acid Sequence:
SEQ ID No. 78
(VL)
DVQMTQSPSTLSASVGDRVTITCQASENIYRVLAWYQQKPGKRPKLLIYGASNLESGVPSRFSGSGSGTEYTL
TISSLQPDDFATYYCQGVVYNSDDSAFGGGTKVEIK
CDR1: ENIYRV
CDR2: GAS
CDR3: QGVVYNSDDSA
Humanized 38D5-VH domain
Nucleic Acid Sequence:
SEQ ID No. 79
(VH)
GAGCAGCAGCTGCTGGAGTCCGGCGGAGGCCTGGTGCAGCCAGGAGGAAGCCTGAGACTGAGCTGCGCCGCCA
GCGGCATCGACTTCAATAATTACGGCATCTCCTGGGTGAGACAGGCCCCTGGCAAGGGCCTGGAGTGGATCGC
CTACAAGTACCCCGGCTTCGGCATCAGAAATTACGCCAATAGCGTGAAGGGCAGATTCACCATCTCCTCCGAT
AACAGCAAGAACACCGTGTACCTGCAGATGAATAGCCTGAGGGCCGAGGATACCGCCGTGTACTTTTGCGCCA
GGGGCGCCAGATACAGACACGATGACTACGGCGCCCTGAATCTGTGGGGCCCCGGCACACTGGTGACCGTGTC
CAGC
CDR1: GGCATCGACTTCAATAATTACGGC
CDR2: AAGTACCCCGGCTTCGGCATCAGA
CDR3: GCCAGGGGCGCCAGATACAGACACGATGACTACGGCGCCCTGAATCTG
Amino Acid Sequence:
SEQ ID No. 80
(VH)
EQQLLESGGGLVQPGGSLRLSCAASGIDFNNYGISWVRQAPGKGLEWIAYKYPGFGIRNYANSVKGRFTISSD
NSKNTVYLQMNSLRAEDTAVYFCARGARYPADDYGALNLWGPGTLVTVSS
CDR1: GIDFNNYG
CDR2: KYPGFGIR
CDR3: ARGARYRHDDYGALNL
Humanized 38D5-VL domain
Nucleic Acid Sequence:
SEQ ID No. 81
(VL)
GACATCCAGATGACCCAGTCCCCCAGCACACTGTCCGCCAGCGTGGGCGACAGAGTGACAATCACCTGCCAGG
CCAGCCAGTCCGTGACCAATCTGCTGGCCTGGTACCAGCAGAAGCCCGGCAAGCCCCCTAAGCTGCTGATCTA
CGGCGCCTCCAATCTGGAGAGCGGCGTGCCCAGCAGGTTTTCCGGCTCCGGCAGCGGCACAGAGTTTACACTG
ACCATCTCCTCCCTGCAGCCTGATGACTTCGCCACCTACTACTGTCAGAGCGGCTACTACAGCGCCAACACCT
TTGGCCCCGGCACCAAGGTGGATATCAAG
CDR1: CAGTCCGTGACCAATCTG
CDR2: GGCGCCTCC
CDR3: CAGAGCGGCTACTACAGCGCCAACACC
Amino Acid Sequence:
SEQ ID No. 82
(VL)
DIQMTQSPSTLSASVGDRVTITCQASQSVTNLLAWYQQKPGKPPKLLIYGASNLESGVPSRFSGSGSGTEFTL
TISSLQPDDFATYYCQSGYYSANTFGPGTKVDIK
CDR1: QSVTNL
CDR2: GAS
CDR3: QSGYYSANT
For use in human patients, it will be desirable to humanize these antibodies, replacing both
the constant regions of the heavy and light chains with human constant regions, as well as
replacing the framework regions of the variable regions with human antibody framework
regions. In some embodiments, the anti-NTPDase3 antibody or antigen-binding fragment
thereof, is a humanized version of a rabbit antibody.
```

In some embodiments, the anti-NTPDase3 antibody or antigen-binding fragment thereof comprises at least one heavy chain variable is at least 60% identical to SEQ ID No. 2, 10, 14, 18, 22, 26, 30, 34, 38, 42, 76, 80, or sequences listed in Table 2A, 2B, 2C, 2D, or 3 and even more preferably at least 65%, 70%, 75%, 80%, 85% or even 90% identical to SEQ ID No. 2, 10, 14, 18, 22, 26, 30, 34, 38, 42, 76, 80, or sequences listed in Table 2A, 2B, 2C, 2D, or 3, and able to specifically bind human NTPDase3.

In some embodiments, the anti-NTPDase3 antibody or antigen-binding fragment thereof comprises at least one light chain variable is at least 60% identical to SEQ ID No. 4, 12, 16, 20, 24, 28, 32, 36, 40, 44, 78, 82, or sequences listed in Table 2A, 2B, 2C, 2D, or 3, and even more preferably at least 65%, 70%, 75%, 80%, 85% or even 90% identical to SEQ ID No. 4, 12, 16, 20, 24, 28, 32, 36, 40, 44, 78, 82, or sequences listed in Table 2A, 2B, 2C, 2D, or 3, and able to specifically bind human NTPDase3.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

In certain embodiments, the anti-NTPDase3 antibody is a humanized antibody comprising a VH domain having human framework sequences associated with CDRs of a VH domain selected from SEQ ID No. 2, 10, 14, 18, 22, 26, 30, 34, 38, 42, 76, 80, and sequences listed in Tables 2A, 2B, 2C, 2D, and 3, and the CDRs of the corresponding VL domain selected from SEQ ID No. 4, 12, 16, 20, 24, 28, 32, 36, 40, 44, 78, 82, and sequences listed in Tables 2A, 2B, 2C, 2D, and 3. The CDRs are preferably identical, but may vary by 1, 2 or 3 amino acids across each CDR so long as the resulting antibody specifically binds human NTPDase3.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J Biol. Chem. 271:22611-22618 (1996)).

In certain embodiments, an anti-NTPDase3 antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

For instance, human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE technology; U.S. Pat. No. 5,770,429 describing HuMAB technology; U.S. Pat. No. 7,041,870 describing K-M MOUSE technology, and U.S. Patent Application Publication No. US 2007/0061900, describing VELOCIMOUSE technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage, yeast or bacterial display libraries. Such variable domain sequences may then be combined with a desired human constant domain. Techniques for selecting human antibodies from antibody libraries are described below.

To illustrate, anti-NTPDase3 antibodies encompassed by the present invention may be isolated by screening combinatorial libraries for antibodies with the desired activity or activities. For example, a variety of methods are known in the art for generating phage or yeast display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

As an example of phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

FcγRIII binding can also be increased by methods according to the state of the art, e.g. by modifying the amino acid sequence of the Fc part or the glycosylation of the Fc part of the antibody (see e.g. EP2235061). In certain embodiments, the subject antibodies are produced by cells in which, when glycosylated, less than 50% of the oligosaccharide chains on the antibody contain α-1,6-fucose. Typically, less than about 40%, less than about 30%, less than about 20%, less than about 10%, or less than 5% or less than 1% of the oligosaccharide chains contain α-1,6-fucose in a “hypofucosylated” antibody preparation. An “afucosylated” antibody lacks α-1,6-fucose in the carbohydrate attached to the CH2 domain of the IgG heavy chain. Mori, K et al., Cytotechnology 55 (2007) 109 and Satoh M, et al., Expert Opin Biol Ther. 6 (2006) 1161-1173 relate to a FUT8 (α-1,6-fucosyltransferase) gene knockout CHO line for the generation of afucosylated antibodies.

### IV. Expression Vectors

In certain embodiments, a recombinant expression vector is used to amplify and express DNA encoding the anti-NTPDase3 antibody described herein. For example, a recombinant expression vector can be a replicable DNA construct which has synthetic or cDNA-derived DNA fragments encoding the polypeptide chains of the anti-NTPDase3 antibody operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In other embodiments, where recombinant protein is expressed without a leader or transport sequence, it can include an N-terminal methionine residue. This residue can optionally be subsequently cleaved from the expressed recombinant protein to provide a final product.

The choice of an expression control sequence and an expression vector depends upon the choice of host cell. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

Suitable host cells for expression of the polypeptide chains of the anti-NTPDase3 antibody (or a protein to use as a target) include prokaryotes, yeast cells, insect cells, or higher eukaryotic cells under the control of appropriate promoters. Prokaryotes include gram-negative or gram-positive organisms, for example E. coli or Bacillus. Higher eukaryotic cells include established cell lines of mammalian origin as described below. Cell-free translation systems may also be employed. Appropriate cloning and expression vectors for use with bacterial, fungal, yeast, and mammalian cellular hosts are well known by those skilled in the art.

Various mammalian cell culture systems are used to express recombinant polypeptides. Expression of recombinant proteins in mammalian cells can be preferred because such proteins are generally correctly folded, appropriately modified, and biologically functional. Examples of suitable mammalian host cell lines include COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), and HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

In certain embodiments, the polynucleotide comprises a polynucleotide encoding an antibody heavy chain comprising a variable region at least 60% identical to a heavy chain variable region described herein, such as SEQ ID No. 2, and even more preferably at least 65%, 70%, 75%, 80%, 85% or even 90% identical to a heavy chain variable region described herein, such as SEQ ID No. 2, and able to specifically bind human NTPDase3.

In certain embodiments, the polynucleotide comprises a polynucleotide encoding an antibody light chain comprising a variable region at least 60% identical to a light chain variable region described herein, such as SEQ ID No. 4, and even more preferably at least 65%, 70%, 75%, 80%, 85% or even 90% identical to a light chain variable region described herein, such as SEQ ID No. 4, and able to specifically bind human NTPDase3.

### V. Encoded Anti-NTPDase3 Antibodies for In Vivo Delivery

Therapeutic vectors for delivering the coding sequence for an anti-NTPDase3 antibody to be expressed in the patient can be viral, non-viral, or physical. See, for example, Rosenberg et al., Science, 242:1575-1578, 1988, and Wolff et al., Proc. Natl. Acad. Sci. USA 86:9011-9014 (1989). Discussion of methods and compositions for use in gene therapy include Eck et al., in Goodman & Gilman's The Pharmacological Basis of Therapeutics, Ninth Edition, Hardman et al., eds., McGraw-Hill, New York, (1996), Chapter 5, pp. 77-101; Wilson, Clin. Exp. Immunol. 107 (Suppl. 1):31-32, 1997; Wivel et al., Hematology/Oncology Clinics of North America, Gene Therapy, S. L. Eck, ed., 12(3):483-501, 1998; Romano et al., Stem Cells, 18:19-39, 2000, and the references cited therein. U.S. Pat. No. 6,080,728 also provides a discussion of a wide variety of gene delivery methods and compositions. The routes of delivery include, for example, systemic administration and administration in situ. Well-known viral delivery techniques include the use of adenovirus, retrovirus, lentivirus, foamy virus, herpes simplex virus, vaccinia virus and adeno-associated virus vectors.

a. Viral Vectors

Preferred viral vectors are based on non-cytopathic eukaryotic viruses in which non-essential genes have been replaced with the nucleic acid construct carrying the nucleic acid sequences encoding the epitopes and targeting sequences of interest. Preferred viruses for certain embodiments encompassed by the present invention are the adenoviruses and adeno-associated (AAV) viruses, which are double-stranded DNA viruses that have already been approved for human use in gene therapy. In addition, preferred vectors for tolerizing do not include immune-stimulating sequences.

Adenovirus Vectors

One illustrative method for in vivo delivery of one or more nucleic acid sequences involves the use of an adenovirus expression vector. “Adenovirus expression vector” is meant to include those constructs containing adenovirus sequences sufficient to (a) support packaging of the construct and (b) to express a polynucleotide that has been cloned therein in a sense or antisense orientation. Of course, in the context of an antisense construct, expression does not require that the gene product be synthesized. In a specific embodiment, the delivery vector pertains to commercially available ORF of cytochrome b5 reductase 3 (CYB5R3), transcript variant 1 in adenoviral vector pAd, with C terminal Flag and His tag, (Vigene Biosciences Product code AH889428). WIPO Patent Application WO/2015/050364 also teaches vectors with expression constructs including a Cyb5r3 gene.

Adenoviral vectors are highly immunogenic and therefore are less preferred for administration to induce tolerance by presenting antigens, or in the case of autoimmune diseases. These vectors can be used, however to induce immunity, for example in treatment of infectious diseases and the like, include, for example, influenza, HBV, HCV and HIV.

Adeno-Associated Virus Vectors (AAV)

AAV is a good choice of delivery vehicles due to its safety, i.e., genetically engineered (recombinant) does not integrate into the host genome. Likewise, AAV is not pathogenic and not associated with any disease. The removal of viral coding sequences minimizes immune reactions to viral gene expression, and therefore, rAAV does not evoke an inflammatory response. According to a specific embodiment, an AAV vector containing an epitope sequence containing nucleic acid construct described herein is useful for transducing APCs.

Typically, viral vectors containing an epitope containing nucleic acid construct are assembled from polynucleotides encoding the desired epitopes, suitable regulatory elements and elements necessary for epitope expression which mediate cell transduction. In one embodiment, adeno-associated viral (AAV) vectors are employed. In a more specific embodiment, the AAV vector is an AAV1, AAV6, or AAV8.

The AAV expression vector which harbors the DNA molecule of interest bounded by AAV ITRs, can be constructed by directly inserting the selected sequence(s) into an AAV genome which has had the major AAV open reading frames (“ORFs”) excised therefrom. Examples of constitutive promoters which may be included in the AAV of this invention include, without limitation, the exemplified CMV immediate early enhancer/chicken β-actin (CBA) promoter.

For eukaryotic cells, expression control sequences typically include a promoter, an enhancer, such as one derived from an immunoglobulin gene, SV40, cytomegalovirus, etc., and a polyadenylation sequence which may include splice donor and acceptor sites. The polyadenylation sequence generally is inserted following the transgene sequences and before the 3′ ITR sequence. In one embodiment, the bovine growth hormone polyA may be used.

Selection of these and other common vector and regulatory elements are conventional, and many such sequences are available. See, e.g., Sambrook et al., and references cited therein at, for example, pages 3.18-3.26 and 16.17-16.27 and Ausubel et al., Current Protocols in Molecular Biology, John Wiley & Sons, New York, 1989). Of course, not all vectors and expression control sequences will function equally well to express all of the transgenes of this invention. However, one of skill in the art may make a selection among these expression control sequences without departing from the scope of this invention. Suitable promoter/enhancer sequences may be selected by one of skill in the art using the guidance provided by this application. Such selection is a routine matter and is not a limitation of the molecule or construct.

Retrovirus Vectors

In certain embodiments, the viral vector may be a retroviral vector. “Retroviruses” are viruses having an RNA genome. In particular embodiments, a retroviral vector contains all of the cis-acting sequences necessary for the packaging and integration of the viral genome, i.e., (a) a long terminal repeat (LTR), or portions thereof, at each end of the vector; (b) primer binding sites for negative and positive strand DNA synthesis; and (c) a packaging signal, necessary for the incorporation of genomic RNA into virions. More detail regarding retroviral vectors can be found in Boesen, et al., 1994, Biotherapy 6:291-302; Clowes, et ai, 1994, J. Clin. Invest. 93:644-651; Kiem, et al., 1994, Blood 83: 1467-1473; Salmons and Gunzberg, 1993, Human Gene Therapy 4: 129-141; Miller, et al., 1993, Meth. Enzymol. 217:581-599; and Grossman and Wilson, 1993, Curr. Opin. in Genetics and Devel. 3: 110-1 14.

“Gammaretroviruses” refers to a genus of the retroviridae family. Exemplary gammaretroviruses include mouse stem cell virus, murine leukemia virus, feline leukemia virus, feline sarcoma virus, and avian reticuloendotheliosis viruses.

Widely used retroviral vectors include those based upon murine leukemia virus (MuLV), gibbon ape leukemia virus (GaLV), simian immunodeficiency virus (SIV), human immunodeficiency virus (HIV), and combinations thereof (see, e.g., Buchscher et al., J. Virol. 66:2731-2739, 1992; Johann et al., J. Virol. 66: 1635-1640, 1992; Sommerfelt et al., Virol. 176:58-59, 1990; Wilson et al., J. Virol. 63:2374-2378, 1989; Miller et al., J. Virol. 65:2220-2224, 1991; and PCT/US94/05700).

Lentiviral vectors refer to a genus of retroviruses that are capable of infecting dividing and non-dividing cells and typically produce high viral titers. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

In particular embodiments, other retroviral vectors can be used. These include, e.g., vectors based on human foamy virus (HFV) or other viruses in the Spumavirus genera. Foamy viruses (FVes) are the largest retroviruses known today and are widespread among different mammals, including all non-human primate species, however are absent in humans. This complete apathogenicity qualifies FV vectors as ideal gene transfer vehicles for genetic therapies in humans and clearly distinguishes FV vectors as gene delivery system from HIV-derived and also gammaretrovirus-derived vectors.

Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are known to those of skill in the art.

The retroviral genome contains three genes, gag, pol, and env that code for capsid proteins, polymerase enzyme, and envelope components, respectively. A sequence found upstream from the gag gene contains a signal for packaging of the genome into virions. Retroviral vectors are gene transfer plasmids wherein the heterologous nucleic acid resides between two retroviral LTRs. Retroviral vectors typically contain appropriate packaging signals that enable the retroviral vector, or RNA transcribed using the retroviral vector as a template, to be packaged into a viral virion in an appropriate packaging cell line (see, e.g., U.S. Pat. No. 4,650,764). These two long terminal repeat (LTR) sequences are present at the 5′ and 3′ ends of the viral genome. These contain strong promoter and enhancer sequences and are also required for integration in the host cell genome (Coffin, 1990). In order to construct a retroviral vector, a nucleic acid encoding one or more oligonucleotide or polynucleotide sequences of interest is inserted into the viral genome in the place of certain viral sequences to produce a virus that is replication-defective. Also included are episomal or non-integrating forms of retroviral vectors based on lentiviruses (e.g., a type of retrovirus).

Lentiviral vectors are useful when stable expression is needed, but lentiviral vectors can be immunogenic, and possibly have other undesirable effects. Therefore, although lentiviral vectors are convenient for research, care should be taken when using them for human administration, particularly where it is desired to induce tolerance rather than immunity. Lentiviruses are suitable for engineering T cells or dendritic cells or other antigen presenting cells ex vivo for cancer therapy, although mRNA electroporation is more safe. However, two recent advances have made the use of lentiviruses safer and more clinically translatable. First, the coexpression of a suicide gene along with the antigens whose products become functional when a drug is administered. A typical example is Herpes simplex virus thymidine kinase (HSV-Tk). Cells that express these genes can metabolize the drug ganciclovir into a cytotoxic product that induces cell death. Thus, in case some transduced cells become malignant, they can be eradicated. About a dozen such systems exist (Duarte et al., Cancer Letters, 324:160-170, 2012). Second, there are now non-integrating lentiviral vectors being developed that are therefore non-oncogenic (Nightingale et al., 2006, Mol. Ther., 13:1121-1132). These methods can be used with the invention according to the judgement of the person of skill in the art.

Suitable retroviral vectors for use herein are described, for example, in U.S. Pat. Nos. 5,399,346 and 5,252,479; and in WIPO publications WO 92/07573, WO 90/06997, WO 89/05345, WO 92/05266 and WO 92/14829, which provide a description of methods for efficiently introducing nucleic acids into human cells using such retroviral vectors. Other retroviral vectors include, for example, mouse mammary tumor virus vectors (e.g., Shackleford et al., Proc. Natl. Acad. Sci. U.S.A. 85:9655-9659, 1998), lentiviruses, and the like. An exemplary viral vector is plentilox-IRES-GFP.

Additional retroviral viral delivery systems that can be readily adapted for delivery of a transgene encoding an Anti-NTPDase3 antibody Agent include, merely to illustrate Published PCT Applications WO/2010/045002, WO/2010/148203, WO/2011/126864, WO/2012/058673, WO/2014/066700, WO/2015/021077, WO/2015/148683, WO/2017/040815—the specifications and figures of each of which are incorporated by reference herein.

In certain embodiments, the retrovirus is a recombinant replication competent retrovirus comprising: a nucleic acid sequence encoding a retroviral GAG protein; a nucleic acid sequence encoding a retroviral POL protein; a nucleic acid sequence encoding a retroviral envelope; an oncoretroviral polynucleotide sequence comprising Long-Terminal Repeat (LTR) sequences at the 5′ and 3′ end of the oncoretroviral polynucleotide sequence; a cassette comprising an internal ribosome entry site (IRES) operably linked to a coding sequence for an Anti-NTPDase3 antibody Agent, wherein the cassette is positioned 5′ to the U3 region of the 3′ LTR and 3′ to the sequence encoding the retroviral envelope; and cis-acting sequences for reverse transcription, packaging and integration in a target cell.

In certain embodiments, the retrovirus is a recombinant replication competent retrovirus comprising: a retroviral GAG protein; a retroviral POL protein; a retroviral envelope; a retroviral polynucleotide comprising Long-Terminal Repeat (LTR) sequences at the 3′ end of the retroviral polynucleotide sequence, a promoter sequence at the 5′ end of the retroviral polynucleotide, the promoter being suitable for expression in a mammalian cell, a gag nucleic acid domain, a pol nucleic acid domain and an env nucleic acid domain; a cassette comprising an Anti-NTPDase3 antibody Agent coding sequence operably linked to a heterologous polynucleotide, wherein the cassette is positioned 5′ to the 3′ LTR and is operably linked and 3′ to the env nucleic acid domain encoding the retroviral envelope; and cis-acting sequences necessary for reverse transcription, packaging and integration in a target cell.

In certain preferred embodiments of the recombinant replication competent retrovirus, the envelope is chosen from one of amphotropic, polytropic, xenotropic, 10A1, GALV, Baboon endogenous virus, RD114, rhabdovirus, alphavirus, measles or influenza virus envelopes.

In certain preferred embodiments of the recombinant replication competent retrovirus, the retroviral polynucleotide sequence is engineered from a virus selected from the group consisting of murine leukemia virus (MLV), Moloney murine leukemia virus (MoMLV), Feline leukemia virus (FeLV), Baboon endogenous retrovirus (BEV), porcine endogenous virus (PERV), the cat derived retrovirus RD114, squirrel monkey retrovirus, Xenotropic murine leukemia virus-related virus (XMRV), avian reticuloendotheliosis virus (REV), or Gibbon ape leukemia virus (GALV).

In certain preferred embodiments of the recombinant replication competent retrovirus, retrovirus is a gammaretrovirus.

In certain preferred embodiments of the recombinant replication competent retrovirus, there is a second cassette comprising a coding sequence for a second therapeutic protein, such as another checkpoint inhibitor polypeptide, a co-stimulatory polypeptide and/or an immunostimulatory cytokine (merely as examples), e.g., downstream of the cassette. In certain instances, the second cassette can include an internal ribosome entry site (IRES) or a minipromoter or a polIIII promoter operably linked to the coding sequence for the second therapeutic protein.

In certain preferred embodiments of the recombinant replication competent retrovirus, it is a nonlytic, amphotropic retroviral replicating vector which, preferably, selectively infects and replicates in the cells of the tumor microenvironment.

Other Viral Vectors as Expression Constructs

Other viral vectors may be employed as expression constructs in embodiments encompassed within the present invention for the delivery of oligonucleotide or polynucleotide sequences to a host cell. Vectors derived from viruses such as vaccinia virus, polioviruses and herpes viruses may be employed. They offer several attractive features for various mammalian cells. Also included are hepatitis B viruses.

b. Non-Viral Vectors

Plasmid Vectors

Other vectors include plasmid vectors. Plasmid vectors have been extensively described in the art and are well known to those of skill in the art. See e.g. Sambrook et al., 1989, cited above. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide epitope encoded by nucleic acid within the plasmid. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

Thus, in one aspect, a plasmid is provided for expression of the epitope containing nucleic acid construct which includes an expression cassette; also referred to as a transcription unit. When a plasmid is placed in an environment suitable for epitope expression, the transcriptional unit will express the polynucleotide including a sequence encoding the epitopes, ETS and MHCII activator sequence, or sequence encoding the epitopes and secretion signal sequence, and anything else encoded in the construct. The transcription unit includes a transcriptional control sequence, which is transcriptionally linked with a cellular immune response element coding sequence. Transcriptional control sequence may include promoter/enhancer sequences such as cytomegalovirus (CMV) promoter/enhancer sequences. However, those skilled in the art will recognize that a variety of other promoter sequences suitable for expression in eukaryotic cells are known and can similarly be used in the constructs disclosed herein. The level of expression of the nucleic acid product will depend on the associated promoter and the presence and activation of an associated enhancer element.

In certain embodiments, a sequence encoding the desired epitopes and targeting sequence can be cloned into an expression plasmid which contains the regulatory elements for transcription, translation, RNA stability and replication (i.e., including a transcriptional control sequence). Such expression plasmids are well known in the art and one of ordinary skill would be capable of designing an appropriate expression construct with a polynucleotide including a sequence encoding a cellular immune response element or fragment thereof in such a manner that the cellular immune response element is expressible. There are numerous examples of suitable expression plasmids into which a polynucleotide including a sequence could be cloned such as pCI-neo, pUMVC or pcDNA3.

Large quantities of a bacterial host harboring a plasmid for expression of cellular immune response element or fragment thereof may be fermented and the plasmid can be purified for subsequent use. Current human clinical trials using plasmids utilize this approach. Recombinant DNA Advisory Committee Data Management Report, Human Gene Therapy 6: 535-548, 1994. Current DNA isolation methods known in the art include removal of lipopolysaccharides (endotoxins) that are contaminants from the bacteria used to propagate the plasmids. This step is most preferably taken for use of tolerogenic DNA vaccines as endotoxins act as strong adjuvants and can produce undesired immune stimulation.

The purpose of the plasmid is the efficient delivery of nucleic acid sequences to and expression of therapeutic epitopes in a cell or tissue. In particular, the purpose of the plasmid may be to achieve high copy number, avoid potential causes of plasmid instability and provide a means for plasmid selection. As for expression, the nucleic acid cassette contains the necessary elements for expression of the nucleic acid within the cassette. Expression includes the efficient transcription of an inserted gene, nucleic acid sequence, or nucleic acid cassette with the plasmid. Expression products may be proteins, polypeptides or RNA. The nucleic acid sequence can be contained in a nucleic acid cassette. Expression of the nucleic acid can be continuous or regulated.

Minicircle

Embodiments of nucleic acid constructs described herein may be processed in the form of minicircle DNA. Minicircle DNA pertains to small (2-4 kb) circular plasmid derivatives that have been freed from all prokaryotic vector parts. Since minicircle DNA vectors contain no bacterial DNA sequences, they are less likely to be perceived as foreign and destroyed. (Typical transgene delivery methods involve plasmids, which contain foreign DNA.) As a result, these vectors can be expressed for longer periods of time (in order of weeks or months) compared to conventional plasmids (days to weeks). The smaller size of minicircles also extends their cloning capacity and facilitates their delivery into cells. Kits for producing minicircle DNA are known in the art and are commercially available (System Biosciences, Inc., Palo Alto, Calif.). Information on minicircle DNA is provided in Dietz et al., Vector Engineering and Delivery Molecular Therapy (2013); 21 8, 1526-1535 and Hou et al., Molecular Therapy—Methods & Clinical Development, Article number: 14062 (2015) doi:10.1038/mtm.2014.62. More information on Minicircles is provided in Chen Z Y, He C Y, Ehrhardt A, Kay M A. Mol Ther. 2003 September; 8(3):495-500 and Minicircle DNA vectors achieve sustained expression reflected by active chromatin and transcriptional level. Gracey Maniar L E, Maniar J M, Chen Z Y, Lu J, Fire A Z, Kay M A. Mol Ther. 2013 January; 21(1):131-8

As an initial step in the process of ultimately obtaining expression of a product encoded by a nucleic acid, is to effect the uptake of the nucleic acid by cells. Uptake of nucleic acid by cells is dependent on a number of factors, one of which is the length of time during which a nucleic acid is in proximity to a cellular surface. For instance, after intramuscular (i.m.) administration of plasmid DNA in buffer, a marked reduction in gene expression was observed if the muscle is massaged, presumably due to DNA leakage out of the muscle either directly or via lymphatic vessels (Human Gene Therapy 4:151-159; 1993). Accordingly, it may be desirable to formulate nucleic acids with compounds which would retard the rate at which nucleic acids diffuse or are carried away from a site at which cellular uptake of the nucleic acid is desired. Further, these compounds could be suitable for administration to an organism by means such as injection while maintaining or regaining the physical characteristics necessary to increase cellular uptake of nucleic acids.

In order to effect expression of oligonucleotide or polynucleotide sequences, the expression construct must be delivered into a cell. In certain embodiments encompassed by the present invention, an expression construct comprising one or more oligonucleotide or polynucleotide sequences may simply consist of naked recombinant DNA or plasmids.

To prime immunity, DNA vaccine vectors of any type preferably are engineered to be CpG-rich (to stimulate TLR9 on immune cells) or conversely are engineered to remove CpG, and when possible, replace CpG motifs with GpG motifs (Ho et al., J. Immunol. 71(9):4920-6, 2003; Ho et al., J. Immunol. 175(9):6226-34, 2005). DNA vaccines can be engineered to contain the antigen(s)/epitope(s), and also can contain additional genes for co-expression with the antigens to act as adjuvants or immunomodulators (multiple promoter vectors. These DNA vaccines have been found to be safe clinically, for example in T1D patients (Roep et al., Sci. Transl. Med. 5(191):191ra82, 2013).

Mechanical Delivery Systems

Additional non-viral delivery methods include but are not limited to mechanical delivery systems that can be used in vitro such as the approach described in Woffendin et al., Proc. Natl. Acad. Sci. USA 91(24):11581, 1994; deposition of photopolymerized hydrogel materials or use of ionizing radiation (see, e.g., U.S. Pat. No. 5,206,152 and WO 92/11033); the use of a hand-held gene transfer particle gun (see, e.g., U.S. Pat. No. 5,149,655); and the use of ionizing radiation for activating transferred gene (see, e.g., U.S. Pat. No. 5,206,152 and WO 92/11033). Delivery devices can also be biocompatible, and may also be biodegradable. The formulation preferably provides a relatively constant level of active component release. On the other hand, a more rapid rate of release immediately upon administration may be desired. The formulation of such compositions is well within the level of ordinary skill in the art using known techniques.

Physical methods to enhance delivery include electroporation (where short pulses of high voltage carries the nucleic acid across the membrane), a gene gun (where DNA is loaded onto gold particles and forced to achieve penetration of the DNA into the cells), sonoporation, magnetofection, hydrodynamic delivery and the like, all of which are known to those of skill in the art. DNA also can be encapsulated in liposomes, preferably cationic liposomes, or polymersomes (synthetic liposomes) which can interact with the cell membrane and fuse or undergo endocytosis to effect DNA transfer into the cell. The DNA also can be formed into complexes with polymers (polyplexes) or with dendrimers which can directly release their load into the cytoplasm of a cell.

Illustrative carriers useful in this regard include microparticles of poly(lactide-co-glycolide), polyacrylate, latex, starch, cellulose, dextran and the like. Other illustrative delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701 and WO 96/06638). The amount of active agent contained within a sustained release formulation depends upon the site of implantation, the rate and expected duration of release and the nature of the condition to be treated or prevented.

Biodegradable microspheres (e.g., polylactate polyglycolate) may be employed as carriers for compositions. Suitable biodegradable microspheres are disclosed, for example, in U.S. Pat. Nos. 4,897,268; 5,075,109; 5,928,647; 5,811,128; 5,820,883; 5,853,763; 5,814,344, 5,407,609 and 5,942,252. Modified hepatitis B core protein carrier systems such as described in WO/99 40934, and references cited therein, will also be useful for many applications. Another illustrative carrier/delivery system employs a carrier comprising particulate-protein complexes, such as those described in U.S. Pat. No. 5,928,647, which can have the added benefit when used intratumorally to deliver the coding sequence for an Anti-NTPDase3 antibody Agent of being capable of inducing an MHC I-restricted cytotoxic T lymphocyte responses targeted tumor tissues of the patient.

Biodegradable polymeric nanoparticles facilitate nonviral nucleic acid transfer to cells. Small (approximately 200 nm), positively charged (approximately 10 mV) particles are formed by the self-assembly of cationic, hydrolytically degradable poly(beta-amino esters) and plasmid DNA.

Polynucleotides may also be administered to cells by direct microinjection, temporary cell permeabilizations (e.g., co-administration of repressor and/or activator with a cell permeabilizing agent), fusion to membrane translocating peptides, and the like.

In certain particular embodiments of the present disclosure, the gene construct is introduced into target cells via electroporation. Electroporation involves the exposure of cells (or tissues) and DNA (or a DNA complex) to a high-voltage electric discharge. In vivo electroporation is a gene delivery technique that has been used successfully for efficient delivery of plasmid DNA to many different tissues. Studies have reported the administration of in vivo electroporation for delivery of plasmid DNA to B16 melanomas and other tumor tissues. Systemic and local expression of a gene or cDNA encoded by a plasmid can be obtained with administration of in vivo electroporation. Use of in vivo electroporation enhances plasmid DNA uptake in tumor tissue, resulting in expression within the tumor, and delivers plasmids to muscle tissue, resulting in systemic expression of secreted proteins, such as cytokines (see, e.g., U.S. Pat. No. 8,026,223). Exemplary techniques, vectors and devices for electroporating Anti-NTPDase3 antibody Agent transgenes into cells in vivo include PCT Publications WO/2017/106795, WO/2016/161201, WO/2016/154473, WO/2016/112359 and WO/2014/066655.

U.S. Pat. No. 7,245,963 describes modular electrode systems and their use for facilitating the introduction of a biomolecule into cells of a selected tissue in a body or plant. The modular electrode systems comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the ceil between the plurality of electrodes. The entire content of U.S. Pat. No. 7,245,963 is hereby incorporated by reference.

U.S. Patent Pub. 2005/0052630 describes an electroporation device which may be used to effectively facilitate the introduction of a biomolecule into ceils of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk (see, e.g., U.S. Patent Pub. 2005/0052630) is hereby incorporated by reference.

The electrode arrays and methods described in U.S. Pat. No. 7,245,963 and U.S. Patent Pub. 2005/0052630 are adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes.

Typically, the electric fields needed for in vivo cell electroporation are generally similar in magnitude to the fields required for cells in vitro. In one embodiment, the magnitude of the electric field range from approximately, 10 V/cm to about 1500 V/cm, preferably from about 300 V/cm to 1500 V/cm and preferably from about 1000 V/cm to 1500 V/cm. Alternatively, lower field strengths (from about 10 V/cm to 100 V/cm, and more preferably from about 25 V/cm to 75 V/cm) the pulse length is long. For example, when the nominal electric field is about 25-75 V/cm, if is preferred that the pulse length is about 10 msec.

The pulse length can be about 10 s to about 100 ms. There can be any desired number of pulses, typically one to 100 pulses per second. The delay between pulses sets can be any desired time, such as one second. The waveform, electric field strength and pulse duration may also depend upon the type of cells and the type of molecules that are to enter the cells via electroporation.

Also encompassed are electroporation devices incorporating electrochemical impedance spectroscopy (“EIS”). Such devices provide real-time information on in vivo, in particular, intratumoral electroporation efficiency, allowing for the optimization of conditions. Examples of electroporation devices incorporating EIS can be found, e.g., in WO2016/161201, which is hereby incorporated by reference.

Uptake of the non-viral delivery vectors encompassed by the present invention may also be enhanced by plasma electroporation also termed avalanche transfection. Briefly, microsecond discharges create cavitation microbubbles at electrode surface. The mechanical force created by the collapsing microbubbles combined with the magnetic field serve to increase transport efficiency across the cell membrane as compared with the diffusion mediated transport associated with conventional electroporation. The technique of plasma electroporation is described in U.S. Pat. Nos. 7,923,251 and 8,283,171. This technique may also be employed in vivo for the transformation of cells. Chaiberg, et al. (2006) Investigative Ophthalmology & Visual Science 47:4083-4090; Chaiberg, et al. U.S. Pat. No. 8,101,169 Issued Jan. 24, 2012.

Other alternative electroporation technologies are also contemplated. In vivo plasmid delivery can also be performed using cold plasma. Plasma is one of the four fundamental states of matter, the others being solid, liquid, and gas. Plasma is an electrically neutral medium of unbound positive and negative particles (i.e. the overall charge of a plasma is roughly zero). A plasma can be created by heating a gas or subjecting it to a strong electromagnetic field, applied with a laser or microwave generator. This decreases or increases the number of electrons, creating positive or negative charged particles called ions (Luo, et al. (1998) Phys. Plasma 5:2868-2870) and is accompanied by the dissociation of molecular bonds, if present.

Cold plasmas (i.e., non-thermal plasmas) are produced by the delivery of pulsed high voltage signals to a suitable electrode. Cold plasma devices may take the form of a gas jet device or a dielectric barrier discharge (DBD) device. Cold temperature plasmas have attracted a great deal of enthusiasm and interest by virtue of their provision of plasmas at relatively low gas temperatures. The provision of plasmas at such a temperature is of interest to a variety of applications, including wound healing, anti-bacterial processes, various other medical therapies and sterilization. As noted earlier, cold plasmas (i.e., non-thermal plasmas) are produced by the delivery of pulsed high-voltage signals to a suitable electrode. Cold plasma devices may take the form of a gas jet device, a dielectric barrier discharge (DBD) device or multi-frequency harmonic-rich power supply.

Dielectric barrier discharge device relies on a different process to generate the cold plasma. A dielectric barrier discharge (DBD) device contains at least one conductive electrode covered by a dielectric layer. The electrical return path is formed by the ground that can be provided by the target substrate undergoing the cold plasma treatment or by providing an in-built ground for the electrode. Energy for the dielectric barrier discharge device can be provided by a high voltage power supply, such as that mentioned above. More generally, energy is input to the dielectric barrier discharge device in the form of pulsed DC electrical voltage to form the plasma discharge. By virtue of the dielectric layer, the discharge is separated from the conductive electrode and electrode etching and gas heating is reduced. The pulsed DC electrical voltage can be varied in amplitude and frequency to achieve varying regimes of operation. Any device incorporating such a principle of cold plasma generation (e.g., a DBD electrode device) falls within the scope of various embodiments encompassed by the present invention.

Cold plasma has been employed to transfect cells with foreign nucleic acids. In particular, transfection of tumor cells (see, e.g., Connolly, et al. (2012) Human Vaccines & Immune-therapeutics 8: 1729-1733; and Connolly et al (2015) Bioelectrochemistry 103: 15-21).

In certain illustrative embodiments, the transgene construct encoding the anti-NTPDase3 antibody agent encompassed by the present invention is delivered using an electroporation device comprising: an applicator; a plurality of electrodes extending from the applicator, the electrodes being associated with a cover area; a power supply in electrical communication with the electrodes, the power supply configured to generate one or more electroporating signals to cells within the cover area; and a guide member coupled to the electrodes, wherein the guide member is configured to adjust the cover area of the electrodes. At least a portion of the electrodes can be positioned within the applicator in a conical arrangement. The one or more electroporating signals may be each associated with an electric field. The device may further comprise a potentiometer coupled to the power supply and electrodes. The potentiometer may be configured to maintain the electric field substantially within a predetermined range.

The one or more electroporating signals may be each associated with an electric field. The device may further comprise a potentiometer coupled to the power supply and the electrodes. The potentiometer may be configured to maintain the electric field within a predetermined range so as to substantially prevent permanent damage in the cells within the cover area and/or substantially minimize pain. For instance, potentiometer may be configured to maintain the electric field to about 1300 V/cm.

The power supply may provide a first electrical signal to a first electrode and a second electrical signal to a second electrode. The first and second electrical signals may combine to produce a wave having a beat frequency. The first and second electrical signals may each have at least one of a unipolar waveform and a bipolar waveform. The first electrical signal may have a first frequency and a first amplitude. The second electrical signal may have a second frequency and a second amplitude. The first frequency may be different from or the same as the second frequency. The first amplitude may be different from or the same as the second amplitude.

In certain embodiments, the present invention provides a method for treating a subject having a tumor, the method comprising: injecting the tumor with an effective dose of plasmid coding for an Anti-NTPDase3 antibody Agent; and administering electroporation therapy to the tumor. In certain embodiments, the electroporation therapy further comprises the administration of at least one voltage pulse of about 200 V/cm to about 1500 V/cm over a pulse width of about 100 microseconds to about 20 milliseconds.

In certain embodiments, the plasmid (or a second electroporated plasmid) further encodes at least one immunostimulatory cytokine, such as selected from the group encoding IL-12, IL-15, and a combination of IL-12 and IL-15.

Lipids and Polycationic Molecules for Delivering Anti-NTPDase3 Antibody Encoding Nucleic Constructs

Lipid-mediated nucleic acid delivery and expression of foreign nucleic acids, including mRNA, in vitro and in vivo has been very successful. Lipid based non-viral formulations provide an alternative to adenoviral gene therapies. Current in vivo lipid delivery methods use subcutaneous, intradermal, intratumoral, or intracranial injection. Advances in lipid formulations have improved the efficiency of gene transfer in vivo (see PCT Application WO 98/07408). For instance, a lipid formulation composed of an equimolar ratio of 1,2-bis(oleoyloxy)-3-(trimethyl ammonio)propane (DOTAP) and cholesterol can significantly enhances systemic in vivo gene transfer. The DOTAP:cholesterol lipid formulation forms unique structure termed a “sandwich liposome”. This formulation is reported to “sandwich” DNA between an invaginated bi-layer or ‘vase’ structure. Beneficial characteristics of these lipid structures include a positive p, colloidal stabilization by cholesterol, two dimensional nucleic acid packing and increased serum stability.

Cationic liposome technology is based on the ability of amphipathic lipids, possessing a positively charged head group and a hydrophobic lipid tail, to bind to negatively charged DNA or RNA and form particles that generally enter cells by endocytosis. Some cationic liposomes also contain a neutral co-lipid, thought to enhance liposome uptake by mammalian cells. Similarly, other polycations, such as poly-I-lysine and polyethylene-imine, complex with nucleic acids via charge interaction and aid in the condensation of DNA or RNA into nanoparticles, which are then substrates for endosome-mediated uptake.[8] Several of these cationic-nucleic acid complex technologies have been developed as potential clinical products, including complexes with plasmid DNA (pDNA), oligodeoxynucleotides, and various forms of synthetic RNA.

The nucleic acid constructs disclosed herein may be associated with polycationic molecules that serve to enhance uptake into cells. Complexing the nucleic acid construct with polycationic molecules also helps in packaging the construct such their size is reduced, which is believed to assist with cellular uptake. Once in the endosome, the complex dissociates due to the lower pH, and the polycationic molecules can disrupt the endosome's membrane to facilitate DNA escape into the cytoplasm before it can be degraded. Preliminary data shows that the nucleic acid construct embodiments had enhanced uptake into SCs over DCs when complexed with the polycationic molecules polylysine or polyethyleneimine.

One example of polycationic molecules useful for complexing with nucleic acid constructs includes cell penetrating peptides (CPP), examples include polylysine (described above), polyarginine and Tat peptides. Cell penetrating peptides (CPP) are small peptides which can bind to DNA and, once released, penetrate cell membranes to facilitate escape of the DNA from the endosome to the cytoplasm. Another example of a CPP pertains to a 27 residue chimeric peptide, termed MPG, was shown some time ago to bind ss- and ds-oligonucleotides in a stable manner, resulting in a non-covalent complex that protected the nucleic acids from degradation by DNase and effectively delivered oligonucleotides to cells in vitro (Mahapatro A, et al., J Nanobiotechnol, 2011, 9:55). The complex formed small particles of approximately 150 nm to 1 um when different peptide:DNA ratios were examined, and the 10:1 and 5:1 ratios (150 nm and 1 um respectively). Another CPP pertains to a modified tetrapeptide [tetralysine containing guanidinocarbonylpyrrole (GCP) groups (TL-GCP)], which was reported to bind with high affinity to a 6.2 kb plasmid DNA resulting in a positive charged aggregate of 700-900 nm (Li et al., Agnew Chem Int Ed Enl 2015; 54(10):2941-4). RNA can also be complexed by such polycationic molecules for in vivo delivery.

Other examples of polycationic molecules that may be complexed with the nucleic acid constructs described herein include polycationic polymers commercially available as JETPRIME® and In Vivo JET (Polypus-transfection, S.A., Illkirch, France).

### VI. Methods of Use and Pharmaceutical Compositions

The anti-NTPDase3 antibodies encompassed by the present invention are useful in a variety of applications including, but not limited to, therapeutic treatment methods, such as immunotherapy for cancer. In certain embodiments, an anti-NTPDase3 antibody described herein is useful for activating, promoting, increasing, and/or enhancing an immune response, inhibiting tumor growth, reducing tumor volume, inducing tumor regression, increasing tumor cell apoptosis, and/or reducing the tumorigenicity of a tumor. In certain embodiments, the anti-NTPDase3 antibody and derivatives encompassed by the present invention are also useful for immunotherapy against pathogens, such as viruses. In certain embodiments, an anti-NTPDase3 antibody described herein is useful for inhibiting viral infection, reducing viral infection, increasing virally-infected cell apoptosis, and/or increasing killing of virus-infected cells. The methods of use may be in vitro, ex vivo, or in vivo methods.

The present invention provides methods for activating an immune response in a subject using an anti-NTPDase3 antibody described herein. In some embodiments, the invention provides methods for promoting an immune response in a subject using an anti-NTPDase3 antibody described herein. In some embodiments, the invention provides methods for increasing an immune response in a subject using an anti-NTPDase3 antibody described herein. In some embodiments, the invention provides methods for enhancing an immune response in a subject using an anti-NTPDase3 antibody described herein. In some embodiments, the activating, promoting, increasing, and/or enhancing of an immune response comprises increasing cell-mediated immunity.

In some embodiments, the activating, promoting, increasing, and/or enhancing of an immune response comprises decreasing the number of M2 or M2-like macrophage in the tumor. In some embodiments, the activating, promoting, increasing, and/or enhancing of an immune response comprises decreasing M2 macrophage activity. In some embodiments, the activating, promoting, increasing, and/or enhancing of an immune response comprises increasing M1 macrophage activity.

In some embodiments, the activating, promoting, increasing, and/or enhancing of an immune response comprises decreasing the level of adenosine in the tumor.

In some embodiments, the activating, promoting, increasing, and/or enhancing of an immune response comprises increasing Th1-type responses. In some embodiments, the activating, promoting, increasing, and/or enhancing of an immune response comprises increasing T-cell activity. In some embodiments, the activating, promoting, increasing, and/or enhancing of an immune response comprises increasing CD4+ T-cell activity. In some embodiments, the activating, promoting, increasing, and/or enhancing of an immune response comprises increasing CD8+ T-cell activity. In some embodiments, the activating, promoting, increasing, and/or enhancing of an immune response comprises increasing CTL activity. In some embodiments, the activating, promoting, increasing, and/or enhancing of an immune response comprises increasing NK cell activity. In some embodiments, the activating, promoting, increasing, and/or enhancing of an immune response comprises increasing T-cell activity and increasing NK cell activity. In some embodiments, the activating, promoting, increasing, and/or enhancing of an immune response comprises increasing CU activity and increasing NK cell activity. In some embodiments, the activating, promoting, increasing, and/or enhancing of an immune response comprises inhibiting or decreasing the suppressive activity of Treg cells. In some embodiments, the activating, promoting, increasing, and/or enhancing of an immune response comprises inhibiting or decreasing the suppressive activity of myeloid derived suppressor cells (MDSCs). In some embodiments, the activating, promoting, increasing, and/or enhancing of an immune response comprises increasing the number of the percentage of memory T-cells. In some embodiments, the activating, promoting, increasing, and/or enhancing of an immune response comprises increasing long-term immune memory function. In some embodiments, the activating, promoting, increasing, and/or enhancing of an immune response comprises increasing long-term memory. In some embodiments, the activating, promoting, increasing, and/or enhancing of an immune response comprises no evidence of substantial side effects and/or immune-based toxicities. In some embodiments, the activating, promoting, increasing, and/or enhancing of an immune response comprises no evidence of cytokine release syndrome (CRS) or a cytokine storm. In some embodiments, the immune response is a result of antigenic stimulation. In some embodiments, the antigenic stimulation is a tumor cell. In some embodiments, the antigenic stimulation is cancer. In some embodiments, the antigenic stimulation is a pathogen. In some other embodiments, the antigenic stimulation may be a virally-infected cell.

In vivo and in vitro assays for determining whether an anti-NTPDase3 antibody modulates, activates, or inhibits an immune response are known in the art or are being developed.

In certain embodiments of the methods described herein, a method of inducing a persistent or long-term immunity which inhibits tumor relapse or tumor regrowth comprises administering to a subject a therapeutically effective amount of an anti-NTPDase3 antibody.

In some embodiments, the tumor is a solid tumor. In certain embodiments, the tumor is a tumor selected from the group consisting of: colorectal tumor, pancreatic tumor, lung tumor, ovarian tumor, liver tumor, breast tumor, kidney tumor, prostate tumor, neuroendocrine tumor, gastrointestinal tumor, melanoma, cervical tumor, bladder tumor, glioblastoma, lymphoma and head and neck tumor. In certain embodiments, the tumor is a colorectal tumor. In certain embodiments, the tumor is an ovarian tumor. In some embodiments, the tumor is a lung tumor. In certain embodiments, the tumor is a pancreatic or pancreatic islet tumor. In certain embodiments, the tumor is a melanoma tumor. In some embodiments, the tumor is a bladder or urothelial tumor.

In some embodiments, the tumor is a liquid tumor. In certain embodiments, the tumor is a leukemia, such as myelogenous or granulocytic leukemia, lymphatic, lymphocytic, or lymphoblastic leukemia, and polycythemia vera or erythrocytosis.

In some embodiments, the tumor expresses or overexpresses a tumor antigen targeted by the anti-NTPDase3 antibody, such as a bispecific agent which comprises an antigen-binding site that specifically binds the tumor antigen.

The present invention further provides methods for treating cancer in a subject comprising administering to the subject a therapeutically effective amount of an anti-NTPDase3 antibody described herein. In some embodiments, the anti-NTPDase3 antibody and inhibits or limits growth of the cancer.

The present invention provides for methods of treating cancer comprising administering to a subject (e.g., a subject in need of treatment) a therapeutically effective amount of an anti-NTPDase3 antibody described herein. In certain embodiments, the subject is a human. In certain embodiments, the subject has a cancerous tumor. In certain embodiments, the subject has had a tumor removed.

In certain embodiments, the cancer is a cancer selected from the group consisting of colorectal cancer, pancreatic cancer, lung cancer, ovarian cancer, liver cancer, breast cancer, kidney cancer, prostate cancer, gastrointestinal cancer, melanoma, cervical cancer, neuroendocrine cancer, bladder cancer, brain cancer, glioblastoma, and head and neck cancer. In certain embodiments, the cancer is pancreatic cancer. In certain embodiments, the cancer is ovarian cancer. In certain embodiments, the cancer is colorectal cancer. In certain embodiments, the cancer is breast cancer. In certain embodiments, the cancer is prostate cancer. In certain embodiments, the cancer is lung cancer. In certain embodiments, the cancer is melanoma. In some embodiments, the cancer is bladder cancer.

The present invention provides compositions comprising an anti-NTPDase3 antibody described herein. The present invention also provides pharmaceutical compositions comprising an anti-NTPDase3 antibody described herein and a pharmaceutically acceptable vehicle. In some embodiments, the pharmaceutical compositions find use in immunotherapy. In some embodiments, the pharmaceutical compositions find use in immuno-oncology. In some embodiments, the compositions find use in inhibiting tumor growth. In some embodiments, the pharmaceutical compositions find use in inhibiting tumor growth in a subject (e.g., a human patient). In some embodiments, the compositions find use in treating cancer. In some embodiments, the pharmaceutical compositions find use in treating cancer in a subject (e.g., a human patient).

Formulations are prepared for storage and use by combining a purified agent encompassed by the present invention with a pharmaceutically acceptable vehicle (e.g., a carrier or excipient). Those of skill in the art generally consider pharmaceutically acceptable carriers, excipients, and/or stabilizers to be inactive ingredients of a formulation or pharmaceutical composition.

In some embodiments, the anti-NTPDase3 antibody is lyophilized and/or stored in a lyophilized form. In some embodiments, a formulation comprising an anti-NTPDase3 antibody described herein is lyophilized.

Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22.sup.nd Edition, 2012, Pharmaceutical Press, London.).

The pharmaceutical compositions encompassed by the present invention can be administered in any number of ways for either local or systemic treatment. Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intraarterial, intratumoral, subcutaneous, intraperitoneal, intramuscular (e.g., injection or infusion), or intracranial (e.g., intrathecal or intraventricular).

The therapeutic formulation can be in unit dosage form. Such formulations include tablets, pills, capsules, powders, granules, solutions or suspensions in water or non-aqueous media, or suppositories. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and diluents (e.g., water). These can be used to form a solid preformulation composition containing a homogeneous mixture of a compound encompassed by the present invention, or a non-toxic pharmaceutically acceptable salt thereof. The solid preformulation composition is then subdivided into unit dosage forms of a type described above. The tablets, pills, etc. of the formulation or composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials include a number of polymeric acids and mixtures of polymeric acids with such materials as shellac, cetyl alcohol and cellulose acetate.

The anti-NTPDase3 antibody can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22.sup.nd Edition, 2012, Pharmaceutical Press, London.

In certain embodiments, pharmaceutical formulations include an anti-NTPDase3 antibody complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In certain embodiments, sustained-release preparations comprising the anti-NTPDase3 antibody can be produced. Suitable examples of sustained-release preparations include semi-permeable matrices of solid hydrophobic polymers containing an anti-NTPDase3 antibody, where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT (injectable microspheres composed of lactic acid-glycolic acid copolymer and leuprolide acetate), sucrose acetate isobutyrate, and poly-D-(−)-3-hydroxybutyric acid.

In certain embodiments, in addition to administering an anti-NTPDase3 antibody, the method or treatment further comprises administering at least one additional immune response stimulating agent. In some embodiments, the additional immune response stimulating agent includes, but is not limited to, a colony stimulating factor (e.g., granulocyte-macrophage colony stimulating factor (GM-CSF), macrophage colony stimulating factor (M-CSF), granulocyte colony stimulating factor (G-CSF), stem cell factor (SCF)), an interleukin (e.g., IL-1, IL2, IL-3, IL-7, IL-12, IL-15, IL-18), a checkpoint inhibitor, an antibody that blocks immunosuppressive functions (e.g., an anti-CTLA-4 antibody, anti-CD28 antibody, anti-CD3 antibody), a toll-like receptor (e.g., TLR4, TLR7, TLR9), or a member of the B7 family (e.g., CD80, CD86). An additional immune response stimulating agent can be administered prior to, concurrently with, and/or subsequently to, administration of the anti-NTPDase3 antibody. Pharmaceutical compositions comprising an anti-NTPDase3 antibody and the immune response stimulating agent(s) are also provided. In some embodiments, the immune response stimulating agent comprises 1, 2, 3, or more immune response stimulating agents.

In certain embodiments, in addition to administering an anti-NTPDase3 antibody, the method or treatment further comprises administering at least one additional therapeutic agent. An additional therapeutic agent can be administered prior to, concurrently with, and/or subsequently to, administration of the anti-NTPDase3 antibody. Pharmaceutical compositions comprising an anti-NTPDase3 antibody and the additional therapeutic agent(s) are also provided. In some embodiments, the at least one additional therapeutic agent comprises 1, 2, 3, or more additional therapeutic agents.

Combination therapy with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the anti-NTPDase3 antibody. Combination therapy may decrease the likelihood that resistant cancer cells will develop. In some embodiments, combination therapy comprises a therapeutic agent that affects the immune response (e.g., enhances or activates the response) and a therapeutic agent that affects (e.g., inhibits or kills) the tumor/cancer cells.

In some embodiments of the methods described herein, the combination of an anti-NTPDase3 antibody and at least one additional therapeutic agent results in additive or synergistic results. In some embodiments, the combination therapy results in an increase in the therapeutic index of the anti-NTPDase3 antibody. In some embodiments, the combination therapy results in an increase in the therapeutic index of the additional therapeutic agent(s). In some embodiments, the combination therapy results in a decrease in the toxicity and/or side effects of the anti-NTPDase3 antibody. In some embodiments, the combination therapy results in a decrease in the toxicity and/or side effects of the additional therapeutic agent(s).

Useful classes of therapeutic agents include, for example, anti-tubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, anti-folates, anti-metabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the second therapeutic agent is an alkylating agent, an antimetabolite, an antimitotic, a topoisomerase inhibitor, or an angiogenesis inhibitor.

Therapeutic agents that may be administered in combination with the anti-NTPDase3 antibody described herein include chemotherapeutic agents. Thus, in some embodiments, the method or treatment involves the administration of an anti-NTPDase3 antibody in combination with a chemotherapeutic agent or in combination with a cocktail of chemotherapeutic agents. Treatment with an anti-NTPDase3 antibody can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book, 4.sup.th Edition, 2008, M. C. Perry, Editor, Lippincott, Williams & Wilkins, Philadelphia, Pa.

Chemotherapeutic agents useful according to embodiments compassed within the present invention include, but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); and pharmaceutically acceptable salts, acids or derivatives of any of the above.

Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON); and anti-androgens such as flutamide, nilutamide, bicalutamide, leuprolide, and goserelin; and pharmaceutically acceptable salts, acids or derivatives of any of the above. In certain embodiments, the additional therapeutic agent is cisplatin. In certain embodiments, the additional therapeutic agent is carboplatin.

In certain embodiments of the methods described herein, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these.

In certain embodiments of the methods described herein, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (nab-paclitaxel; ABRAXANE), DHA-paclitaxel, or PG-paclitaxel. In certain alternative embodiments, the antimitotic agent comprises a vinca alkaloid, such as vincristine, vinblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or PIk1.

In certain embodiments of the methods described herein, it is anticipated that the subject anti-NTPDase3 antibody will have a greater combinatorial effect (perhaps even synergy) with those chemotherapeutic agents that induce the release of ATP in the tumor and/or cause upregulation of NTPDase3 or CD73 intratumorally. There are a wide range of chemotherapeutic agents that cause the release of ATP into the extracellular space as they induce tumor cell death, such as (but not limited to) anthracyclines (such as doxorubicin, daunorubicin, epirubicin and idarubicin), platinum-based drugs (such as cisplatin, carboplatin, and oxaliplatin), and proteasome inhibitors (such as bortezomib). Radiotherapy and photodynamic therapy (PDT) may also result in ATP release and/or upregulation of intratumoral levels of NTPDase3, CD73 and/or CD39.

In some embodiments of the methods described herein, an additional therapeutic agent comprises an agent such as a small molecule. For example, treatment can involve the combined administration of an anti-NTPDase3 antibody with a small molecule that acts as an inhibitor against tumor-associated antigens including, but not limited to, EGFR, HER2 (ErbB2), and/or VEGF. In some embodiments, an anti-NTPDase3 antibody is administered in combination with a protein kinase inhibitor selected from the group consisting of: gefitinib (IRESSA), erlotinib (TARCEVA), sunitinib (SUTENT), lapatanib, vandetanib (ZACTIMA), AEE788, CI-1033, cediranib (RECENTIN), sorafenib (NEXAVAR), and pazopanib (GW786034B). In some embodiments, an additional therapeutic agent comprises an mTOR inhibitor.

In certain embodiments of the methods described herein, the additional therapeutic agent is a small molecule that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an inhibitor of the Hippo pathway. In some embodiments, the additional therapeutic agent is an inhibitor of the mTOR/AKR pathway. In some embodiments, the additional therapeutic agent is an inhibitor of the RSPO/LGR pathway.

In some embodiments of the methods described herein, an additional therapeutic agent comprises a biological molecule, such as an antibody. For example, treatment can involve the combined administration of an anti-NTPDase3 antibody with antibodies against tumor-associated antigens including, but not limited to, antibodies that bind EGFR, HER2/ErbB2, and/or VEGF. In certain embodiments, the additional therapeutic agent is an antibody specific for a cancer stem cell marker. In some embodiments, the additional therapeutic agent is an antibody that binds a component of the Notch pathway. In some embodiments, the additional therapeutic agent is an antibody that binds a component of the Wnt pathway. In certain embodiments, the additional therapeutic agent is an antibody that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an antibody that inhibits β-catenin signaling. In certain embodiments, the additional therapeutic agent is an antibody that is an angiogenesis inhibitor (e.g., an anti-VEGF or VEGF receptor antibody). In certain embodiments, the additional therapeutic agent is bevacizumab (AVASTIN), ramucirumab, trastuzumab (HERCEPTIN), pertuzumab (OMNITARG), panitumumab (VECTIBIX), nimotuzumab, zalutumumab, or cetuximab (ERBITUX).

I/O Combinations—Representative Checkpoint Inhibitors and Co-stimulatory Agonists In some embodiments of the methods described herein, the additional therapeutic agent is an antibody that modulates the immune response. In some embodiments, the additional therapeutic agent is an anti-PD-1 antibody, an anti-PD-L1 antibody, an anti-CTLA-4 antibody, an anti-LAG-3 antibody, and anti-TIM-3 antibody, an anti-TIGIT antibody or an anti-Siglec-15 antibody.

For instance, the therapy can further include administering an inhibitor of immune checkpoint molecule or an activator of a costimulatory molecule, or a combination thereof. Exemplary inhibitors of immune checkpoints include inhibitors of one or more of PD-1, CTLA-4, TIM-3, LAG-3, CEACAM, VISTA, BTLA, TIGIT, LAIR1, CD160, 2B4, NLRP1, NRLP3, STING, TGFR beta or Siglec-15. Exemplary activators of costimulatory molecules include agonists of one or more of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand. Exemplary inhibitor of immune checkpoints and exemplary activators of costimulatory molecules can be found in PCT Publication WO 2016/054555, which is incorporated by reference herein.

PD-1 Antagonists

The PD-1 gene is a 55 kDa type I transmembrane protein that is part of the Ig gene superfamily (Agata et al. (1996) Int Immunol 8:765-72). PD-1 contains a membrane proximal immunoreceptor tyrosine inhibitory motif (ITIM) and a membrane distal tyrosine-based switch motif (ITSM) (Thomas, M. L. (1995) J Exp Med 181:1953-6; Vivier, E and Daeron, M (1997) Immunol Today 18:286-91). Two ligands for PD-1 have been identified, PD-L1 and PD-L2, that have been shown to downregulate T cell activation upon binding to PD-1 (Freeman et al. (2000) J Exp Med 192: 1027-34; Latchman et al. (2001) Nat Immunol 2:261-8; Carter et al. (2002) Eur J Immunol 32:634-43). Both PD-L1 and PD-L2 are B7 homologs that bind to PD-1, but do not bind to other CD28 family members. PD-L1 is abundant in a variety of human cancers (Dong et al. (2002) Nat. Med. 8:787-9). The interaction between PD-1 and PD-L1 results in a decrease in tumor infiltrating lymphocytes, a decrease in T-cell receptor mediated proliferation, and immune evasion by the cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1, and the effect is additive when the interaction of PD-1 with PD-L2 is blocked as well (Iwai et al. (2002) Proc. Nat'l. Acad. Sci. USA 99:12293-7; Brown et al. (2003) J. Immunol. 170: 1257-66)

As used herein, the terms “Programmed Death 1,” “Programmed Cell Death 1,” “Protein PD-1,” “PD-1,” PD1,” “PDCD1,” “hPD-1” and “hPD-1” are used interchangeably, and include variants, isoforms, species homologs of human PD-1, and analogs having at least one common epitope with human PD-1. The complete human PD-1 sequence can be found under GenBank Accession No. U64863.

As used herein, the terms “Programmed Cell Death 1 Ligand 1”, “PD-L1”, “PDL1”, “PDCD1L1”, “PDCD1LG1”, “CD274”, “B7 homolog 1”, “B7-H1”, “B7-H”, and “B7H1” are used interchangeably, and include variants, isoforms, species homologs of human PDL-1, and analogs having at least one common epitope with human PDL-1. The complete human PD-L1 amino acid sequence—isoform a precursor—can be found under GenBank Accession No. NP_054862.1. The complete human PD-L1 amino acid sequence-isoform b precursor—can be found under GenBank Accession No. NP_001254635.1. Fencode. The term “PD-1 axis binding antagonist” refers to a molecule that inhibits the interaction of a PD-1 axis binding partner with either one or more of its binding partner, so as to remove T-cell dysfunction resulting from signaling on the PD-1 signaling axis with a result being to restore or enhance T-cell function (e.g., proliferation, cytokine production, target cell killing). As used herein, a PD-1 axis binding antagonist includes a PD-1 binding antagonist, a PD-L1 binding antagonist and a PD-L2 binding antagonist.

The term “PD-1 binding antagonists” is a molecule that decreases, blocks, inhibits, abrogates or interferes with signal transduction resulting from the interaction of PD-1 with one or more of its binding partners, such as PD-L1, PD-L2. In some embodiments, the PD-1 binding antagonist is a molecule that inhibits the binding of PD-1 to its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one embodiment, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 binding antagonist is an anti-PD-1 antibody. In a specific aspect, a PD-1 binding antagonist is MDX-1106 described herein. In another specific aspect, a PD-1 binding antagonist is Merck 3745 described herein. In another specific aspect, a PD-1 binding antagonist is CT-011 described herein.

The term “PD-L1 binding antagonists” is a molecule that decreases, blocks, inhibits, abrogates or interferes with signal transduction resulting from the interaction of PD-L1 with either one or more of its binding partners, such as PD-1, B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In one embodiment, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some embodiments, a PD-L1 binding antagonist is an anti-PD-L1 antibody. In a specific aspect, an anti-PD-L1 antibody is YW243.55.S70 described herein. In another specific aspect, an anti-PD-L1 antibody is MDX-1105 described herein. In still another specific aspect, an anti-PD-L1 antibody is MPDL3280A described herein.

The term “PD-L2 binding antagonists” is a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one embodiment, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some embodiments, a PD-L2 binding antagonist is an immunoadhesin.

PD-1 pathway: Members of the PD-1 pathway are all proteins which are associated with PD-1 signaling. On the one hand these might be proteins which induce PD-1 signaling upstream of PD-1 as e.g., the ligands of PD-1 PD-L1 and PD-L2 and the signal transduction receptor PD-1. On the other hand, these might be signal transduction proteins downstream of PD-1 receptor. Particularly preferred as members of the PD-1 pathway in the context encompassed by the present invention are PD-1, PD-L1 and PD-L2.

PD-1 pathway inhibitor: In the context encompassed by the present invention, a PD-1 pathway inhibitor is preferably defined herein as a compound capable to impair the PD-1 pathway signaling, preferably signaling mediated by the PD-1 receptor. Therefore, the PD-1 pathway inhibitor may be any inhibitor directed against any member of the PD-1 pathway capable of antagonizing PD-1 pathway signaling. In this context, the inhibitor may be an antagonistic antibody as defined herein, targeting any member of the PD-1 pathway, preferably directed against PD-1 receptor, PD-L1 or PD-L2. This antagonistic antibody may also be encoded by a nucleic acid. Such encoded antibodies are also called “intrabodies” as defined herein. Also, the PD-1 pathway inhibitor may be a fragment of the PD-1 receptor or the PD1-receptor blocking the activity of PD1 ligands. B7-1 or fragments thereof may act as PD1-inhibiting ligands as well. Furthermore, the PD-1 pathway inhibitor may be siRNA (small interfering RNA) or antisense RNA directed against a member of the PD-1 pathway, preferably PD-1, PD-L1 or PD-L2. Additionally, a PD-1 pathway inhibitor may be a protein comprising (or a nucleic acid coding for) an amino acid sequence capable of binding to PD-1 but preventing PD-1 signaling, e.g., by inhibiting PD-1 and B7-H1 or B7-DL interaction. Additionally, a PD-1 pathway inhibitor may be a small molecule inhibitor capable of inhibiting PD-1 pathway signaling, e.g., a PD-1 binding peptide or a small organic molecule.

In certain embodiments, PD-1 antagonists encompassed by the present invention include agents that bind to ligands of PD-1 and interfere with, reduce, or inhibit the binding of one or more ligands to the PD-1 receptor, or bind directly to the PD-1 receptor, without engaging in signal transduction through the PD-1 receptor. In one embodiment, the PD-1 antagonist binds directly to PD-1 and blocks PD-1 inhibitory signal transduction. In another embodiment, the PD-1 antagonist binds to one or more ligands of PD-1 (e.g., PD-L1 and PD-L2) and reduces or inhibits the ligand(s) from triggering inhibitory signal transduction through the PD-1. In one embodiment, the PD-1 antagonist binds directly to PD-L1, inhibiting or preventing PD-L1 from binding to PD-1, thereby blocking PD-1 inhibitory signal transduction.

PD-1 antagonists used in the methods and compositions encompassed by the present invention include PD-1 binding scaffold proteins and include, but are not limited to, PD-ligands, antibodies and multivalent agents. In a particular embodiment, the antagonist is a fusion protein, such as AMP-224. In another embodiment, the antagonist is an anti-PD-1 antibody (“PD-1 antibody”). Anti-human-PD-1 antibodies (or VH and/or VL domains derived therefrom) suitable for use in the invention can be generated using methods well known in the art. Alternatively, art recognized anti-PD-1 antibodies can be used. For example, antibodies MK-3475 or CT-011 can be used. Additionally, monoclonal antibodies 5C4, 17D8, 2D3, 4H1, 4A11, 7D3, and 5F4, described in WO 2006/121168, the teachings of which are hereby incorporated by reference, can be used. Antibodies that compete with any of these art-recognized antibodies for binding to PD-1 also can be used.

In another embodiment, the PD-1 antagonist is an anti-PD-L1 antibody. Anti-human-PD-L1 antibodies (or VH and/or VL domains derived therefrom) suitable for use in the invention can be generated using methods well known in the art. Alternatively, art recognized anti-PD-L1 antibodies can be used. For example, MEDI4736 (also known as Anti-B7-H1) or MPDL3280A (also known as RG7446) can be used. Additionally, monoclonal antibodies 12A4, 3G10, 10A5, 5F8, 10H10, 1B12, 7H1, 11E6, 12B7, and 13G4 described in WO 2007/005874 and U.S. Pat. No. 7,943,743, the teachings of which are hereby incorporated by reference, can be used. Antibodies that compete with any of these art-recognized antibodies for binding to PD-L1 also can be used.

An exemplary anti-PD-L1 antibody is 12A4 (WO 2007/005874 and U.S. Pat. No. 7,943,743). Anti-PD-1 or anti-PD-L1 antibodies may bind to PD-1 or PD-L1, respectively, with a KD of 10⁻⁷ M, 5×10⁻⁸ M, 10⁻⁸ M, 5×10⁻⁹ M, 10⁻⁹ M, 5×10⁻¹⁰ M, 10⁻¹⁰ M or less.

In one embodiment, the PD-1 inhibitor is an anti-PD-1 antibody chosen from Nivolumab, Pembrolizumab or Pidilizumab. A preferred PD-1 inhibitor is Nivolumab.

In some embodiments, the anti-PD-1 antibody is Nivolumab. Alternative names for Nivolumab include MDX-1106, MDX-1106-04, ONO-4538, or BMS-936558. In some embodiments, the anti-PD-1 antibody is Nivolumab (CAS Registry Number: 946414-94-4). Nivolumab is a fully human IgG4 monoclonal antibody which specifically blocks PDI. Nivolumab (clone 5C4) and other human monoclonal antibodies that specifically bind to PDI are disclosed in U.S. Pat. No. 8,008,449 (incorporated by reference) and WO 2006/121168 (incorporated by reference). In other embodiments, the anti-PD-1 antibody is Pembrolizumab. Pembrolizumab (Trade name KEYTRUDA® formerly Lambrolizumab, also known as Merck 3745, MK-3475 or SCH-900475) is a humanized IgG4 monoclonal antibody that binds to PD1. Pembrolizumab is disclosed, e.g., in Hamid, O. et al. (2013) New England Journal of Medicine 369 (2): 134-44, WO 2009/114335 (incorporated by reference), and U.S. Pat. No. 8,354,509 (incorporated by reference).

In some embodiments, the anti-PD-1 antibody is Pidilizumab. Pidilizumab (CT-011; Cure Tech) is a humanized IgG1k monoclonal antibody that binds to PD1. Pidilizumab and other humanized anti-PD-1 monoclonal antibodies are disclosed in WO2009/101611. Other anti-PD1 antibodies are disclosed in U.S. Pat. No. 8,609,089, US 2010028330, and/or US 20120114649. Other anti-PDI antibodies include AMP 514 (Amplimmune).

In some embodiments, the PD-1 inhibitor is an immunoadhesin {e.g., an immunoadhesin comprising an extracellular or PD-1 binding portion of PD-L1 or PD-L2 fused to a constant region {e.g., an Fc region of an immunoglobulin sequence). In some embodiments, the PD-1 inhibitor is AMP-224. In some embodiments, the PD-L1 inhibitor is anti-PD-L1 antibody. In some embodiments, the anti-PD-L1 inhibitor is YW243.55.570, MPDL3280A, MEDI-4736, MSB-0010718C, or MDX-1105.

In one embodiment, the PD-L1 inhibitor is MDX-1105. MDX-1105, also known as BMS-936559, is an anti-PD-L1 antibody described in WO 2007/005874. In one embodiment, the PD-L1 inhibitor is YW243.55.570. The YW243.55.570 antibody is an anti-PD-L1 described in WO 2010/077634 (incorporated by reference).

In one embodiment, the PD-L1 inhibitor is MDPL3280A (Genentech/Roche). MDPL3280A is a human Fc optimized IgG1 monoclonal antibody that binds to PD-L1. M DPL3280A and other human monoclonal antibodies to PD-L1 are disclosed in U.S. Pat. No. 7,943,743 (incorporated by reference) and U.S Publication No.: 2012/0039906 (incorporated by reference). In other embodiments, the PD-L2 inhibitor is AMP-224. AMP-224 is a PD-L2 Fc fusion soluble receptor that blocks the interaction between PD1 and B7-H1 (B7-DCIg; Amplimmune; e.g., disclosed in WO 2010/027827 (incorporated by reference) and WO 2011/066342 (incorporated by reference)).

In certain embodiments, the PD-1 pathway inhibitor is a small molecule antagonist of PD-1 pathway signaling. Such small molecule antagonists include those agents that bind to one or more of PD-1, PD-1L and/or PD-1L2 and inhibits the interaction of PD-1 with PD-1L1 and/or PD-1L2.

Exemplary small molecule antagonist of PD-1 pathway signaling can be found in, inter alia, published US applications 2014/0294898 and 2014/0199334, and published PCT Applications WO 2013/132317 and WO 2012/168944, each of which is incorporated by reference herein.

Merely to illustrate, the subject combination therapy can be practiced with small molecule antagonist selected from the group consisting of

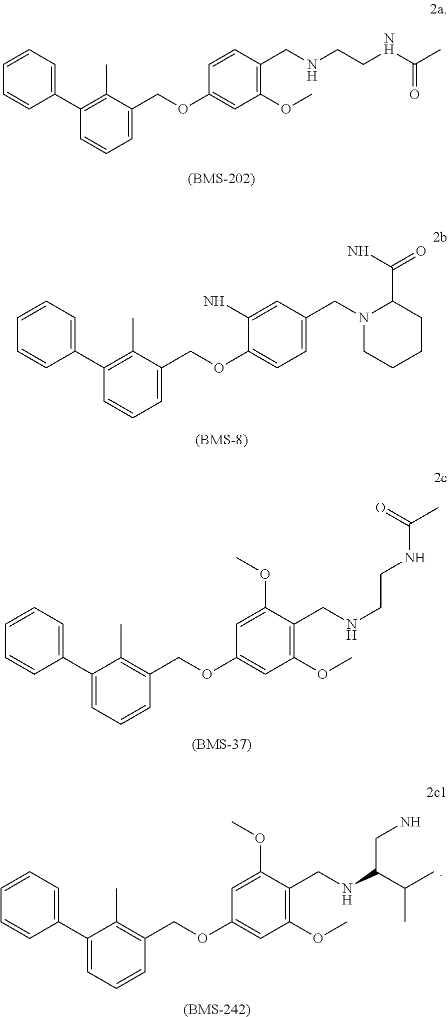

In other embodiments, the small molecule antagonist is represented in the general formula

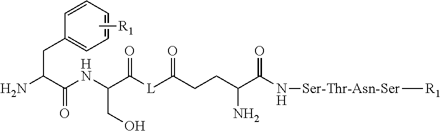

wherein,
R1 is free C-terminal or amidated C-terminal of Ser;
L is a linker selected from —NH(CH₂)ₙNH— or —NH(CH₂CH₂O)ₙNH—;
R4 is selected from hydrogen, amino(C₁-C₂₀)alkyl, —NHCOCH₃ or —NHCONH₂;
or retro analogue or a pharmaceutically acceptable stereoisomer or a pharmaceutically acceptable salt thereof.

In still other embodiments, the small molecule antagonist is represented in the general formula

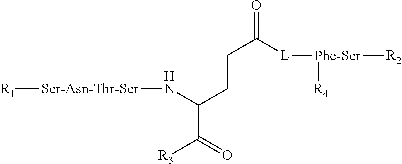

wherein,
R₁ is N-terminal of Ser; or (C₁-C₂₀)acyl substituted with either hydroxyl group or amino group of Ser;
L is a linker selected from —NH(CH₂)ₙNH—, —NH(CH₂)ₙCH(NH₂)CO—, —OOC(CH₂)ₘCOO—, —NH(CH₂)ₙCO—, —NH(CH₂CH₂O)ₙNH—, —NH(CH₂CH₂O)ₙCO— or —CO(CH₂CH₂O)ₙCO—;
R₂ is free C-terminal, amidated C-terminal or N-terminal of Am₂; or Y—R₅;
Y is an optional linker selected from —OOC(CH₂)ₘCOO—, —CO(CH₂)ₙNH—, —CO(CH₂CH₂O)ₙNH— or —COCH₂(OCH₂CH₂)ₙNH—;
R₅ is an albumin binding moiety such as maleimido propionic acid;
R₃ is OH or NH₂;
R₄ is a substituent on phenyl group of Phe and is selected from hydrogen, amino(C₁-C₂₀)alkyl, —NHCOCH₃ or —NHCONH₂;
n is an integer having values selected from 2 to 10, both inclusive;
m is an integer having values selected from 0 to 8, both inclusive; and one of the peptide bond (—CONH—) of Ser-Asn, Asn-Thr or Thr-Ser may be replaced with a modified peptide bond of

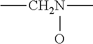

wherein Q is hydrogen, —CO(C₁-C₂₀)alkyl or —COO(C₁-C₂₀)alkyl group; wherein one or more or all amino acids may be in the D-configuration;
or retro analogue or a pharmaceutically acceptable stereoisomer or a pharmaceutically acceptable salt thereof.

For instance, the small molecule antagonist can be selected from the group consisting of

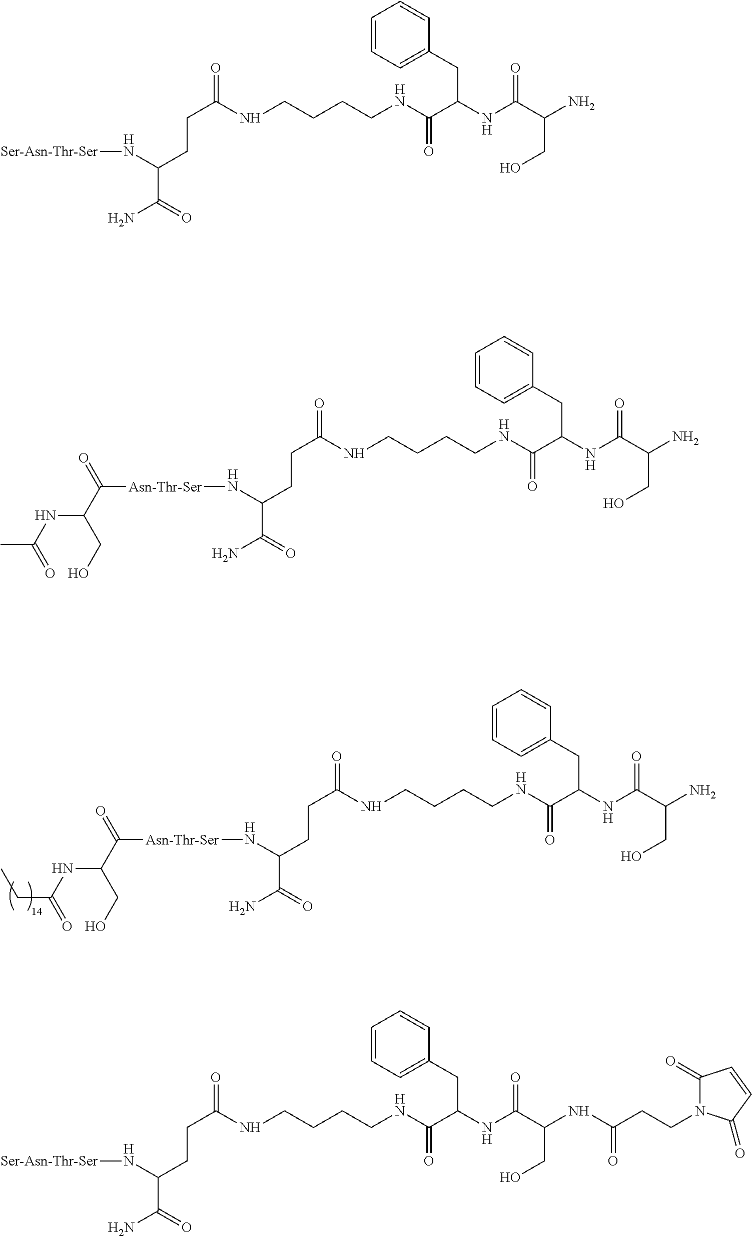

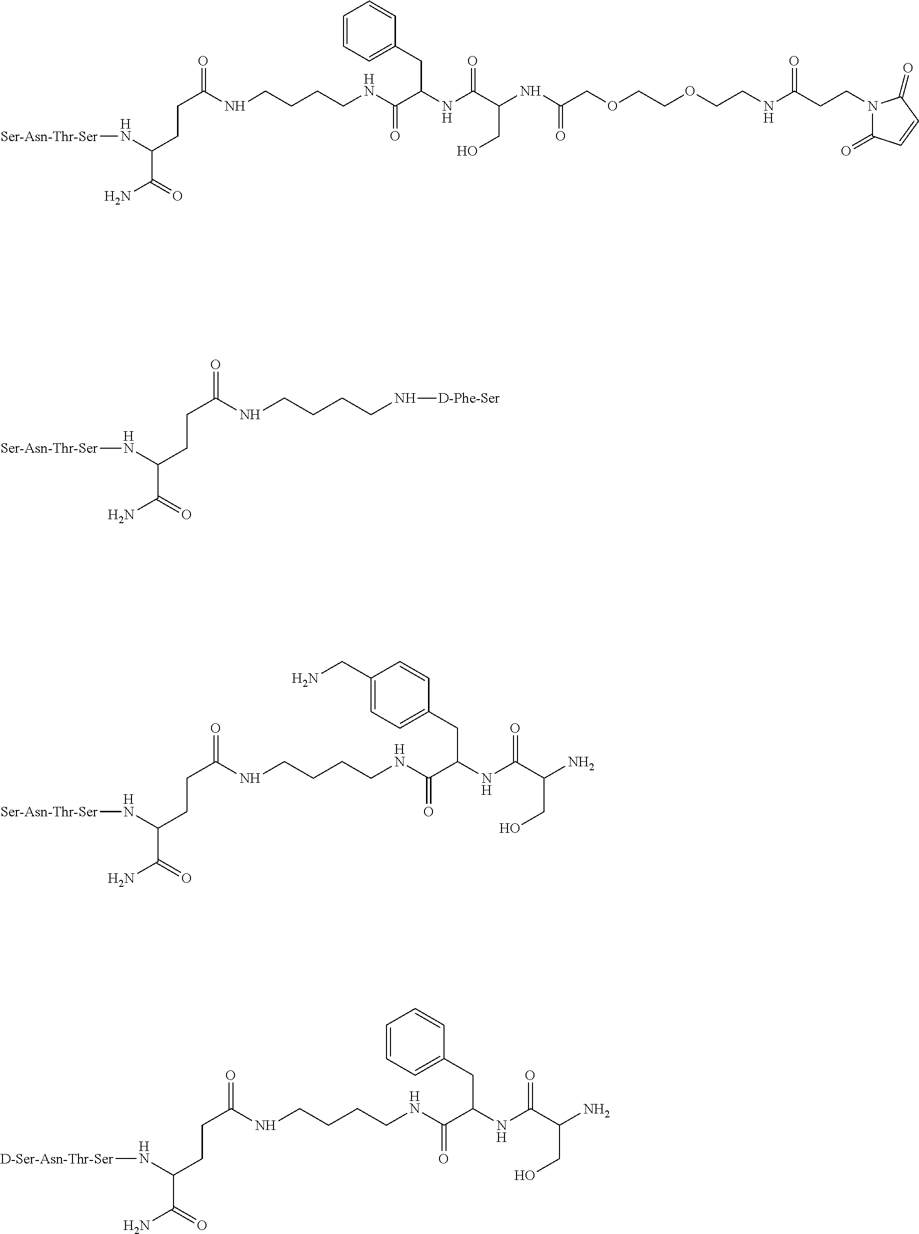

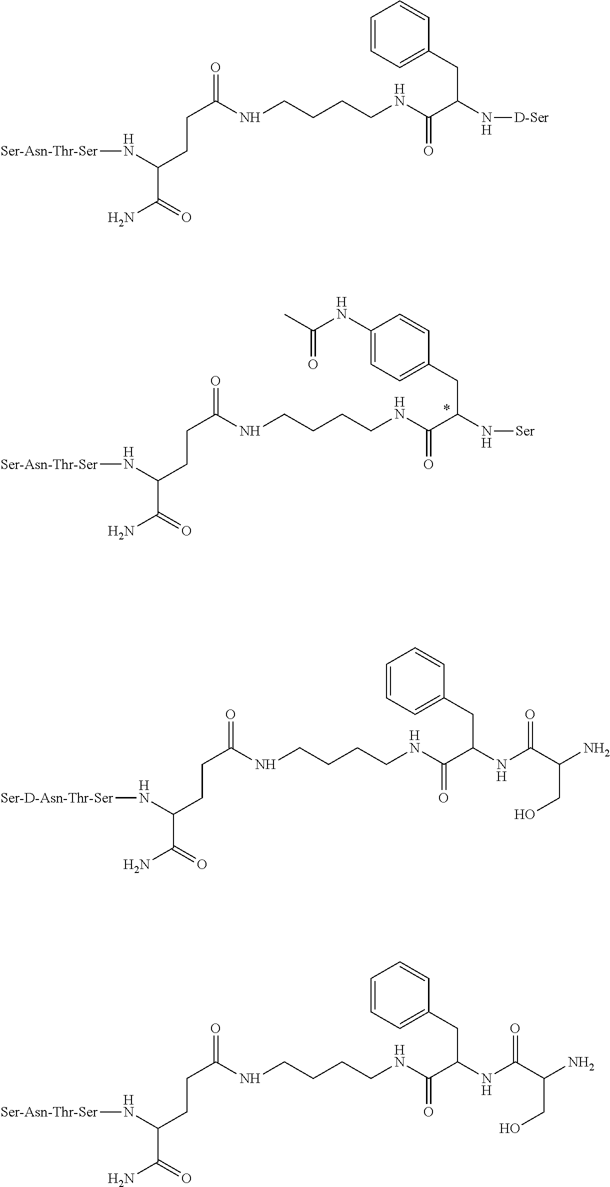

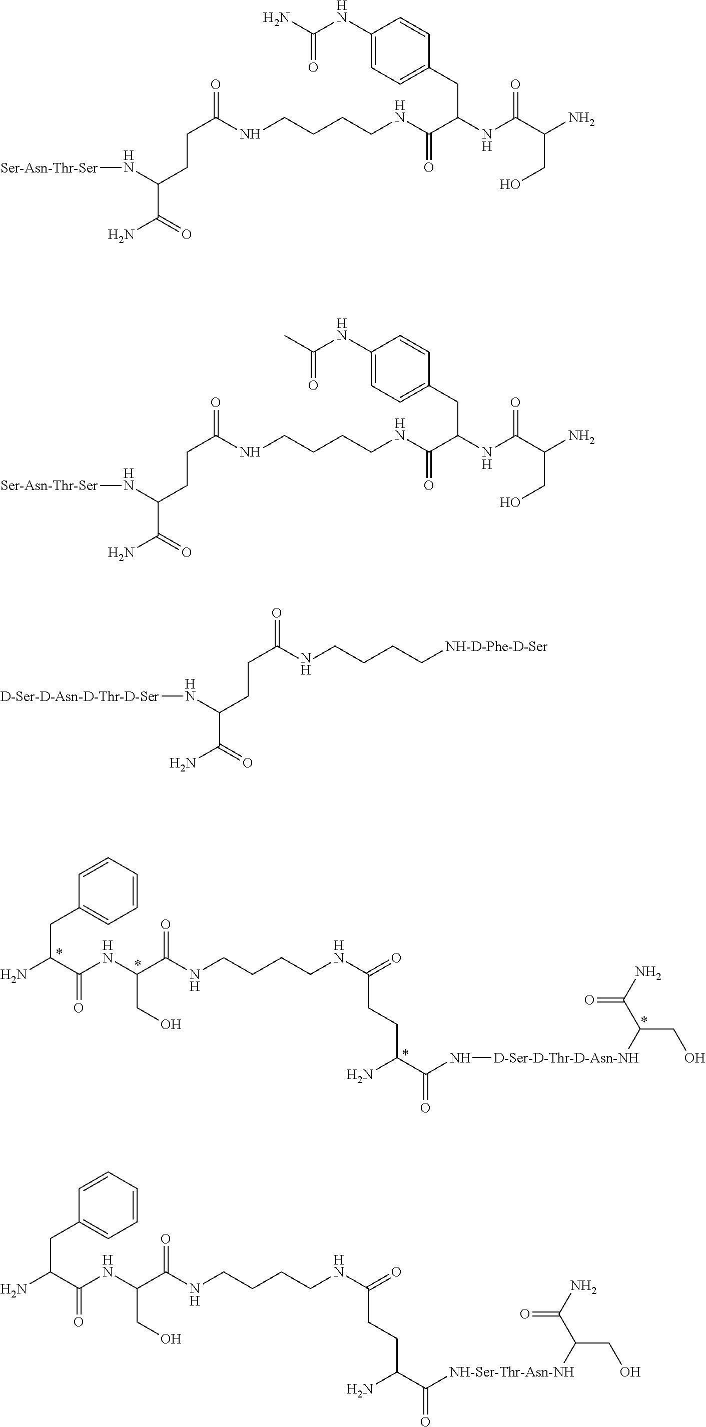

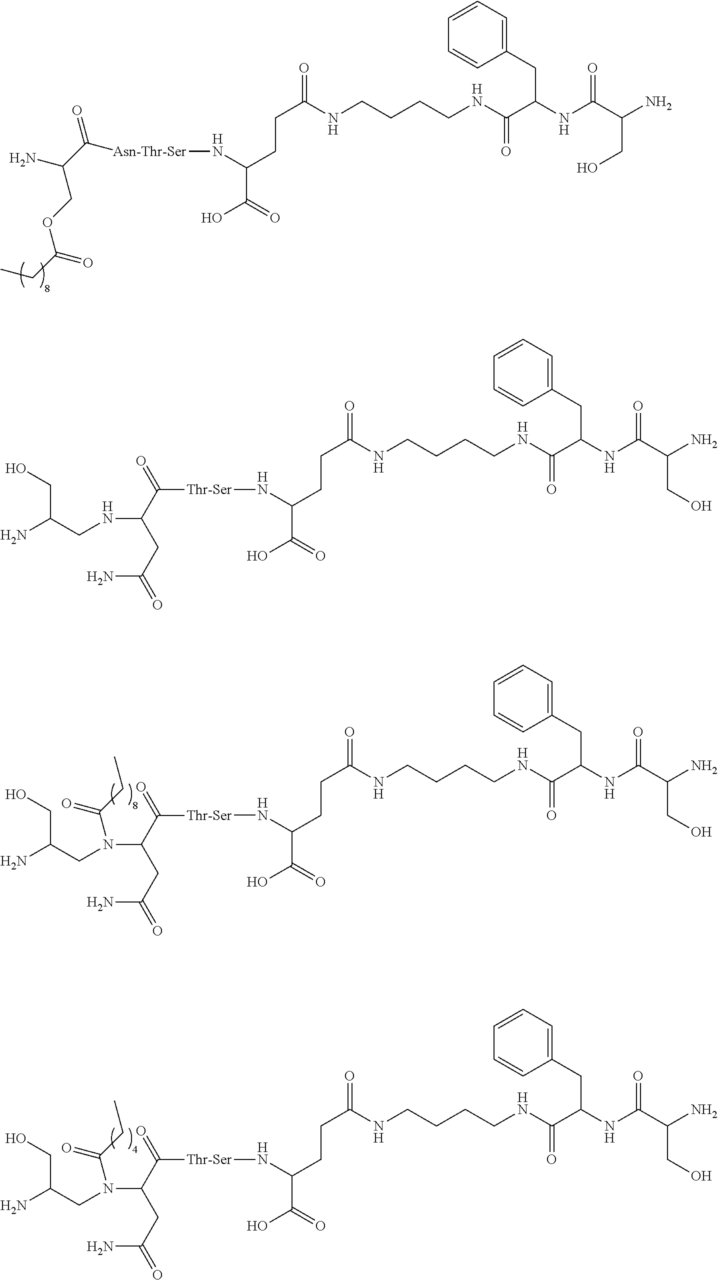

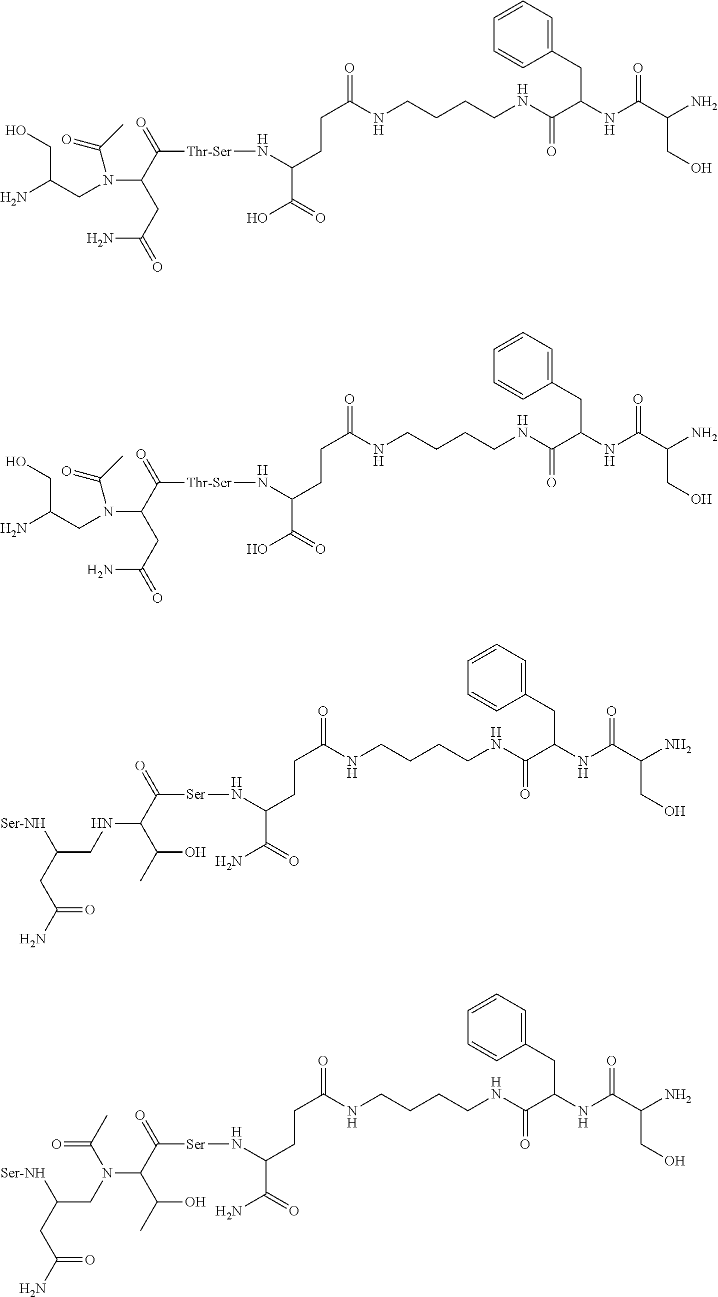

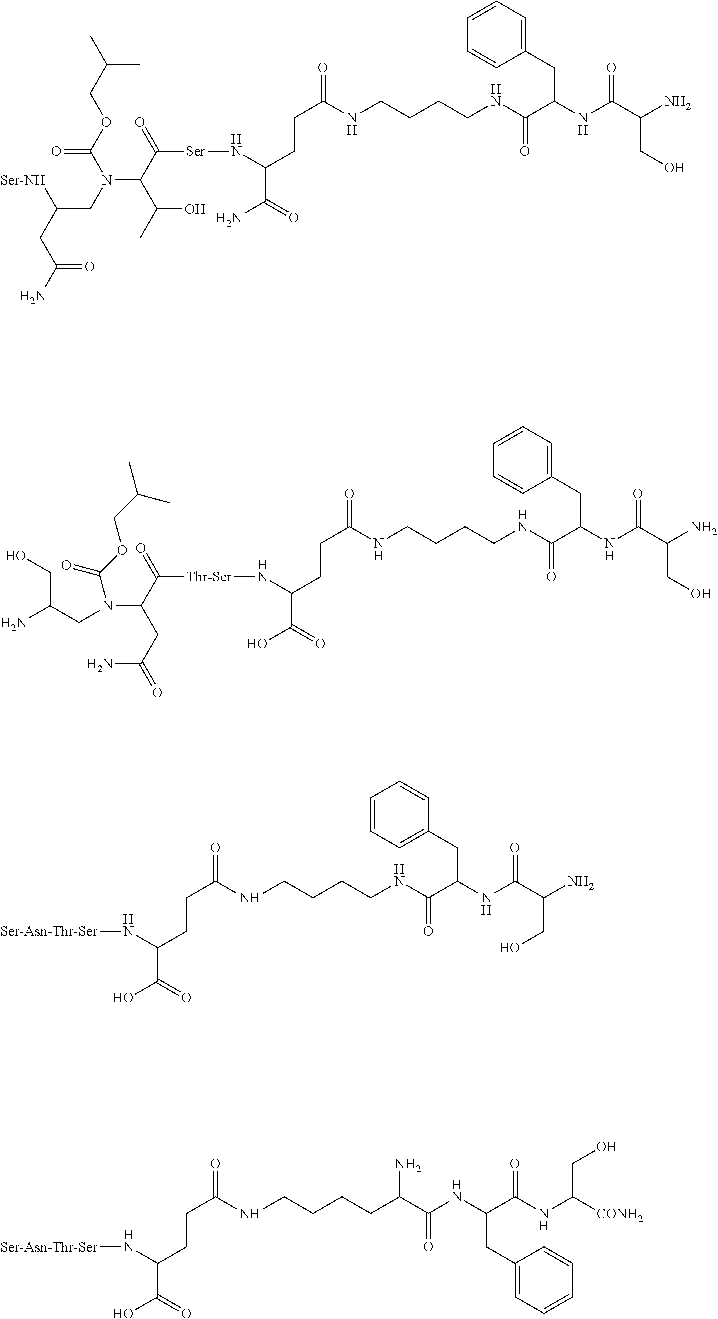

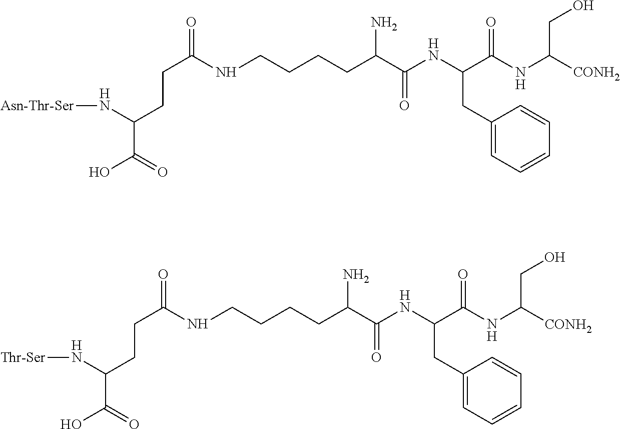

CTLA-4 Antagonists

In certain embodiments, a combination described herein also includes a CTLA-4 inhibitor. Exemplary anti-CTLA-4 antibodies include Tremelimumab (IgG2 monoclonal antibody available from Pfizer, formerly known as ticilimumab, CP-675,206); and Ipilimumab (CTLA-4 antibody, also known as MDX-010, CAS No. 477202-00-9).

Information regarding tremelimumab (or antigen-binding fragments thereof) for use in the methods provided herein can be found in U.S. Pat. No. 6,682,736 (incorporated by reference) (where it is referred to as 11.2.1), the disclosure of which is incorporated herein by reference in its entirety. Tremelimumab (also known as CP-675,206, CP-675, CP-675206, and ticilimumab) is a human IgG2 monoclonal antibody that is highly selective for CTLA-4 and blocks binding of CTLA-4 to CD80 (B7.1) and CD86 (B7.2). It has been shown to result in immune activation in vitro and some patients treated with tremelimumab have shown tumor regression.

Tremelimumab for use in the methods provided herein comprises a heavy chain and a light chain or a heavy chain variable region and a light chain variable region. In a specific aspect, tremelimumab or an antigen-binding fragment thereof for use in the methods provided herein comprises a light chain variable region comprising the amino acid sequences shown herein above and a heavy chain variable region comprising the amino acid sequence shown herein above. In a specific aspect, tremelimumab or an antigen-binding fragment thereof for use in the methods provided herein comprises a heavy chain variable region and a light chain variable region, wherein the heavy chain variable region comprises the Kabat-defined CDR1, CDR2, and CDR3 sequences shown herein above, and wherein the light chain variable region comprises the Kabat-defined CDR1, CDR2, and CDR3 sequences shown herein above. Those of ordinary skill in the art would easily be able to identify Chothia-defined, Abm-defined or other CDR definitions known to those of ordinary skill in the art. In a specific aspect, tremelimumab or an antigen-binding fragment thereof for use in the methods provided herein comprises the variable heavy chain and variable light chain CDR sequences of the antibody as disclosed in U.S. Pat. No. 6,682,736, which is herein incorporated by reference in its entirety.

The present invention also contemplates utilizing small molecule inhibitors of CTLA-4, such as described by Huxley et al. 2004 Cell Chemical Biology 11:1651-1658, which includes compounds of the formula:

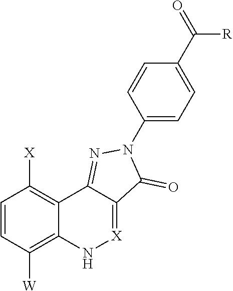

| Compound | W | Z | X | R |
|---|---|---|---|---|
| 1 | F | H | CH | OH |
| 2 | F | H | CH | NHCH₂CH₂CH₂NMe₂ |
| 3 | H | H | N | 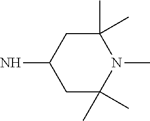 |
| 4 | F | H | N | 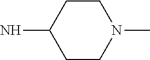 |
| 5 | F | H | N | 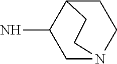 |
| 6 | F | F | N | 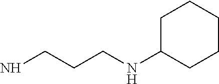 |

Other small molecule CTLA-4 antagonists include

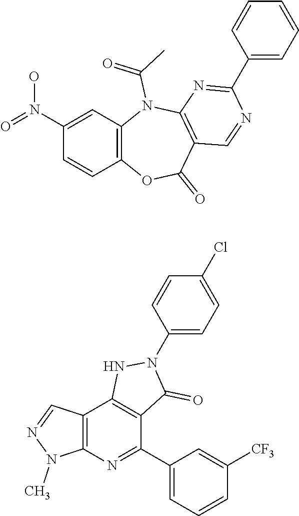

In one embodiment, the combination includes an immuno-DASH inhibitor, an anti-PD-1 antibody molecule, e.g., as described herein, and an anti-CTLA-4 antibody, e.g., ipilimumab. Exemplary doses that can be use include a dose of anti-PD-1 antibody molecule of about 1 to 10 mg/kg, e.g., 3 mg/kg, and a dose of an anti-CTLA-4 antibody, e.g., ipilimumab, of about 3 mg/kg.

Other exemplary anti-CTLA-4 antibodies are disclosed, e.g., in U.S. Pat. No. 5,811,097.

In some embodiments of the methods described herein, the additional therapeutic agent is an antibody that modulates the immune response. In some embodiments, the additional therapeutic agent is an anti-PD-1 antibody, an anti-LAG-3 antibody, an anti-CTLA-4 antibody, an anti-TIM-3 antibody, an anti-TIGIT antibody or an anti-Siglec-15 antibody.

In some embodiments, the LAG3 antibody is IMP701, IMP731, BMS-986016, LAG525, and GSK2831781. In some embodiments, the LAG3 antagonist includes a soluble LAG3 receptor, for example, IMP321.

In some embodiments, an immune response stimulating agent is selected from the group consisting of: a CD28 agonist, a 4-1BB agonist, an OX40 agonist, a CD27 agonist, a CD80 agonist, a CD86 agonist, a CD40 agonist, and a GITR agonist. In some embodiments, the OX40 agonist includes OX40 ligand, or an OX40-binding portion thereof. For example, the OX40 agonist may be MED16383. In some embodiments, the OX40 agonist is an antibody that specifically binds OX40. In some embodiments, the antibody that binds OX40 is MED16469, MED10562, or MOXR0916 (RG7888). In some embodiments, the OX40 agonist is a vector (e.g., an expression vector or virus, such as an adenovirus) capable of expressing OX40 ligand. In some embodiments the OX40-expressing vector is Delta-24-RGDOX or DNX2401.

In some embodiments, the 4-1BB (CD137) agonist is a binding molecule, such as an anticalin. In some embodiments, the anticalin is PRS-343. In some embodiments, the 4-1BB agonist is an antibody that specifically binds 4-1BB. In some embodiments, antibody that binds 4-1BB is PF-2566 (PF-05082566) or urelumab (BMS-663513).

In some embodiments, the CD27 agonist is an antibody that specifically binds CD27. In some embodiments, the antibody that binds CD27 is varlilumab (CDX-1127).

In some embodiments, the GITR agonist comprises GITR ligand or a GITR-binding portion thereof. In some embodiments, the GITR agonist is an antibody that specifically binds GITR. In some embodiments, the antibody that binds GITR is TRX518, MK-4166, or INBRX-110.

In certain embodiments, an anti-NTPDase3 antibody is combined with STING agonist, preferably as part of a pharmaceutical composition. The cyclic-di-nucleotides (CDNs) cyclic-di-AMP (produced by Listeria monocytogenes and other bacteria) and its analogs cyclic-di-GMP and cyclic-GMP-AMP are recognized by the host cell as a pathogen associated molecular pattern (PAMP), which bind to the pathogen recognition receptor (PRR) known as Stimulator of Interferon Genes (STING). STING is an adaptor protein in the cytoplasm of host mammalian cells which activates the TANK binding kinase (TBK1)-IRF3 and the NF-κB signaling axis, resulting in the induction of IFN-β and other gene products that strongly activate innate immunity. It is now recognized that STING is a component of the host cytosolic surveillance pathway (Vance et al., 2009), that senses infection with intracellular pathogens and in response induces the production of IFN-β, leading to the development of an adaptive protective pathogen-specific immune response consisting of both antigen-specific CD4+ and CD8+ T cells as well as pathogen-specific antibodies. U.S. Pat. Nos. 7,709,458 and 7,592,326; PCT Publication Nos. WO2007/054279, WO2014/093936, WO2014/179335, WO2014/189805, WO2015/185565, WO2016/096174, WO2016/145102, WO2017/027645, WO2017/027646, and WO2017/075477; and Yan et al., Bioorg. Med. Chem Lett. 18:5631-4, 2008.

Exemplary Combinations

In a preferred embodiment, the invention is directed to the combination of an anti-NTPDase3 antibody with an antitumor platinum coordination complex in the treatment of cancer, and more particularly in the treatment of a cancer selected from lung cancer, sarcoma, malignant melanoma, prostate cancer, pancreas carcinoma, gastric carcinoma, ovarian cancer, hepatoma, breast cancer, colorectal cancer, kidney cancer, brain cancer and lymphoma. This chemotherapeutic group includes, but is not limited to cisplatin, oxaliplatin, carboplatin, triplatin tetranitrate (BBR3464), satraplatin, tetraplatin, ormiplatin, iproplatin, nedaplatin and lobaplatin. Particularly preferred is the combination of an anti-NTPDase3 antibody with cisplatin, oxaliplatin, carboplatin, triplatin tetranitrate, satraplatin, tetraplatin, ormiplatin, iproplatin, nedaplatin and lobaplatin, and even more preferred is the combination with cisplatin and oxaliplatin in the treatment of cancer, and more particularly in the treatment of a cancer selected from lung cancer, sarcoma, malignant melanoma, prostate cancer, pancreas carcinoma, gastric carcinoma, ovarian cancer, hepatoma, breast cancer, colorectal cancer, kidney cancer and brain cancer. In another preferred embodiment, the invention is directed to the combination of an anti-NTPDase3 antibody with an antimetabolite in the treatment of cancer, and more particularly in the treatment of a cancer selected from lung cancer, sarcoma, malignant melanoma, bladder carcinoma, prostate cancer, pancreas carcinoma, gastric carcinoma, ovarian cancer, hepatoma, breast cancer, colorectal cancer, kidney cancer, esophageal cancer, brain cancer, anal cancer, leukaemia and lymphoma. This chemotherapeutic group includes, but is not limited to 5-fluorouracil, gemcitabine, cytarabine, capecitabine, decitabine, floxuridine, fludarabine, aminopterin, methotrexate, pemetrexed, raltitrexed, cladribine, clofarabine, mercaptopurine, pentostatin, and thioguanine. Particularly preferred is the combination of an anti-NTPDase3 antibody with 5-fluorouracil, gemcitabine, cytarabine, capecitabine, decitabine, floxuridine, fludarabine, aminopterin, methotrexate, pemetrexed, raltitrexed, cladribine, clofarabine, mercaptopurine, pentostatin, and thioguanine, and even more preferred is the combination with 5-fluorouracil, gemcitabine, cytarabine and methotrexate in the treatment of cancer, and more particularly in the treatment of a cancer selected from lung cancer, sarcoma, malignant melanoma, prostate cancer, pancreas carcinoma, gastric carcinoma, ovarian cancer, hepatoma, breast cancer, colorectal cancer, kidney cancer, brain cancer, leukemia and lymphoma.

In another preferred embodiment, the invention is directed to the combination of an anti-NTPDase3 antibody with a mitotic inhibitor in the treatment of cancer, and more particularly in the treatment of a cancer selected from lung cancer, sarcoma, prostate cancer, gastric carcinoma, ovarian cancer, hepatoma, breast cancer, colorectal cancer, kidney cancer, brain cancer, leukemia, and lymphoma. This chemotherapeutic group includes, but is not limited to paclitaxel, docetaxel, vinblastine, vincristine, vindesine, and vinorelbine. Particularly preferred is the combination of an anti-NTPDase3 antibody with paclitaxel, docetaxel, vinblastine, vincristine, vindesine, and vinorelbine, and even more preferred is the combination with paclitaxel, docetaxel, vincristine and vinorelbine in the treatment of cancer, and more particularly in the treatment of a cancer selected from lung cancer, sarcoma, prostate cancer, gastric carcinoma, ovarian cancer, hepatoma, breast cancer, colorectal cancer, kidney cancer and brain cancer.

In another preferred embodiment, the invention is directed to the combination of anti-NTPDase3 antibody with an anticancer antibiotic in the treatment of cancer, and more particularly in the treatment of lung cancer, sarcoma, malignant melanoma, bladder carcinoma, prostate cancer, pancreas carcinoma, thyroid cancer, gastric carcinoma, ovarian cancer, hepatoma, breast cancer, colorectal cancer, kidney cancer, neuroblastoma, brain cancer, anal cancer, testicular cancer, leukemia, multiple myeloma and lymphoma. This chemotherapeutic group includes, but is not limited to daunorubicin, doxorubicin, epirubicin, idarubicin, mitoxantrone, pixantrone, valrubicin, mitomycin C, bleomycin, actinomycin A and mithramycin. Particularly preferred is the combination of an anti-NTPDase3 antibody with daunorubicin, doxorubicin, epirubicin, idarubicin, mitoxantrone, pixantrone, valrubicin, mitomycin C, bleomycin, actinomycin D and mithramycin, and even more preferred is the combination with daunorubicin, doxorubicin, mitomycin C and actinomycin D in the treatment of cancer, and more particularly in the treatment of lung cancer, sarcoma, malignant melanoma, prostate cancer, pancreas carcinoma, gastric carcinoma, ovarian cancer, hepatoma, breast cancer, colorectal cancer, kidney cancer, brain cancer, leukemia and lymphoma.

In another preferred embodiment, the invention is directed to the combination of anti-NTPDase3 antibody with a topoisomerase I and/or II inhibitor in the treatment of cancer, and more particularly in the treatment of lung cancer, sarcoma, malignant melanoma, prostate cancer, pancreas carcinoma, gastric carcinoma, ovarian cancer, hepatoma, breast cancer, colorectal cancer, kidney cancer, neuroblastoma, brain cancer, cervical cancer, testicular cancer, leukemia and lymphoma. This chemotherapeutic group includes, but is not limited to topotecan, SN-38, irinotecan, camptothecin, rubitecan, etoposide, amsacrine and teniposide. Particularly preferred is the combination of PM00104, or a pharmaceutically acceptable salt thereof, with topotecan, SN-38, irinotecan, camptothecin, rubitecan, etoposide, amsacrine and teniposide, and even more preferred is the combination with topotecan, irinotecan and etoposide in the treatment of cancer, and more particularly in the treatment of lung cancer, sarcoma, malignant melanoma, prostate cancer, pancreas carcinoma, gastric carcinoma, ovarian cancer, hepatoma, breast cancer, colorectal cancer, kidney cancer, and brain cancer.

In another preferred embodiment, the invention is directed to the combination of an anti-NTPDase3 antibody with a proteosome inhibitor in the treatment of cancer, and more particularly in the treatment of lung cancer, prostate cancer, pancreas carcinoma, gastric carcinoma, hepatoma, colorectal cancer, brain cancer, multiple myeloma and lymphoma. This chemotherapeutic group includes, but is not limited to bortezomib, disulfiram, epigallocatechin gallate, and salinosporamide A. Particularly preferred is the combination of an anti-NTPDase3 antibody with bortezomib, disulfiram, epigallocatechin gallate, and salinosporamide A, and even more preferred is the combination with bortezomib in the treatment of cancer, and more particularly in the treatment of lung cancer, prostate cancer, pancreas carcinoma, gastric carcinoma, hepatoma, colorectal cancer and brain cancer.

In another preferred embodiment, the invention is directed to the combination of an anti-NTPDase3 antibody with a histone deacetylase inhibitor in the treatment of cancer, and more particularly in the treatment of lung cancer, sarcoma, prostate cancer, pancreas carcinoma, gastric carcinoma, ovarian cancer, breast cancer, colorectal cancer, kidney cancer, brain cancer and lymphoma. This chemotherapeutic group includes, but is not limited to romidepsin, panobinostat, vorinostat, mocetinostat, belinostat, entinostat, resminostat, PCI-24781, AR-42, CUDC-101, and valproic acid. Particularly preferred is the combination of an anti-NTPDase3 antibody with romidepsin, panobinostat, vorinostat, mocetinostat, belinostat, entinostat, resminostat, PCI-24781, AR-42, CUDC-101, and valproic acid, and even more preferred is the combination with vorinostat in the treatment of cancer, and more particularly in the treatment of lung cancer, sarcoma, prostate cancer, pancreas carcinoma, gastric carcinoma, ovarian cancer, breast cancer, colorectal cancer, kidney cancer and brain cancer.

In another preferred embodiment, the invention is directed to the combination of an anti-NTPDase3 antibody with a nitrogen mustard alkylating agent in the treatment of cancer, and more particularly in the treatment of lung cancer, sarcoma, bladder carcinoma, gastric carcinoma, ovarian cancer, hepatoma, breast cancer, colorectal cancer, kidney cancer, leukemia, multiple myeloma and lymphoma. This chemotherapeutic group includes, but is not limited to melphalan, ifosfamide, chlorambucil, cyclophosphamide, mechlorethamine, uramustine, estramustine and bendamustine. Particularly preferred is the combination of an anti-NTPDase3 antibody with melphalan, ifosfamide, chlorambucil, cyclophosphamide, mechlorethamine, uramustine, estramustine and bendamustine, and even more preferred is the combination with cyclophosphamide in the treatment of cancer, and more particularly in the treatment of lung cancer, sarcoma, gastric carcinoma, ovarian cancer, hepatoma, breast cancer, colorectal cancer and kidney cancer. In another preferred embodiment, the invention is directed to the combination of an anti-NTPDase3 antibody with a nitrosourea alkylating agent in the treatment of cancer, and more particularly in the treatment of lung cancer, ovarian cancer, breast cancer, brain cancer, multiple myeloma and lymphoma. This chemotherapeutic group includes, but is not limited to lomustine, semustine, carmustine, fotemustine and streptozotocin. Particularly preferred is the combination of an anti-NTPDase3 antibody with lomustine, semustine, carmustine, fotemustine and streptozotocin, and even more preferred is the combination with carmustine in the treatment of cancer, and more particularly in the treatment of lung cancer, ovarian cancer and breast cancer.

In another preferred embodiment, the invention is directed to the combination of an anti-NTPDase3 antibody with a nonclassical alkylating agent in the treatment of cancer, and more particularly in the treatment of lung cancer, sarcoma, malignant melanoma, pancreas carcinoma, gastric carcinoma, ovarian cancer, breast cancer, colorectal cancer, kidney cancer, brain cancer, leukemia and lymphoma. This chemotherapeutic group includes, but is not limited to procarbazine, dacarbazine, temozolomide and altretamine. Particularly preferred is the combination of an anti-NTPDase3 antibody with procarbazine, dacarbazine, temozolomide and altretamine, and even more preferred is the combination with dacarbazine and tezolomide in the treatment of lung cancer, sarcoma, malignant melanoma, gastric carcinoma, ovarian cancer, breast cancer, colorectal cancer, kidney cancer and brain cancer. In another preferred embodiment, the invention is directed to the combination of an anti-NTPDase3 antibody with an estrogen antagonist in the treatment of cancer, and more particularly in the treatment of breast cancer. This chemotherapeutic group includes, but is not limited to toremifene, fulvestrant, tamoxifen and nafoxidine. Particularly preferred is the combination of an anti-NTPDase3 antibody with toremifene, fulvestrant, tamoxifen and nafoxidine, and even more preferred is the combination with tamoxifen in the treatment of breast cancer.

In another preferred embodiment, the invention is directed to the combination of an anti-NTPDase3 antibody with an androgen antagonist in the treatment of cancer, and more particularly in the treatment of prostate cancer. This chemotherapeutic group includes, but is not limited to bicalutamide, flutamide, MDV3100 and nilutamide. Particularly preferred is the combination of an anti-NTPDase3 antibody with bicalutamide, flutamide, MDV3100 and nilutamide, and even more preferred is the combination with flutamide in the treatment of prostate cancer.

In another preferred embodiment, the invention is directed to the combination of an anti-NTPDase3 antibody with a mTOR inhibitor in the treatment of cancer, and more particularly in the treatment of lung cancer, sarcoma, malignant melanoma, prostate cancer, pancreas carcinoma, gastric carcinoma, ovarian cancer, breast cancer, colorectal cancer, kidney cancer and brain cancer. This chemotherapeutic group includes, but is not limited to sirolimus, temsirolimus, everolimus, ridaforolimus, KU-0063794 and WYE-354. Particularly preferred is the combination of an anti-NTPDase3 antibody with sirolimus, temsirolimus, everolimus, ridaforolimus, KU-0063794 and WYE-354, and even more preferred is the combination with temsirolimus in the treatment of lung cancer, sarcoma, malignant melanoma, prostate cancer, pancreas carcinoma, gastric carcinoma, ovarian cancer, breast cancer, colorectal cancer and brain cancer.

In another preferred embodiment, the invention is directed to the combination of an anti-NTPDase3 antibody with a tyrosine kinase inhibitor in the treatment of cancer, and more particularly in the treatment of a cancer selected from lung cancer, sarcoma, prostate cancer, pancreas carcinoma, gastric carcinoma, ovarian cancer, hepatoma, breast cancer, colorectal cancer, kidney cancer and brain cancer. This chemotherapeutic group includes, but is not limited to erlotinib, sorafenib, axitinib, bosutinib, cediranib, crizotinib, dasatinib, gefitinib, imatinib, canertinib, lapatinib, lestaurtinib, neratinib, nilotinib, semaxanib, sunitinib, vatalanib and vandetanib. Particularly preferred is the combination of an anti-NTPDase3 antibody with erlotinib, sorafenib, axitinib, bosutinib, cediranib, crizotinib, dasatinib, gefitinib, imatinib, canertinib, lapatinib, lestaurtinib, neratinib, nilotinib, semaxanib, sunitinib, vatalanib and vandetanib, and even more preferred is the combination with erlotinib in the treatment of cancer, and more particularly in the treatment of a cancer selected from lung cancer, sarcoma, prostate cancer, pancreas carcinoma, gastric carcinoma, ovarian cancer, hepatoma, breast cancer, colorectal cancer, kidney cancer and brain cancer.

Another aspect encompassed by the present invention relates to any one of the foregoing methods, further comprising administering to the patient a MAP kinase pathway inhibitor or a WNT pathway inhibitor.

In some embodiments, the MAP kinase pathway inhibitor is selected from the group consisting of a BRAF inhibitor, a MEK inhibitor, a PI3K inhibitor and a c-KIT inhibitor.

In some embodiments, the BRAF inhibitor is selected from the group consisting of GDC-0879, PLX-4720, sorafenib tosylate, dabrafenib and LGX818.

In some embodiments, the MEK inhibitor is selected from the group consisting of GSK1120212, selumetinib and MEK162.

In some embodiments, the WNT pathway inhibitor is a [symbol] -catenin inhibitor or a frizzled inhibitor.

In some embodiments, the β-catenin inhibitor is selected from the group consisting of niclosamide, XAV-939, FH 535 and ICG 001.

Another aspect encompassed by the present invention relates to any one of the foregoing methods, further comprising administering to the patient a cancer vaccine. In some embodiments, the cancer vaccine is a dendritic cell vaccine.

Another aspect encompassed by the present invention relates to any one of the foregoing methods, further comprising administering to the patient an adoptive cell transfer.

In some embodiments, the adoptive cell transfer is a CAR-T cell therapy.

Another aspect encompassed by the present invention relates to any one of the foregoing methods, further comprising administering to the patient an antibody therapy.

Another aspect encompassed by the present invention relates to any one of the foregoing methods, wherein administration of the anti-NTPDase3 antibody enhances antibody-dependent cell-mediated cytotoxicity of the antibody therapy.

In some embodiment, the antibody therapy is selected from the group consisting of trastuzumab, cetuximab, bevacizumab, and rituximab.

Furthermore, treatment with the anti-NTPDase3 antibody can include combination treatment with other biologic molecules, such as one or more cytokines (e.g., lymphokines, interleukins, tumor necrosis factors, and/or growth factors) or can be accompanied by surgical removal of tumors, removal of cancer cells, or any other therapy deemed necessary by a treating physician. In some embodiments, the additional therapeutic agent is an immune response stimulating agent.

In some embodiments of the methods described herein, the anti-NTPDase3 antibody can be combined with a growth factor selected from the group consisting of: adrenomedullin (AM), angiopoietin (Ang), BMPs, BDNF, EGF, erythropoietin (EPO), FGF, GDNF, G-CSF, GM-CSF, GDF9, HGF, HDGF, IGF, migration-stimulating factor, myostatin (GDF-8), NGF, neurotrophins, PDGF, thrombopoietin, TGF-α, TGF-β, TNF-α, VEGF, P1GF, IL-1, IL-2, IL-3, IL-4, IL-5, IL-6, IL-7, IL-12, IL-15, and IL-18.

In some embodiments of the methods described herein, the additional therapeutic agent is an immune response stimulating agent. In some embodiments, the immune response stimulating agent is selected from the group consisting of granulocyte-macrophage colony stimulating factor (GM-CSF), macrophage colony stimulating factor (M-CSF), granulocyte colony stimulating factor (G-CSF), interleukin 3 (IL-3), interleukin 12 (IL-12), interleukin 1 (IL-1) or interleukin 2 (IL-2).

Administration Scheduling

In certain embodiments of the methods described herein, the treatment involves the administration of an anti-NTPDase3 antibody in combination with radiation therapy. Treatment with an anti-NTPDase3 antibody can occur prior to, concurrently with, or subsequent to administration of radiation therapy. Dosing schedules for such radiation therapy can be determined by the skilled medical practitioner.

In certain embodiments of the methods described herein, the treatment involves the administration of an anti-NTPDase3 antibody in combination with anti-viral therapy. Treatment with an anti-NTPDase3 antibody can occur prior to, concurrently with, or subsequent to administration of antiviral therapy. The anti-viral drug used in combination therapy will depend upon the virus the subject is infected with.

Combined administration can include co-administration, either in a single pharmaceutical formulation or using separate formulations, or consecutive administration in either order but generally within a time period such that all active agents can exert their biological activities simultaneously.

It will be appreciated that the combination of an anti-NTPDase3 antibody and at least one additional therapeutic agent may be administered in any order or concurrently. In some embodiments, the anti-NTPDase3 antibody will be administered to patients that have previously undergone treatment with a second therapeutic agent. In certain other embodiments, the anti-NTPDase3 antibody and a second therapeutic agent will be administered substantially simultaneously or concurrently. For example, a subject may be given an anti-NTPDase3 antibody while undergoing a course of treatment with a second therapeutic agent (e.g., chemotherapy). In certain embodiments, an anti-NTPDase3 antibody will be administered within 1 year of the treatment with a second therapeutic agent. In certain alternative embodiments, an anti-NTPDase3 antibody will be administered within 10, 8, 6, 4, or 2 months of any treatment with a second therapeutic agent. In certain other embodiments, an anti-NTPDase3 antibody will be administered within 4, 3, 2, or 1 weeks of any treatment with a second therapeutic agent. In some embodiments, an anti-NTPDase3 antibody will be administered within 5, 4, 3, 2, or 1 days of any treatment with a second therapeutic agent. It will further be appreciated that the two (or more) agents or treatments may be administered to the subject within a matter of hours or minutes (i.e., substantially simultaneously).

For the treatment of a disease, the appropriate dosage of an anti-NTPDase3 antibody depends on the type of disease to be treated, the severity and course of the disease, the responsiveness of the disease, whether the anti-NTPDase3 antibody is administered for therapeutic or preventative purposes, previous therapy, the patient's clinical history, and so on, all at the discretion of the treating physician. The anti-NTPDase3 antibody can be administered one time or over a series of treatments lasting from several days to several months, or until a cure is effected or a diminution of the disease state is achieved (e.g., reduction in tumor size). Optimal dosing schedules can be calculated from measurements of drug accumulation in the body of the patient and will vary depending on the relative potency of an individual agent. The administering physician can determine optimum dosages, dosing methodologies, and repetition rates. In certain embodiments, dosage is from 0.01 μg to 100 mg/kg of body weight, from 0.1 pig to 100 mg/kg of body weight, from 1 μg to 100 mg/kg of body weight, from 1 mg to 100 mg/kg of body weight, 1 mg to 80 mg/kg of body weight from 10 mg to 100 mg/kg of body weight, from 10 mg to 75 mg/kg of body weight, or from 10 mg to 50 mg/kg of body weight. In certain embodiments, the dosage of the anti-NTPDase3 antibody is from about 0.1 mg to about 20 mg/kg of body weight. In some embodiments, the dosage of the anti-NTPDase3 antibody is about 0.1 mg/kg of body weight. In some embodiments, the dosage of the anti-NTPDase3 antibody is about 0.25 mg/kg of body weight. In some embodiments, the dosage of the anti-NTPDase3 antibody is about 0.5 mg/kg of body weight. In some embodiments, the dosage of the anti-NTPDase3 antibody is about 1 mg/kg of body weight. In some embodiments, the dosage of the anti-NTPDase3 antibody is about 1.5 mg/kg of body weight. In some embodiments, the dosage of the anti-NTPDase3 antibody is about 2 mg/kg of body weight. In some embodiments, the dosage of the anti-NTPDase3 antibody is about 2.5 mg/kg of body weight. In some embodiments, the dosage of the anti-NTPDase3 antibody is about 5 mg/kg of body weight. In some embodiments, the dosage of the anti-NTPDase3 antibody is about 7.5 mg/kg of body weight. In some embodiments, the dosage of the anti-NTPDase3 antibody is about 10 mg/kg of body weight. In some embodiments, the dosage of the anti-NTPDase3 antibody is about 12.5 mg/kg of body weight. In some embodiments, the dosage of the anti-NTPDase3 antibody is about 15 mg/kg of body weight. In certain embodiments, the dosage can be given once or more daily, weekly, monthly, or yearly. In certain embodiments, the anti-NTPDase3 antibody is given once every week, once every two weeks, once every three weeks, or once every four weeks.

In some embodiments, an anti-NTPDase3 antibody may be administered at an initial higher “loading” dose, followed by one or more lower doses. In some embodiments, the frequency of administration may also change. In some embodiments, a dosing regimen may comprise administering an initial dose, followed by additional doses (or “maintenance” doses) once a week, once every two weeks, once every three weeks, or once every month. For example, a dosing regimen may comprise administering an initial loading dose, followed by a weekly maintenance dose of, for example, one-half of the initial dose. Or a dosing regimen may comprise administering an initial loading dose, followed by maintenance doses of, for example one-half of the initial dose every other week. Or a dosing regimen may comprise administering three initial doses for 3 weeks, followed by maintenance doses of, for example, the same amount every other week.

As is known to those of skill in the art, administration of any therapeutic agent may lead to side effects and/or toxicities. In some cases, the side effects and/or toxicities are so severe as to preclude administration of the particular agent at a therapeutically effective dose. In some cases, drug therapy must be discontinued, and other agents may be tried. However, many agents in the same therapeutic class often display similar side effects and/or toxicities, meaning that the patient either has to stop therapy, or if possible, suffer from the unpleasant side effects associated with the therapeutic agent.

In some embodiments, the dosing schedule may be limited to a specific number of administrations or “cycles”. In some embodiments, the anti-NTPDase3 antibody is administered for 3, 4, 5, 6, 7, 8, or more cycles. For example, the anti-NTPDase3 antibody is administered every 2 weeks for 6 cycles, the anti-NTPDase3 antibody is administered every 3 weeks for 6 cycles, the anti-NTPDase3 antibody is administered every 2 weeks for 4 cycles, the anti-NTPDase3 antibody is administered every 3 weeks for 4 cycles, etc. Dosing schedules can be decided upon and subsequently modified by those skilled in the art.

Thus, the present invention provides methods of administering to a subject the anti-NTPDase3 antibody described herein comprising using an intermittent dosing strategy for administering one or more agents, which may reduce side effects and/or toxicities associated with administration of an anti-NTPDase3 antibody, chemotherapeutic agent, etc. In some embodiments, a method for treating cancer in a human subject comprises administering to the subject a therapeutically effective dose of an anti-NTPDase3 antibody in combination with a therapeutically effective dose of a chemotherapeutic agent, wherein one or both of the agents are administered according to an intermittent dosing strategy. In some embodiments, the intermittent dosing strategy comprises administering an initial dose of an anti-NTPDase3 antibody to the subject, and administering subsequent doses of the anti-NTPDase3 antibody about once every 2 weeks. In some embodiments, the intermittent dosing strategy comprises administering an initial dose of an anti-NTPDase3 antibody to the subject, and administering subsequent doses of the anti-NTPDase3 antibody about once every 3 weeks. In some embodiments, the intermittent dosing strategy comprises administering an initial dose of an anti-NTPDase3 antibody to the subject, and administering subsequent doses of the anti-NTPDase3 antibody about once every 4 weeks.

In some embodiments, the anti-NTPDase3 antibody is administered using an intermittent dosing strategy and the chemotherapeutic agent is administered weekly.

Anti-Infective Therapeutic Combinations

In an embodiment, the invention provides methods for treating subjects using an anti-NTPDase3 antibody, wherein the subject suffers from a viral infection. In one embodiment, the viral infection is infection with a virus selected from the group consisting of human immunodeficiency virus (HIV), hepatitis virus (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus or arboviral encephalitis virus.

In an embodiment, the invention provides methods for treating subjects using an anti-NTPDase3 antibody, wherein the subject suffers from a bacterial infection. In one embodiment, the bacterial infection is infection with a bacterium selected from the group consisting of Chlamydia, rickettsial bacteria, mycobacteria, staphylococci, streptococci, pneumonococci, meningococci and gonococci, klebsiella, proteus, serratia, pseudomonas, Legionella, Corynebacterium diphtheriae, Salmonella, bacilli, Vibrio cholerae, Clostridium tetani, Clostridium botulinum, Bacillus anthricis, Yersinia pestis, Mycobacterium leprae, Mycobacterium lepromatosis, and Borriella.

In an embodiment, the invention provides methods for treating subjects using an anti-NTPDase3 antibody, wherein the subject suffers from a fungal infection. In one embodiment, the fungal infection is infection with a fungus selected from the group consisting of Candida (albicans, krusei, glabrata, tropicalis, etc.), Cryptococcus neoformans, Aspergillus (fumigatus, niger, etc.), Genus Mucorales (mucor, absidia, rhizopus), Sporothrix schenkii, Blastomyces dermatitidis, Paracoccidioides brasiliensis, Coccidioides immitis and Histoplasma capsulatum.

In an embodiment, the invention provides methods for treating subjects using an anti-NTPDase3 antibody, wherein the subject suffers from a parasitic infection. In one embodiment, the parasitic infection is infection with a parasite selected from the group consisting of Entamoeba histolytica, Balantidium coli, Naegleria fowleri, Acanthamoeba, Giardia lambia, Cryptosporidium, Pneumocystis carinii, Plasmodium vivax, Babesia microti, Trypanosoma brucei, Trypanosoma cruzi, Leishmania donovani, Toxoplasma gondii and Nippostrongylus brasiliensis.

### VII. Anti-NTPDase3 Antibody Conjugates

The anti-NTPDase3 antibodies disclosed herein may also be conjugated to a chemical moiety. The chemical moiety may be, inter alia, a polymer, a radionuclide or a cytotoxic factor.

For instance, the present invention provides an anti-NTPDase3 antibody conjugated to a therapeutic moiety, i.e. a drug. The therapeutic moiety can be, e.g., a cytotoxin, a chemotherapeutic agent, a cytokine, an immunosuppressant, an immune stimulator, a lytic peptide, or a radioisotope. Such conjugates are referred to herein as an “antibody-drug conjugates” or “ADCs”. Accordingly, in one aspect, the anti-NTPDase3 antibody according to any above-described aspect or embodiment is conjugated to a therapeutic moiety.

Exemplary therapeutic moieties include a cytotoxic moiety, a radioisotope, a cytokine, and a lytic peptide.

In certain embodiments, the anti-NTPDase3 antibody is capable of inducing cytotoxicity in NTPDase3-expressing cells by internalization of the antibody conjugated to or associated with a cytotoxic moiety. The cytotoxic moiety may, for example, be selected from the group consisting of taxol; cytochalasin B; gramicidin D; ethidium bromide; emetine; mitomycin; etoposide; tenoposide; vincristine; vinblastine; colchicine; doxorubicin; daunorubicin; dihydroxy anthracin dione; a tubulin-inhibitor such as maytansine or an analog or derivative thereof; an antimitotic agent such as monomethyl auristatin E or F or an analog or derivative thereof; dolastatin 10 or 15 or an analogue thereof; irinotecan or an analogue thereof; mitoxantrone; mithramycin; actinomycin D; 1-dehydrotestosterone; a glucocorticoid; procaine; tetracaine; lidocaine; propranolol; puromycin; calicheamicin or an analog or derivative thereof; an antimetabolite such as methotrexate, 6 mercaptopurine, 6 thioguanine, cytarabine, fludarabin, 5 fluorouracil, decarbazine, hydroxyurea, asparaginase, gemcitabine, or cladribine; an alkylating agent such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C; a platinum derivative such as cisplatin or carboplatin; duocarmycin A, duocarmycin SA, rachelmycin (CC-1065), or an analog or derivative thereof; an antibiotic such as dactinomycin, bleomycin, daunorubicin, doxorubicin, idarubicin, mithramycin, mitomycin, mitoxantrone, plicamycin, anthramycin (AMC)); pyrrolo[2,1-c][1,4]-benzodiazepines (PDB); diphtheria toxin and related molecules such as diphtheria A chain and active fragments thereof and hybrid molecules, ricin toxin such as ricin A or a deglycosylated ricin A chain toxin, cholera toxin, a Shiga-like toxin such as SLT I, SLT II, SLT IIV, LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins such as PAPI, PAPII, and PAP-S, Momordica charantia inhibitor, curcin, crotin, Sapaonaria officinalis inhibitor, gelonin, mitogellin, restrictocin, phenomycin, and enomycin toxins; ribonuclease (RNase); DNase I, Staphylococcal enterotoxin A; pokeweed antiviral protein; diphtherin toxin; and Pseudomonas endotoxin.

In one embodiment, the anti-NTPDase3 antibody is conjugated to an auristatin or a peptide analog, derivative or prodrug thereof. Auristatins have been shown to interfere with microtubule dynamics, GTP hydrolysis and nuclear and cellular division (Woyke et al. (2001) Antimicrob. Agents and Chemother. 45(12): 3580-3584) and have anti-cancer (U.S. Pat. No. 5,663,149) and anti-fungal activity (Pettit et al., (1998) Antimicrob. Agents and Chemother. 42:2961-2965). For example, auristatin E can be reacted with para-acetyl benzoic acid or benzoylvaleric acid to produce AEB and AEVB, respectively. Other typical auristatin derivatives include AFP, MMAF (monomethyl auristatin F), and MMAE (monomethyl auristatin E). Suitable auristatins and auristatin analogs, derivatives and prodrugs, as well as suitable linkers for conjugation of auristatins to Abs, are described in, e.g., U.S. Pat. Nos. 5,635,483, 5,780,588 and 6,214,345 and in International patent application publications WO02088172, WO2004010957, WO2005081711, WO2005084390, WO2006132670, WO03026577, WO200700860, WO207011968 and WO205082023.

In another embodiment, the anti-NTPDase3 antibody is conjugated to pyrrolo[2,1-c][1,4]-benzodiazepine (PDB) or an analog, derivative or prodrug thereof. Suitable PDBs and PDB derivatives, and related technologies are described in, e.g., Hartley J. A. et al., Cancer Res 2010; 70(17): 6849-6858; Antonow D. et al., Cancer J 2008; 14(3):154-169; Howard P. W. et al., Bioorg Med Chem Lett 2009; 19: 6463-6466 and Sagnou et al., Bioorg Med Chem Lett 2000; 10(18): 2083-2086.

In another embodiment, the anti-NTPDase3 antibody is conjugated to a cytotoxic moiety selected from the group consisting of an anthracycline, maytansine, calicheamicin, duocarmycin, rachelmycin (CC-1065), dolastatin 10, dolastatin 15, irinotecan, monomethyl auristatin E, monomethyl auristatin F, a PDB, or an analog, derivative, or prodrug of any thereof.

In a particular embodiment, the anti-NTPDase3 antibody is conjugated to an anthracycline or an analog, derivative or prodrug thereof. In another particular embodiment, the antibody is conjugated to maytansine or an analog, derivative or prodrug thereof. In another particular embodiment, the antibody is conjugated to calicheamicin or an analog, derivative or prodrug thereof. In another particular embodiment, the antibody is conjugated to duocarmycin or an analog, derivative or prodrug thereof. In another particular embodiment, the antibody is conjugated to rachelmycin (CC-1065) or an analog, derivative or prodrug thereof. In another particular embodiment, the antibody is conjugated to dolastatin 10 or an analog, derivative or prodrug thereof. In another particular embodiment, the antibody is conjugated to dolastatin 15 or an analog, derivative or prodrug thereof. In another particular embodiment, the antibody is conjugated to monomethyl auristatin E or an analog, derivative or prodrug thereof. In another particular embodiment, the antibody is conjugated to monomethyl auristatin F or an analog, derivative or prodrug thereof. In another particular embodiment, the antibody is conjugated to pyrrolo[2,1-c][1,4]-benzodiazepine or an analog, derivative or prodrug thereof. In another particular embodiment, the antibody is conjugated to irinotecan or an analog, derivative or prodrug thereof.

In one embodiment, an anti-NTPDase3 antibody encompassed by the present invention is conjugated to a nucleic acid or nucleic acid-associated molecule. In one such embodiment, the conjugated nucleic acid is a cytotoxic ribonuclease (RNase) or deoxy-ribonuclease (e.g., DNase 1), an antisense nucleic acid, an inhibitory RNA molecule (e.g., a siRNA molecule) or an immunostimulatory nucleic acid (e.g., an immunostimulatory CpG motif-containing DNA molecule). In another embodiment, a NTPDase3-specific antibody encompassed by the present invention is conjugated to an aptamer or a ribozyme.

In one embodiment, an anti-NTPDase3 antibody encompassed by the present invention is conjugated, e.g., as a fusion protein, to a lytic peptide such as CLIP, Magainin 2, mellitin, Cecropin and P18.

In one embodiment, the anti-NTPDase3 antibody is conjugated to a cytokine, such as, e.g., IL-2, IL-4, IL-6, IL-7, IL-10, IL-12, IL-13, IL-15, IL-18, IL-23, IL-24, IL-27, IL-28a, IL-28b, IL-29, KGF, IFNα, IFNβ, IFNγ, GM-CSF, CD40L, Flt3 ligand, stem cell factor, ancestim, and TNFα.

In certain embodiments, the chemical moiety is a polymer which increases the half-life of the antibody or fragment in the body of a subject. Suitable polymers include, but are not limited to, hydrophilic polymers which include but are not limited to polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee, et al., (1999) (Bioconj. Chem. 10:973-981) discloses PEG conjugated single-chain antibodies. Wen, et al., (2001) (Bioconj. Chem. 12:545-553) disclose conjugating antibodies with PEG which is attached to a radiometal chelator (diethylenetriaminpentaacetic acid (DTPA)).

The anti-NTPDase3 antibodies may also be conjugated with labels such as ⁹⁹Tc, ⁹⁰Y, ¹¹¹In, ³²P, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷CU, ²¹⁷Ci, ²¹¹At, ²¹²Pb ⁴⁷Sc, ¹⁰⁹Pd, ²³⁴Th, ⁴⁰K, ¹⁵⁷Gd, ⁵⁵Mn, ⁵²Tr, and ⁵⁶Fe.

The anti-NTPDase3 antibodies may also be conjugated with fluorescent or chemiluminescent labels, including fluorophores such as rare earth chelates, fluorescein and its derivatives, rhodamine and its derivatives, isothiocyanate, phycoerythrin, phycocyanin, allophycocyanin, o-phthalaldehyde, fluorescamine, ¹⁵²Eu, dansyl, umbelliferone, luciferin, luminal label, isoluminal label, an aromatic acridinium ester label, an imidazole label, an acridinium salt label, an oxalate ester label, an aequorin label, 2,3-dihydrophthalazinediones, biotin/avidin, spin labels and stable free radicals.

Any method known in the art for conjugating the antibodies and antigen-binding fragments thereof encompassed by the present invention to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating antibodies and fragments are conventional and very well known in the art.

### VIII. Pharmaceutical Compositions

Anti-NTPDase3 antibodies, antibody fragments, nucleic acids, or vectors encompassed by the present invention can be formulated in compositions, especially pharmaceutical compositions. Such compositions comprise a therapeutically or prophylactically effective amount of an anti-NTPDase3 antibody, antibody fragment, nucleic acid, or vector encompassed by the present invention in admixture with a suitable carrier, e.g., a pharmaceutically acceptable agent. Typically, anti-NTPDase3 antibodies, antibody fragments, nucleic acids, or vectors encompassed by the present invention are sufficiently purified for administration to an animal before formulation in a pharmaceutical composition.

Pharmaceutically acceptable agents for use in the present pharmaceutical compositions include carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants.

Neutral buffered saline or saline mixed with serum albumin are exemplary appropriate carriers. The pharmaceutical compositions can include antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics, or polyethylene glycol (PEG). Also by way of example, suitable tonicity enhancing agents include alkali metal halides (preferably sodium or potassium chloride), mannitol, sorbitol, and the like. Suitable preservatives include benzalkonium chloride, thimerosal, phenethyl alcohol, methylparaben, propylparaben, chlorhexidine, sorbic acid and the like. Hydrogen peroxide also can be used as preservative. Suitable cosolvents include glycerin, propylene glycol, and PEG. Suitable complexing agents include caffeine, polyvinylpyrrolidone, beta-cyclodextrin or hydroxy-propyl-beta-cyclodextrin. Suitable surfactants or wetting agents include sorbitan esters, polysorbates such as polysorbate 80, tromethamine, lecithin, cholesterol, tyloxapal, and the like. The buffers can be conventional buffers such as acetate, borate, citrate, phosphate, bicarbonate, or Tris-HCl. Acetate buffer may be about pH 4-5.5, and Tris buffer can be about pH 7-8.5. Additional pharmaceutical agents are set forth in Remington's Pharmaceutical Sciences, 18th Edition, A. R. Gennaro, ed., Mack Publishing Company, 1990.

The composition can be in liquid form or in a lyophilized or freeze-dried form and may include one or more lyoprotectants, excipients, surfactants, high molecular weight structural additives and/or bulking agents (see for example U.S. Pat. Nos. 6,685,940, 6,566,329, and 6,372,716). In one embodiment, a lyoprotectant is included, which is a non-reducing sugar such as sucrose, lactose or trehalose. The amount of lyoprotectant generally included is such that, upon reconstitution, the resulting formulation will be isotonic, although hypertonic or slightly hypotonic formulations also may be suitable. In addition, the amount of lyoprotectant should be sufficient to prevent an unacceptable amount of degradation and/or aggregation of the protein upon lyophilization. Exemplary lyoprotectant concentrations for sugars (e.g., sucrose, lactose, trehalose) in the pre-lyophilized formulation are from about 10 mM to about 400 mM. In another embodiment, a surfactant is included, such as for example, nonionic surfactants and ionic surfactants such as polysorbates (e.g. polysorbate 20, polysorbate 80); poloxamers (e.g. poloxamer 188); poly (ethylene glycol) phenyl ethers (e.g. Triton); sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, Hnoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl ofeyl-taurate; and the MONAQUAT™. series (Mona Industries, Inc., Paterson, NJ.), polyethyl glycol, polypropyl glycol, and copolymers of ethylene and propylene glycol (e.g. Pluronics, PF68 etc.). Exemplary amounts of surfactant that may be present in the pre-lyophilized formulation are from about 0.001-0.5%. High molecular weight structural additives (e.g. fillers, binders) may include for example, acacia, albumin, alginic acid, calcium phosphate (dibasic), cellulose, carboxymethylcellulose, carboxymethylcellulose sodium, hydroxyethylcellulose, hydroxypropylcellulose, hydroxypropylmethylcellulose, microcrystalline cellulose, dextran, dextrin, dextrates, sucrose, tylose, pregelatinized starch, calcium sulfate, amylose, glycine, bentonite, maltose, sorbitol, ethylcellulose, disodium hydrogen phosphate, disodium phosphate, disodium pyrosulfite, polyvinyl alcohol, gelatin, glucose, guar gum, liquid glucose, compressible sugar, magnesium aluminum silicate, maltodextrin, polyethylene oxide, polymethacrylates, povidone, sodium alginate, tragacanth microcrystalline cellulose, starch, and zein. Exemplary concentrations of high molecular weight structural additives are from 0.1% to 10% by weight. In other embodiments, a bulking agent (e.g., mannitol, glycine) may be included.

Compositions can be suitable for parenteral administration. Exemplary compositions are suitable for injection or infusion into an animal by any route available to the skilled worker, such as intraarticular, subcutaneous, intravenous, intramuscular, intraperitoneal, intracerebral (intraparenchymal), intracerebroventricular, intramuscular, intraocular, intraarterial, or intralesional routes. A parenteral formulation typically will be a sterile, pyrogen-free, isotonic aqueous solution, optionally containing pharmaceutically acceptable preservatives.

Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringers' dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present, such as, for example, anti-microbials, anti-oxidants, chelating agents, inert gases and the like. See generally, Remington's Pharmaceutical Science, 16th Ed., Mack Eds., 1980, which is incorporated herein by reference.

Pharmaceutical compositions described herein can be formulated for controlled or sustained delivery in a manner that provides local concentration of the product (e.g., bolus, depot effect) and/or increased stability or half-life in a particular local environment. The compositions can include the formulation of anti-NTPDase3 antibodies, antibody fragments, nucleic acids, or vectors encompassed by the present invention with particulate preparations of polymeric compounds such as polylactic acid, polyglycolic acid, etc., as well as agents such as a biodegradable matrix, injectable microspheres, microcapsular particles, microcapsules, bioerodible particles beads, liposomes, and implantable delivery devices that provide for the controlled or sustained release of the active agent which then can be delivered as a depot injection. Techniques for formulating such sustained- or controlled-delivery means are known and a variety of polymers have been developed and used for the controlled release and delivery of drugs. Such polymers are typically biodegradable and biocompatible. Polymer hydrogels, including those formed by complexation of enantiomeric polymer or polypeptide segments, and hydrogels with temperature or pH sensitive properties, may be desirable for providing drug depot effect because of the mild and aqueous conditions involved in trapping bioactive protein agents (e.g., antibodies). See, for example, the description of controlled release porous polymeric microparticles for the delivery of pharmaceutical compositions in PCT Application Publication WO 93/15722.

Suitable materials for this purpose include polylactides (see, e.g., U.S. Pat. No. 3,773,919), polymers of poly-(a-hydroxycarboxylic acids), such as poly-D-(−)-3-hydroxybutyric acid (EP 133,988A), copolymers of L-glutamic acid and gamma ethyl-L-glutamate (Sidman et al., Biopolymers, 22: 547-556 (1983)), poly (2-hydroxyethyl-methacrylate) (Langer et al., J. Biomed. Mater. Res., 15: 167-277 (1981), and Langer, Chem. Tech., 12: 98-105 (1982)), ethylene vinyl acetate, or poly-D(˜)˜3-hydroxybutyric acid. Other biodegradable polymers include poly(lactones), poly(acetals), poly(orthoesters), and poly(orthocarbonates). Sustained-release compositions also may include liposomes, which can be prepared by any of several methods known in the art (see, e.g., Eppstein et al, Proc. Natl. Acad. Sci. USA, 82: 3688-92 (1985)). The carrier itself, or its degradation products, should be nontoxic in the target tissue and should not further aggravate the condition. This can be determined by routine screening in animal models of the target disorder or, if such models are unavailable, in normal animals. [00196] Microencapsulation of recombinant proteins for sustained release has been performed successfully with human growth hormone (rhGH), interferon-(rhlFN-), interleukin-2, and MNrgpl20. Johnson et al., Nat. Med., 2:795-799 (1996); Yasuda, Biomed. Ther., 27:1221-1223 (1993); Hora et al., Bio/Technology. 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010. The sustained-release formulations of these proteins were developed using poly-lactic-coglycolic acid (PLGA) polymer due to its biocompatibility and wide range of biodegradable properties. The degradation products of PLGA, lactic and glycolic acids can be cleared quickly within the human body. Moreover, the degradability of this polymer can be depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41. Additional examples of sustained release compositions include, for example, EP 58,48 IA, U.S. Pat. No. 3,887,699, EP 158,277A, Canadian Patent No. 1176565, U. Sidman et al., Biopolymers 22, 547 [1983], R. Langer et al., Chem. Tech. 12, 98 [1982], Sinha et al., J. Control. Release 90, 261 [2003], Zhu et al., Nat. Biotechnol. 18, 24 [2000], and Dai et al., Colloids Surf B Biointerfaces 41, 117 [2005].

Bioadhesive polymers are also contemplated for use in or with compositions encompassed by the present invention. Bioadhesives are synthetic and naturally occurring materials able to adhere to biological substrates for extended time periods. For example, Carbopol and polycarbophil are both synthetic cross-linked derivatives of poly (acrylic acid). Bioadhesive delivery systems based on naturally occurring substances include for example hyaluronic acid, also known as hyaluronan. Hyaluronic acid is a naturally occurring mucopolysaccharide consisting of residues of D-glucuronic and N-acetyl-D-glucosamine. Hyaluronic acid is found in the extracellular tissue matrix of vertebrates, including in connective tissues, as well as in synovial fluid and in the vitreous and aqueous humor of the eye. Esterified derivatives of hyaluronic acid have been used to produce microspheres for use in delivery that are biocompatible and biodegradable (see for example, Cortivo et al., Biomaterials (1991) 12:727-730; European Publication No. 517,565; International Publication No. WO 96/29998; Ilium et al., J. Controlled Rel. (1994) 29:133-141). Exemplary hyaluronic acid containing compositions encompassed by the present invention comprise a hyaluronic acid ester polymer in an amount of approximately 0.1% to about 40% (w/w) of an IL-1/3 binding antibody or fragment to hyaluronic acid polymer. [00198] Both biodegradable and non-biodegradable polymeric matrices can be used to deliver compositions encompassed by the present invention, and such polymeric matrices may comprise natural or synthetic polymers. Biodegradable matrices are preferred. The period of time over which release occurs is based on selection of the polymer. Typically, release over a period ranging from between a few hours and three to twelve months is most desirable. Exemplary synthetic polymers which can be used to form the biodegradable delivery system include: polymers of lactic acid and glycolic acid, polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyanhydrides, polyurethanes and co-polymers thereof, poly(butic acid), poly(valeric acid), alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), polyethylene oxide), polyethylene terephthalate), poly(vinyl alcohols), polyvinyl acetate, poly vinyl chloride, polystyrene and polyvinylpyrrolidone. Exemplary natural polymers include alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The polymer optionally is in the form of a hydrogel (see for example WO 04/009664, WO 05/087201, Sawhney, et al., Macromolecules, 1993, 26, 581-587,) that can absorb up to about 90% of its weight in water and further, optionally is cross-linked with multi-valent ions or other polymers.

Delivery systems also include non-polymer systems that are lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the product is contained in a form within a matrix such as those described in U.S. Pat. Nos. 4,452,775, 4,675,189 and 5,736,152 and (b) diffusional systems in which a product permeates at a controlled rate from a polymer such as described in U.S. Pat. Nos. 3,854,480, 5,133,974 and 5,407,686. Liposomes containing the product may be prepared by methods known methods, such as for example (DE 3,218,121; Epstein et al., Proc. Natl. Acad. Sci. USA, 82: 3688-3692 (1985); Hwang et al., Proc. Natl. Acad. Sci. USA, 77: 4030-4034 (1980); EP 52,322; EP 36,676; EP 88,046; EP 143,949; EP 142,641; Japanese patent application 83-118008; U.S. Pat. Nos. 4,485,045 and 4,544,545; and EP 102,324).

Alternatively or additionally, the compositions can be administered locally via implantation into the affected area of a membrane, sponge, or other appropriate material on to which an anti-NTPDase3 antibody, antibody fragment, nucleic acid, or vector encompassed by the present invention has been absorbed or encapsulated. Where an implantation device is used, the device can be implanted into any suitable tissue or organ, and delivery of an anti-NTPDase3 antibody, antibody fragment, nucleic acid, or vector encompassed by the present invention can be directly through the device via bolus, or via continuous administration, or via catheter using continuous infusion.

A pharmaceutical composition comprising an anti-NTPDase3 antibody, antibody fragment, nucleic acid, or vector encompassed by the present invention can be formulated for inhalation, such as for example, as a dry powder. Inhalation solutions also can be formulated in a liquefied propellant for aerosol delivery. In yet another formulation, solutions may be nebulized. Additional pharmaceutical composition for pulmonary administration include, those described, for example, in PCT Application Publication WO 94/20069, which discloses pulmonary delivery of chemically modified proteins. For pulmonary delivery, the particle size should be suitable for delivery to the distal lung. For example, the particle size can be from 1 μm to 5 μm; however, larger particles may be used, for example, if each particle is fairly porous.

Certain formulations containing anti-NTPDase3 antibody, antibody fragments, nucleic acids, or vectors encompassed by the present invention can be administered orally. Formulations administered in this fashion can be formulated with or without those carriers customarily used in the compounding of solid dosage forms such as tablets and capsules. For example, a capsule can be designed to release the active portion of the formulation at the point in the gastrointestinal tract when bioavailability is maximized and pre-systemic degradation is minimized. Additional agents can be included to facilitate absorption of a selective binding agent. Diluents, flavorings, low melting point waxes, vegetable oils, lubricants, suspending agents, tablet disintegrating agents, and binders also can be employed.

Another preparation can involve an effective quantity of an anti-NTPDase3 antibody, antibody fragment, nucleic acid, or vector encompassed by the present invention in a mixture with non-toxic excipients which are suitable for the manufacture of tablets. By dissolving the tablets in sterile water, or another appropriate vehicle, solutions can be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

### IX. Exemplary Methods

Materials and Methods

Reagents

All chemical reagents were purchased from Sigma-Aldrich (St. Louis, MO), cell culture media from Life Technologies (Carlsbad, CA), cell culture consumables from CELLTREAT® Scientific Products (Shirley, MA), and commercial antibodies from Biolegend (San Diego, CA), unless otherwise stated. Secondary antibodies including Alexa Fluor® 488-conjugated AffinityPure Donkey anti-human IgG (Fc specific) (#709-545-098), Alexa Fluor® 488-conjugated anti-rabbit IgG (H+L) (#711-545-152), Biotin-SP-conjugated AffinPure F(ab′)2 Fragment Donkey Anti-Rabbit IgG(H+L) (#711-066-152), Biotin-SP-conjugated AffinPure F(ab′)2 Fragment Donkey Anti-Human IgG(H+L) (#709-066-149) were obtained from Jackson ImmunoResearch (West Grove, PA), CellTiter-Glo® (#G7571) and Bio-Glo™ (#G7941) from Promega (Madison, WI), mouse anti-hENTPD3 clone hN3-B3s from Ectonocleotidases-ab (Cat #hN3-B3s; Quebec, Canada).

Cell Culture

Human ENTPD3 stably transfected Chinese Hamster Ovary cells (CHO-hENTPD3) were maintained in DMEM supplemented with 10% fetal bovine serum (FBS), 1% penicillin-streptomycin. Human bladder carcinoma cells endogenously expressing hENTPD3 (RT4; ECACC; Sigma #91091914) were grown in McCoy's 5a plus 10% FBS, 1% penicillin-streptomycin. All cell lines were maintained in culture flasks at 37° C. in a 5% CO₂ atmosphere at 100% humidity, except for Jurkat cells/NFAT-luc+FcγRIIIA (Promega Cat #: G7011), which were thawed in water bath at 37° C. prior to use for experiments.

Monoclonal Antibody Affinity to Cell Lines Expressing Human ENTPD3

Cells transfected with or endogenously expressing hENTPD3 (1×10⁵ cells) were incubated with serially diluted monoclonal antibodies for 30 minutes at 4° C., followed by two washes with cell staining buffer. Cells were then incubated with anti-human IgG (Fc specific) Alexa Fluor® 488 (1:5000) for 30 minutes at 4° C., followed by two washes with cell staining buffer and analysis by Cytek™ Aurora flow cytometry (Cytek Biosciences, Fremont, CA). Alexa Fluor® 488 (AF488) median fluorescence intensity (MFI) was detected and data was analyzed by FCS Express 7 software (De Novo Software, Los Angeles, CA).

Inhibition of human ENTPD3 enzyme activity on intact cells CHO-hENTPD3 cells (8×10⁴ cells/well) and RT4 bladder cancer cells (3×10⁵ cells/well) were trypsinized, counted and plated in a 96-well plate-U bottom. Suspension cells were then washed twice with Modified Ringer Buffer (RB) (120 mM NaCl, 5 mM KCl, 2.5 mM CaCl2), 1.2 mM MgSO4, 25 mM NaHCO₃, 10 mM dextrose, 80 mM Tris-HCl, pH 7.4) and incubated with monoclonal antibodies for 30 minutes at 37° C. CHO-hENTPD3 cells were then incubated with 250 μM of ATP for 15 minutes at room temperature, while RT4 were incubated with 50 μM of ATP for 45 minutes at 37° C. Supernatants were finally collected to a 96-well opaque-walled multiwell plate (BRANDplates #781968) and ATP levels were detected by luminescence using CellTiter-Glo®. Luminescence values were read on a Synergy™ Neo2 Multi-Mode Reader (BioTeK Instruments Inc., Winooski, VT) and are directly correlated to ATP levels. Cells with no antibody (Cells+ATP) or ATP alone in the absence of cells served as controls. Results were either expressed as relative luminescence unit (RLU) or % of enzyme activity inhibition calculated by: [(Cells+ATP+Ab)−(Cells+ATP)/(ATP)−(Cells+ATP)]×100. All steps were performed in RB.

Enzyme activity for attached RT4 cells were performed the same way, except cells (1.5×10⁵ cells/well) were plated in a 96-well plate-flat bottom, incubated overnight and exposed to 25 μM of ATP for 45 minutes at 37° C. For the sake of clarity, all percentage values of NTPD3 enzyme activity inhibition described herein indicate the percentage inhibition relative to 100% (e.g., a value of 41 as shown in FIG. 5 indicates a decrease of enzymatic activity of 41% as compared to a baseline of 100% activity).

NFAT Luciferase Reporter Jurkat System (ADCC Assay)

Target cells (CHO-hENTPD3) were seeded in a 96-well plate (8×10³ cells/100 μl/well) (BRANDplates #781965) and grown for 24 hours. Cells were then washed twice with ADCC assay buffer (DMEM medium supplemented with 4% ultra-low IgG serum) and incubated with serially diluted monoclonal antibodies for 30 minutes at 37° C. Effector cells (Jurkat cells/NFAT-luc+FcγRIIIA) (3×10⁶ cells/ml) were then added to the wells and the mixture (T:E=1:6) were incubated for 6 hours at 37° C. Bio-Glo™ was finally added into wells and luminescence value was read at 5, 15, and 30 minutes using a Synergy™ Neo2 Multi-Mode Reader (BioTeK Instruments Inc.). ADCC activity was indicated by an increase of luciferase activity over background.

Epitope Competition Assay

Anti-hENTPD3 monoclonal antibodies (clones 3E9, 38D5, 38D12, 44H5 and PBI #30) were conjugated with Alexa Fluor® 647 using Antibody Labeling Kit according to the manufacturer's instructions (Thermo Fisher Scientific #A20186). Unconjugated human IgG1 isotype control or anti-hENTPD3 monoclonal antibodies (20 μg/mL) were incubated with CHO-hENTPD3 cells (1×10⁵ cells) for 30 minutes at 4° C. Next, Alexa Fluor® 647-conjugated anti-hENTPD3 monoclonal antibodies (3E9 1:1000; 38D5, 38D12 and 44H5 1:800; and PBI #30 1:400) were added to each well and incubated for 30 minutes at 4° C. Cells were then washed twice with cell staining buffer and analyzed by Cytek™ Aurora flow cytometry (Cytek Biosciences). Alexa Fluor® 647 (AF647) median fluorescence intensity (MFI) was detected and data was analyzed by FCS Express 7 software (De Novo Software). Fold change in AF647 MFI detection in relation to isotype control was calculated (No epitope overlap=1).

Animal Studies

C57BL6 hENTPD3 KI mice were licensed from Beth Israel Deaconess Medical Center. Six to 8-week-old female mice were used for tumor inoculation. Syngeneic murine MC38 colorectal cancer cells were maintained in RPMI 1640 medium supplemented with 10% FBS, penicillin (100 units/mL) and streptomycin (100 μg/mL). 5×10⁵ of MC38 cells were harvested by trypsinization and resuspended with 150 μl of RPMI 1640 supplemented with 10% FBS for injection. MC38 cells were injected subcutaneously into the right flank of hENTPD3 KI mice. Mice were then randomized into three groups (n=6). At day 4, tumor-bearing mice received 20 mg/kg of chimeric antibody 38D5 or 8E1, or 200 μl of saline, via intraperitoneal injection. On days 7, 10 and 14, tumor-bearing mice received 10 mg/kg of 38D5 or 8E1 antibody, or 200 μl of saline. Tumor length (L) and width (W) were measured using a digital caliper twice weekly. Tumor volume (mm³) was determined as L*W*W*0.52.

Statistical Analyses

Statistical analyses were performed using GraphPad Prism 8 (GraphPad Software, San Diego, CA).

Additional assay methods, including assaying functionality of select anti-ENTPD3 antibody functionality such as humanized, affinity matured and/or isotype switched variants of parental clones like clone PBI #30, 8E1 and 38D5, were further used and supplemented experimental procedures are detailed as follows:

Reagents

Normal Human Serum (#A113) was purchased from Quidel Corporatio (San Siego, CA) fo the CDC assay.

Cell Culture

Human ENTPD3 stably transfected Chinese Hamster Ovary (CHO-hENTPD3), COS7 (COS7-hENTPD3), and HEK293T (HEK293T-hENTPD3) cells were maintained in DMEM supplemented with 10% fetal bovine serum (FBS), 1% penicillin-streptomycin. All cell lines were maintained in culture flasks at 37° C. in a 5% CO₂ atmosphere at 100% humidity. These three cell lines express differential levels of hENTPD3: HEK293T-hENTPD3 (very high) >CHO-hENTPD3 (high) >COS7-hENTPD3 (moderate). HEK293T-hENTPD3 cells contain very high levels of hENTPD3 enzyme activity thus degrading exogenous ATP very quickly, which are not considered to be physiologically relevant. hENTPD3 expression/enzymatic activity levels on COS7-hENTPD3 cells are similar to those on RT4 human bladder cancer cells (endogenously expressing hENTPD3). Due to the technical difficulty with culturing/working with RT4 cells, two physiologically relevant cell lines COS7-hENTPD3 and CHO-hENTPD3 cells matched for ENTPD3 expression/enzyme activity were therefore chosen for in vitro binding and functional assays, representative of ENTPD3+ and ENTPD3^{high} cells in the tumor microenvironment, respectively.

Monoclonal Antibody Affinity to Cell Lines Expressing Human ENTPD3

Cells transfected with hENTPD3 (1×10⁵ cells) were incubated with serially diluted monoclonal antibodies for 2 hours at 37° C., followed by two washes with cell staining buffer. All subsequent procedures and analysis were the same as described above.

Inhibition of Human ENTPD3 Enzyme Activity on Intact Cells

Cells were trypsinized, counted and plated (8×10⁴ cells/well) in a 96-well plate-U bottom. Suspension cells were then incubated with monoclonal antibodies for 2 h at 37° C. in complete culture media. All subsequent procedures and analysis were the same as described above.

Complement-Dependent Cytotoxicity (CDC) Assay

CHO-hENTPD3 cells were washed twice with serum-free RPMI 1640 medium, resuspended in CDC assay buffer (RPMI 1640 medium with 4% ultra-low IgG FBS) at the final concentration of 2×10⁶/ml and rested on ice for 2-3 hours. Cells were then incubated with serially diluted monoclonal antibodies for 30 minutes at 37° C. in 5% CO₂. Normal Human Serum (NHS 10%) were then added to the cells and incubated for 2 hours at 37° C. in 5% CO₂. After incubation, cells were stained with Propidium Iodide (P/I) (200 ng/mL) for 10 minutes at room temperature and target cell death was analyzed by Cytek Aurora flow cytometry (Cytek Biosciences). Results were expressed as % of cytotoxicity (P/I^{+ cells) and % of maximal cytotoxicity over background (% of maximal cytotoxicity at} 1 μg/mL-% of background cytotoxicity).

Stable Immune Complex Assay

FIG. 50 for exemplary anti-ENTPD3 monoclonal antibodies in this invention, COS7-hENTPD3 cells were trypsinized, counted, plated (3×10⁴ cells/well) in a 48-well plate and grown overnight. Attached cells were then incubated with anti-human ENTPD3 antibodies (6 μg/ml) or left untreated for 24 hours at 37° C. in 5% CO₂. The following day, untreated cells were exposed to the same panel of monoclonal antibodies (6 μg/ml) but for 2 hours at 37° C. to obtain the basal level of ENTPD3 expression. Cells were then washed twice with cell staining buffer and stained with anti-human IgG (Fc specific) Alexa Fluor 488 (1:2000) for 30 minutes at 4° C., followed by two additional washes with PBS 1× and trypsinization. Once cells were detached, culture media was added to the plate and cells were transferred to a 96 U-well untreated plate (Corning #3365). Lastly, cells were spined down at 1200 rpm for 5 min, resuspend in cell staining buffer, and analyzed by Cytek Aurora flow cytometry (Cytek Biosciences). Alexa Flour 488 (AF488) MFI was detected, and data was analyzed by FCS Express 7 software (De Novo Software). The percentage of human ENTPD3 loss on cell membrane at 24 hours was calculated as: [(2 hours MFI−24 h MFI/2 hours MFI)]×100. FIG. 51 for the other subject anti-ENTPD3 monoclonal antibodies in this invention, CHO-hENTPD3 cells were trypsinized, counted and plated (5×10⁵ cells/mL) in a 96 U-well untreated plate (Corning #3365) to avoid cell attachment. Cells were then incubated with anti-human ENTPD3 Human/Rabbit chimeric antibodies (2 μg/ml) or left untreated for 24 hours at 37° C. in 5% CO₂. The following day, untreated cells were exposed to the same panel of monoclonal antibodies (2 μg/ml) but for 20 minutes at 4° C. to obtain the basal level of ENTPD3 expression. Cells were then washed twice with cell staining buffer and stained with anti-human IgG (Fc specific) Alexa Fluor 488 (1:2000) for 30 minutes at 4° C., followed by two additional washes and fixation with paraformaldehyde (PFA, 2%) for 10 minutes at room temperature. Lastly, cells were washed twice and analyzed by Cytek Aurora flow cytometry (Cytek Biosciences). Alexa Flour 488 (AF488) MFI was detected, and data was analyzed by FCS Express 7 software (De Novo Software). The percentage of human ENTPD3 loss on cell membrane at 24 hours was calculated as: [(20 min MFI−24 h MFI/20 min MFI)]×100.

Detection of Free Antibody in Plasma of Tumor-Bearing Mice after Repeated Antibody Treatment

For PBI #30 maturation variants: C57BL6 hENTPD3 KI female mice (13- to 16-week-old) were inoculated subcutaneously with MC38 colorectal cancer cells (1×10⁵) in 150 μl of RPMI 1640 media. Tumor-bearing mice received 3 mg/kg of fully human anti-hENTPD3 antibody PBI #3af4 hIgG1 or PBI #3af4 hIgG4, on days 8, 12, 15, 18, and 21 via intraperitoneal injection. Plasma samples were collected on days 20, 21, 22 and 24 and kept at −80° C. until further analysis. Plasma was collected before dose administration on dosing days and two different animals were subjected to sample analysis per collection day (sample name was designated as animal number+collection date). The estimated free antibody level in plasma was detected by incubating serially diluted plasma with COS7-hENTPD3 cells and analyzing cell binding activity as described in the new section of “Monoclonal antibody affinity to cell lines expressing human ENTPD3” above.

For humanized 38D5 hIgG1: C57BL6 hENTPD3 KI female mice (9-week-old) were inoculated subcutaneously with MC38 colorectal cancer cells (1×10⁵) in 150 μl of RPMI 1640 media. Tumor-bearing mice received 3 mg/kg of 38D5 hIgG1, on days 12, 15, 18, and 21 via intraperitoneal injection. Plasma samples were collected on days 19, 21 and 23, and kept at −80° C. until further analysis. Plasma was collected before dose administration on dosing days and two to three different animals were subjected to sample analysis per collection day (sample name was designated as animal number+collection date). The estimate free antibody level in plasma was detected in the same manner as for PBI #30 maturation variants described above.

Detection of Free Antibody in Plasma of Tumor-Free Mice after Single Dose Antibody Treatment

C57BL6 hENTPD3 KI tumor-free female mice (9-week-old) received one dose of 1 or 10 mg/kg of 38D5 hIgG1 via intraperitoneal injection. A total of two mice were used in this experiment, one for each dose. Plasma samples were then collected from each mouse 24 h and 48 h later and kept at −80° C. until further analysis (sample name was designated as: dose injected+collection time). The estimated free antibody level in plasma was detected as described in the section above.

Animal Studies

Additional in vivo studies were performed for PBI #30 hIgG1 and its affinity maturation variants PBI #30af4 hIgG1 and PBI #3af4 hIgG4, as well as humanized 38D5 hIgG1 and 38D5 hIgG4. C57BL6 hENTPD3 KI mice were licensed from Beth Israel Deaconess Medical Center and bred in-house at the Purinomia Animal Facility. Syngeneic murine MC38 colorectal cancer cells were maintained in RPMI 1640 medium supplemented with 10% FBS, penicillin (100 units/mL) and streptomycin (100 μg/mL).

For PBI #3af4 hIgG1 and PBI #3af4 hIgG4 maturation variants: 13- to 16-week-old C57BL6 hENTPD3 KI female mice were inoculated subcutaneously with MC38 colorectal cancer cells (1×10⁵) in 150 μl of RPMI 1640 media. Mice were then randomized into three groups (n=8 per group). Tumor-bearing mice received 3 mg/kg of fully human anti-hENTPD3 antibody PBI #3af4 hIgG1 or PBI #3af4 hIgG4, or 200 μl of saline, on days 8, 12, 15, 18 and 21, via intraperitoneal injection. Tumor length (L) and width (W) were measured using a digital caliper every two days. Tumor volume (mm³) was determined as L*W*W*0.52.

For PBI #30 hIgG1 and humanized 38D5 hIgG1 and 38D5 hIgG4 antibodies: Six- to 8-week-old C57BL6 hENTPD3 KI female mice were inoculated subcutaneously with MC38 colorectal cancer cells (5×10⁵) in 150 μl of RPMI 1640 media. Mice were then randomized into two groups (n=5 per group). On day 4, tumor-bearing mice received 20 mg/kg of PBI #30 hIgG1 or humanized 38D5 hIgG1 or 38D5 hIgG4 antibody, or 200 μl of saline, via intraperitoneal injection. On days 7, 10 and 14, tumor-bearing mice received 10 mg/kg of the respective anti-ENTPD3 antibody, or 200 μl of saline. Tumor length (L) and width (W) were measured using a digital caliper twice weekly. Tumor volume (mm³) was determined as L*W*W*0.52.

TABLE 4
Detection antibodies

| Name | Vendor/Cat# | Dilution |
|---|---|---|
| Donkey Anti-human IgG, Fc specific | Jackson ImmunoResearch | 1:5000 |
| Alexa Fluor ® 488 | Cat#709-545-098 | (FACS) |
| AffinityPure Donkey Anti-rabbit IgG | Jackson ImmunoResearch | 1:5000 |
| (H + L) Alexa Fluor ® 488 | Cat#711-545-152 | |
| Biotin-SP-conjugated AffinPure | Jackson ImmunoResearch | 1:800 |
| F(ab′)2 Fragment Donkey Anti- | Cat#711-066-152 | |
| rabbit IgG(H + L) | | |
| Biotin-SP-conjugated AffinPure | Jackson ImmunoResearch | 1:800 |
| F(ab′)2 Fragment Donkey Anti- | Cat#709-066-149 | |
| human IgG(H + L) | | |
| Rabbit Anti-human ENTPD3 Clone | Purinomia Biotech Inc. | 1:1000 |
| 3E9 Alexa Fluor ® 647 | | |
| Human/Rabbit Anti-human ENTPD3 | Purinomia Biotech Inc. | 1:800 |
| Clone 38D5 Alexa Fluor ® 647 | | |
| Human/Rabbit Anti-human ENTPD3 | Purinomia Biotech Inc. | 1:800 |
| Clone 38D12 Alexa Fluor ® 647 | | |
| Human/Rabbit Anti-human ENTPD3 | Purinomia Biotech Inc. | 1:800 |
| Clone 44H5 Alexa Fluor ® 647 | | |
| Human Anti-human ENTPD3 Clone | Purinomia Biotech Inc. | 1:400 |
| PBI#30 Alexa Fluor ® 647 | | |

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web and/or the National Center for Biotechnology Information (NCBI) on the World Wide Web.

### EQUIVALENTS AND SCOPE

The details of one or more embodiments encompassed by the present invention are set forth in the description above. Although the preferred materials and methods have been described above, any materials and methods similar or equivalent to those described herein may be used in the practice or testing of embodiments encompassed by the present invention. Other features, objects and advantages related to the present invention are apparent from the description. Unless defined otherwise, all technical and scientific terms used herein have the same meaning as commonly understood by one of ordinary skill in the art to which this invention belongs. In the case of conflict, the present description provided above will control.

Those skilled in the art will recognize or be able to ascertain using no more than routine experimentation, many equivalents to the specific embodiments encompassed by the present invention described herein. The scope encompassed by the present invention is not intended to be limited to the description provided herein and such equivalents are intended to be encompassed by the appended claims.

The articles “a” and “an” are used herein to refer to one or to more than one (i.e. to at least one) of the grammatical object of the article unless indicated to the contrary or otherwise evident from the context. By way of example, “an element” means one element or more than one element. Claims or descriptions that include “or” between one or more members of a group are considered satisfied if one, more than one, or all of the group members are present in, employed in, or otherwise relevant to a given product or process unless indicated to the contrary or otherwise evident from the context. The present invention includes embodiments in which exactly one member of the group is present in, employed in, or otherwise relevant to a given product or process. The present invention also includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges may assume any specific value or subrange within the stated ranges in different embodiments encompassed by the present invention, to the tenth of the unit of the lower limit of the range, unless the context clearly dictates otherwise.

In addition, it is to be understood that any particular embodiment encompassed by the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions encompassed by the present invention (e.g., any antibiotic, therapeutic or active ingredient; any method of production; any method of use; etc.) may be excluded from any one or more claims, for any reason, whether or not related to the existence of prior art.

It is to be understood that the words which have been used are words of description rather than limitation, and that changes may be made within the purview of the appended claims without departing from the true scope and spirit encompassed by the present invention in its broader aspects.

While the present invention has been described at some length and with some particularity with respect to several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope encompassed by the present invention.

1. An anti-NTPDase3 antibody, or antigen-binding fragment thereof, comprising at least one antigen binding domain that binds ectonucleoside triphosphate diphosphohydrolase-3 (NTPDase3) at a site such that the anti-NTPDase3 antibody forms a stable immune complex, and
(a) an FcγRIIIa binding moiety that binds FcγRIIIa receptor and confers antibody-dependent cellular cytotoxicity (ADCC) activity against NTPDase3+ cells to the anti-NTPDase3 antibody; and/or
(b) wherein the anti-NTPDase3 antibody, or antigen-binding fragment thereof, inhibits NTPDase3 enzymatic activity;
optionally wherein
(i) the anti-NTPDase3 antibody, or antigen-binding fragment thereof, has an EC50 of at least 2×10⁻⁶ molar (M) or lower in an in vitro ADCC assay, preferably wherein the EC50 is 1×10⁻⁶ M or lower, 0.5×10⁻⁶ M or lower, 1×10⁻⁷ M or lower, 7.5×10⁻⁸ M or lower, 5×10⁻⁸ M or lower, 2.5×10⁻⁸ M, 1×10⁻⁸ M or lower, 7.5×10⁻⁹ M or lower, 5×10⁻⁹ M or lower, 2.5×10⁻⁹ M or lower, 1×10⁻⁹ M or lower, 7.5×10⁻¹⁰ M or lower, 5×10⁻¹⁰ M or lower, 2.5×10⁻¹⁰ M or lower, 1×10⁻¹⁰ M or lower, 7.5×10⁻¹¹ M or lower, 5×10⁻¹¹ M or lower, 2.5×10⁻¹¹ M or lower, 1×10⁻¹¹ M or lower, 7.5×10⁻¹² M or lower, 5×10⁻¹² M or lower, 2.5×10⁻¹² M or lower, or 1×10⁻¹² M or lower, or any range in between, inclusive, including 1×10⁻⁶ M to 1×10⁻¹² M, 5×10⁻⁷ to 5×10⁻⁹ M, and 1×10⁻⁷ to 1×10⁻⁹ M;
(ii) the anti-NTPDase3 antibody, or antigen-binding fragment thereof, has an EC50 of at least 2×10⁻⁶ M or lower in an in vitro NTPD3 enzymatic activity inhibition assay, preferably wherein the EC50 is 1×10⁻⁶ M or lower, 0.5×10⁻⁶ M or lower, 1×10⁻⁷ M or lower, 7.5×10⁻⁸ M or lower, 5×10⁻⁸ M or lower, 2.5×10⁻⁸ M, 1×10⁻⁸ M or lower, 7.5×10⁻⁹ M or lower, 5×10⁻⁹ M or lower, 2.5×10⁻⁹ M or lower, 1×10⁻⁹ M or lower, 7.5×10⁻¹⁰ M or lower, 5×10⁻¹⁰ M or lower, 2.5×10⁻¹⁰ M or lower, 1×10⁻¹⁰ M or lower, 7.5×10⁻¹¹ M or lower, 5×10⁻¹¹ M or lower, 2.5×10⁻¹¹ M or lower, 1×10⁻¹¹ M or lower, 7.5×10⁻¹² M or lower, 5×10⁻¹² M or lower, 2.5×10⁻¹² M or lower, or 1×10⁻¹² M or lower, or any range in between, inclusive, including 1×10⁻⁶ M to 1×10⁻¹² M, 5×10⁻⁷ to 5×10⁻⁹ M, and 1×10⁻⁷ to 1×10⁻⁹ M, and with a maximal inhibition potency of at least 30%, 35%, 40%, 45%, 50%, 55%, 60%, 65%, 70%, 75%, 80%, 85%, 90%, 95%, 99%, or higher, or any range in between, inclusive, including 30% to 99%, as determined by an in vitro NTPD3 enzymatic activity inhibition assay; or
(iii) the anti-NTPDase3 antibody, or antigen-binding fragment thereof, has an EC50 of at least 4×10⁻⁶ M or lower in an in vitro ADCC assay, preferably wherein the EC50 is 2×10⁻⁶ M or lower, 1×10⁻⁶ M or lower, 0.5×10⁻⁶ M or lower, 1×10⁻⁷ M or lower, 7.5×10⁻⁸ M or lower, 5×10⁻⁸ M or lower, 2.5×10⁻⁸ M, 1×10⁻⁸ M or lower, 7.5×10⁻⁹ M or lower, 5×10⁻⁹ M or lower, 2.5×10⁻⁹ M or lower, 1×10⁻⁹ M or lower, 7.5×10⁻¹⁰ M or lower, 5×10⁻¹⁰ M or lower, 2.5×10⁻¹⁰ M or lower, 1×10⁻¹⁰ M or lower, 7.5×10⁻¹¹ M or lower, 5×10⁻¹¹ M or lower, 2.5×10⁻¹¹ M or lower, 1×10⁻¹¹ M or lower, 7.5×10⁻¹² M or lower, 5×10⁻¹² M or lower, 2.5×10⁻¹² M or lower, or 1×10⁻¹² M or lower, or any range in between, inclusive, including 1×10⁻⁶ M to 1×10⁻¹² M, 5×10⁻⁷ to 5×10⁻⁹ M, and 1×10⁻⁷ to 1×10⁻⁹ M; plus having an EC50 of at least 4×10⁻⁶ M or lower in an in vitro enzymatic activity inhibition assay, preferably wherein the EC50 is 2×10⁻⁶ M or lower, 1×10⁻⁶ M or lower, 0.5×10⁻⁶ M or lower, 1×10⁻⁷ M or lower, 7.5×10⁻⁸ M or lower, 5×10⁻⁸ M or lower, 2.5×10⁻⁸ M, 1×10⁻⁸ M or lower, 7.5×10⁻⁹ M or lower, 5×10⁻⁹ M or lower, 2.5×10⁻⁹ M or lower, 1×10⁻⁹ M or lower, 7.5×10⁻¹⁰ M or lower, 5×10⁻¹⁰ M or lower, 2.5×10⁻¹⁰ M or lower, 1×10⁻¹⁰ M or lower, 7.5×10⁻¹¹ M or lower, 5×10⁻¹¹ M or lower, 2.5×10⁻¹¹ M or lower, 1×10⁻¹¹ M or lower, 7.5×10⁻¹² M or lower, 5×10⁻¹² M or lower, 2.5×10⁻¹² M or lower, or 1×10⁻¹² M or lower, or any range in between, inclusive, including 1×10⁻⁶ M to 1×10⁻¹² M, 5×10⁻⁷ to 5×10⁻⁹ M, and 1×10⁻⁷ to 1×10⁻⁹ M, and with a maximal inhibition potency of at least 5%, 10%, 15%, 20%, 25%, 30%, 35%, 40%, 45%, 50%, 55%, 60%, 65%, 70%, 75%, 80%, 85%, 90%, 95%, 99%, or higher, or any range in between, inclusive, including 30% to 99%, as determined by an in vitro NTPD3 enzymatic activity inhibition assay.

2. The anti-NTPDase3 antibody, or antigen-binding fragment thereof, of claim 1, wherein the anti-NTPDase3 antibody, or antigen-binding fragment thereof, is a therapeutic antibody, and/or further promotes:
(i) complement dependent cytotoxicity (CDC) activity against NTPDase3+ cells; and/or
(ii) ADCC activity against NTPDase3+ intratumoral cells and/or NTPDase3+ pericytes and/or fibroblastic cells around the tumor vessels; and/or
(iii) antibody-mediated target cytosis of NTPDase3 on NTPDase3+ immune cells (preferably M2 macrophage); and/or
(iv) binding to NTPDase3 in a manner that is competitive, non-competitive, or partially competitive with an NTPDase3 monoclonal antibody clone binding to NTPDase3, wherein the NTPDase3 monoclonal antibody clone is selected from the group consisting of PBI #30 and its affinity maturation variants, 3E9, 4F9, 8E1 and its humanized counterparts, 16D4, 37H1, 38D5 and its humanized counterparts with or without point mutation in their backbone sequences, 38D12, 42D8, and 44H5;
optionally wherein:
the FcγRIIIa binding moiety is selected from the group consisting of an Fc domain, an antibody or fragment thereof that binds to FcγRIIIa, and an FcγRIIIa binding peptide;
the antigen binding domain is selected from the group consisting of a Fab, Fab′, F(ab′)2, Fv or single chain Fv (scFv), Fav, dsFv, sc(Fv)2, Fde, sdFv, single domain antibody (dAb), and diabodies fragments and/or wherein the anti-NTPDase3 antibody, or antigen-binding fragment, is monoclonal; and/or
the anti-NTPDase3 antibody, or antigen-binding fragment thereof, is conjugated to an agent, further optionally wherein the agent is selected from the group consisting of a binding protein, an enzyme, a drug, a chemotherapeutic agent, a biologic agent, a toxin, a radionuclide, an immunomodulatory agent, a detectable moiety, and a tag.

3-5. (canceled)

6. The anti-NTPDase3 antibody, or antigen-binding fragment thereof, of claim 1, wherein the anti-NTPDase3 antibody, or antigen-binding fragment thereof, has a VH domain with an amino acid sequence that can be encoded by a nucleic acid that hybridizes under stringent conditions to the nucleic acid of SEQ ID No. 1, 9, 13, 17, 21, 25, 29, 33, 37, 41, 75, 79, or sequences listed in Table 2A, 2B, 2C, 2D, or 3, and a VL domain with an amino acid sequence that can be encoded by a nucleic acid that hybridizes under stringent conditions to the nucleic acid of SEQ ID No. 3, 11, 15, 19, 23, 27, 31, 35, 39, 43, 77, 81, or sequences listed in Table 2A, 2B, 2C, 2D, or 3;
optionally wherein:
the anti-NTPDase3 antibody, or antigen-binding fragment thereof, comprises a heavy chain having CDRs at least 60% identical to the CDRs of SEQ ID No. 2, 10, 14, 18, 22, 26, 30, 34, 38, 42, 76, 80, or sequences listed in Table 2A, 2B, 2C, 2D, or 3, and a light chain having CDRs at least 60% identical to the CDRs of SEQ ID No. 4, 12, 16, 20, 24, 28, 32, 36, 40, 44, 78, 82, or sequences listed in Table 2A, 2B, 2C, 2D, or 3;
the anti-NTPDase3 antibody, or antigen-binding fragment thereof, comprises a variable heavy (VH) chain at least 60% identical to SEQ ID No. 2, 10, 14, 18, 22, 26, 30, 34, 38, 42, 76, 80, or sequences listed in Table 2A, 2B, 2C, 2D, or 3, and a variable light (VL) chain at least 60% identical to SEQ ID No. 4, 12, 16, 20, 24, 28, 32, 36, 40, 44, 78, 82, or sequences listed in Table 2A, 2B, 2C, 2D, or 3; and/or
the anti-NTPDase3 antibody, or antigen-binding fragment thereof, comprises:
(i) a heavy chain having a CDR1 amino acid sequence at least 80% identical to SEQ ID No. 45, a CDR2 amino acid sequence at least 80% identical to SEQ ID No. 46, and a CDR3 amino acid sequence at least 80% identical to SEQ ID No. 47; and
(ii) a light chain having a CDR1 amino acid sequence at least 80% identical to SEQ ID No. 48, a CDR2 amino acid sequence at least 80% identical to SEQ ID No. 49, and a CDR3 amino acid sequence at least 80% identical to SEQ ID No. 50.

7-9. (canceled)

10. The anti-NTPDase3 antibody, or antigen-binding fragment thereof, of claim 1, wherein the anti-NTPDase3 antibody, or antigen-binding fragment thereof, comprises a heavy chain having CDRs selected from the group consisting of CDRs of SEQ ID No. 2, 10, 14, 18, 22, 26, 30, 34, 38 42, 76, 80, and sequences listed in Tables 2A, 2B, 2C, 2D, and 3, and a light chain having CDRs selected from the group consisting of CDRs of SEQ ID No. 4, 12, 16, 20, 24, 28, 32, 36, 40, 44, 78, 82, and sequences listed in Tables 2A, 2B, 2C, 2D, and 3, and human framework sequences to form humanized heavy and light chains with an antigen binding site able to specifically bind human NTPDase3;
optionally wherein:
the anti-NTPDase3 antibody, or antigen-binding fragment thereof, comprises an Fc domain of an IgG1, IgG3, IgG2, or IgG4 isotype, further optionally wherein the Fc domain is human, preferably the isotype is an IgG1 or IgG3 that is ADCC active;
the anti-NTPDase3 antibody, or antigen-binding fragment thereof, is hypo-fucosylated or afucosylated;
the anti-NTPDase3 antibody, or antigen-binding fragment thereof, is human or is humanized; and/or
the anti-NTPDase3 antibody, or antigen-binding fragment thereof, is a bispecific comprising at least one additional antigen binding site for a tumor antigen, immune checkpoint, or costimulatory receptor, wherein if the additional antigen binding site is for an immune checkpoint it functions as a checkpoint inhibitor and wherein if the additional antigen binding site is for a costimulatory receptor it functions as a costimulatory agonist, further optionally wherein:
the additional antigen binding site binds to a checkpoint protein selected from the group consisting of PD-1, PD-L1, CTLA-4/B7-1/B7-2, PD-L2, NKG2A, KIR, LAG-3, TIM-3, CD96, VISTA, TIGIT, CD39 and Siglec-15:
the additional antigen binding site binds a checkpoint protein upregulated on T-cells and associated with T-cell exhaustion:
the additional antigen binding site binds to an immune costimulatory receptors selected from the group consisting of MHCI molecules, BTLA receptor, OX40, CD27, CD28, CDS, ICAM-1, LFA-1 (CD11a/CD18), ICOS (CD278) and 4-1BB (CD137); and/or
the additional antigen binding site binds to CD47, SIRPα, CD24 or Siglec-10.

11-18. (canceled)

19. A pharmaceutical preparation comprising a therapeutically effective amount of at least one anti-NTPDase3 antibody, or antigen-binding fragment thereof, of claim 1, and one or more pharmaceutically acceptable excipients, buffers or solutions,
optionally wherein the pharmaceutical preparation is for improving anti-tumor T cell immunity and suitable for administration to a subject having a tumor, comprising an effective amount of the anti-NTPDase3 antibody, or antigen-binding fragment thereof, and one or more pharmaceutically acceptable excipients, buffers or solutions, wherein administration of the anti-NTPDase3 antibody to the subject results in a reduction in numbers of intratumoral NTPDase3+ cells (such as M2 and/or M2-like macrophages, pericytes, and/or fibroblasts) and, optionally, i) enhances T-cell infiltration into the tumor, ii) decreases T-cell exhaustion in the tumor, and/or iii) disrupts tumor-associated vasculature thereby resulting in tumor starvation.

20. (canceled)

21. An isolated nucleic acid molecule that
(i) hybridizes, under stringent conditions, with the complement of a nucleic acid encoding an immunoglobulin heavy and/or light chain polypeptide of the anti-NTPDase3 antibody, or antigen-binding fragment thereof, of claim 1;
(ii) has a sequence with at least about 90% identity across its full length to a nucleic acid encoding an immunoglobulin heavy and/or light chain polypeptide of the anti-NTPDase3 antibody, or antigen-binding fragment thereof, of claim 1; and/or
(iii) encodes an immunoglobulin heavy and/or light chain polypeptide of the anti-NTPDase3 antibody, or antigen-binding fragment thereof, of claim 1.

22. An isolated immunoglobulin heavy and/or light chain polypeptide encoded by the isolated nucleic acid of claim 21.

23. A vector comprising the isolated nucleic acid of claim 21, optionally wherein the vector is an expression vector.

24. A host cell which comprises the isolated nucleic acid of claim 21, and optionally:
a) expresses the anti-NTPDase3 antibody, or antigen-binding fragment thereof;
b) comprises the immunoglobulin heavy and/or light chain polypeptide; and/or
c) comprises a vector comprising the isolated nucleic acid, further optionally wherein the vector is an expression vector.

25. A device or kit comprising at least one anti-NTPDase3 antibody, or antigen-binding fragment thereof, of claim 1, said device or kit optionally comprising a label to detect at least one anti-NTPDase3 antibody, or antigen-binding fragment thereof, or a complex comprising the anti-NTPDase3 antibody, or antigen-binding fragment thereof.

26. A device or kit comprising the isolated nucleic acid molecule of claim 21.

27. A method of producing at least one anti-NTPDase3 antibody, or antigen-binding fragment thereof, of claim 1, which method comprises the steps of: (i) culturing a transformed host cell which has been transformed by a nucleic acid comprising a sequence encoding at least one anti-NTPDase3 antibody, or antigen-binding fragment thereof, under conditions suitable to allow expression of said anti-NTPDase3 antibody, or antigen-binding fragment thereof; and (ii) recovering the expressed anti-NTPDase3 antibody, or antigen-binding fragment thereof.

28. A method of detecting the presence or level of NTPDase3 polypeptide comprising obtaining a sample and detecting said polypeptide in the sample by use of at least one anti-NTPDase3 antibody, or antigen-binding fragment thereof, of claim 1:
optionally wherein at least one anti-NTPDase3 antibody, or antigen-binding fragment thereof, forms a complex with the NTPDase3 polypeptide and the complex is detected in the form of an enzyme linked immunosorbent assay (ELISA), radioimmune assay (RIA), immunochemical assay, Western blot, mass spectrometry assay, nuclear magnetic resonance assay, or using an intracellular flow assay.

29. (canceled)

30. A method for improving anti-tumor T cell immunity by depleting intratumoral NTPDase3+ cells, comprising administering to a subject having a tumor an effective amount of a pharmaceutical composition of an anti-NTPDase3 antibody, or antigen-binding fragment thereof, of claim 1,
wherein administration of the anti-NTPDase3 antibody, or antigen-binding fragment thereof, results in a reduction in numbers of intratumoral NTPDase3+ cells (such as M2 and M2-like macrophages, pericytes, and/or fibroblasts) and enhances T-cell infiltration into the tumor or decreases T-cell exhaustion in the tumor or both; and optionally, disrupts tumor-associated vasculature causing tumor starvation.

31. A method for promoting immune cell infiltration into tumors, comprising administering to a subject having a tumor an effective amount of a pharmaceutical composition of an anti-NTPDase3 antibody, or antigen-binding fragment thereof, of claim 1,
wherein administration of the anti-NTPDase3 antibody, or antigen-binding fragment thereof, results in ablation and reduction of NTPDase3+ cells in the tumor.

32. A method for reducing type II NKT cells suppression of intratumoral immune cell function, comprising administering to a subject having a tumor an effective amount of a pharmaceutical composition of an anti-NTPDase3 antibody, or antigen-binding fragment thereof, of claim 1, optionally
wherein administration of the anti-NTPDase3 antibody, or antigen-binding fragment thereof, results in ablation and reduction of M2 macrophage in the tumor.

33. A method for reducing regulatory T cells (Treg) suppression of intratumoral immune cell function, comprising administering to a subject having a tumor an effective amount of a pharmaceutical composition of an anti-NTPDase3 antibody, or antigen-binding fragment thereof, of claim 1, optionally
wherein administration of the anti-NTPDase3 antibody, or antigen-binding fragment thereof, results in reduced immunosuppressive activity of M2 macrophage in the tumor.

34. A method for promoting an anti-tumor immune response comprising administering to a subject having a tumor an anti-NTPDase3 antibody, or antigen-binding fragment thereof, of claim 1, in an amount sufficient to result in a reduction of NTPDase3 expressing cells in the tumor.

35. A method for promoting antitumor immune function in a tumor of a subject, comprising
(i) identifying a cancer subject having a degree of tumor infiltrated tumor-reactive lymphocytes that is below a predetermined threshold so as to be characterized as being a non-infiltrated or under-infiltrated tumor phenotype; and
(ii) administering to the subject an anti-NTPDase3 antibody, or antigen-binding fragment thereof, of claim 1 in an amount that decreases the cell number or immunosuppressive activity of M2 macrophage in the tumor.

36. The method of claim 30, wherein the anti-NTPDase3 antibody, or antigen-binding fragment thereof is:
administered as part of an antitumor therapy;
administered as part of an antitumor therapy for treating a solid tumor, optionally wherein the solid tumor is pancreatic cancer, liver cancer, lung cancer, stomach cancer, esophageal cancer, head and neck squamous cell carcinoma, prostate cancer, colon cancer, breast cancer, lymphoma, gallbladder cancer, renal cancer, multiple myeloma, ovarian cancer, cervical cancer or glioma;
administered as part of an antitumor therapy for treating a liquid tumor, further optionally wherein the liquid tumor is a leukemia;
administered as part of a therapy involving one or more chemotherapeutic agents, anti-angiogenetic agents, immuno-oncology agents and/or radiation; and/or
administered as a part of therapy comprising a tumor vaccine, adoptive cell therapy, antitumor gene therapy, inhibitory nucleic acid therapy and/or oncolytic viral therapy;
further optionally wherein the therapy comprises:
administered as one or more inhibitors (antagonists) of one or more checkpoint molecules, further optionally wherein the one or more checkpoint molecules is selected from the group consisting of a PD-1 antagonists, a CTLA-4 antagonist, a LAG-3 antagonist, a TIM-3 antagonist, a TIGIT antagonist and a Siglec-15 antagonist;
administered as one or more activators (agonists) of one or more costimulatory molecules, further optionally wherein the one or more costimulatory molecules is selected from the group consisting of a GITR agonist, a CD27 agonist, a 4-1BB agonist, an OX40 agonist, a CD137 agonist, an ICOS agonist and a CD28 agonist;
administered as one or more of a VEGFR or VEGF antagonist, an EGFR or EGF antagonist, an IDO inhibitor, an IDO1 inhibitor, an HDAC inhibitor, a PI3K delta inhibitor, an IL-15 agonist, a CXCR4 antagonist, a CXCL12 antagonist, a DNMT inhibitor, interleukin-21, an anti-KIR antibody, an anti-CSF-1R antibody, an anti-CCR4 antibody, GMCSF, an anti-PS antibody, an anti-CD30 antibody-auristatin E conjugate, an anti-CD19 antibody, an anti-CEA IL-2 antibody, an anti-NY-ESO-1 antibody, an anti-NKG2A antibody, a STING agonist, a TRL7/8 agonist, a RIG-1 agonist and/or NRLP3 inhibitor, an anti-CD73 antibody (such as MEDI9447), a P2X7 antagonist, an adenosine A2A receptor antagonist or an anti-CD39 antibody; and/or
administered as one or more innate immune inducers, further optionally wherein the one or more innate immune inducers is selected from the group consisting of an inhibitor of the CD47-SIRPα axis, an inhibitor of the CD24-Siglec-10 axis, an NGK2A checkpoint inhibitor that blocks HLA-E driven inhibition of NK and CD8+ cells, a STING agonist, a TLR7/8 agonist and an RIG-I agonist.

37-44. (canceled)


SEQUENCE LISTING (146 sequences)

SEQ ID NO: 1 - DNA, 384 bp, Homo sapiens
  CDS (1)..(384)
caggtgcagctgcaggagtcggggggaggtttgatacagcctgggggatccctgagactctcctgcgcagcctctggattcacctctagttctagtgattatgccatgagttgggtccgccaggctccagggaaggggctggagtgggtctcgtctattagtggaagtggcggtagcacatactacgcagactccgtgaagggccgcttcaccatctccagagacaattccaagaacacactctatgtgcaaatgaccagcctgacagccgaggacacggccgtatattactgtgcgaaagtcgtcggatacagtatgtacgactactactaccactacgctttggacgtctggggccaagggaccacggtcaccgtctcctca

SEQ ID NO: 2 - protein, 128 aa, Homo sapiens
QVQLQESGGGLIQPGGSLRLSCAASGFTSSSSDYAMSWVRQAPGKGLEWVSSISGSGGSTYYADSVKGRFTISRDNSKNTLYVQMTSLTAEDTAVYYCAKVVGYSMYDYYYHYALDVWGQGTTVTVSS

SEQ ID NO: 3 - DNA, 324 bp, Homo sapiens
  CDS (1)..(324)
cagcctgtgctgactcagccaccttcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctcaccatctctggactgaagactgaggacgaggctggctactactgtcagtcttatgataccagcaatgtggtattcggcggagggaccaaggtgaccgtcctc

SEQ ID NO: 4 - protein, 108 aa, Homo sapiens
QPVLTQPPSASGTPGQRVTISCSGSSSNIGSNTVNWYQQLPGTAPKLLIYSNNQRPSGVPDRFSGSKSGTSASLTISGLKTEDEAGYYCQSYDTSNVVFGGGTKVTVL

SEQ ID NO: 5 - DNA, 1377 bp, Homo sapiens
  CDS (1)..(1377)
caggtgcagctgcaggagtcggggggaggtttgatacagcctgggggatccctgagactctcctgcgcagcctctggattcacctctagttctagtgattatgccatgagttgggtccgccaggctccagggaaggggctggagtgggtctcgtctattagtggaagtggcggtagcacatactacgcagactccgtgaagggccgcttcaccatctccagagacaattccaagaacacactctatgtgcaaatgaccagcctgacagccgaggacacggccgtatattactgtgcgaaagtcgtcggatacagtatgtacgactactactaccactacgctttggacgtctggggccaagggaccacggtcaccgtctcctcagcctccactaagggcccatccgtgttcccactggcaccctctagtaagagcacatctgggggtactgccgctctgggatgtctggtgaaggattacttcccagagccagtcaccgtgtcctggaacagcggggccctgacttccggtgtccatacctttccagctgtgctgcagtcatccggcctgtacagcctgagctctgtggtcaccgtccccagttcatccctgggaacacagacttatatctgcaacgtgaatcacaagccatccaatacaaaagtcgacaagaaagtggaacccaagagctgtgataaaacccatacatgccccccttgtcctgctccagagctgctgggaggaccatccgtgttcctgtttccacccaagcctaaagacactctgatgatttctcgaacccccgaagtcacatgcgtggtcgtggacgtgtcccacgaggatcctgaagtcaagttcaactggtacgtggatggcgtcgaggtgcataatgccaagacaaaaccacgagaggaacagtacaacagtacctatcgtgtcgtgtcagtcctgacagtgctgcaccaggactggctgaacgggaaggaatataagtgcaaagtgagcaataaggcactgcccgcccctatcgagaaaacaatttctaaggctaaaggacagcctagggaaccacaggtgtacactctgcctccatcacgggacgagctgacaaagaaccaggtcagtctgacttgtctggtgaaagggttctatccttctgatatcgccgtggagtgggaaagtaatggtcagccagagaacaattacaagaccacaccccctgtcctggactctgatgggagtttctttctgtattccaagctgaccgtggataaaagccggtggcagcagggtaatgtctttagttgttcagtgatgcacgaggcactgcacaatcactacacccagaaatcactgtcactgtcaccaggtaaatga

SEQ ID NO: 6 - protein, 458 aa, Homo sapiens
QVQLQESGGGLIQPGGSLRLSCAASGFTSSSSDYAMSWVRQAPGKGLEWVSSISGSGGSTYYADSVKGRFTISRDNSKNTLYVQMTSLTAEDTAVYYCAKVVGYSMYDYYYHYALDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 645 bp, Homo sapiens
  CDS (1)..(645)
cagcctgtgctgactcagccaccttcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctcaccatctctggactgaagactgaggacgaggctggctactactgtcagtcttatgataccagcaatgtggtattcggcggagggaccaaggtgaccgtcctcggccagcctaaagccaacccaacagtgactctgtttccaccctccagcgaggaactgcaggctaataaggcaactctggtctgtctgatttctgacttctaccctggagctgtgaccgtcgcatggaaggctgatggttcccccgtgaaagcaggcgtcgagaccacaaagccttctaaacagagtaacaataagtacgccgcttctagttatctgtctctgacaccagaacagtggaaaagtcataggtcctatagctgtcaggtcactcacgaaggctcaactgtggaaaaaactgtggctcctaccgaatgctcatag

SEQ ID NO: 8 - protein, 214 aa, Homo sapiens
QPVLTQPPSASGTPGQRVTISCSGSSSNIGSNTVNWYQQLPGTAPKLLIYSNNQRPSGVPDRFSGSKSGTSASLTISGLKTEDEAGYYCQSYDTSNVVFGGGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 9 - DNA, 369 bp, Artificial Sequence
  human-rabbit hybrid
  CDS (1)..(369)
cagtcgttggaggagtccgggggagacctggtcaagcctggggcatccctgacactcacctgcacggcctctggattctccttcagtagcagcgactggatatgctgggtccgccaggctccagggaaggggctggagtggatcgcatgcatttatgctggtagtattggtggcgcttacttcgcgagctgggtgaatggccgattcaccatctccagagcctcgtcgaccacggtgactctgcaaatgaccagtctgacagccgcggacacggccacctatttctgtgcgagatcaagtagtaatggtcgtttgaaattggatcaattcgacttgtggggcccaggcaccctggtcaccgtctcctca

SEQ ID NO: 10 - protein, 123 aa, Artificial Sequence
  Synthetic Construct
QSLEESGGDLVKPGASLTLTCTASGFSFSSSDWICWVRQAPGKGLEWIACIYAGSIGGAYFASWVNGRFTISRASSTTVTLQMTSLTAADTATYFCARSSSNGRLKLDQFDLWGPGTLVTVSS

SEQ ID NO: 11 - DNA, 330 bp, Artificial Sequence
  human-rabbit hybrid
  CDS (1)..(330)
gcctatgatatgacccagactccagcctctgtggaggtagctgtgggaggcacagtcaccatcaattgccaggccagtcagagcattggcggttggttatcctggtatcggcagaaaccagggcagcctcccagcctcctgatctacaaggcttccactctggcatctggggtctcatcgcggttcagcggcactggatctaggacacacttcactctcaccatcaacggcgtgcagtgtgccgatgctgccacttactactgtcaacagggtagtgctgctgatgttgctaatactttcggcggagggaccgaggtggtggtcaaaggt

SEQ ID NO: 12 - protein, 110 aa, Artificial Sequence
  Synthetic Construct
AYDMTQTPASVEVAVGGTVTINCQASQSIGGWLSWYRQKPGQPPSLLIYKASTLASGVSSRFSGTGSRTHFTLTINGVQCADAATYYCQQGSAADVANTFGGGTEVVVKG

SEQ ID NO: 13 - DNA, 363 bp, Artificial Sequence
  human-rabbit hybrid
  CDS (1)..(363)
cagtcggtggaggagtccgggggtcgcctggtcacgcctgggacacccctgacactcacctgcacagtctctggattctccctcagtagctatgcaatgggctgggtccgccaggctccagggaaggggctggaattcatcggaatcattacttataatggtaacacatactacgcgagttgggcgaaaggccgattccccatctccaagacctcgtcgaccacggtggatctgaaaatgaccagtccgacaaccgaagacacggccacttatttctgtgccagagcccgttatggtggttatagtactaattcctactatcttaatatctggggcccaggcaccctggtcaccgtctcctca

SEQ ID NO: 14 - protein, 121 aa, Artificial Sequence
  Synthetic Construct
QSVEESGGRLVTPGTPLTLTCTVSGFSLSSYAMGWVRQAPGKGLEFIGIITYNGNTYYASWAKGRFPISKTSSTTVDLKMTSPTTEDTATYFCARARYGGYSTNSYYLNIWGPGTLVTVSS

SEQ ID NO: 15 - DNA, 333 bp, Artificial Sequence
  human-rabbit hybrid
  CDS (1)..(333)
gccgacgtcgtgatgacccagactccagcctccgtgtctgcagctgtgggcggcacagtcaccatcaactgccaggccagtgagaatatttataggattttggcctggtatcagcagaaaccagggcagcgtcccaaactcctgatctatggtgcatccaatctggaaactggggtcccatcacggttcaaaggcagtggatctgggacagaatacactctcaccatcagcgacctggagtgtgacgatgctgccacttactattgtcaaggtgttctttataatagtaatgatagtactttcggcggagggaccgaggtggtggtcaaaggt

SEQ ID NO: 16 - protein, 111 aa, Artificial Sequence
  Synthetic Construct
ADVVMTQTPASVSAAVGGTVTINCQASENIYRILAWYQQKPGQRPKLLIYGASNLETGVPSRFKGSGSGTEYTLTISDLECDDAATYYCQGVLYNSNDSTFGGGTEVVVKG

SEQ ID NO: 17 - DNA, 363 bp, Artificial Sequence
  human-rabbit hybrid
  CDS (1)..(363)
cagtcggtggaggagtccgggggtcgcctggtcacgcctgggacacccctgacactcacctgcacagtctctggattctccctcagtagctatgcaatgggctgggtccgccaggctccagggaaggggctggaattcatcggaatcattagttatagtggtaacacatactacgcgagttgggcgaaaggccgattccccatctccaaaacctcgtcgaccacggtggatctgaaaatgaccagtccgacaaccgaagacacggccaattatttctgtgccagagcccgttatggtggttatagtactaattcctactatcttaatatctggggcccaggcaccctggtcaccgtctcctca

SEQ ID NO: 18 - protein, 121 aa, Artificial Sequence
  Synthetic Construct
QSVEESGGRLVTPGTPLTLTCTVSGFSLSSYAMGWVRQAPGKGLEFIGIISYSGNTYYASWAKGRFPISKTSSTTVDLKMTSPTTEDTANYFCARARYGGYSTNSYYLNIWGPGTLVTVSS

SEQ ID NO: 19 - DNA, 333 bp, Artificial Sequence
  human-rabbit hybrid
  CDS (1)..(333)
gccgacgtcgtgatgacccagactccagcctccgtggaggcagctgtgggaggcacagtcaccatcaactgccaggccagtgagaatatttatagagttttggcctggtatcagcagaaaccagggcagcgtcccaaactcctgatctatggtgcatccaatctggaatctggggtcccatcacggttcaaaggcagtggatctgggacagactacactctcaccatcagcgacctggagtgtgacgatgctgccacttactattgtcaaggtgttgtttataatagtgatgatagtgctttcggcggagggaccgaggtggtggtcaaaggt

SEQ ID NO: 20 - protein, 111 aa, Artificial Sequence
  Synthetic Construct
ADVVMTQTPASVEAAVGGTVTINCQASENIYRVLAWYQQKPGQRPKLLIYGASNLESGVPSRFKGSGSGTDYTLTISDLECDDAATYYCQGVVYNSDDSAFGGGTEVVVKG

SEQ ID NO: 21 - DNA, 363 bp, Artificial Sequence
  human-rabbit hybrid
  CDS (1)..(363)
cagtcggtggaggagtccgggggtcgcctggtcacgcctgggacacccctgacactcacctgcacagtctctggattctccctcagtagctatgcaatgggctgggtccgccaggctccagggaaggggctggaattcatcggaatcattacttataatggtggcacatactacgcgagttgggcgaaaggccgattccccatctccaagacctcgtcgaccacggtggatctgaaaatgaccagtccgacaaccgaagacacggccacttatttctgtgccagagcccgttatggtggttatagtactaattcctactatcttaatatctggggcccaggcaccctggtcaccgtctcctca

SEQ ID NO: 22 - protein, 121 aa, Artificial Sequence
  Synthetic Construct
QSVEESGGRLVTPGTPLTLTCTVSGFSLSSYAMGWVRQAPGKGLEFIGIITYNGGTYYASWAKGRFPISKTSSTTVDLKMTSPTTEDTATYFCARARYGGYSTNSYYLNIWGPGTLVTVSS

SEQ ID NO: 23 - DNA, 333 bp, Artificial Sequence
  human-rabbit hybrid
  CDS (1)..(333)
gccgacgtcgtgatgacccagactccagcctccgtgtctgcagctgtgggcggcacagtcaccatcaactgccaggccagtgagaatatttatagggttttggcctggtatcagcagaaaccagggcagcgtcccaaactcctgatctatggtgcatccaatctggaaactggggtcccatcacggttcaaaggcagtggatctgggacagaatacactctcaccatcagcgacctggagtgtgacgatgctgccacttactattgtcaaggtgttctttatgataataatgatagtactttcggcggagggaccgaggtggtggtcaaaggt

SEQ ID NO: 24 - protein, 111 aa, Artificial Sequence
  Synthetic Construct
ADVVMTQTPASVSAAVGGTVTINCQASENIYRVLAWYQQKPGQRPKLLIYGASNLETGVPSRFKGSGSGTEYTLTISDLECDDAATYYCQGVLYDNNDSTFGGGTEVVVKG

SEQ ID NO: 25 - DNA, 363 bp, Artificial Sequence
  human-rabbit hybrid
  CDS (1)..(363)
cagtcggtggaggagtccgggggtcgcctggtcacgcctgggacacccctgacactcacctgcacagtctctggattctccctcagtagctatgcggtgggctgggtccgccaggctccagggaaggggctggaattcatcggaatcatcacttataatggtgccacatactacgcgagttgggcggaaggccgattccccatctccaagacctcgtcgaccacggtggatctgaaaatgaccagtccgacaaccgaagacacggccacttatttctgtgccagagcccgttatggtggttatagtactaattcctactatcttaatatctggggcccaggcaccctggtcaccgtctcctca

SEQ ID NO: 26 - protein, 121 aa, Artificial Sequence
  Synthetic Construct
QSVEESGGRLVTPGTPLTLTCTVSGFSLSSYAVGWVRQAPGKGLEFIGIITYNGATYYASWAEGRFPISKTSSTTVDLKMTSPTTEDTATYFCARARYGGYSTNSYYLNIWGPGTLVTVSS

SEQ ID NO: 27 - DNA, 333 bp, Artificial Sequence
  human-rabbit hybrid
  CDS (1)..(333)
gccgacgtcgtgatgacccagactccagcctccgtgtctgcagctgtgggcggcacagtcaccatcaactgccaggccagtgagaatatttataggattttggcctggtatcagcagaaaccagggcagcgtcccaagctcctgatctatggtgcatccaatctggaaactggggtcccatcacggttcaaaggcagtggatctgggacagaatacactctcaccatcagcgacctggagtgtgacgatgctgccacttactattgtcaaggtgttctttggaatagtaatgatagtactttcggcggagggaccgaggtggtggtcaaaggt

SEQ ID NO: 28 - protein, 111 aa, Artificial Sequence
  Synthetic Construct
ADVVMTQTPASVSAAVGGTVTINCQASENIYRILAWYQQKPGQRPKLLIYGASNLETGVPSRFKGSGSGTEYTLTISDLECDDAATYYCQGVLWNSNDSTFGGGTEVVVKG

SEQ ID NO: 29 - DNA, 366 bp, Artificial Sequence
  human-rabbit hybrid
  CDS (1)..(366)
caggaactagtggagtctggagggggtctggtccagccggggggatccctgaaactctcctgcaaagcctctggaatcgacttcaataactatggcattagctgggtccgccaggctccagggaaggggctggagtggatcgcatacaagtatcctggttttggtatcagaaactacgcgaactctgtgaagggccgattcaccatctccagcgacaacgcccagaacacggtgtttctgcaaatgaccagtctggcagcctcggacacggccacctatttctgtgcaagaggagcgcgatataggcacgatgactatggtgctttgaacttgtggggcccaggcaccctggtcaccgtctcctca

SEQ ID NO: 30 - protein, 122 aa, Artificial Sequence
  Synthetic Construct
QELVESGGGLVQPGGSLKLSCKASGIDFNNYGISWVRQAPGKGLEWIAYKYPGFGIRNYANSVKGRFTISSDNAQNTVFLQMTSLAASDTATYFCARGARYRHDDYGALNLWGPGTLVTVSS

SEQ ID NO: 31 - DNA, 324 bp, Artificial Sequence
  human-rabbit hybrid
  CDS (1)..(324)
gacatcgtgatgacccagactccaccctctgtgtctgcagctgtgggaggcacagtcaccatcaattgtcaggccagtcagagtgttaccaacctcttagcctggtatcagcagaaaccagggcagcctcccaagctcctgatttatggtgcatctaatctggaatctggggtcccatcgcgtttccgtggcagtggatctgggacagagttcactctcaccatcagtggcatgaaggctgaagatgctgccacttattactgtcaaagtggttattatagtgctaatactttcggtgcggggaccaaggtggggatcaaacgt

SEQ ID NO: 32 - protein, 108 aa, Artificial Sequence
  Synthetic Construct
DIVMTQTPPSVSAAVGGTVTINCQASQSVTNLLAWYQQKPGQPPKLLIYGASNLESGVPSRFRGSGSGTEFTLTISGMKAEDAATYYCQSGYYSANTFGAGTKVGIKR

SEQ ID NO: 33 - DNA, 357 bp, Artificial Sequence
  human-rabbit hybrid
  CDS (1)..(357)
cagtcggtggaggagtccgggggtcgcctggtcacgcctgggacacccctgacactcacctgcacagtctctggattctccctcagtagttatgtaataaattgggtccgccaggctccagggaaggggctggaatggatcggaaccattagtagtagtggtagcacatactacgcgagctgggcgaaaggccgattcaccatctccacaacctcgtcgaccacggtggatctgaaaatcaccagtccgacaaccgaggacacggccacctatttctgtgccagaggggctgatggtgcttttattagtgactactttaacatctggggcccaggcaccctggtcaccgtctcctca

SEQ ID NO: 34 - protein, 119 aa, Artificial Sequence
  Synthetic Construct
QSVEESGGRLVTPGTPLTLTCTVSGFSLSSYVINWVRQAPGKGLEWIGTISSSGSTYYASWAKGRFTISTTSSTTVDLKITSPTTEDTATYFCARGADGAFISDYFNIWGPGTLVTVSS

SEQ ID NO: 35 - DNA, 336 bp, Artificial Sequence
  human-rabbit hybrid
  CDS (1)..(336)
gccgacatcgtgatgacccagactccatcctccgtgtctgcagctgtgggaggcacagtcaccatcaagtgccaggccaatgagaatattgacagttggttagcctggtatcagcagaaaccagggcagcgtcccaagctcctaatctattatgcatccactctggcatctggggtcccatcgcggttcaaaggcagtggatctgggacagagtacactctcaccatcagcgacctggagtgtgccgatggtgccacttactactgtcaaagctatgatactattagtgactatggtgttggtttcggcggagggaccgaggtggtggtcaaaggt

SEQ ID NO: 36 - protein, 112 aa, Artificial Sequence
  Synthetic Construct
ADIVMTQTPSSVSAAVGGTVTIKCQANENIDSWLAWYQQKPGQRPKLLIYYASTLASGVPSRFKGSGSGTEYTLTISDLECADGATYYCQSYDTISDYGVGFGGGTEVVVKG

SEQ ID NO: 37 - DNA, 363 bp, Artificial Sequence
  human-rabbit hybrid
  CDS (1)..(363)
cagtcggtggaggagtccgggggtcgcctggtcacgcctgggacacccctgacactcacctgcacagtctctggattctccctcagtagctatgcaatgggctgggtccgccaggctccagggaaggggctggaattcatcggaatcatcacttataatagtaacacatactacgcgaattgggcgaaaggccgattccccatctccaagacctcgtcgaccacggtggatctgaaaatgaccagtccgacaaccgaagacacggccacttatttctgtgccagagcccgttatggtggttatagtactaattcctactatcttaatatctggggcccaggcaccctggtcaccgtctcctca

SEQ ID NO: 38 - protein, 121 aa, Artificial Sequence
  Synthetic Construct
QSVEESGGRLVTPGTPLTLTCTVSGFSLSSYAMGWVRQAPGKGLEFIGIITYNSNTYYANWAKGRFPISKTSSTTVDLKMTSPTTEDTATYFCARARYGGYSTNSYYLNIWGPGTLVTVSS

SEQ ID NO: 39 - DNA, 333 bp, Artificial Sequence
  human-rabbit hybrid
  CDS (1)..(333)
gccgacgtcgtgatgacccagactccagcctccgtgtctgcagctgtgggcggcacagtcaccatcaactgccaggccagtgagaatatttataggattttggcctggtatcagcagaaaccagggcagcctcccaagctcctgatctatggtgcatccaatctggaaactggggtcccatcacggttcaaaggcagtggatctgggacagaatatactctcaccatcagcgacctggagtgtgacgatgctgccacttactattgtcaaggtgttctttataatagtaatgatagtactttcggcggagggaccgaggtggtggtcaaaggt

SEQ ID NO: 40 - protein, 111 aa, Artificial Sequence
  Synthetic Construct
ADVVMTQTPASVSAAVGGTVTINCQASENIYRILAWYQQKPGQPPKLLIYGASNLETGVPSRFKGSGSGTEYTLTISDLECDDAATYYCQGVLYNSNDSTFGGGTEVVVKG

SEQ ID NO: 41 - DNA, 363 bp, Artificial Sequence
  human-rabbit hybrid
  CDS (1)..(363)
caggagcagctggaggagtccgggggagacctggtcaagcctgagggatccctgacactcacctgcactgcctctggatttgactccagtagcacctactacatgtgctgggtccgccaggctccagggaaggggctggagtggagcggatgcatttatactggtggtcttacatactacgcgagctgggcgaaaggccgattcaccatctccaaaacctcgtcgaccacggtgactctgcaaatgaccagtctgacagccgcggacacggccacctatttctgtgcgagttatattggtagtcgtttttatatgcccggttttagcttgtggggcccaggcaccctggtcaccgtctcctca

SEQ ID NO: 42 - protein, 121 aa, Artificial Sequence
  Synthetic Construct
QEQLEESGGDLVKPEGSLTLTCTASGFDSSSTYYMCWVRQAPGKGLEWSGCIYTGGLTYYASWAKGRFTISKTSSTTVTLQMTSLTAADTATYFCASYIGSRFYMPGFSLWGPGTLVTVSS

SEQ ID NO: 43 - DNA, 339 bp, Artificial Sequence
  human-rabbit hybrid
  CDS (1)..(339)
gccatcgtgatgacccagactccatcttccaagtctgtccctgtgggagacacagtcaccatcaattgccaggccagtgagagtatttacagtaacaatcgcttagcctggtttcaacaaaaaccagggcagcctcccaagctcctgatctatctggcatccactctggcatctggggtcccatcgcggttcaaaggcagtggatctgggacacagttcactctcaccatcagcgatgtggtgtgtgacgatgctgccacttactactgtgcaggatataaaggtagtagtactgatggtactgcgttcggcggagggaccgaggtggtggtcaaaggt

SEQ ID NO: 44 - protein, 113 aa, Artificial Sequence
  Synthetic Construct
AIVMTQTPSSKSVPVGDTVTINCQASESIYSNNRLAWFQQKPGQPPKLLIYLASTLASGVPSRFKGSGSGTQFTLTISDVVCDDAATYYCAGYKGSSTDGTAFGGGTEVVVKG

SEQ ID NO: 45 - protein, 10 aa, Homo sapiens
GFTSSSSDYA

SEQ ID NO: 46 - protein, 8 aa, Homo sapiens
ISGSGGST

SEQ ID NO: 47 - protein, 19 aa, Homo sapiens
AKVVGYSMYDYYYHYALDV

SEQ ID NO: 48 - protein, 8 aa, Homo sapiens
SSNIGSNT

SEQ ID NO: 49 - protein, 3 aa, Homo sapiens
SNN

SEQ ID NO: 50 - protein, 9 aa, Homo sapiens
QSYDTSNVV

SEQ ID NO: 51 - protein, 10 aa, Homo sapiens
GFTSSSSDYA

SEQ ID NO: 52 - protein, 8 aa, Homo sapiens
ISGSGVST

SEQ ID NO: 53 - protein, 19 aa, Homo sapiens
AKVVGYSVYDYYYHYALDV

SEQ ID NO: 54 - protein, 8 aa, Homo sapiens
SSNIGSNT

SEQ ID NO: 55 - protein, 3 aa, Homo sapiens
SDN

SEQ ID NO: 56 - protein, 9 aa, Homo sapiens
QSDDTSNVV

SEQ ID NO: 57 - protein, 10 aa, Homo sapiens
GFTSSSSDYA

SEQ ID NO: 58 - protein, 8 aa, Homo sapiens
ISGSGGST

SEQ ID NO: 59 - protein, 19 aa, Homo sapiens
AKVFGYSMYDYYYHYALDV

SEQ ID NO: 60 - protein, 8 aa, Homo sapiens
SSNIGSNT

SEQ ID NO: 61 - protein, 3 aa, Homo sapiens
SNN

SEQ ID NO: 62 - protein, 9 aa, Homo sapiens
QSDDTSNVV

SEQ ID NO: 63 - protein, 10 aa, Homo sapiens
GFTTSSSDYA

SEQ ID NO: 64 - protein, 8 aa, Homo sapiens
ISGSGGST

SEQ ID NO: 65 - protein, 19 aa, Homo sapiens
AKDVGYSTYDYYYHYALDV

SEQ ID NO: 66 - protein, 8 aa, Homo sapiens
SSNVGSNT

SEQ ID NO: 67 - protein, 3 aa, Homo sapiens
SDD

SEQ ID NO: 68 - protein, 9 aa, Homo sapiens
QSYDTSNVV

SEQ ID NO: 69 - protein, 10 aa, Homo sapiens
GFQSDSSDYA

SEQ ID NO: 70 - protein, 8 aa, Homo sapiens
ISGTGGST

SEQ ID NO: 71 - protein, 19 aa, Homo sapiens
AKVVGFSTYDYYYHYALDV

SEQ ID NO: 72 - protein, 8 aa, Homo sapiens
SSNIGSNT

SEQ ID NO: 73 - protein, 3 aa, Homo sapiens
SDD

SEQ ID NO: 74 - protein, 9 aa, Homo sapiens
QSYDTSNVV

SEQ ID NO: 75 - DNA, 369 bp, Homo sapiens
gagcagcagctgctggagagcggcggaggcctggtgcagccaggaggaagcctgaggctgtcctgcgccgtgagcggcttttccctgagcagctacgccatgggctgggtgaggcaggcccctggaaagggcctggagttcatcggcatcatctcctactccggcaacacatactacgccagctgggccaagggcaggttcacaatcagcaaggatagctccaagaacaccgtgtacctgcagatgaacagcctgagggccgaggacacagccgtgtacttctgtgccagggccaggtacggcggctactccaccaatagctactacctgaacatctggggccccggcacactggtgacagtgagctcc

SEQ ID NO: 76 - protein, 124 aa, Homo sapiens
EQQLLESGGGLVQPGGSLRLSCAVSGFSLSSYAMGWVRQAPGKGLEFIGIISYSGNTYYASWAKGRFTISKDSSKNTVYLQMNSLRAEDTAVYFCARARYGGYSTNSYYLNIWCGPGTLVTVSS

SEQ ID NO: 77 - DNA, 327 bp, Homo sapiens
gatgtgcagatgacccagtccccttccaccctgtccgccagcgtgggcgatagagtgacaatcacctgtcaggccagcgagaatatctacagggtgctggcctggtaccagcagaagcctggcaagaggcctaagctgctgatctacggcgccagcaatctggagtccggcgtgcccagcagattttccggctccggcagcggcaccgagtacacactgacaatcagctccctgcagcctgacgatttcgccacctactactgccagggcgtggtgtacaacagcgacgatagcgccttcggcggcggcacaaaggtggagatcaag

SEQ ID NO: 78 - protein, 109 aa, Homo sapiens
DVQMTQSPSTLSASVGDRVTITCQASENIYRVLAWYQQKPGKRPKLLIYGASNLESGVPSRFSGSGSGTEYTLTISSLQPDDFATYYCQGVVYNSDDSAFGGGTKVEIK

SEQ ID NO: 79 - DNA, 369 bp, Homo sapiens
gagcagcagctgctggagtccggcggaggcctggtgcagccaggaggaagcctgagactgagctgcgccgccagcggcatcgacttcaataattacggcatctcctgggtgagacaggcccctggcaagggcctggagtggatcgcctacaagtaccccggcttcggcatcagaaattacgccaatagcgtgaagggcagattcaccatctcctccgataacagcaagaacaccgtgtacctgcagatgaatagcctgagggccgaggataccgccgtgtacttttgcgccaggggcgccagatacagacacgatgactacggcgccctgaatctgtggggccccggcacactggtgaccgtgtccagc

SEQ ID NO: 80 - protein, 123 aa, Homo sapiens
EQQLLESGGGLVQPGGSLRLSCAASGIDFNNYGISWVRQAPGKGLEWIAYKYPGFGIRNYANSVKGRFTISSDNSKNTVYLQMNSLRAEDTAVYFCARGARYRHDDYGALNLWGPGTLVTVSS

SEQ ID NO: 81 - DNA, 321 bp, Homo sapiens
gacatccagatgacccagtcccccagcacactgtccgccagcgtgggcgacagagtgacaatcacctgccaggccagccagtccgtgaccaatctgctggcctggtaccagcagaagcccggcaagccccctaagctgctgatctacggcgcctccaatctggagagcggcgtgcccagcaggttttccggctccggcagcggcacagagtttacactgaccatctcctccctgcagcctgatgacttcgccacctactactgtcagagcggctactacagcgccaacacctttggccccggcaccaaggtggatatcaag

SEQ ID NO: 82 - protein, 107 aa, Homo sapiens
DIQMTQSPSTLSASVGDRVTITCQASQSVTNLLAWYQQKPGKPPKLLIYGASNLESGVPSRFSGSGSGTEFTLTISSLQPDDFATYYCQSGYYSANTFGPGTKVDIK

SEQ ID NO: 83 - DNA, 384 bp, Homo sapiens
caggtgcagctgcaggagtctggaggtggcttgatacagcctggtggatctctgagactctcctgtgcagcctctggattcacctctagttctagtgattatgccatgagttgggtcaggcaggctccagggaaggggctggagtgggtctcgtccatcagtggtagtggcgttagcacatactacgcagactccgtgaagggtcgcttcaccatctccagagacaattccaagaacacactctatgtgcaaatgaccagcctgacagccgaggacacggccgtgtactactgtgcgaaggtcgtcggttacagtgtttacgactactactaccactacgctttggacgtctggggccaagggaccacggtcaccgtctcctca

SEQ ID NO: 84 - DNA, 30 bp, Homo sapiens
ggattcacctctagttctagtgattatgcc

SEQ ID NO: 85 - DNA, 24 bp, Homo sapiens
atcagtggtagtggcgttagcaca

SEQ ID NO: 86 - DNA, 57 bp, Homo sapiens
gcgaaggtcgtcggttacagtgtttacgactactactaccactacgctttggacgtc

SEQ ID NO: 87 - protein, 128 aa, Homo sapiens
QVQLQESGGGLIQPGGSLRLSCAASGFTSSSSDYAMSWVRQAPGKGLEWVSSISGSGVSTYYADSVKGRFTISRDNSKNTLYVQMTSLTAEDTAVYYCAKVVGYSVYDYYYHYALDVWGQGTTVTVSS

SEQ ID NO: 88 - DNA, 324 bp, Homo sapiens
cagcctgtgctgactcagccaccttcagcgtctgggacccccgggcagagggtcaccatctcttgttctggaagcagctccaacatcggtagtaatactgtgaactggtaccagcagcttccaggaacggctcctaagctcctcatctatagtgataatcagagaccctcaggtgtccctgaccgattctctggctccaagtctggcacctcagcctccctcaccatctctggactgaagactgaggacgaggctggctactactgtcagtctgatgataccagcaatgtggtattcggtggaggcaccaaggtgaccgtcctc

SEQ ID NO: 89 - DNA, 24 bp, Homo sapiens
agctccaacatcggtagtaatact

SEQ ID NO: 90 - DNA, 9 bp, Homo sapiens
agtgataat

SEQ ID NO: 91 - DNA, 27 bp, Homo sapiens
cagtctgatgataccagcaatgtggta

SEQ ID NO: 92 - protein, 108 aa, Homo sapiens
QPVLTQPPSASGTPGQRVTISCSGSSSNIGSNTVNWYQQLPGTAPKLLIYSDNQRPSGVPDRFSGSKSGTSASLTISGLKTEDEAGYYCQSDDTSNVVFGGGTKVTVL

SEQ ID NO: 93 - DNA, 384 bp, Homo sapiens
caggtgcagctgcaggagtctggaggtggcttgatacagcctggtggatctctgagactctcctgtgcagcctctggattcacctctagttctagtgattatgccatgagttgggtcaggcaggctccagggaaggggctggagtgggtctcgtccatcagtggtagtggcggtagcacatactacgcagactccgtgaagggtcgcttcaccatctccagagacaattccaagaacacactctatgtgcaaatgaccagcctgacagccgaggacacggccgtgtactactgtgcgaaggtcttcggttacagtatgtacgactactactaccactacgctttggacgtctggggccaagggaccacggtcaccgtctcctca

SEQ ID NO: 94 - DNA, 30 bp, Homo sapiens; identical to SEQ ID NO: 84

SEQ ID NO: 95 - DNA, 24 bp, Homo sapiens
atcagtggtagtggcggtagcaca

SEQ ID NO: 96 - DNA, 57 bp, Homo sapiens
gcgaaggtcttcggttacagtatgtacgactactactaccactacgctttggacgtc

SEQ ID NO: 97 - protein, 128 aa, Homo sapiens
QVQLQESGGGLIQPGGSLRLSCAASGFTSSSSDYAMSWVRQAPGKGLEWVSSISGSGGSTYYADSVKGRFTISRDNSKNTLYVQMTSLTAEDTAVYYCAKVFGYSMYDYYYHYALDVWGQGTTVTVSS

SEQ ID NO: 98 - DNA, 324 bp, Homo sapiens
cagcctgtgctgactcagccaccttcagcgtctgggacccccgggcagaggatcaccatctcttgttctggaagcagctccaacatcggtagtaatactgtgaactggtaccagcagcttccaggaacggctcctaagctcctcatctatagtaataatcagagaccctcaggtgtccctgaccgattctctggctccaagtctggcacctcagcctccctcaccatctctggactgaagactgaggacgaggctggctactactgtcagtctgatgataccagcaatgtggtattcggtggaggcaccaaggtgaccgtcctc

SEQ ID NO: 99 - DNA, 24 bp, Homo sapiens
agctccaacatcggtagtaatact

SEQ ID NO: 100 - DNA, 9 bp, Homo sapiens
agtaataat

SEQ ID NO: 101 - DNA, 27 bp, Homo sapiens
cagtctgatgataccagcaatgtggta

SEQ ID NO: 102 - protein, 108 aa, Homo sapiens
QPVLTQPPSASGTPGQRITISCSGSSSNIGSNTVNWYQQLPGTAPKLLIYSNNQRPSGVPDRFSGSKSGTSASLTISGLKTEDEAGYYCQSDDTSNVVFGGGTKVTVL

SEQ ID NO: 103 - DNA, 384 bp, Homo sapiens
caggtgcagctgcaggagtctggaggtggcttgatacagcctggtggatctctgagactctcctgtgcagcctctggattcaccactagttctagtgattatgccatgagttgggtcaggcaggctccagggaaggggctggagtgggtctcgtccatcagtggtagtgggggtagcacatactacgcagactccgtgaagggtcgcttcaccatctccagagacaattccaagaacacactctatgtgcaaatgaccagcctgacagccgaggacacggccgtgtactactgtgcgaaggacgtcggttacagtacgtacgactactactaccactacgctttggacgtctggggccaagggaccacggtcaccgtctcctca

SEQ ID NO: 104 - DNA, 30 bp, Homo sapiens
ggattcaccactagttctagtgattatgcc

SEQ ID NO: 105 - DNA, 24 bp, Homo sapiens
atcagtggtagtgggggtagcaca

SEQ ID NO: 106 - DNA, 57 bp, Homo sapiens
gcgaaggacgtcggttacagtacgtacgactactactaccactacgctttggacgtc

SEQ ID NO: 107 - protein, 128 aa, Homo sapiens
QVQLQESGGGLIQPGGSLRLSCAASGFTTSSSDYAMSWVRQAPGKGLEWVSSISGSGGSTYYADSVKGRFTISRDNSKNTLYVQMTSLTAEDTAVYYCAKDVGYSTYDYYYHYALDVWGQGTTVTVSS

SEQ ID NO: 108 - DNA, 324 bp, Homo sapiens
cagcctgtgctgactcagccaccttcagcgtctgggacccccgggcagagggtcaccatctcttgttctggaagcagctccaacgtcggtagtaatactgtgaactggtaccagcagcttccaggaacggctcctaagctcctcatctatagtaataatcagagaccctcaggtgtccctgaccgattctctggctccaagtctggcacctcagcctccctcaccatctctggactgaagactgaggacgaggctggctactactgtcagtcttatgataccagcaatgtggtattcggtggaggcaccaaggtgaccgtcctc

SEQ ID NO: 109 - DNA, 24 bp, Homo sapiens
agctccaacgtcggtagtaatact

SEQ ID NO: 110 - DNA, 9 bp, Homo sapiens
agtaataat

SEQ ID NO: 111 - DNA, 27 bp, Homo sapiens
cagtcttatgataccagcaatgtggta

SEQ ID NO: 112 - protein, 108 aa, Homo sapiens
QPVLTQPPSASGTPGQRVTISCSGSSSNVGSNTVNWYQQLPGTAPKLLIYSNNQRPSGVPDRFSGSKSGTSASLTISGLKTEDEAGYYCQSYDTSNVVFGGGTKVTVL

SEQ ID NO: 113 - DNA, 384 bp, Homo sapiens
caggtgcagctgcaggagtctggaggtggcttgatacagcctggtggatctctgagactctcctgtgcagcctctggattccagtctgattctagtgattatgccatgagttgggtcaggcaggctccagggaaggggctggagtgggtctcgtccatcagtggtactggcggtagcacatactacgcagactccgtgaagggtcgcttcaccatctccagagacaattccaagaacacactctatgtgcaaatgaccagcctgacagccgaggacacggccgtgtactactgtgcgaaggtggtcggtttcagtacgtacgactactactaccactacgctttggacgtctggggccaagggaccacggtcaccgtctcctca

SEQ ID NO: 114 - DNA, 30 bp, Homo sapiens
ggattccagtctgattctagtgattatgcc

SEQ ID NO: 115 - DNA, 24 bp, Homo sapiens
atcagtggtactggcggtagcaca

SEQ ID NO: 116 - DNA, 57 bp, Homo sapiens
gcgaaggtggtcggtttcagtacgtacgactactactaccactacgctttggacgtc

SEQ ID NO: 117 - protein, 128 aa, Homo sapiens
QVQLQESGGGLIQPGGSLRLSCAASGFQSDSSDYAMSWVRQAPGKGLEWVSSISGTGGSTYYADSVKGRFTISRDNSKNTLYVQMTSLTAEDTAVYYCAKVVGFSTYDYYYHYALDVWGQGTTVTVSS

SEQ ID NO: 118 - DNA, 324 bp, Homo sapiens
cagcctgtgctgactcagccaccttcagcgtctgggacccccgggcagagggtcaccatctcttgttctggaagcagctccaacatcggtagtaatactgtgaactggtaccagcagcttccaggaacggctcctaagctcctcatctatagtgatgatcagagaccctcaggtgtccctgaccgattctctggctccaagtctggcacctcagcctccctcaccatctctggactgaagactgaggacgaggctggctactactgtcagtcttatgataccagcaatgtggtattcggtggaggcaccaaggtgaccgtcctc

SEQ ID NO: 119 - DNA, 24 bp, Homo sapiens
agctccaacatcggtagtaatact

SEQ ID NO: 120 - DNA, 9 bp, Homo sapiens
agtgatgat

SEQ ID NO: 121 - DNA, 27 bp, Homo sapiens
cagtcttatgataccagcaatgtggta

SEQ ID NO: 122 - protein, 108 aa, Homo sapiens
QPVLTQPPSASGTPGQRVTISCSGSSSNIGSNTVNWYQQLPGTAPKLLIYSDDQRPSGVPDRFSGSKSGTSASLTISGLKTEDEAGYYCQSYDTSNVVFGGGTKVTVL

SEQ ID NO: 123 - DNA, 24 bp, Homo sapiens
ggcttttccctgagcagctacgcc

SEQ ID NO: 124 - DNA, 21 bp, Homo sapiens
atctcctactccggcaacaca

SEQ ID NO: 125 - DNA, 51 bp, Homo sapiens
gccagggccaggtacggcggctactccaccaatagctactacctgaacatc

SEQ ID NO: 126 - protein, 8 aa, Homo sapiens
GFSLSSYA

SEQ ID NO: 127 - protein, 7 aa, Homo sapiens
ISYSGNT

SEQ ID NO: 128 - protein, 17 aa, Homo sapiens
ARARYGGYSTNSYYLNI

SEQ ID NO: 129 - DNA, 18 bp, Homo sapiens
gagaatatctacagggtg

SEQ ID NO: 130 - DNA, 9 bp, Homo sapiens
ggcgccagc

SEQ ID NO: 131 - DNA, 33 bp, Homo sapiens
cagggcgtggtgtacaacagcgacgatagcgcc

SEQ ID NO: 132 - protein, 6 aa, Homo sapiens
ENIYRV

SEQ ID NO: 133 - protein, 3 aa, Homo sapiens
GAS

SEQ ID NO: 134 - protein, 11 aa, Homo sapiens
QGVVYNSDDSA

SEQ ID NO: 135 - DNA, 24 bp, Homo sapiens
ggcatcgacttcaataattacggc

SEQ ID NO: 136 - DNA, 24 bp, Homo sapiens
aagtaccccggcttcggcatcaga

SEQ ID NO: 137 - DNA, 48 bp, Homo sapiens
gccaggggcgccagatacagacacgatgactacggcgccctgaatctg

SEQ ID NO: 138 - protein, 8 aa, Homo sapiens
GIDFNNYG

SEQ ID NO: 139 - protein, 8 aa, Homo sapiens
KYPGFGIR

SEQ ID NO: 140 - protein, 16 aa, Homo sapiens
ARGARYRHDDYGALNL

SEQ ID NO: 141 - DNA, 18 bp, Homo sapiens
cagtccgtgaccaatctg

SEQ ID NO: 142 - DNA, 9 bp, Homo sapiens
ggcgcctcc

SEQ ID NO: 143 - DNA, 27 bp, Homo sapiens
cagagcggctactacagcgccaacacc

SEQ ID NO: 144 - protein, 6 aa, Homo sapiens
QSVTNL

SEQ ID NO: 145 - protein, 3 aa, Homo sapiens
GAS

SEQ ID NO: 146 - protein, 9 aa, Homo sapiens
QSGYYSANT